(19) United States
(12) Patent Application Publication
(10) Pub. No.: US 2025/0092086 A1
(43) Pub. Date: Mar. 20, 2025

(54) CELASTROL SALTS, CRYSTALLINE FORMS, AND USES THEREOF

(71) Applicant: ERX Pharmaceuticals Inc., Cambridge, MA (US)

(72) Inventor: Teoman Uysal, Cambridge, MA (US)

(21) Appl. No.: 18/945,768

(22) Filed: Nov. 13, 2024

Related U.S. Application Data
(63) Continuation of application No. PCT/US23/80122, filed on Nov. 16, 2023.
(60) Provisional application No. 63/384,153, filed on Nov. 17, 2022.

(51) Int. Cl. C07J 63/00; A61K 31/56

(52) U.S. Cl. CPC C07J 63/008; A61K 31/56

(57) ABSTRACT

The invention relates to solid forms of Celastrol or a salt thereof, including crystalline forms of Celastrol or a salt thereof, and methods of preparation and use thereof.

### RELATED APPLICATIONS

This application is a bypass continuation and claims priority to PCT/US2023/080122, filed on Nov. 16, 2023, which claims priority to, and the benefit of, U.S. Provisional Application No. 63/384,153, filed on Nov. 17, 2022, the content of which is incorporated herein by reference in their entireties.

### FIELD OF THE INVENTION

The invention relates to crystalline forms of celastrol, or a salt thereof, methods of preparing the crystalline forms, and use thereof.

### BACKGROUND

In 2008, the World Health Organization (WHO) estimated that 1.4 billion adults worldwide were overweight; of these, 200 million men and 300 million women were obese. It is predicted that more than one billion people in the world will be obese by 2030. Obesity is a major cause for the development of debilitating conditions such as type 2 diabetes, cardiovascular disease, osteoarthritis (a health problem causing pain, swelling, and stiffness in one or more joints), stroke, hypertension, cancer (breast, colon, endometrial (related to the uterine lining), and kidney), and non-alcoholic steatohepatitis, all of which reduce life quality as well as lifespan.

Amongst healthcare experts around the world, there is now agreement that the global epidemic of obesity will be one of the leading causes of morbidity and mortality for current and future generations, unless the inexorable rise in the prevalence of this disorder is reversed. Once considered to be a problem mainly in Western cultures, developing nations have now joined the ranks of countries burdened by obesity. A 1999 United Nations study found obesity to be present in all developing regions and growing rapidly, even in countries where hunger also existed. Obesity is defined by the World Health Organization (WHO) as a subject who has a body mass index (BMI=weight in kg/height in m²) value of >30 kg m⁻² (normal BMI=20-25 kg m⁻²).

Excessive weight and obesity result from an energy imbalance. The body needs a certain amount of energy (calories) from food to keep up basic life functions. Body weight tends to remain the same when the number of calories eaten equals the number of calories the body uses or “burns.” Over time, when people eat and drink more calories than they burn, the energy balance tips toward weight gain, overweight, and obesity.

A possible explanation for the rapid increase in obesity is that it is being driven by a combination of genetic, social and environmental factors. Although a significant proportion of people manage very successfully to maintain a healthy bodyweight by following a careful diet and having a reasonable level of physical exercise, for many others this plan has not resulted in the desired healthy outcome. For some of the obese population, pharmacotherapy will be required to provide the requisite adjunctive support to diet, exercise and lifestyle modification that will deliver a clinically beneficial bodyweight reduction of >5%.

There is no single cause of all excessive weight and obesity. There is no single approach that can help prevent or treat excessive weight and obesity. Treatment may include a mix of behavioral treatment, diet, exercise, and sometimes weight-loss drugs. In some cases of extreme obesity, weight-loss surgery may be an option. Over the last 15 years, only four new drugs, i.e. dexfenfluramine (Redux®), sibutramine (Meridia®, Reductil®), orlistat (Xenical®) and rimonabant (Acomplia®), have been registered for the treatment of obesity. Of these drugs, only three, dexfenfluramine, sibutramine and orlistat, have achieved global (with the exception of Japan) registration. There is a great need to develop additional anti-obesity drugs, which are safe and effective.

### SUMMARY

Celastrol, a naturally occurring quinone methide triterpene derived from the Thunder of God Vine, has been identified as a pleiotropic compound showing anti-tumor, anti-inflammatory, anti-hypertensive, and anti-diabetic activity in numerous cellular and in vivo models. Of particular relevance to Glioblastoma (GBM) treatment, celastrol inhibits the growth of human glioma xenografts in mice and was selected from a screen of over 2000 natural products for its potential to synergistically enhance the anti-cancer response to temozolomide (TMZ). Moreover, in a recent Harvard study, the herbal extracts of the Thunder of God Vine has been shown to function as an appetite suppressant in rodents and may potentially become the key ingredient in a powerful obesity drug. A weight loss drug containing Celastrol could be effective due to a hormone called leptin.

Provided are, inter alia, solid forms (e.g. crystallines) of Compound I (celastrol, or 3-hydroxy-9β,13α-dimethyl-2-oxo-24,25,26-trinoroleana-1(10),3,5,7-tetraen-29-oic acid) or a salt of Compound I (celastrol, or 3-hydroxy-9β,13α-dimethyl-2-oxo-24,25,26-trinoroleana-1(10),3,5,7-tetraen-29-oic acid).

In an aspect, provided is a crystalline Form III of Compound I,

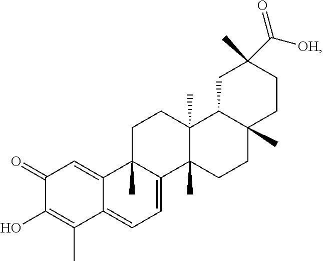

which is characterized by an x-ray powder diffraction pattern comprising peaks at 2θ angles (±0.2) of 9.51°, 14.81°, and 16.84°.

In an aspect, provided is a crystalline Form I of Compound I,

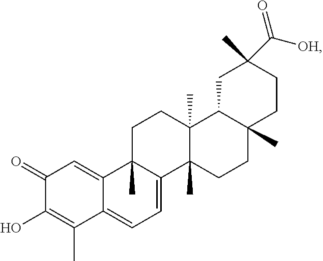

which is characterized by an x-ray powder diffraction pattern comprising peaks at 2θ angles (±0.2) of 9.53°, 13.29°, and 16.13°.

In an aspect, provided is a crystalline Form II of Compound I,

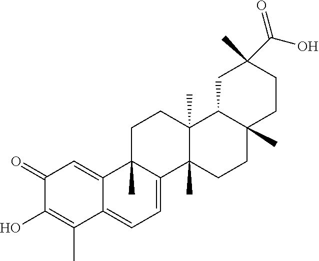

characterized by an X-ray powder diffraction pattern comprising peaks at 2θ angles (±0.2) of 11.68°, 16.07°, and 17.04°.

In an aspect, provided is a crystalline Form IV of Compound I,

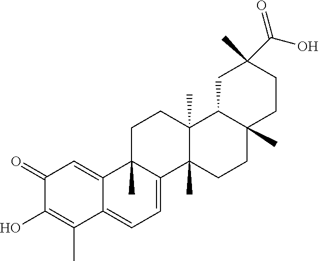

characterized by an X-ray powder diffraction pattern comprising peaks at 2θ angles (±0.2) of 13.4°, 14.6°, and 17.4°.

In an aspect, provided is a pharmaceutical composition comprising the crystalline form (e.g., Form III, Form I, or Form II) as described herein, and a pharmaceutically acceptable excipient. In some embodiments, the crystalline form is Form III.

In an aspect, provide is a method of treating obesity in a subject in need thereof comprising administering to the subject an effective amount of the crystalline form (e.g., Form III, Form I, or Form II) as described herein, or the pharmaceutical composition described herein. In some embodiments, the crystalline form is Form III.

In an aspect, provide is a method of treating an obesity-related disease or disorder comprising administering to a subject suffering from or at risk of suffering from an obesity-related disease or disorder the crystalline form (e.g., Form III, Form I, or Form II) as described herein, or the pharmaceutical composition described herein. In some embodiments, the crystalline form is Form III.

In an aspect, provided is a method of treating a malignancy-related disease or disorder comprising administering to a subject suffering from or at risk of suffering from a malignancy-related disease or disorder the crystalline form (e.g., Form III, Form I, or Form II) as described herein, or the pharmaceutical composition described herein. In some embodiments, the crystalline form is Form III.

In an aspect, provided is a process for preparing a crystalline Form III of Compound I as described herein.

In an aspect, provided is a method of purifying Compound I or a crystalline form of Compound I, comprising the making a crystalline Form III of Compound I by a process as described herein.

In an aspect, provided is a crystalline Form III of Compound I, made by a process described herein.

In an aspect, provided is a synthetic intermediate for making a crystalline Form III of Compound I described herein, wherein the synthetic intermediate is crystalline Form IV of Compound I.

In an aspect, provided herein is a pharmaceutical composition comprising:
(i) a pharmaceutical agent, wherein the pharmaceutical agent is a crystalline form of Compound I as described herein;
(ii) an emulsifier or surfactant; and
(iii) a mucoadhesive or viscosifier;
wherein the pharmaceutical composition is in the form of a liquid suspension.

In an aspect, provided herein is a pharmaceutical composition comprising:
(i) a pharmaceutical agent, wherein the pharmaceutical agent is a crystalline form of Compound I as described herein;
(ii) a polysorbate; and
(iii) a cellulose;
wherein the pharmaceutical composition is in the form of a liquid suspension.

In an aspect, provided herein is a pharmaceutical composition comprising:
(i) a pharmaceutical agent, wherein the pharmaceutical agent is a crystalline form of Compound I as described herein;
(ii) hydroxypropyl-beta-cyclodextrin (HPBCD) or trehalose; and
(iii) hydroxypropyl methyl cellulose (HPMC);
In an aspect, provided herein is a pharmaceutical composition comprising:
(i) a pharmaceutical agent, wherein the pharmaceutical agent is a crystalline form of Compound I as described herein in an amount of from about 1 wt. % to about 5 wt. %;
(ii) hydroxypropyl-beta-cyclodextrin (HPBCD) or trehalose in an amount of from about 60 wt. % to about 70 wt. %; and
(iii) hydroxypropyl methyl cellulose (HPMC) in an amount of from about 10 wt. % to about 40 wt. %;
wherein the pharmaceutical composition is in the form of a powder.

Other aspects of the invention are disclosed infra.

### BRIEF DESCRIPTION OF THE DRAWINGS

The patent or application file contains at least one drawing in color. Copies of this patent or patent publication with color drawing(s) will be provided by the Office upon request and payment of necessary fee.

FIG. 1 is a photograph by optical microscopy (obj.×10) of the trometamol salt of celastrol (Compound 2).

FIG. 2 is a photograph by SEM (G×800) of the trometamol salt of celastrol (Compound 2).

FIG. 3 is a graph of the X-ray diffraction pattern obtained for trometamol salt of celastrol (Compound 2).

FIG. 4 is a graph of TGA and DSC data for Compound 2.

FIG. 5 is a graph of the DVS profile for Compound 2.

FIGS. 6A-6C are graphs of XRPD (X-ray powder diffraction) patterns representing the influence of temperature on the trometamol salt Compound 2 (FIG. 6A=25° C.; FIG. 6B=60° C.; and FIG. 6C=100° C.).

FIGS. 7A-7C are graphs of the XRPD pattern showing the influence of RH at 40° C. on trometamol salt Compound 2 (FIG. 7A=10%; FIG. 7B=30%; and FIG. 7C=80%).

FIG. 8 is a photograph by optical microscopy (obj.×10) of Compound 1.

FIG. 9 is a photograph by SEM (G×800) of Compound 1.

FIG. 10 is a graph of the X-ray diffraction pattern of neutral Compound 1.

FIG. 11 is a graph of thermal analysis (DSC and TGA) of neutral Compound 1.

FIG. 12 is a graph of a DVS profile showing water sorption/desorption of neutral Compound 1 at 37° C. from 0% to 95% then to 0%.

FIGS. 13A-13C are graphs of the X-ray diffraction pattern (XRPD) of neutral Compound 1 showing the influence of temperature: FIG. 13A is a graph of the XRPD at 25° C.; FIG. 13B is a graph of XRPD at 100° C.; and FIG. 13C is a graph of the XRPD at 50° C.

FIG. 14 is a graph of the XRPD pattern of neutral Compound 1 representing the influence of RH at 40° C.

FIG. 15A is a photograph by optical microscope (obj.×10, left) and a SEM (G×300, right) of Compound 1 Form III.

FIG. 15B is a graph of the XRPD pattern for Compound 1 Form III.

FIG. 16 is a graph of thermal analyses (DSC and TGA) of Compound 1 Form III.

FIG. 17 is a graph of a DVS profile showing the water sorption/desorption of Compound 1 Form III at 37° C. from 0% to 95% then to 0%.

FIG. 18 is a graph of solubility curves corresponding to the 2 solvents namely ethanol and methanol.

FIG. 19 is a graph of the XRPD pattern of Compound 1 Form I.

FIG. 20 is a graph of the XRPD pattern of Compound 1 Form II.

FIG. 21 is a graph of the XRPD pattern of Compound 1 Form III.

FIG. 22A is a graph of the XRPD pattern of Compound 1 Form IV.

FIG. 22B is a graph of the NMR spectrum of Compound 1 Form IV produced by dissolving in dichloromethane and some ethyl acetate as anti-solvent.

FIG. 23A is a graph of the XRPD pattern of Compound 1 Form V.

FIG. 23B is a graph of the NMR spectrum of Compound 1 Form V produced by slurry in propan-2-ol.

FIG. 24A is a graph of the XRPD pattern of Compound 1 Form VI.

FIG. 24B is a graph of the NMR spectrum of Compound 1 Form VI produced by slurry in MTBE.

FIG. 25A is a photograph by an optical microscope (obj.×10, left) and a scanning electron microscope (G×300, right) of Compound 1 Form I.

FIG. 25B is a graph of the XRPD of Compound 1 Form I.

FIGS. 26A-26B are graphs of data from DSC and TGA analyses of Compound 1 Form I.

FIGS. 27A-27B are graphs of DVS profile showing water sorption/desorption of Compound Form I at 37° C. from 0% to 95% then to 50%.

FIG. 28 is a graph of the XRPD pattern representing the influence of temperature on Compound 1 Form I.

FIG. 29 is a graph of the XRPD pattern representing the influence of RH at 40° C. on Compound 1 Form I.

FIG. 30A is a photograph by optical microscope (obj.×10, left) and SEM (×300, right) of Compound 1 Form II.

FIG. 30B is a graph of the XRPD pattern of Compound 1 Form II.

FIG. 31 is a graph of thermal analyses (DSC, TGA) of Compound 1 Form II.

FIG. 32 is a graph of a DVS profile showing water sorption/desorption of Compound 1 Form II at 37° C. from 0% to 95% then to 0%.

FIG. 33 is a graph of an XRPD pattern representing the influence of temperature on Compound 1 Form II.

FIG. 34 is a graph of an XRPD pattern representing the influence of RH at 40° C. on Compound 1 Form II.

FIG. 35 compares the XRPD patterns of Compound I Form III, Compound I Form IV, the crystalline form of Compound I obtained after one of the crystallization purification steps of the process of Example 13, and the crystalline form of Compound I obtained after the crystal transformation of the process of Example 13.

FIG. 36 is a graph summarizing the concentration of celastrol in plasma of male Sprague Dawley rats following intranasal dosing of a dry powder intranasal formulation (Dry A as described in Example 15) at a dose concentration of 2 mg/kg. The numbers identify the tested rats (rats number 13, 14, 15, and 16).

FIG. 37 is a graph summarizing the concentration of celastrol in plasma of male Sprague Dawley rats following intranasal dosing of a dry powder intranasal formulation (Dry A as described in Example 15) at a dose concentration of 2 mg/kg. The numbers identify the tested rats (rats number 17, 18, 19, and 20).

FIG. 38 is a graph summarizing the concentration of celastrol in plasma of male Sprague Dawley rats following intranasal dosing of a liquid intranasal formulation (Liquid A as described in Example 15) at a dose concentration of 2 mg/kg. The numbers identify the tested rats (rats number 5, 6, 7, and 8).

FIG. 39 is a graph summarizing the concentration of celastrol in plasma of male Sprague Dawley rats following intranasal dosing of a liquid intranasal formulation (Liquid B as described in Example 15) at a dose concentration of 2 mg/kg. The numbers identify the tested rats (rats number 9, 10, 11, and 12).

FIG. 40 is a graph summarizing the concentration of celastrol in plasma of male Sprague Dawley rats following oral dosing of celastrol at a dose concentration of 2 mg/kg. The numbers identify the tested rats (rats number 1, 2, 3, and 4).

FIG. 41 is a graph comparing the mean concentrations of celastrol in plasma of male Sprague Dawley rats (average of tested rats) following intranasal administration of formulations Dry A, Dry B, Liquid A, and Liquid B (as described in Example 15), and oral dosing of celastrol at a dose concentration of 2 mg/kg.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

As used herein, “Celastrol” refers to a compound that is Compound I,

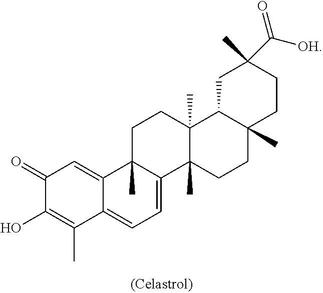

The terms “Compound 1,” “(2R,4aS,6aS,12bR,14aS,14bR)-10-hydroxy-2,4a,6a,9,12b,14a-hexamethyl-11-oxo-1,2,3,4,4a,5,6,6a,11,12b,13,14,14a,14b-tetradecahydropicene-2-carboxylic acid,” and “(9β,13α,14β,20α)-3-hydroxy-9,13-dimethyl-2-oxo-24,25,26-trinoroleana-1(10),3,5,7-tetraen-29-oic acid” are also synonymous to celastrol.

The term “active pharmaceutical ingredient” or “API” refers to the substance in a pharmaceutical drug that is biologically active. In some embodiments, the API described herein is celastrol.

The term “antisolvent” is used to refer to a solvent in which compounds described herein (e.g., celastrol and solid forms thereof) are poorly soluble.

The terms “crystalline form”, “single crystalline form” and “polymorph” are synonymous and refer to a solid form having a highly regular chemical structure. A crystalline form can have distinguishing, characteristic properties (e.g., XRPD data or thermal data such as DSC and TGA profiles). The term “polymorph” also includes pseudo-polymorphs, which are typically different solvates of a material, and thus their properties differ from one another. Thus, each distinct polymorph and pseudo-polymorph described herein is considered to be a distinct single crystalline form herein.

The term “about” refers to being within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

“Substantially crystalline” refers to a solid form that may be at least a particular weight percent crystalline. Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. In some embodiments, substantially crystalline refers to solid forms that are at least 70% crystalline or at least 90% crystalline.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate or solvated” means a physical association of a compound of this invention with one or more solvent molecules. A physical association includes hydrogen bonding. In some embodiments, a solvate will be capable of isolation, for example when one or more solvent molecules are incorporated in the crystal lattice of the crystalline solid. “Solvate or solvated” encompasses both solution-phase and isolable solvates. Representative solvates include, for example, a hydrate, ethanolates or a methanolate.

The term “hydrate” is a solvate wherein the solvent molecule is H₂O, which is present in a defined stoichiometric amount. Exemplary hydrates include hemihydrate, monohydrate, dihydrate, or trihydrate.

The term “molecular complex” refers to a solid form comprising two or more different molecules, comprising (e.g., a co-crystal that comprises an API (e.g., Celastrol) and a coformer) or ions (in the case of a salt of Celastrol and a counterion). In some embodiments, the molecular complex is composed of an API (e.g., Celastrol) with a neutral guest compound (also referred to as a conormer) in the crystal lattice. In some embodiments, the molecular complex comprises an API (e.g., Celastrol) and a coformer, wherein the API and coformer are bonded through non-covalent and non-ionic interactions (e.g., by hydrogen bonding, pi-stacking, guest-host complexation and/or van der Waals interactions). The term also includes solvates (e.g., hydrates) thereof.

The term “coformer” refers to a compound that is a component of a molecular complex with an API, wherein the compound and the API are bonded through non-covalent and non-ionic interactions (e.g., by hydrogen bonding, pi-stacking, guest-host complexation and/or van der Waals interactions). In some embodiments, a molecular complex comprises an API and a coformer that is an amino acid.

The term “mixture” is used to refer to the combined elements of the mixture regardless of the phase-state of the combination (for example, liquid or liquid/crystalline).

As used herein, the term “salt” refers to ionic compounds that result from the neutralization reaction of an acid and a base. They are composed of related numbers of cations (positively charged ions) and anions (negative ions) so that the product is electrically neutral (without a net charge). These component ions can be inorganic, such as chloride (Cl−), or organic, such as acetate (C₂H₃O₂−); and can be monatomic, such as fluoride (F−), or polyatomic, such as sulfate (SO₄²−).

The term “seeding” is used to refer to the addition of a crystalline material to initiate recrystallization or crystallization.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present invention, whether radioactive or not, are encompassed within the scope of the present invention.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat diseases associated with weight gain such as obesity.

An “effective amount” is an amount sufficient for a compound to accomplish a stated purpose relative to the absence of the compound (e.g. achieve the effect for which it is administered, treat a disease, reduce enzyme activity, increase enzyme activity, reduce signaling pathway, reduce one or more symptoms of a disease or condition. An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “associated” or “associated with” in the context of a substance or substance activity or function associated with a disease (e.g. obesity) means that the disease is caused by (in whole or in part), or a symptom of the disease is caused by (in whole or in part) the substance or substance activity or function. As used herein, what is described as being associated with a disease, if a causative agent, could be a target for treatment of the disease. For example, a disease associated with weight gain such as obesity may be treated with an agent (e.g. compound as described herein) effective for decreasing weight gain.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

As defined herein, the term “inhibition”, “inhibit”, “inhibiting” and the like in reference to a protein-inhibitor (e.g. antagonist) interaction means negatively affecting (e.g. decreasing) the level of activity or function of the protein relative to the level of activity or function of the protein in the absence of the inhibitor. In some embodiments inhibition refers to reduction of a disease or symptoms of disease. Thus, inhibition may include, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g. agonist) interaction means positively affecting (e.g. increasing) the activity or function of the protein relative to the activity or function of the protein in the absence of the activator (e.g. compound described herein). Thus, activation may include, at least in part, partially or totally increasing stimulation, increasing or enabling activation, or activating, sensitizing, or up-regulating signal transduction or enzymatic activity or the amount of a harmful mediator/substance decreased in a disease. Activation may include, at least in part, partially or totally increasing stimulation, increasing or enabling activation, or activating, sensitizing, or up-regulating signal transduction or enzymatic activity or the amount of a harmful mediator/substance.

“Anti-obesity agent” refers to the property of a substance or treatment that reduces weight gain and promotes weight loss. Non-limiting examples of anti-obesity agents include Sibutramine, Phentermine, Mazindol, Diethylpropion, Leptin, Orlistat, Beta-3 agonists, and Rimonabant.

The term “obese” is used therein, refers to a patient having a body mass index of greater than 30 kg/m². “Overweight” and “pre-obese”, as used herein, refer to patients having a body mass index of greater than 25 kg/m². “Morbidly obese”, as used herein, refers to a patient having a BMI of greater than 40 mg/m², a BMI of greater than 35 kg/m² in combination with one ore more co-morbidities, a BMI of greater than 30 kg/m² in combination with uncontrollable diabetes, or combinations thereof.

The term “prodrug” refers to a pharmacological substance such as a drug that is administered to a subject in an inactive (or significantly less active) form. Once administered, the prodrug is metabolized in the body (in vivo) into a compound having the desired pharmacological activity.

“Patient” or “subject in need thereof” refers to a living organism suffering from or prone or susceptible to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. In various embodiments, a patient or subject has not been genetically modified (e.g., does not comprise a transgene or a germline mutation that has been intentionally and artificially introduced into the subject).

“Disease” or “condition” refer to a state of being or health status of a patient or subject capable of being treated with a compound, pharmaceutical composition, or method provided herein. In some embodiments, the disease is a disease having an increase in body weight. In some embodiments, the disease is obesity. Obesity may be the primary cause of the disease and/or disorder to be treated or may also by a result of the primary disease and/or disorder.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-obesity agent). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### II. Compounds

Provided herein, inter alia, are solid forms, including crystalline and amorphous forms, of Compound I,

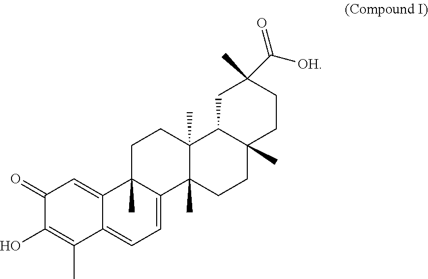

The invention also features salts and molecular complexes of Compound I (Celastrol). In some embodiments, a salt and/or a molecular complex of Compound I (Celastrol) is amorphous. In some embodiments, a salt and/or a molecular complex of Compound I is crystalline

Celastrol can be obtained from commercial sources, or isolated from plants, e.g. Tripterygium, by methods known in the art (Kutney et al, Can. J. Chem. 59:2677, 1981) and Zhang et al, Acta Pharm. Sin. 212: 592, 1986). Celastrol can be modified to render compounds of Formula (I) as disclosed in U.S. Patent Application Publication No. 2015-0250753, published Sep. 10, 2015, which is incorporated herein by reference in its entirety.

Without wishing to be bound by theory, Compound I isolated from plants or obtained by other sources may contain an undesirable amount of impurities. For example, the purity of crude Compound I may be ≤98%, ≤97%, ≤96%, ≤95%, ≤94%, ≤93%, ≤92%, ≤91%, or ≤90%. In some aspects, the disclosure features methods of purifying celastrol by making crystalline forms of celastrol. In some embodiments, a highly pure form of Compound I is Form III of Compound I as described herein.

In an aspect is provided a crystalline Form III of Compound I, which is characterized by an x-ray powder diffraction pattern comprising two or more characteristic peaks at 2θ angles selected from the group consisting of 9.51°, 13.60°, 14.65°, 14.81°, 16.84°, 18.74°, and 19.10°.

In some embodiments, the crystalline Form III, is characterized by an x-ray powder diffraction pattern comprising three, four, five, or six characteristic peaks at 2θ angles selected from the group consisting of 9.51°, 13.60°, 14.65°, 14.81°, 16.84°, 18.74°, and 19.10°.

In an aspect is provided a crystalline Form III of Compound I, which is characterized by an x-ray powder diffraction pattern comprising peaks at 2θ angles (±0.2) of 9.51°, 14.81°, and 16.84°.

In some embodiments, the crystalline Form III of Compound I is characterized by an X-ray powder diffraction pattern comprising one or more peaks at 2θ angles (±0.2) selected from the group consisting of 13.60°, 14.65°, 18.74°, and 19.10°.

In some embodiments, the crystalline Form III of Compound I is characterized by a differential scanning calorimetry (DSC) profile with no endothermic peak and an exothermic peak at about 215° C. (onset).

In some embodiments, the crystalline Form III of Compound I is characterized by a thermal gravimetric analysis (TGA) profile with about 0.5% weight loss at about the exothermic peak at about 215° C. (onset).

In some embodiments, the crystalline Form III of Compound I is anhydrous (e.g., an anhydrous Form III crystalline solid form).

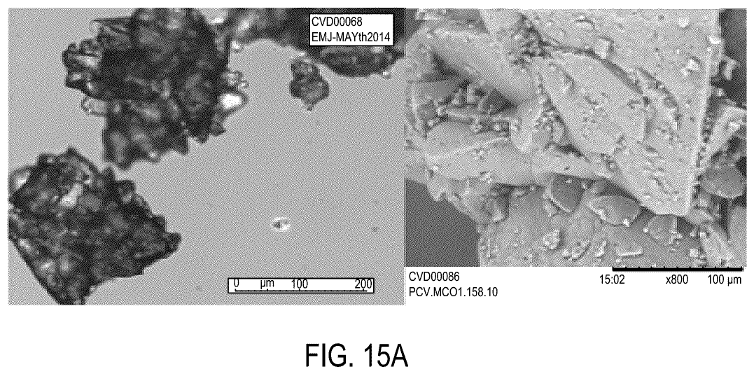

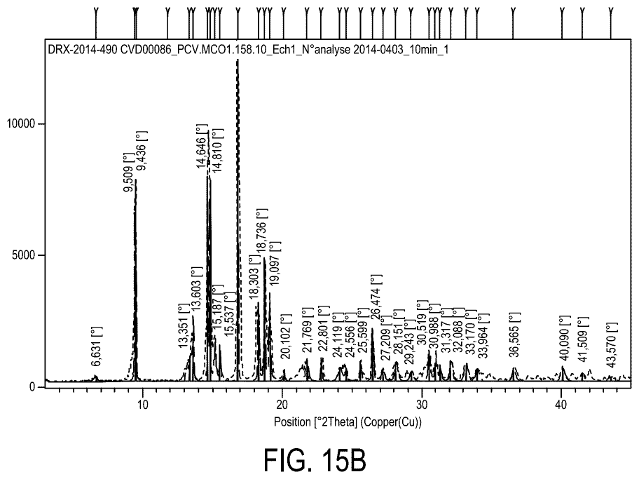

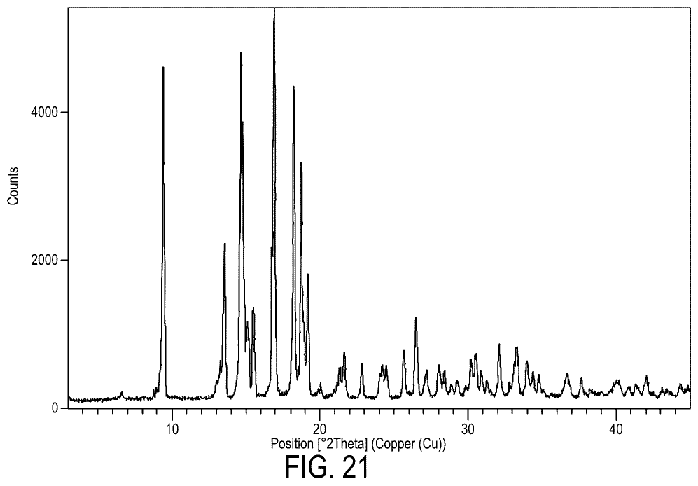

In some embodiments, the crystalline Form III of Compound I (e.g., a Form III crystalline solid form, e.g., an anhydrous Form III crystalline solid form) is characterized by an X-ray powder diffraction pattern substantially similar to FIG. 21 or FIG. 15B. In some embodiments, the crystalline Form III of Compound I (e.g., Form III crystalline solid form, e.g., an anhydrous Form III crystalline solid form) is characterized by an X-ray powder diffraction pattern that is about the same or is the same as the pattern shown in FIG. 21 or FIG. 15B.

In some embodiments, the crystalline Form III of Compound I (e.g., a Form III crystalline solid form, e.g., an anhydrous Form III crystalline solid form) is characterized by an X-ray powder diffraction pattern comprising one or more peaks (e.g., one, two, or three peaks) at 2θ angles (±0.2) selected from the group consisting of 9.51°, 14.81°, and 16.84°.

In some embodiments, the crystalline Form III of Compound I (e.g., a Form III crystalline solid form, e.g., an anhydrous Form III crystalline solid form) is characterized by an X-ray powder diffraction pattern comprising peaks at 2θ angles (±0.2) of 9.51°, 14.81°, and 16.84°.

In some embodiments, the crystalline Form III of Compound I (e.g., a Form III crystalline solid form, e.g., an anhydrous Form III crystalline solid form) is characterized by an X-ray powder diffraction pattern comprising one or more peaks (e.g., one, two, three, or four peaks) at 2θ angles (±0.2) selected from the group consisting of 13.60°, 14.65°, 18.74°, and 19.10°.

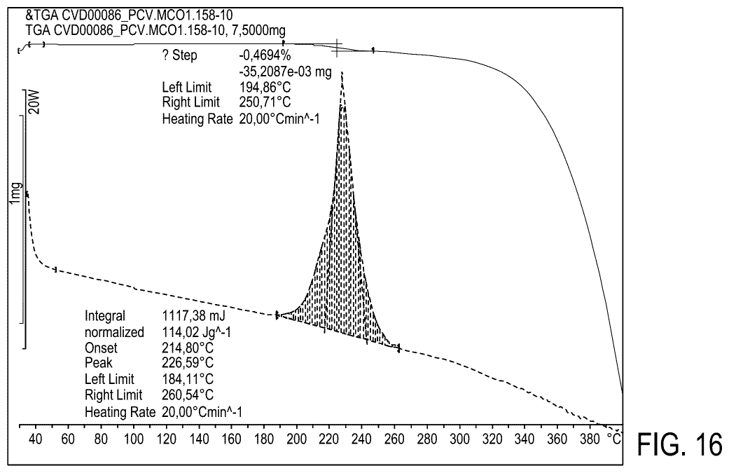

In some embodiments, the crystalline Form III of Compound I (e.g., a Form III crystalline solid form, e.g., an anhydrous Form III crystalline solid form) is characterized by a differential scanning calorimetry (DSC) profile substantially similar to FIG. 16. In some embodiments, the crystalline Form III of Compound I (e.g., a Form III crystalline solid form, e.g., an anhydrous Form III crystalline solid form) is characterized by a DSC that is about the same or is the same as the profile shown in FIG. 16.

In some embodiments, the crystalline Form III of Compound I (e.g., a Form III crystalline solid form, e.g., an anhydrous Form III crystalline solid form) is characterized by a thermal gravimetric analysis (TGA) profile substantially similar to FIG. 16. In some embodiments, the crystalline Form III of Compound I (e.g., a Form III crystalline solid form, e.g., an anhydrous Form III crystalline solid form) is characterized by a TGA profile substantially that is about the same or is the same as the profile shown in FIG. 16.

In some embodiments, the crystalline Form III of Compound I (e.g., a Form III crystalline solid form, e.g., an anhydrous Form III crystalline solid form) is characterized by a differential scanning calorimetry (DSC) profile with no endothermic peak. In some embodiments, the crystalline Form III of Compound I (e.g., a Form III crystalline solid form, e.g., an anhydrous Form III crystalline solid form) is characterized by a differential scanning calorimetry (DSC) profile with an exothermic peak at about 215° C. (onset). In some embodiments, the crystalline Form III of Compound I (e.g., a Form III crystalline solid form, e.g., an anhydrous Form III crystalline solid form) is characterized by a differential scanning calorimetry (DSC) profile with no endothermic peak but with an exothermic peak at about 215° C. (onset).

In some embodiments, the crystalline Form III of Compound I (e.g., a Form III crystalline solid form, e.g., an anhydrous Form III crystalline solid form) is characterized by a thermal gravimetric analysis (TGA) profile with about 0.5% weight loss at about the exothermic peak (e.g., at about 215° C. (onset)).

In some embodiments, the crystalline Form III of Compound I (e.g., a Form III crystalline solid form, e.g., an anhydrous Form III crystalline solid form) is characterized by
(i) a differential scanning calorimetry (DSC) profile with no endothermic peak and an exothermic peak at about 215° C. (onset); and
(ii) a thermal gravimetric analysis (TGA) profile with about 0.5% weight loss at about the exothermic peak at about 215° C. (onset).

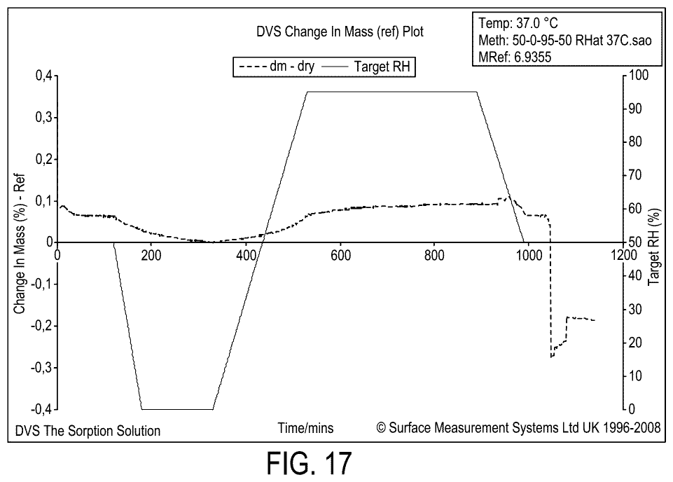

In some embodiments, the crystalline Form III of Compound I (e.g., a Form III crystalline solid form, e.g., an anhydrous Form III crystalline solid form) is characterized by a dynamic vapor sorption profile substantially similar to FIG. 17. In some embodiments, the crystalline Form III of Compound I (e.g., a Form III crystalline solid form, e.g., an anhydrous Form III crystalline solid form) is characterized by a dynamic vapor sorption that is about the same or is the same as the profile shown in FIG. 17.

In an aspect is provided a crystalline Form I of Compound I, which is characterized by an x-ray powder diffraction pattern comprising peaks at 2θ angles (±0.2) of 9.53°, 13.29°, and 16.13°.

In some embodiments, the crystalline Form I of Compound I is characterized by an X-ray powder diffraction pattern comprising one or more peaks at 2θ angles (±0.2) selected from the group consisting of 8.34°, 11.12°, 15.08°, 15.35°, 20.86°, and 22.95°.

In some embodiments, the crystalline Form I of Compound I is characterized by a differential scanning calorimetry (DSC) profile with an endothermic peak at about 156° C. (onset) and an exothermic peak at about 222° C. (onset).

In some embodiments, the crystalline Form I of Compound I is characterized by a thermal gravimetric analysis (TGA) profile with about 4.7% weight loss at about 155° C.

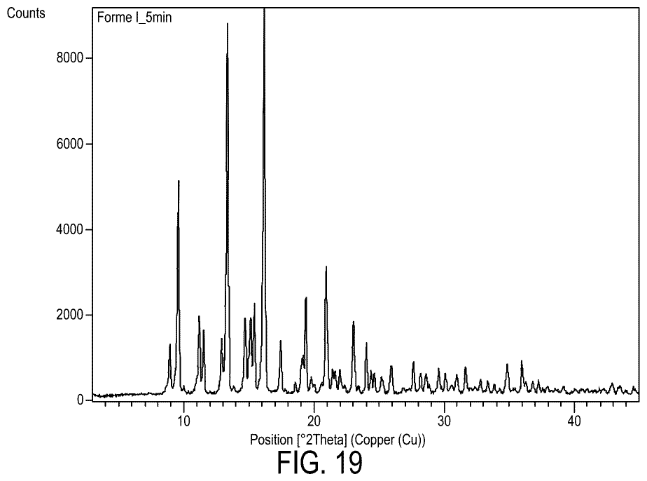

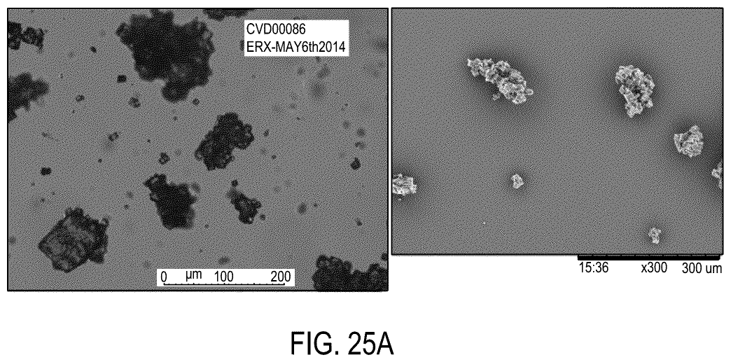

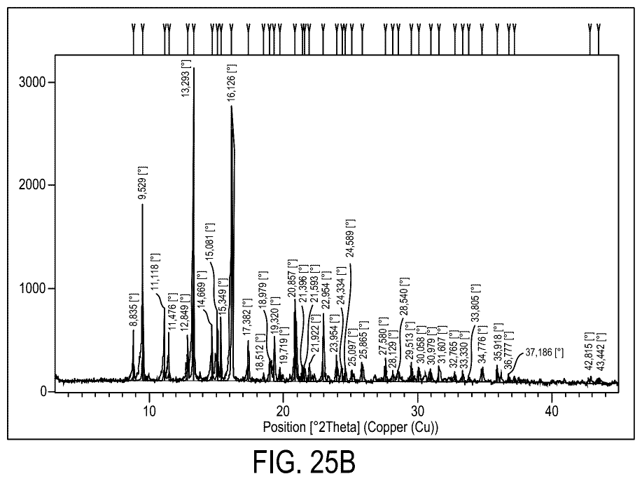

In some embodiments, the crystalline Form I of Compound I (e.g., a Form I crystalline solid form) is characterized by an X-ray powder diffraction pattern substantially similar to FIG. 19 or FIG. 25B. In some embodiments, the crystalline Form I of Compound I (e.g., a Form I crystalline solid form) is characterized by an X-ray powder diffraction pattern that is about the same or is the same as the pattern shown in FIG. 19 or FIG. 25B. In some embodiments, the crystalline Form I of Compound I (e.g., a Form I crystalline solid form) is characterized by an X-ray powder diffraction pattern including one or more peaks (e.g., 1, 2, or 3 peaks) at 2θ angles (±0.2) selected from the group consisting of 9.53°, 13.29°, and 16.13°.

In some embodiments, the crystalline Form I of Compound I (e.g., a Form I crystalline solid form) is characterized by an X-ray powder diffraction pattern including peaks at 2θ angles (±0.2) of 9.53°, 13.29°, and 16.13°.

In some embodiments, the crystalline Form I of Compound I (e.g., a Form I crystalline solid form) is characterized by one or more peaks (e.g., one, two, three, four, five, or six peaks) at 2θ angles (±0.2) selected from the group consisting of 8.34°, 11.12°, 15.08°, 15.35°, 20.86°, and 22.95°.

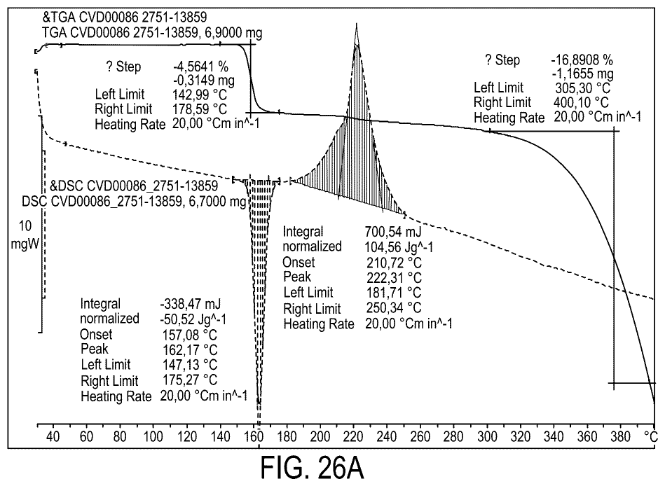

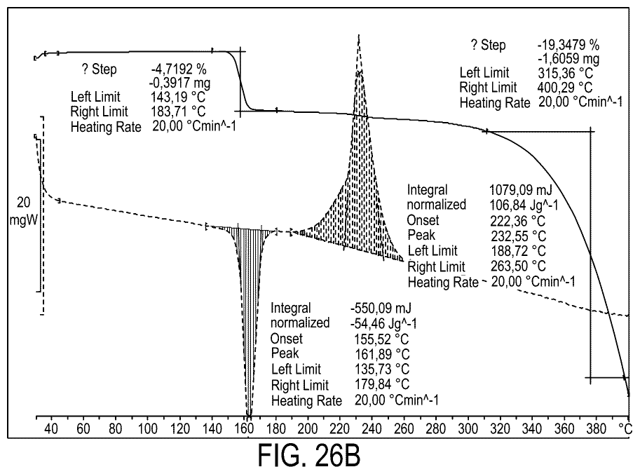

In some embodiments, the crystalline Form I of Compound I (e.g., a Form I crystalline solid form) is characterized by a differential scanning calorimetry (DSC) profile substantially similar to FIG. 26. In some embodiments, the crystalline Form I of Compound I (e.g., a Form I crystalline solid form) is characterized by a DSC that is about the same or is the same as the profile shown in FIG. 26B.

In some embodiments, the crystalline Form I of Compound I (e.g., a Form I crystalline solid form) is characterized by a thermal gravimetric analysis (TGA) profile substantially similar to FIG. 26B. In some embodiments, the crystalline Form I of Compound I (e.g., a Form I crystalline solid form) is characterized by a TGA profile substantially that is ab out the same or is the same as the profile shown in FIG. 26B.

In some embodiments, the crystalline Form I of Compound I (e.g., a Form I crystalline solid form) is characterized by a differential scanning calorimetry (DSC) profile with an endothermic peak at about 155° C. (onset). In some embodiments, the crystalline Form I of Compound I (e.g., a Form I crystalline solid form) is characterized by a differential scanning calorimetry (DSC) profile with an exothermic peak at about 222° C. (onset). In some embodiments, the crystalline Form I of Compound I (e.g., a Form I crystalline solid form) is characterized by a differential scanning calorimetry (DSC) profile with an endothermic peak at about 155° C. (onset) and an exothermic peak at about 222° C. (onset).

In some embodiments, the crystalline Form I of Compound I (e.g., a Form I crystalline solid form) is characterized by a thermal gravimetric analysis (TGA) profile with about 4.7% weight loss at about 155° C.

In some embodiments, the crystalline Form I of Compound I (e.g., a Form I crystalline solid form) is characterized by
(i) a differential scanning calorimetry (DSC) profile with an endothermic peak at about 155° C. (onset) and an exothermic peak at about 222° C. (onset); and
(ii) a thermal gravimetric analysis (TGA) profile with about 4.7% weight loss at about 155° C.

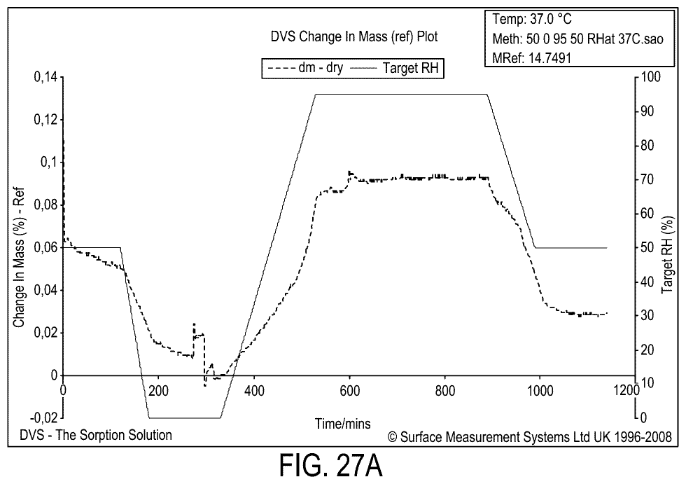

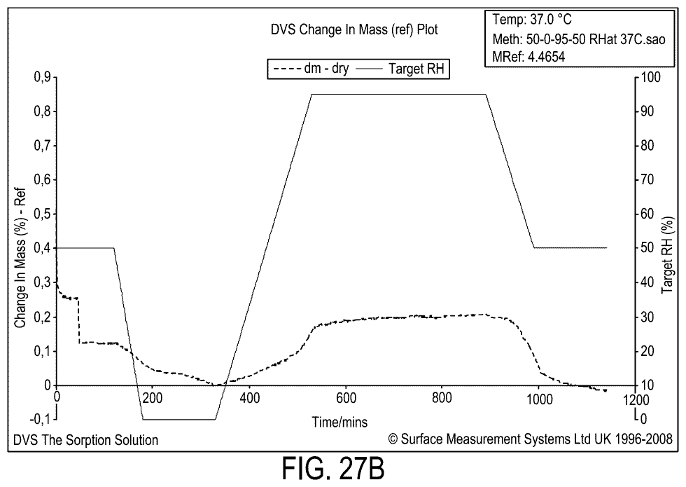

In some embodiments, the crystalline Form I of Compound I (e.g., a Form I crystalline solid form) is characterized by a dynamic vapor sorption profile substantially similar to FIG. 27A. In some embodiments, the crystalline Form I of Compound I (e.g., a Form I crystalline solid form) is characterized by a dynamic vapor sorption that is about the same or is the same as the profile shown in FIG. 27A.

In an aspect is provided a crystalline Form II of Compound I (e.g., a Form II crystalline solid form), which is characterized by an X-ray powder diffraction pattern comprising peaks at 2θ angles (±0.2) of 11.68°, 16.07°, and 17.04°.

In some embodiments, the crystalline Form II of Compound I (e.g., a Form II crystalline solid form) is characterized by an X-ray powder diffraction pattern comprising one or more peaks at 2θ angles (±0.2) selected from the group consisting of 8.48°, 14.05°, 14.68°, 19.30°, 24.18, and 26.87°.

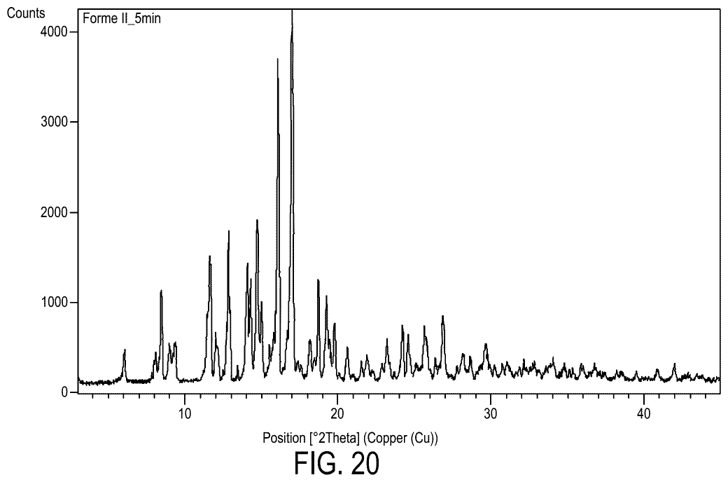

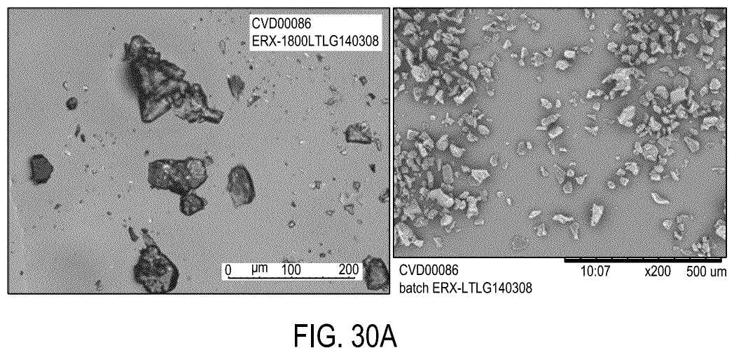

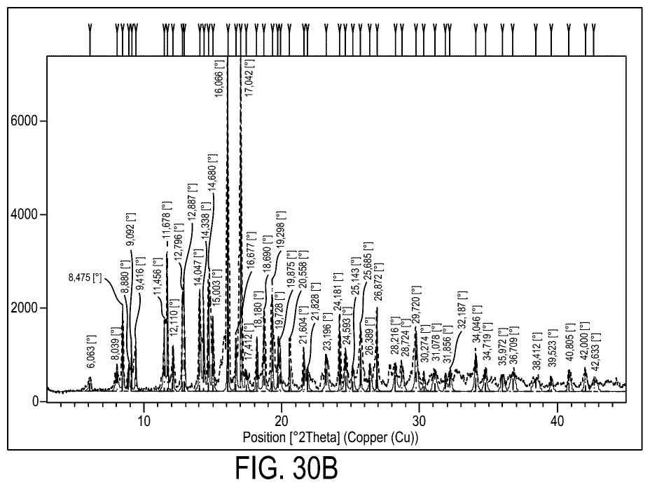

In some embodiments, the crystalline Form II of Compound I (e.g., a Form II crystalline solid form) is characterized by an X-ray powder diffraction pattern substantially similar to FIG. 20 or FIG. 30B. In some embodiments, the crystalline Form II of Compound I (e.g., a Form II crystalline solid form) is characterized by an X-ray powder diffraction pattern that is about the same or is the same as the pattern shown in FIG. 20 or FIG. 30B.

In some embodiments, the crystalline Form II of Compound I (e.g., a Form II crystalline solid form) is characterized by an X-ray powder diffraction pattern comprising one or more peaks (e.g., one, two, or three peaks) at 2θ angles (±0.2) selected from the group consisting of 11.68°, 16.07°, and 17.04°.

In some embodiments, the crystalline Form II of Compound I (e.g., a Form II crystalline solid form) is characterized by an X-ray powder diffraction pattern comprising peaks at 2θ angles (±0.2) of 11.68°, 16.07°, and 17.04°.

In some embodiments, the crystalline Form II of Compound I (e.g., a Form II crystalline solid form) is characterized by one or more peaks (e.g., one, two, three, four, five, or six peaks) at 2θ angles (±0.2) selected from the group consisting of 8.48°, 14.05°, 14.68°, 19.30°, 24.18, and 26.87°.

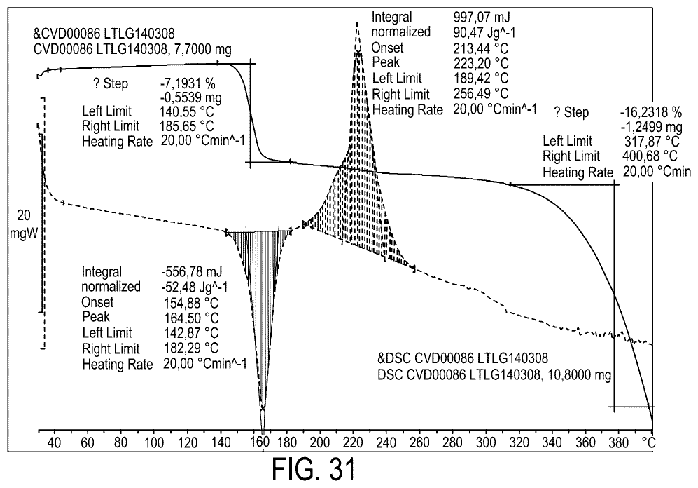

In some embodiments, the crystalline Form II of Compound I (e.g., a Form II crystalline solid form) is characterized by a differential scanning calorimetry (DSC) profile substantially similar to FIG. 31. In some embodiments, the crystalline Form II of Compound I (e.g., a Form II crystalline solid form) is characterized by a DSC that is about the same or is the same as the profile shown in FIG. 31.

In some embodiments, the crystalline Form II of Compound I (e.g., a Form II crystalline solid form) is characterized by a thermal gravimetric analysis (TGA) profile substantially similar to FIG. 31. In some embodiments, the crystalline Form II of Compound I (e.g., a Form II crystalline solid form) is characterized by a TGA profile substantially that is about the same or is the same as the profile shown in FIG. 31.

In some embodiments, the crystalline Form II of Compound I (e.g., a Form II crystalline solid form) is characterized by a differential scanning calorimetry (DSC) profile with an endothermic peak at about 155° C. (onset). In some embodiments, the crystalline Form II of Compound I (e.g., a Form II crystalline solid form) is characterized by a differential scanning calorimetry (DSC) profile with an exothermic peak at about 213° C. (onset). In some embodiments, the crystalline Form II of Compound I (e.g., a Form II crystalline solid form) is characterized by a differential scanning calorimetry (DSC) profile with an endothermic peak at about 155° C. (onset) and an exothermic peak at about 213° C. (onset).

In some embodiments, the crystalline Form II of Compound I (e.g., a Form II crystalline solid form) is characterized by a differential scanning calorimetry (DSC) profile with a thermal gravimetric analysis (TGA) profile with about 7% weight loss at about 155° C.

In some embodiments, the crystalline Form II of Compound I (e.g., a Form II crystalline solid form) is characterized by
(i) a differential scanning calorimetry (DSC) profile with an endothermic peak at about 155° C. (onset) and an exothermic peak at about 213° C. (onset); and
(ii) a thermal gravimetric analysis (TGA) profile with about 7% weight loss at about 155° C.

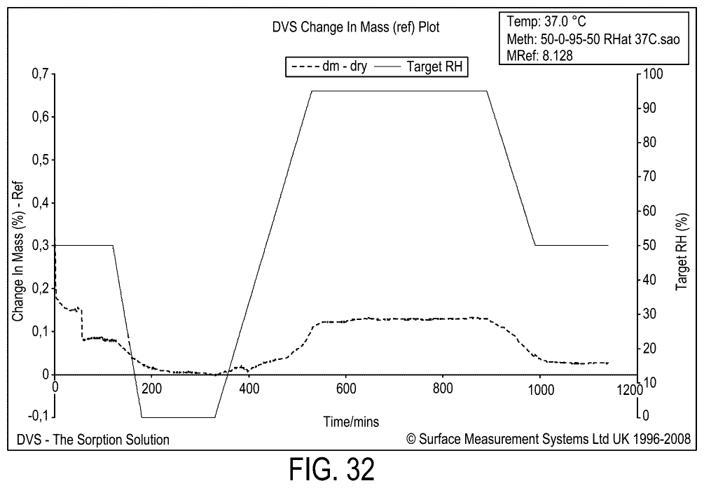

In some embodiments, the crystalline Form II of Compound I (e.g., a Form II crystalline solid form is characterized by a dynamic vapor sorption profile substantially similar to FIG. 32. In some embodiments, the crystalline Form II of Compound I (e.g., a Form II crystalline solid form) is characterized by a dynamic vapor sorption that is about the same or is the same as the profile shown in FIG. 32.

In some embodiments, the crystalline form is Form IV.

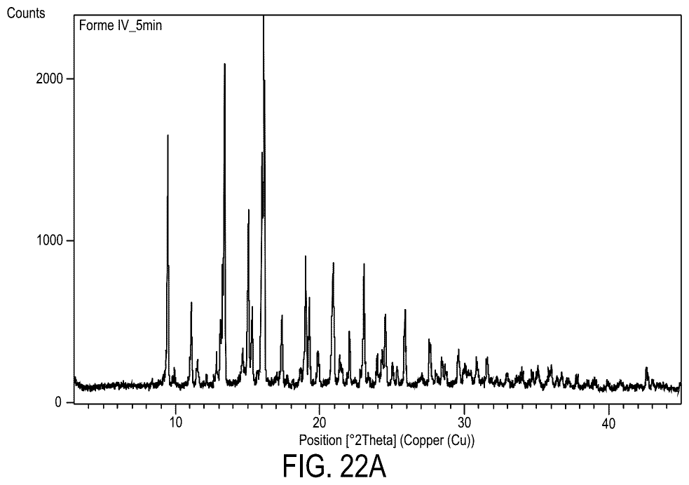

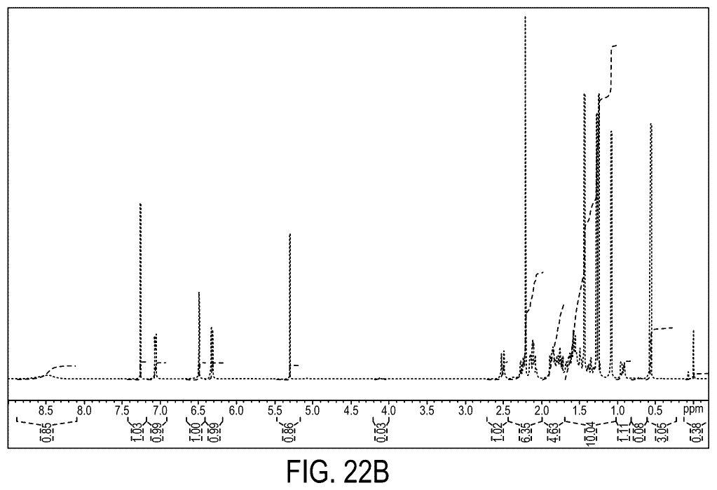

In some embodiments, the crystalline Form IV of Compound I (e.g., a Form IV crystalline solid form) is characterized by an X-ray powder diffraction pattern substantially similar to FIG. 22. In some embodiments, the crystalline Form IV of Compound I (e.g., a Form IV crystalline solid form) is characterized by an X-ray powder diffraction pattern that is about the same or is the same as the pattern shown in FIG. 22A.

In some embodiments, the crystalline Form IV of Compound I (e.g., a Form IV crystalline solid form) is characterized by an X-ray powder diffraction pattern comprising peaks at 2θ angles (±0.2) of 13.4°, 14.6°, and 17.4°.

In some embodiments, the crystalline Form IV of Compound I (e.g., a Form IV crystalline solid form) is characterized by an X-ray powder diffraction pattern comprising peaks at 2θ angles (±0.2) of 11.1°, 16.2°, and 17.4°.

In some embodiments, the crystalline Form IV of Compound I (e.g., a Form IV crystalline solid form) is characterized by an X-ray powder diffraction pattern comprising peaks at 2θ angles (±0.2) of 11.5°, 16.2°, and 17.4°.

In some embodiments, the crystalline Form IV of Compound I (e.g., a Form IV crystalline solid form) is characterized by an X-ray powder diffraction pattern comprising peaks at 2θ angles (±0.2) of 11.1°, 13.4°, 15.0°, 16.2°, and 17.4°.

In some embodiments, the crystalline Form IV of Compound I (e.g., a Form IV crystalline solid form) is characterized by an X-ray powder diffraction pattern comprising peaks at 2θ angles (±0.2) of 11.5°, 13.4°, 15.0°, 16.2°, and 17.4°.

In some embodiments, the crystalline Form IV of Compound I (e.g., a Form IV crystalline solid form) is characterized by an X-ray powder diffraction pattern comprising one or more peaks (e.g., one, two, three, four, or five peaks) at 2θ angles (±0.2) selected from the group consisting of 11.1°, 13.4°, 15.0°, 16.2°, and 17.4°.

In some embodiments, the crystalline Form IV of Compound I (e.g., a Form IV crystalline solid form) is characterized by an X-ray powder diffraction pattern comprising one or more peaks (e.g., one, two, three, four, or five peaks) at 2θ angles (±0.2) selected from the group consisting of 11.5°, 13.4°, 15.0°, 16.2°, and 17.4°.

In some embodiments, the crystalline Form IV of Compound I (e.g., a Form IV crystalline solid form) is characterized by an X-ray powder diffraction pattern comprising peaks at 2θ angles (±0.2) of 9.5, 11.1°, 11.5°, 13.4°, 14.6°, 15.0°, 15.2°, 16.2°, 17.4°, 19.0°, and 19.3°.

In some embodiments, the crystalline Form IV of Compound I (e.g., a Form IV crystalline solid form) is characterized by an X-ray powder diffraction pattern comprising one or more peaks (e.g., one, two, three, four, five, or six peaks) at 2θ angles (±0.2) selected from the group consisting of 9.5, 11.1°, 11.5°, 13.4°, 14.6°, 15.0°, 15.2°, 16.2°, 17.4°, 19.0°, and 19.3°.

In some embodiments, the crystalline Form IV of Compound I (e.g., a Form IV crystalline solid form) is characterized by an X-ray powder diffraction pattern comprising peaks at 2θ angles (±0.2) of 9.5, 11.1°, 11.5°, 13.4°, 14.6°, 15.0°, 15.2°, 16.2°, 17.4°, 19.0°, 19.3°, 20.9°, and 23.0°.

In some embodiments, the crystalline Form IV of Compound I (e.g., a Form IV crystalline solid form) is characterized by an X-ray powder diffraction pattern comprising one or more peaks (e.g., one, two, three, four, five, or six peaks) at 2θ angles (±0.2) selected from the group consisting of 9.5, 11.1°, 11.5°, 13.4°, 14.6°, 15.0°, 15.2°, 16.2°, 17.4°, 19.0°, 19.3°, 20.9°, and 23.0°.

In some embodiments, the crystalline Form IV of Compound I (e.g., a Form IV crystalline solid form) is characterized by an X-ray powder diffraction pattern comprising peaks at 2θ angles (±0.2) of 9.5°, 11.1°, 11.5°, 13.4°, 14.6°, 15.0°, 15.2°, 16.2°, 17.4°, 19.0°, 19.3°, 20.0°, 20.9°, 21.4°, 22.0°, 23.0°, 24.0°, 24.5°, 25.9°, and 26.6°.

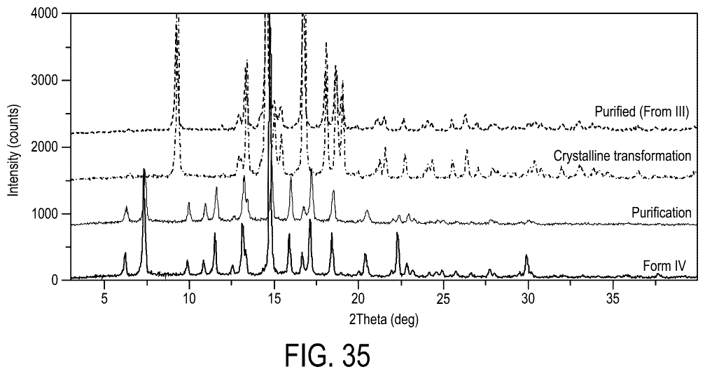

In some embodiments, the crystalline Form IV of Compound I (e.g., a Form IV crystalline solid form) is characterized by an X-ray powder diffraction pattern substantially similar to the bottom two spectra shown in FIG. 35. In some embodiments, the crystalline Form IV of Compound I (e.g., a Form IV crystalline solid form) is characterized by an X-ray powder diffraction pattern that is about the same or is the same as the bottom two spectra shown in FIG. 35.

In some embodiments, the crystalline Form IV of Compound I (e.g., a Form IV crystalline solid form) is characterized by an X-ray powder diffraction pattern comprising peaks at 2θ angles (±0.2) of 13.2°, 14.9, 16.1, and 17.2°.

In some embodiments, the crystalline Form IV of Compound I (e.g., a Form IV crystalline solid form) is characterized by an X-ray powder diffraction pattern comprising peaks at 2θ angles (±0.2) of 10.9°, 13.2°, 14.9, 16.1, and 17.2°.

In some embodiments, the crystalline Form IV of Compound I (e.g., a Form IV crystalline solid form) is characterized by one or more peaks (e.g., one, two, three, or four) at 20 angles (±0.2) selected from the group consisting of 10.0°, 10.9°, 18.5°, and 20.5°.

In some embodiments, the crystalline Form IV of Compound I (e.g., a Form IV crystalline solid form) is characterized by one or more peaks (e.g., one, two, three, four, or five) at 2θ angles (±0.2) selected from the group consisting of 10.0°, 10.9°, 11.6°, 13.2°, 16.1°, and 20.5°.

In some embodiments, the crystalline Form IV of Compound I (e.g., a Form IV crystalline solid form) is characterized by one or more peaks (e.g., one, two, three, four, five, or six peaks) at 2θ angles (±0.2) selected from the group consisting of 10.0°, 10.9°, 11.6°, 13.2°, 14.9°, 16.1°, 17.2°, 18.5°, and 20.5°.

In some embodiments, the crystalline Form IV of Compound I (e.g., a Form IV crystalline solid form) is characterized by one or more peaks (e.g., one, two, three, four, five, or six peaks) at 2θ angles (±0.2) selected from the group consisting of 6.3°, 7.4°, 10.0°, 10.9°, 11.6°, 13.2°, 14.9°, 16.1°, 17.2°, 18.5°, and 20.5°.

In some embodiments, the crystalline form is Form V.

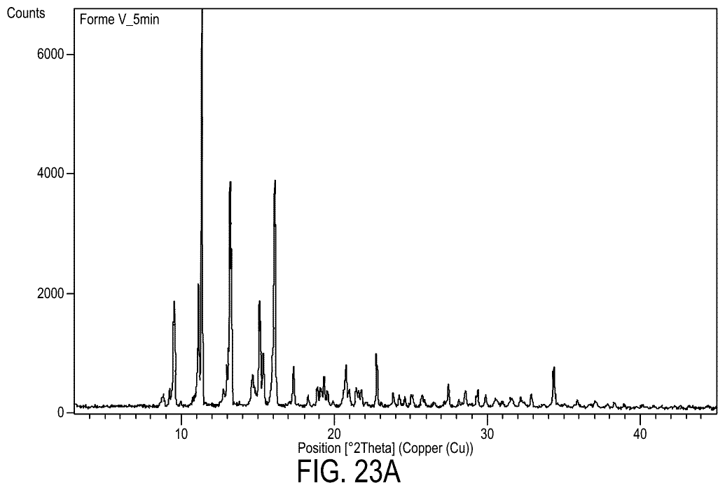

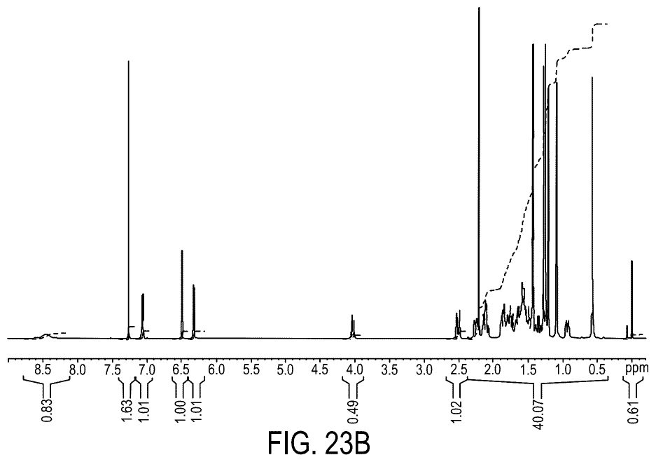

In some embodiments, the crystalline Form V of Compound I (e.g., a Form V crystalline solid form) is characterized by an X-ray powder diffraction pattern substantially similar to FIG. 23. In some embodiments, the crystalline Form V of Compound I (e.g., a Form V crystalline solid form) is characterized by an X-ray powder diffraction pattern that is about the same or is the same as the pattern shown in FIG. 23.

In some embodiments, the crystalline form is Form VI.

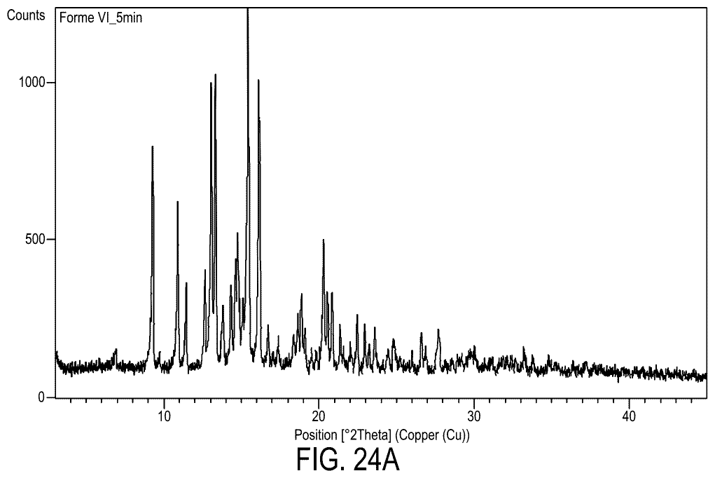

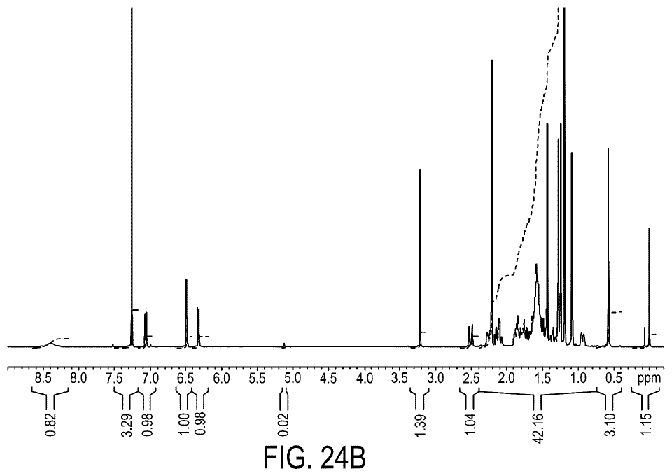

In some embodiments, the crystalline Form VI of Compound I (e.g., a Form VI crystalline solid form) is characterized by an X-ray powder diffraction pattern substantially similar to FIG. 24. In some embodiments, the crystalline Form VI of Compound I (e.g., a Form VI crystalline solid form) is characterized by an X-ray powder diffraction pattern that is about the same or is the same as the pattern shown in FIG. 24.

In some embodiments, a crystalline form described herein is substantially free of methanol, ethanol, and/or dichloromethane. In some embodiments, a crystalline form described herein is substantially free of methanol, ethanol, and dichloromethane.

In an aspect, provided is a pharmaceutical composition including one or more crystalline form of Compound I as described herein, and a pharmaceutically acceptable excipient.

In some embodiments, the crystalline form of Compound I is Form III as described herein. In some embodiments, the crystalline form of Compound I is Form I as described herein. In some embodiments, the crystalline form of Compound I is Form II as described herein.

In some embodiments, the pharmaceutical composition is formulated into a tablet, capsule, suspension, dispersion, injectable, or other pharmaceutical form.

In an aspect, Compound I may be in amorphous solid form. In some embodiments, the amorphous solid form is substantially free of methanol and ethanol.

In an aspect, the invention features a pharmaceutical composition comprising the amorphous solid form of Compound I as described herein, and a pharmaceutically acceptable excipient.

In some embodiments, the pharmaceutical composition is formulated into a tablet, capsule, suspension, dispersion, injectable, or other pharmaceutical form.

In an aspect, provided is a solid form of a complex including Compound I and a co-former (e.g., calcium hydroxide). In some embodiments, the solid form of the complex includes Compound I and Ca(OH)₂.

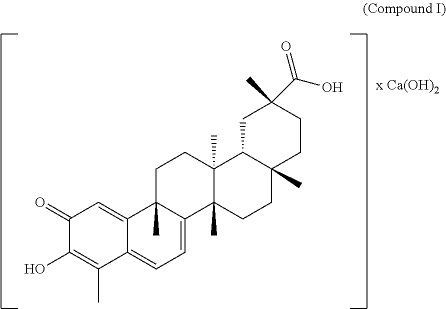

In some embodiments, the solid form of the complex is amorphous. In some embodiments, the solid form of the complex is crystalline.

In an aspect, provided is a solid form of a complex including Compound I and a co-former (e.g., L(+)-lysine). In some embodiments, the solid form of the complex includes Compound I and L(+)-lysine.

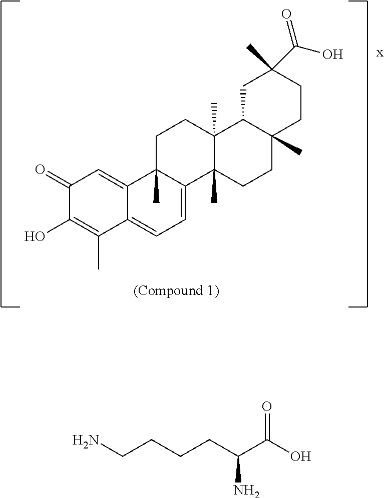

In an aspect, provided is a salt form of Compound 1. In some embodiments, the salt form of Compound I includes trometamol (tromethamine, tris(hydroxymethyl)aminomethane, tris). In some embodiments, provided is a salt form of Compound I and trometamol, e.g., the trometamol salt of Compound I (Compound 2). In some embodiments, the trometamol salt of Compound I includes Compound I and trometamol at a ratio of about 1:1.

In some embodiments, the trometamol salt of Compound I is a solid form. In some embodiments, the trometamol salt of Compound I form is an amorphous solid. In some embodiments, the trometamol salt of Compound I is a crystalline solid.

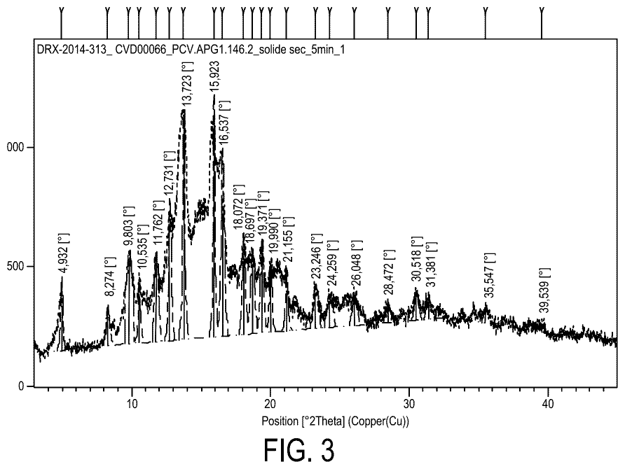

In some embodiments, the salt form of Compound I and trometamol (e.g., the solid salt form of Compound I and trometamol) is characterized by an X-ray powder diffraction pattern substantially similar to FIG. 3. In some embodiments, the salt form of Compound I and trometamol (e.g., the solid salt form of Compound I and trometamol) is characterized by an X-ray powder diffraction pattern that is about the same or is the same as the pattern shown in FIG. 3.

In some embodiments, the salt form of Compound I and trometamol (e.g., the solid salt form of Compound I and trometamol) is characterized by an X-ray powder diffraction pattern comprising one or more peaks (e.g., one, two, or three peaks) at 2θ angles (±0.2) selected from the group consisting of 4.93°, 13.72°, and 15.92°.

In some embodiments, the salt form of Compound I and trometamol (e.g., the solid salt form of Compound I and trometamol) is characterized by an X-ray powder diffraction pattern comprising peaks at 2θ angles (±0.2) of 4.93°, 13.72°, and 15.92°.

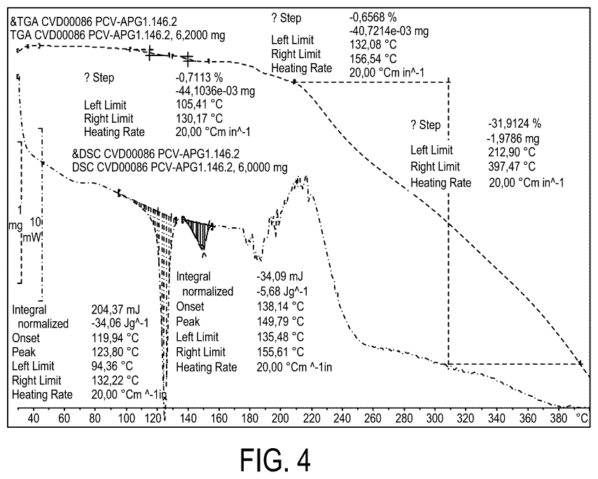

In some embodiments, the salt form of Compound I and trometamol (e.g., the solid salt form of Compound I and trometamol) is characterized by a differential scanning calorimetry (DSC) profile substantially similar to FIG. 4. In some embodiments, the salt form of Compound I and trometamol (e.g., the solid salt form of Compound I and trometamol) is characterized by a DSC profile that is the same or about the same as the profile shown in FIG. 4.

In some embodiments, the salt form of Compound I and trometamol (e.g., the solid salt form of Compound I and trometamol) is characterized by a thermal gravimetric analysis (TGA) profile substantially similar to FIG. 4. In some embodiments, the salt form of Compound I and trometamol (e.g., the solid salt form of Compound I and trometamol) is characterized by a TGA profile that is the same or about the same as the profile shown in FIG. 4.

In some embodiments, the salt form of Compound I and trometamol (e.g., the solid salt form of Compound I and trometamol) is characterized by a differential scanning calorimetry (DSC) profile with an endothermic peak at about 120° C. (onset).

In some embodiments, the salt form of Compound I and trometamol (e.g., the solid salt form of Compound I and trometamol) is characterized by a thermal gravimetric analysis (TGA) profile with about 0.7% weight loss at about the endothermic peak of (i).

In some embodiments, the salt form of Compound I and trometamol (e.g., the solid salt form of Compound I and trometamol) is characterized by
(i) a differential scanning calorimetry (DSC) profile with an endothermic peak at about 120° C. (onset); and
(ii) a thermal gravimetric analysis (TGA) profile with about 0.7% weight loss at about the endothermic peak of (i).

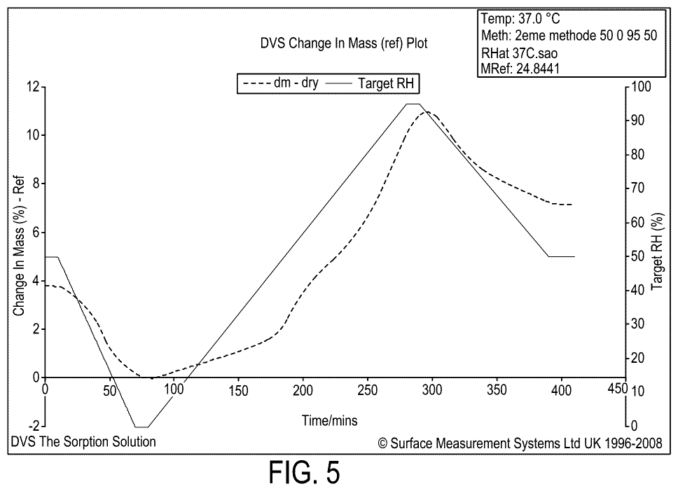

In some embodiments, the salt form of Compound I and trometamol (e.g., the solid salt form of Compound I and trometamol) is characterized by a dynamic vapor sorption profile substantially similar to FIG. 5. In some embodiments, the salt form of Compound I and trometamol (e.g., the solid salt form of Compound I and trometamol) is characterized by a dynamic vapor sorption profile that is the same or about the same as the profile shown in FIG. 5.

In an aspect, provided is a process for preparing a solid form of Compound I.

In some embodiments, a solid form of Compound I or Compound II, described herein (e.g., crystalline Form III of Compound I) is substantially free of methanol, ethanol, and/or dichloromethane, e.g., 1% or less, 0.5% or less, 0.1% or less, 0.05% or less, 0.01% or less, 0.001% or less, 0.0001% or less, 0.00001% or less, 0.000001% or less, or 0.0000001% or less of the solid form is methanol, ethanol, and/or dichloromethane by weight. In some embodiments, the solid form is free of methanol, ethanol, and/or dichloromethane.

In some embodiments, the solid form of Compound I is the crystalline Form I of Compound I form as described herein.

In some embodiments, a solid form of Compound I or Compound II, described herein (e.g., crystalline Form III of Compound, e.g., the anhydrous Form III solid crystalline form) has at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.99%, at least 99.999%, at least 99.9999%, at least 99.99999%, or at least 99.999999% purity.

In some embodiments, a pharmaceutical composition (e.g., a liquid suspension) of the disclosure comprises:
(i) a pharmaceutical agent, wherein the pharmaceutical agent is a crystalline form of Compound I as described herein;
(ii) an emulsifier or surfactant;
(iii) a mucoadhesive or viscosifier; and
(iv) a preservative.

In some embodiments, a pharmaceutical composition (e.g., a liquid suspension) of the disclosure comprises:
(i) a pharmaceutical agent, wherein the pharmaceutical agent is a crystalline form of Compound I as described herein;
(ii) a polysorbate;
(iii) a cellulose; and
(iv) a preservative.

In some embodiments, a pharmaceutical composition (e.g., a liquid suspension) of the disclosure comprises the emulsifier or surfactant at a concentration of from about 5 mg/mL to about 50 mg/mL (e.g., from about 5 mg/mL to about 40 mg/mL, from about 5 mg/mL to about 30 mg/mL, from about 10 mg/mL to about 30 mg/mL, or from about 20 mg/mL to about 30 mg/mL). In some embodiments, a pharmaceutical composition (e.g., a liquid suspension) of the disclosure comprises the emulsifier or surfactant at a concentration of about 25 mg/mL.

In some embodiments, a pharmaceutical composition (e.g., a liquid suspension) of the disclosure comprises the emulsifier or surfactant at a molar concentration of from about 5 mM to about 40 mM, e.g., from about 10 mM to about 30 mM. In some embodiments, a pharmaceutical composition (e.g., a liquid suspension) of the disclosure comprises the emulsifier or surfactant at a molar concentration of about 20 mM.

In some embodiments, a pharmaceutical composition (e.g., a liquid suspension) of the disclosure comprises the mucoadhesive or viscosifier at a concentration of from about 0.1 mg/mL to about 5 mg/mL, e.g., from about 0.1 mg/mL to about 4 mg/mL, from about 0.1 mg/mL to about 3 mg/mL, from about 0.1 mg/mL to about 2 mg/mL, or from about 0.5 mg/mL to about 1.5 mg/mL). In some embodiments, a pharmaceutical composition (e.g., a liquid suspension) of the disclosure comprises the mucoadhesive or viscosifier at a concentration of about 1 mg/mL.

In some embodiments, a pharmaceutical composition (e.g., a liquid suspension) of the disclosure comprises the mucoadhesive or viscosifier at a concentration of from about 5 mg/mL to about 50 mg/mL (e.g., from about 5 mg/mL to about 40 mg/mL, from about 5 mg/mL to about 30 mg/mL, from about 10 mg/mL to about 30 mg/mL, from about 5 mg/mL to about 20 mg/mL, from about 10 mg/mL to about 20 mg/mL, or from about 15 mg/mL to about 20 mg/mL). In some embodiments, a pharmaceutical composition (e.g., a liquid suspension) comprises the mucoadhesive or viscosifier at a concentration of about 18 mg/mL.

In some embodiments, a pharmaceutical composition (e.g., a liquid suspension) of the disclosure, the surfactant is a nonionic surfactant. In some embodiments, a pharmaceutical composition (e.g., a liquid suspension) of the disclosure, the emulsifier or surfactant is a polysorbate. For example, in some embodiments, a pharmaceutical composition (e.g., a liquid suspension) of the disclosure, the emulsifier or surfactant is selected from polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), and polyoxyethylene (20) sorbitan monooleate (polysorbate 80). In some embodiments, a pharmaceutical composition (e.g., a liquid suspension) of the disclosure, the emulsifier or surfactant is polyoxyethylene (20) sorbitan monolaurate (tween 20, polysorbate 20).

In some embodiments of a pharmaceutical composition (e.g., a liquid suspension) of the disclosure, the mucoadhesive or viscosifier is selected from methylcellulose (MC), carboxymethylcellulose (CMC) sodium carboxymethylcellulose (Na-CMC), hydroxypropylmethylcellulose (HPMC), hydroxethylcellulose (HEC), microcrystalline cellulose (MCC), xanthan gum, acacia, tragacanth, alginates, guar gum, colloidal silicon dioxide, and combinations thereof. In some embodiments of a pharmaceutical composition (e.g., a liquid suspension) of the disclosure, the mucoadhesive or viscosifier is selected from methylcellulose (MC), carboxymethylcellulose (CMC) sodium carboxymethylcellulose (Na-CMC), hydroxypropylmethylcellulose (HPMC), hydroxethylcellulose (HEC), microcrystalline cellulose (MCC), and combinations thereof. In some embodiments of a pharmaceutical composition (e.g., a liquid suspension) of the disclosure, the mucoadhesive or viscosifier is selected from microcrystalline cellulose (MCC), sodium carboxymethyl cellulose (Na-CMC) and a combination thereof (MCC-NaCMC). In some embodiments of a pharmaceutical composition (e.g., a liquid suspension) of the disclosure, the mucoadhesive or viscosifier is hydroxypropylmethylcellulose (HPMC). In some embodiments of a pharmaceutical composition (e.g., a liquid suspension) of the disclosure, the mucoadhesive or viscosifier is a combination of microcrystalline cellulose (MCC) and sodium carboxymethyl cellulose (Na-CMC) (MCC-NaCMC).

In some embodiments of a pharmaceutical composition (e.g., a liquid suspension) of the disclosure, a pharmaceutical composition (e.g., a liquid suspension) comprises the preservative at a concentration of up to about 1 mg/mL (e.g., up to about 0.1 mg/mL, up to about 0.2 mg/mL, up to about 0.3 mg/mL, up to about 0.4 mg/mL, up to about 0.5 mg/mL, up to about 0.6 mg/mL, up to about 0.7 mg/mL, up to about 0.8 mg/mL, or up to about 0.9 mg/mL).

In some embodiments of a pharmaceutical composition (e.g., a liquid suspension) of the disclosure, a pharmaceutical composition (e.g., a liquid suspension) comprises the preservative at a concentration of from about 0.01 mg/mL to 1.00 mg/mL (e.g., from about 0.01 mg/mL to about 0.8 mg/mL, from about 0.01 mg/mL to about 0.6 mg/mL, from about 0.01 mg/mL to about 0.4 mg/mL, from about 0.01 mg/mL to about 0.2 mg/mL, from about 0.05 mg/mL to about 0.2 mg/mL, or from about 0.05 mg/mL to about 0.15 mg/mL). In some embodiments of a pharmaceutical composition (e.g., a liquid suspension) of the disclosure, a pharmaceutical composition (e.g., a liquid suspension) comprises the preservative at a concentration of about 0.1 mg/mL.

In some embodiments of a pharmaceutical composition (e.g., a liquid suspension) of the disclosure, a pharmaceutical composition (e.g., a liquid suspension) comprises the preservative at a molar concentration of from about 0.1 mM to about 0.5 mM (e.g., from about 0.2 mM to about 0.4 mM). In some embodiments of a pharmaceutical composition (e.g., a liquid suspension) of the disclosure, a pharmaceutical composition (e.g., a liquid suspension) comprises the preservative at a molar concentration of about 0.3 mM.

In some embodiments of a pharmaceutical composition (e.g., a liquid suspension) of the disclosure, a pharmaceutical composition (e.g., a liquid suspension) comprises the polysorbate at a molar concentration of from about 5 mg/mL to about 50 mg/mL.

In some embodiments of a pharmaceutical composition (e.g., a liquid suspension) of the disclosure, a pharmaceutical composition (e.g., a liquid suspension) comprises the polysorbate at a molar concentration of from about 5 mM to about 40 mM (e.g., from about 10 mM to about 30 mM). In some embodiments of a pharmaceutical composition (e.g., a liquid suspension) of the disclosure, a pharmaceutical composition (e.g., a liquid suspension) comprises the polysorbate at a molar concentration of about 20 mM.

In some embodiments of a pharmaceutical composition (e.g., a liquid suspension) of the disclosure, a pharmaceutical composition (e.g., a liquid suspension) comprises the cellulose at a concentration of from about 0.1 mg/mL to about 5 mg/mL (e.g., from about 5 mg/mL to about 50 mg/mL). In some embodiments of a pharmaceutical composition (e.g., a liquid suspension) of the disclosure, a pharmaceutical composition (e.g., a liquid suspension) comprises cellulose at a concentration of about 18 mg/mL.

In some embodiments, a pharmaceutical composition (e.g., a liquid suspension) of the disclosure comprises the pharmaceutical agent at a concentration of from about 5 mg/mL to about 50 mg/mL (e.g., from about 5 mg/mL to about 40 mg/mL, from about 5 mg/mL to about 30 mg/mL, from about 10 mg/mL to about 30 mg/mL, or from about 15 mg/mL to about 25 mg/mL). In some embodiments, a pharmaceutical composition (e.g., a liquid suspension) of the disclosure comprises the pharmaceutical agent at a concentration of about 20 mg/mL.

In some embodiments, a pharmaceutical composition (e.g., a liquid suspension) of the disclosure comprises the pharmaceutical agent at a molar concentration of from about 10 mM to about 70 mM (e.g., from about 20 mM to about 60 mM, or from about 40 mM to about 50 mM). In some embodiments, a pharmaceutical composition (e.g., a liquid suspension) of the disclosure comprises the pharmaceutical agent at a molar concentration of about 44 mM.

In some embodiments of a pharmaceutical composition (e.g., a liquid suspension) of the disclosure, the preservative is selected from potassium sorbate, chlorobutanol, methyl paraben, propyl paraben, butyl paraben, benzethonium chloride, sodium benzoate, and sorbic acid, and benzalkonium chloride. In some embodiments of a pharmaceutical composition (e.g., a liquid suspension) of the disclosure, the preservative is benzalkonium chloride.

In some embodiments of a pharmaceutical composition (e.g., a liquid suspension) of the disclosure, the cellulose is hydroxypropylmethylcellulose (HPMC) or a combination of microcrystalline cellulose (MCC) and sodium carboxymethyl cellulose (Na-CMC) (MCC-NaCMC).

In some embodiments of a pharmaceutical composition (e.g., a liquid suspension) of the disclosure, the polysorbate is selected from polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), and polyoxyethylene (20) sorbitan monooleate (polysorbate 80). In some embodiments of a pharmaceutical composition (e.g., a liquid suspension) of the disclosure, the polysorbate is polyoxyethylene (20) sorbitan monolaurate (tween 20, polysorbate 20).

In some embodiments of a pharmaceutical composition (e.g., a liquid suspension) of the disclosure, a pharmaceutical composition (e.g., a liquid suspension) comprises the pharmaceutical agent in an amount of about 2 wt. %.

In some embodiments of a pharmaceutical composition (e.g., a liquid suspension) of the disclosure, a pharmaceutical composition (e.g., a liquid suspension) comprises the HPBCD or Trehalose in an amount in an amount of about 68 wt. %.

In some embodiments of a pharmaceutical composition (e.g., a liquid suspension) of the disclosure, a pharmaceutical composition (e.g., a liquid suspension) comprises the HPMC in an amount in an amount of about 30 wt. %.

In some embodiments, a pharmaceutical composition (e.g., a liquid suspension) comprises the pharmaceutical agent and an emulsifier or surfactant in a wt/wt ratio of from about 1:0.6 to about 1:2 (e.g., about 1:0.6, about 1:0.7, about 1:0.8, about 1:0.9, about 1:1.0, about 1:1.1, about 1:1.2, about 1:1.3, about 1:1.4, about 1:1.5, about 1:1.6, about 1:1.7, about 1:1.8, about 1:1.9, or about 1:2.0). In some embodiments, a pharmaceutical composition (e.g., a liquid suspension) comprises the pharmaceutical agent and an emulsifier or surfactant in a wt/wt ratio of about 1:1.3.

In some embodiments, a pharmaceutical composition (e.g., a liquid suspension) comprises the pharmaceutical agent and a preservative in a wt/wt ratio of from about 1:0.001 to about 1:0.01 (e.g., about 1:1:0.001, about 1:0.002, about 1:0.003, about 1:0.004, about 1:0.005, about 1:0.006, about 1:0.007, about 1:0.008, about 1:0.009, or about 1:0.01). In some embodiments, a pharmaceutical composition (e.g., a liquid suspension) comprises the pharmaceutical agent and a preservative in a wt/wt ratio of about 1:0.005.

In some embodiments, a pharmaceutical composition (e.g., a liquid suspension) comprises the pharmaceutical agent, the emulsifier or surfactant and the preservative in a wt/wt ratio of about 1:1.3:0.005.

In some embodiments, a pharmaceutical composition (e.g., a liquid suspension) comprises the pharmaceutical agent and the mucoadhesive or viscosifier in a wt/wt ratio of from about 1:0.01 to about 1:0.1 (e.g., about 1:0.01, about 1:0.02, about 1:0.03, about 1:0.04, about 1:0.05, about 1:0.06, about 1:0.07, about 1:0.08, about 1:0.09, about 1:0.1). In some embodiments, a pharmaceutical composition (e.g., a liquid suspension) comprises the pharmaceutical agent and the mucoadhesive or viscosifier in a wt/wt ratio of about 1:0.05. In some embodiments, a pharmaceutical composition (e.g., a liquid suspension) comprises the pharmaceutical agent and the mucoadhesive or viscosifier in a wt/wt ratio of from about 1:0.3 to about 1:1.5 (e.g., about 1:0.3, about 1:0.4, about 1:0.5, about 1:0.6, about 1:7, about 1:0.8, about 1:0.9, about 1:1, about 1:1.1, about 1:1.2, about 1:1.3, about 1:1.4, or about 1:1.5). In some embodiments, a pharmaceutical composition (e.g., a liquid suspension) comprises the pharmaceutical agent and the mucoadhesive or viscosifier in a wt/wt ratio of about 1:0.9. In some embodiments, a pharmaceutical composition (e.g., a liquid suspension) comprises the pharmaceutical agent, the emulsifier or surfactant and the mucoadhesive or viscosifier in a wt/wt ratio of about 1:1.3:0.05. In some embodiments, a pharmaceutical composition (e.g., a liquid suspension) comprises the pharmaceutical agent, the emulsifier or surfactant and the mucoadhesive or viscosifier in a wt/wt ratio of about 1:1.3:0.9.

In some embodiments, a pharmaceutical composition (e.g., a liquid suspension) comprises the pharmaceutical agent and the polysorbate in a wt/wt ratio of from about 1:0.6 to about 1:2. In some embodiments, a pharmaceutical composition (e.g., a liquid suspension) comprises the pharmaceutical agent and the polysorbate in a wt/wt ratio of about 1:1.3.

In some embodiments, a pharmaceutical composition (e.g., a liquid suspension) comprises the pharmaceutical agent and a preservative in a wt/wt ratio of from about 1:0.001 to about 1:0.01. In some embodiments, a pharmaceutical composition (e.g., a liquid suspension) comprises the pharmaceutical agent and a preservative in a wt/wt ratio of about 1:0.005. In some embodiments, a pharmaceutical composition (e.g., a liquid suspension) comprises the pharmaceutical agent, the polysorbate and the preservative in a wt/wt ratio of about 1:1.3:0.005.

In some embodiments, a pharmaceutical composition (e.g., a liquid suspension) comprises the pharmaceutical agent and the cellulose in a wt/wt ratio of from about 1:0.01 to about 1:0.1. In some embodiments, a pharmaceutical composition (e.g., a liquid suspension) comprises the pharmaceutical agent and the cellulose in a wt/wt ratio of about 1:0.05.

In some embodiments, a pharmaceutical composition (e.g., a liquid suspension) comprises the pharmaceutical agent and the cellulose in a wt/wt ratio of from about 1:0.3 to about 1:1.5 (e.g., about 1:0.3, about 1:0.4, about 1:0.5, about 1:0.6, about 1:7, about 1:0.8, about 1:0.9, about 1:1, about 1:1.1, about 1:1.2, about 1:1.3, about 1:1.4, or about 1:1.5). In some embodiments, a pharmaceutical composition (e.g., a liquid suspension) comprises the pharmaceutical agent and the cellulose in a wt/wt ratio of about 1:0.9.

In some embodiments, a pharmaceutical composition (e.g., a liquid suspension) comprises the pharmaceutical agent, the polysorbate and the cellulose in a wt/wt ratio of about 1:1.3:0.05. In some embodiments, a pharmaceutical composition (e.g., a liquid suspension) comprises the pharmaceutical agent, the polysorbate and the cellulose in a wt/wt ratio of about 1:1.3:0.9.

In some embodiments, the process for preparing the crystalline Form III of Compound I (e.g., a Form III crystalline solid form, e.g., an anhydrous Form III crystalline solid form) comprises a step (a) that comprises:
(a) dissolving the crystalline Form I of Compound I in a volume of methanol to yield a methanol solution.

In some embodiments, the volume of methanol in step (a) is about 20V to about 30V of methanol (e.g., about 25V or about 27V). A unit “V” as used herein refers to a relative volume to the crystalline solid form (1V) in view of the process. For example, “20V” of methanol may refer to 20 mL of methanol per 1 g of crystalline solid.

In some embodiments, the dissolving of step (a) is performed at a temperature that is about 40° C. to about 95° C. (e.g., about 40° C., about 45° C., about 50° C., about 55° C., about 60° C., about 65° C., about 70° C., about 75° C., about 80° C., about 85° C., or about 90° C.). In some embodiments, the temperature is about 45° C., 55° C., or about 90° C.

In some embodiments, the process for preparing the crystalline Form III of Compound I (e.g., a Form III crystalline solid form, e.g., an anhydrous Form III crystalline solid form) comprises, after step (a), a step (b) that comprises:
(b) concentrating the methanol solution of step (a) to a volume that is about 8V to about 15V of methanol to yield a concentrated methanol solution.

In some embodiments, the volume of the methanol solution after concentration in step (b) is about 10V of methanol.

In some embodiments, the process for preparing the crystalline Form III of Compound I (e.g., a Form III crystalline solid form, e.g., an anhydrous Form III crystalline solid form) comprises, after step (b), a step (c) that comprises:
(c) cooling the concentrated methanol solution of step (b) to a temperature that is about 30° C. to about 50° C. to yield a cooled methanol solution.

In some embodiments, in step (c) the methanol solution is cooled to a temperature that is about 40° C.

In some embodiments, the process for preparing the crystalline Form III of Compound I (e.g., a Form III crystalline solid form, e.g., an anhydrous Form III crystalline solid form) comprises, after step (c), a step (d) that comprises:
(d) adding a volume of water to the cooled methanol solution of step (c), wherein the volume of water is from about 3V to about 20V, yielding a mixture.

In some embodiments, the volume of water added in step (d) is about 5V, about 10V, or about 15V.

In some embodiments, the volume ratio of methanol:water after step (d) is about 67:33.

In some embodiments, the process for preparing the crystalline Form III of Compound I (e.g., a Form III crystalline solid form, e.g., an anhydrous Form III crystalline solid form) comprises, after step (d), a step (e) that comprises:
(e) cooling the mixture of step (d) to a temperature that is about 5° C. to about 15° C., wherein the cooling results in precipitation of a solid form of Compound I.

In some embodiments, in step (e) the mixture is cooled to a temperature that is about 10° C.

In some embodiments, in step (e) the mixture is cooled for a period of time that is about 1 to about 15, about 12 to about 24, or about 24 to about 72 hours. In some embodiments, the mixture is cooled for a period of time that is about 1 hour, about 2 hours, about 3 hours, about 4 hours, about 5 hours, about 6 hours, about 7 hours, about 8 hours, about 9 hours, or about 10 hours. In some embodiments, the mixture is cooled for a period of time that is about 3 hours or about 10 hours.

In some embodiments, the process for preparing the crystalline Form III of Compound I (e.g., a Form III crystalline solid form, e.g., an anhydrous Form III crystalline solid form) comprises, after step (e), a step (f) that comprises:
(f) collecting the precipitated solid form of Compound I.

In some embodiments, the collecting of step (f) comprises using vacuum filtration.

In some embodiments, in step (f) the precipitated solid form of Compound I is collected using vacuum filtration.

In some embodiments, the process for preparing the crystalline Form III of Compound I (e.g., a Form III crystalline solid form, e.g., an anhydrous Form III crystalline solid form) comprises, after step (f), a step (g) that comprises:
(g) drying of the precipitated solid form of step (f) to obtain the crystalline Form III of Compound I.

In some embodiments, the drying of step (g) occurs at a temperature that is about 25° C. to about 60° C. In some embodiments, the temperature is about 25° C., 30° C., 35° C., 40° C., 45° C., 50° C., 55° C., or 60° C.

In some embodiments, the process for preparing the crystalline Form III of Compound I (e.g., a Form III crystalline solid form, e.g., an anhydrous Form III crystalline solid form) comprises a step (a′) that comprises:
(a′) dissolving the crystalline Form I of Compound I of Compound I in a volume of methanol to yield a methanol solution of the crystalline Form I of Compound I of Compound I and filtering the methanol solution to yield a filtered methanol solution.

In some embodiments, the volume of methanol in step (a′) is from about 20V to about 30V of methanol (e.g., about 25V or about 27V). A unit “V” as used herein refers to a relative volume to the crystalline solid form (1V) in view of the process. For example, “20V” of methanol may refer to 20 mL of methanol per 1 g of crystalline solid.

In some embodiments, the dissolving of step (a′) is performed at a temperature that is about 40° C. to about 95° C. (e.g., about 40° C., about 45° C., about 50° C., about 55° C., about 60° C., about 65° C., about 70° C., about 75° C., about 80° C., about 85° C., or about 90° C.). In some embodiments, the temperature is about 45° C., 55° C., or about 90° C.

In some embodiments, the process for preparing the crystalline Form III of Compound I (e.g., a Form III crystalline solid form, e.g., an anhydrous Form III crystalline solid form) comprises after step (a′), a step (b′) that comprises:
(b′) cooling the filtered methanol solution of step (a′) to a temperature that is about 30° C. to about 50° C. to yield a cooled methanol solution.

In some embodiments, in step (b′), the cooling of the methanol solution of step (a′) is to a temperature that is about 40° C.

In some embodiments, the process for preparing the crystalline Form III of Compound I (e.g., a Form III crystalline solid form, e.g., an anhydrous Form III crystalline solid form) comprises, after step (b′), a step (c′) that comprises:
(c′) adding a volume of water to the methanol solution of step (b′), wherein the volume of water is from about 3V to about 20V, yielding a mixture.

In some embodiments, the volume of water added in step (c′) is about 5V, about 10V, or about 15V.

In some embodiments, the volume ratio of methanol:water after step (c′) is about 67:33.

In some embodiments, the process for preparing the crystalline Form III of Compound I (e.g., a Form III crystalline solid form, e.g., an anhydrous Form III crystalline solid form) comprises, after step (c′), a step (d′) that comprises:
(d′) cooling the mixture of step (c′) to a temperature that is about 5° C. to about 15° C., wherein the cooling results in precipitation of a solid form of Compound I.

In some embodiments, in step (d′) the mixture of step (c′) is cooled to a temperature that is about 10° C.

In some embodiments, in step (d′) the mixture of step (c′) is cooled for a period of time that is from about 1 to about 15, from about 12 to about 24, or from about 24 to about 72 hours. In some embodiments, the mixture is cooled for a period of time that is from about 8 to about 16 hours or from about 8 to about 12 hours.

In some embodiments, the process for preparing the crystalline Form III of Compound I (e.g., a Form III crystalline solid form, e.g., an anhydrous Form III crystalline solid form) comprises, after step (d′), a step (e′) that comprises: (e′) collecting the precipitated solid form of Compound I.

In some embodiments, the process for preparing the crystalline Form III of Compound I (e.g., a Form III crystalline solid form, e.g., an anhydrous Form III crystalline solid form) comprises, after step (e′), a step (f′) that comprises:
(f) drying of the precipitated solid form of step (e′) and obtaining the Form (III) crystalline of Compound I (e.g., a Form III crystalline solid form, e.g., an anhydrous Form III crystalline solid form).

In some embodiments, the process for preparing the crystalline Form III of Compound I (e.g., a Form III crystalline solid form, e.g., an anhydrous Form III crystalline solid form) comprises:
Step (a) dissolving the crystalline Form I of Compound I in a volume of methanol that is about 25V to about 30V of methanol to yield a methanol solution;
Step (b) concentrating the methanol solution of step (a) to a volume that is about 10V of methanol to yield a concentrated methanol solution, wherein step (b) is after step (a);
Step (c) cooling the concentrated methanol solution of step (b) to a temperature that is about 30° C. to about 50° C. to yield a cooled methanol solution, wherein step (c) is after step (b);
Step (d) adding a volume of water to the cooled methanol solution of step (c), wherein the volume of water is about 3V to about 20V, yielding a mixture, wherein step (d) is after step (c);
Step (e) cooling the mixture of step (d) to a temperature that is about 5° C. to about 15° C., wherein the cooling results in precipitation of a solid form of Compound I, wherein step (e) is after step (d);
Step (f) collecting the precipitated solid form of Compound I, wherein step (f) is after step (e); and
Step (g) drying of the precipitated solid form of step (f) to obtain the Form (III) crystalline form of Compound I, wherein step (g) is after step (f).

In some embodiments, the process for preparing the crystalline Form III of Compound I (e.g., a Form III crystalline solid form, e.g., an anhydrous Form III crystalline solid form) comprises:
Step (a′) dissolving the crystalline Form I of Compound I in a volume of methanol that is about 25V to about 30V of methanol, and
   filtering of the solution of Compound I in methanol to yield a methanol solution;
Step (b′) cooling the methanol solution of step (a′) to a temperature that is about 40° C. to yield a cooled methanol solution, wherein step (b′) is after step (a′);
Step (c′) adding a volume of water to the methanol solution of step (b), wherein the volume of water is about 15V, yielding a mixture, wherein step (c′) is after step (b′);
Step (d′) cooling the mixture of step (c′) to a temperature that is about 10° C., wherein the cooling results in precipitation of a solid form of Compound I, wherein step (d′) is after step (c′);
Step (e′) collecting the precipitated solid form of Compound I, wherein step (e′) is after step (d′); and
Step (f′) drying of the precipitated solid form of step (e′) and obtaining the Form (III) crystalline solid form of Compound I, wherein step (f) is after step (e′).

In some embodiments, the process for preparing a crystalline Form III of Compound I (e.g., a Form III crystalline solid form, e.g., an anhydrous Form III crystalline solid form) comprises a first crystallization purification to yield a first purified crystalline form of Compound I; and (ii) a crystal transformation to arrive at the Form III of Compound I. In some embodiments, the crystalline Form III of Compound I made by the process described herein is a highly pure crystalline form of Compound I.

In some embodiments, the process for preparing a crystalline Form III of Compound I (e.g., a Form III crystalline solid form, e.g., an anhydrous Form III crystalline solid form) comprises (i) a first crystallization purification to yield a first purified crystalline form of Compound I, (ii) a second crystallization purification to yield a second purified crystalline form of Compound I, and (iii) a crystal transformation to arrive at the crystalline Form III. In some embodiments, the crystalline Form III of Compound I made by the process described herein is a highly pure crystalline form of Compound I.

In some embodiments, the first crystallization purification of the process for preparing a crystalline Form III of Compound I (e.g., a Form III crystalline solid form, e.g., an anhydrous Form III crystalline solid form) comprises:

Step 1′: combining a crude crystalline form of Compound I (e.g., a crude crystalline Form I of Compound I or a crude mixture of a crystalline Form I and a crystalline Form III of Compound I) and an organic solvent to form a first mixture;

Step 2′: adding a seed crystal to the first mixture; wherein step 2′ is after step 1′;

Step 3′: adding an anti-solvent to the first mixture (i.e., the seeded mixture; of step 2′) to form a second mixture; wherein step 3′ is after step 2′; and

Step 4′: isolating a first purified crystalline form of Compound I from the second mixture; wherein step 4′ is after step 3′.

In some embodiments, the first crystallization purification of the process for preparing a crystalline Form III of Compound I (e.g., a Form III crystalline solid form, e.g., an anhydrous Form III crystalline solid form) comprises:

Step 1: combining a crude crystalline form of Compound I (e.g., a crude crystalline Form I of Compound I or a crude mixture of a crystalline Form I and a crystalline Form III of Compound I) and an organic solvent to form a first mixture;

Step 2: heating the first mixture to a temperature T¹ to form a first solution; wherein step 2 is after step 1;

Step 3: cooling the first solution to a temperature T²; wherein step 3 is after step 2;

Step 4: adding a seed crystal to the cooled solution of step 3; wherein step 4 is after step 3;

Step 5: adding an anti-solvent to the seeded solution of step 4 to form a second mixture; wherein step 5 is after step 4; and

Step 6: isolating the first purified crystalline form of Compound I from the second mixture; wherein step 6 is after step 5.

In some embodiments, the organic solvent of step 1′ or 1 is selected from methyl ethyl ketone (MEK), methyl isobutyl ketone (MIBK), tetrahydrofuran (THF), 2-methyltetrahydrofuran (2-MeTHF), 1,4-dioxane, dimethylsulfoxide (DMSO), dichloromethane (DCM), anisole, dimethylformamide (DMF), dimethylacetamide (DMAc), and N-methylpyrrolidone (NMP). In some embodiments, the organic solvent of step 1′ or 1 is selected from tetrahydrofuran (THF), 2-methyltetrahydrofuran (2-MeTHF), 1,4-dioxane, dimethylsulfoxide (DMSO), dichloromethane (DCM), anisole, dimethylformamide (DMF), dimethylacetamide (DMAc), and N-methylpyrrolidone (NMP). In some embodiments, the organic solvent of step 1′ or 1 is tetrahydrofuran (THF). In some embodiments, the organic solvent of step 1′ or 1 is 2-methyltetrahydrofuran (2-MeTHF).

In some embodiments, the process further comprises after step 1′ or step 1: step 1-1: adding activated carbon to the first mixture. In some embodiments, the process further comprises after step 1-1: step 1-2: adding an additional amount of the organic solvent.

In some embodiments, the amount of the organic solvent in step 1′ or 1 is from about 5V to about 20 V. In some embodiments, the amount of the organic solvent in step 1′ or 1 is from about 5V to about 10 V. In some embodiments, the amount of the organic solvent in step 1′ or 1 is about 8 V. In some embodiments, the amount of the organic solvent in step 1′ or 1 is from about 10 V to about 20 V. In some embodiments, the amount of the organic solvent in step 1′ or 1 is about 15 V.

In some embodiments, the additional amount of the organic solvent in step 1-2 is from about 5V to about 20 V. In some embodiments, the additional amount of the organic solvent in step 1-2 is from about 5V to about 10 V. In some embodiments, the additional amount of the organic solvent in step 1-2 is about 8 V. In some embodiments, the additional amount of the organic solvent in step 1-2 is from about 10V to about 20 V. In some embodiments, the additional amount of the organic solvent in step 1-2 is about 15 V.

In some embodiments, step 2 further includes adding activated carbon to the first solution.

In some embodiments, the activated carbon in step 1 or 2 is added in an amount of from about 0.01 wt % to about 1 wt %, from about 0.01 wt % to about 0.5 wt %, from about 0.05 wt % to about 0.5 wt %, or from about 0.1 wt % to about 0.4 wt %. In some embodiments, the activated carbon in step 1 or 2 is added in an amount about 0.2 wt %. In some embodiments, the activated carbon is F1600 activated carbon.

In some embodiments, T¹ is from about 40° C. to about 110° C., from about 50° C. to about 100° C., or from about 60° C. to about 90° C., In some embodiments, T¹ is from about 70° C. to about 80° C. (e.g., about 70° C., about 71° C., about 72° C., about 73° C., about 74° C., about 75° C., about 76° C., about 77° C., about 78° C., about 79° C., or about 80° C.). In some embodiments, T¹ is from about 70° C. to about 75° C.

In some embodiments, the process further comprises after step 2: step 2-1: stirring the first solution for a duration t²⁻¹.

In some embodiments, t²⁻¹ is from about 12 h to about 24 h (e.g., about 12 h, about 13 h, about 14 h, about 15 h, about 16 h, about 17 h, about 18 h, about 19 h, about 20 h, about 21 h, about 22 h, about 23 h, or about 24 h). In some embodiments, t²⁻¹ is from about 16 h to about 20 h. In some embodiments, t²⁻¹ is from about 1 h to about 8 h (e.g., about 1 h, about 2 h, about 3 h, about 4 h, about 5 h, about 6 h, about 7 h, or about 8 h). In some embodiments, t²⁻¹ is from about 1 h to about 4 h. In some embodiments, t²⁻¹ is from about 2 h to about 3 h. In some embodiments, t²⁻¹ is from about 2 h to about 8 h. In some embodiments, t²⁻¹ is from about 4 h to about 6 h.

In some embodiments, t²⁻¹ is one hour or less. In some embodiments, t²⁻¹ is from about 10 min to about 40 min. In some embodiments, t²⁻¹ is from about 20 min to about 30 min.

In some embodiments, the process further comprises after step 2 or step 2-1: step 2-2: polish filtering the first solution. In some embodiments, step 2-2 further comprises rinsing the filter with the organic solvent of step 1, and re-heating the filtrate to the temperature T¹.

In some embodiments, in step 2-2, the amount of the organic solvent used to rinse the filter is from about 0.5 V to about 5 V, from about 1 V to about 4 V, or from about 1 V to about 3 V. In some embodiments, in step 2-2, the amount of the organic solvent used to rinse the filter is about 2 V.

In some embodiments, T² is from about 30° C. to about 90° C., from about 40° C. to about 80° C., or from about 50° C. to about 70° C., from about 50° C. to about 60° C., or from about 55° C. to about 60° C. In some embodiments, T² is about 55° C. In some embodiments, T² is about 60° C.

In some embodiments, the seed crystal in step 2′ or 4 is a crystalline Form I of Compound I. In some embodiments, the seed crystal in step 2′ or 4 is a crystalline Form III of Compound I. In some embodiments, the seed crystal in step 2′ or 4 is a mixture of crystalline Form I of Compound I and crystalline Form III of Compound I. In some embodiments, the seed crystal is of the same crystalline form as the first purified crystalline form of Compound I.

In some embodiments, the seed crystal in step 2′ or 4 is added in an amount of from about 0.1 wt % to about 3 wt %, from about 0.1 wt % to about 2 wt %, from about 0.5 wt % to about 3 wt % or from about 0.5 wt % to about 2 wt %. In some embodiments, the seed crystal in step 2′ or 4 is added in an amount of about 1 wt %.

In some embodiments, the process further comprises after step 4: step 4-1: aging the seeded mixture for a duration t⁴⁻¹. In some embodiments, t⁴⁻¹ is 3 h or less. In some embodiments, t⁴⁻¹ is from about 10 min to about 3 h. In some embodiments, t⁴⁻¹ is from about 20 min to about 40 min. In some embodiments, t⁴⁻¹ is about 30 min. In some embodiments, t⁴⁻¹ is from about 1 h to about 3 h. In some embodiments, t⁴⁻¹ is about 2 h.

In some embodiments, the process further comprises after step 4 or step 4-1: step 4-2: cooling the seeded mixture to a temperature T⁴⁻². In some embodiments, step 4-2 further comprises stirring the seeded mixture at temperature T⁴⁻² for 5-10 h.

In some embodiments, T⁴⁻² is from about 0° C. to about 50° C. In some embodiments, T⁴⁻² is from about 0° C. to about 40° C., from about 10° C. to about 40° C., from about 0° C. to about 30° C., from about 10° C. to about 30° C., or from about 20° C. to about 30° C. In some embodiments, T⁴⁻² is about 25° C.

In some embodiments, in step 4-2, the seeded mixture is cooled at a cooling rate of from about 5° C./h to about 20° C./h, or from about 5° C./h to about 15° C./h. In some embodiments, in step 4-2, the seeded mixture is cooled at a cooling rate of 10° C./h.

In some embodiments, the anti-solvent in step 3′ or 5 is added over a period of from about 2 h to about 10 h, from about 4 h to about 8 h, from about 4 h to about 6 h, or from about 5 h to about 7 h. In some embodiments, the anti-solvent in step 3′ or 5 is added over a period of about 6 h.

In some embodiments, the anti-solvent in step 3′ or 5 is selected from, isopropanol (IPA), n-propanol (n-PrOH), 2-butanol, acetone, ethyl acetate (EtOAc), isopropyl acetate (IPAc), methyl tert-butyl ether (MTBE), H₂O, acetonitrile (MeCN), heptane (e.g., n-heptane), and acetic acid (HOAc). In some embodiments, the anti-solvent in step 3′ or 5 is selected from ethyl acetate (EtOAc), acetonitrile (MeCN), and heptane (e.g., n-heptane). In some embodiments, the anti-solvent in step 3′ or 5 is heptane. In some embodiments, the anti-solvent in step 3′ or 5 is n-heptane.

In some embodiments, the amount of anti-solvent in step 3′ or 5 is from about 5 V to about 50 V, from about 10 V to about 50 V, from about 20 V to about 50 V, from about 20 V to about 40 V, or from about 30 V to about 40 V. In some embodiments, the amount of anti-solvent in step 3′ or 5 is from about 30 V to about 35 V. In some embodiments, the amount of anti-solvent in step 3′ or 5 is about 34 V.

In some embodiments, the process further comprises after step 5, step 5-1: cooling the second mixture to a temperature T⁵⁻¹.

In some embodiments, T⁵⁻¹ is from about −10° C. to about 15° C., from about −5° C. to about 15° C., from about −5° C. to about 10° C., or from about 0° C. to about 10° C. In some embodiments, T⁵⁻¹ is about 5° C.

In some embodiments, in step 5-1, the second mixture is cooled at a cooling rate of from about 5° C./h to about 20° C./h, or from about 5° C./h to about 15° C./h. In some embodiments, in step 5-1, the second mixture is cooled at a cooling rate of 10° C./h.

In some embodiments, the process further comprises after step 5-1: step 5-2: stirring the cooled mixture of step 5-1 for a duration t⁵⁻¹ at the temperature T⁵⁻¹. In some embodiments, t⁵⁻¹ is from about 5 h to about 20 h, from about 5 h to about 14 h, from about 8 h to about 14 h, from about 8 h to about 13 h, from about 10 h to about 13 h, or from about 11 h to about 12 h. In some embodiments, t⁵⁻¹ is at least 8 h. In some embodiments, t⁵⁻¹ is from about 8 h to about 16 h, from about 8 h to about 14 h, from about 8 h to about 13 h, or from about 8 h to about 12 h.

In some embodiments, step 4′ or 6 (i.e., isolating the first purified crystalline form) comprises filtering the second mixture. In some embodiments, the filtering further comprises rinsing the solid residue (i.e., the first purified crystalline form) with the anti solvent of step 3′ or 5, and/or drying the solid residue (i.e., the first purified crystalline form) under vacuum. In some embodiments, step 4′ or 6 comprises drying the solid residue (i.e., the first purified crystalline form) under vacuum at a temperature of from about 30° C. to about 40° C.

In some embodiments, in step 4′ or 6, the amount of the anti-solvent used to rinse the filter is from about 0.5 V to about 5 V, from about 1V to about 4 V, or from about 1V to about 3 V. In some embodiments, in step 4′ or 6, the amount of the anti-solvent used to rinse the filter is about 2V.

In some embodiments, the first purified crystalline form of Compound I is Form I of Compound I. In some embodiments, the first purified crystalline form of Compound I is Form III of Compound I. In some embodiments, the first purified crystalline form of Compound I is a mixture of crystalline Form I of Compound I and crystalline Form III of Compound I. In some embodiments, the first purified crystalline form of Compound I is Form II of Compound I. In some embodiments, the first purified crystalline form of Compound I is Form IV of Compound I. In some embodiments, the first purified crystalline form of Compound I is Form V of Compound I.

In some embodiments, the second crystallization purification of the process for preparing a crystalline Form III of Compound I (e.g., a Form III crystalline solid form, e.g., an anhydrous Form III crystalline solid form) comprises:

Step 5′: combining the first purified crystalline form of Compound I (i.e., the purified crystalline form of Compound I obtained in steps 1′-4′ of the first crystallization purification) and an organic solvent to form a third mixture;

Step 6′: adding a seed crystal to the third mixture; wherein step 6′ is after step 5′ Step 7′: adding an anti-solvent to the third mixture (i.e., the seeded solution of step 6′) to form a fourth mixture; wherein step 7′ is after step 8′; and

Step 8′: isolating a second purified crystalline form of Compound I from the fourth mixture; wherein step 8′ is after step 7′.

In some embodiments, the second crystallization purification of the process for preparing a crystalline Form III of Compound I (e.g., a Form III crystalline solid form, e.g., an anhydrous Form III crystalline solid form) comprises:

Step 7: combining the first purified crystalline form of Compound I (i.e., the purified crystalline form of Compound I obtained in steps 1-6 of the first crystallization purification) and an organic solvent to form a third mixture;

Step 8: heating the third mixture to a temperature T³ to form a third solution; wherein step 8 is after step 7;

Step 9: cooling the third solution to a temperature T⁴; wherein step 9 is after step 8;

Step 10: adding a seed crystal to the cooled solution of step 9; wherein step 10 is after step 9;

Step 11: adding an anti-solvent to the seeded solution of step 10 to form a fourth mixture; wherein step 11 is after step 10; and

Step 12: isolating the second purified crystalline form of Compound I from the fourth mixture; wherein step 12 is after step 11.

In some embodiments, the organic solvent of step 5′ or 7 is selected from methyl ethyl ketone (MEK), methyl isobutyl ketone (MIBK), tetrahydrofuran (THF), 2-methyltetrahydrofuran (2-MeTHF), 1,4-dioxane, dimethylsulfoxide (DMSO), dichloromethane (DCM), anisole, dimethylformamide (DMF), dimethylacetamide (DMAc), and N-methylpyrrolidone (NMP). In some embodiments, the organic solvent of step 5′ or 7 is selected from tetrahydrofuran (THF), 2-methyltetrahydrofuran (2-MeTHF), 1,4-dioxane, dimethylsulfoxide (DMSO), dichloromethane (DCM), anisole, dimethylformamide (DMF), dimethylacetamide (DMAc), and N-methylpyrrolidone (NMP). In some embodiments, the organic solvent of step 5′ or 7 is tetrahydrofuran (THF). In some embodiments, the organic solvent of step 5′ or 7 is 2-methyltetrahydrofuran (2-MeTHF).

In some embodiments, the process further comprises after step 7: step 7-1: adding activated carbon to the third mixture. In some embodiments, the process further comprises after step 7-1: step 7-2: adding an additional amount of the organic solvent.

In some embodiments, the amount of the organic solvent in step 5′ or 7 is from about 5 V to about 20 V. In some embodiments, the amount of the organic solvent in step 5′ or 7 is from about 5 V to about 10 V. In some embodiments, the amount of the organic solvent in step 7 is about 8 V. In some embodiments, the amount of the organic solvent in step 5′ or 7 is from about 10 V to about 20 V. In some embodiments, the amount of the organic solvent in step 75′ or is about 15 V.

In some embodiments, the additional amount of the organic solvent in step 7-2 is from about 5 V to about 20 V. In some embodiments, the additional amount of the organic solvent in step 7-2 is from about 5 V to about 10 V. In some embodiments, the additional amount of the organic solvent in step 7-2 is about 8 V. In some embodiments, the additional amount of the organic solvent in step 7-2 is from about 10 V to about 20 V. In some embodiments, the additional amount of the organic solvent in step 7-2 is about 15 V.

In some embodiments, step 8 further includes adding activated carbon to the third solution.

In some embodiments, the activated carbon in step 7 or 8 is added in an amount of from about 0.01 wt % to about 1 wt %, from about 0.05 wt % to about 1 wt %, or from about 0.2 wt % to about 0.8 wt %. In some embodiments, the activated carbon in step 7 or 8 is added in an amount about 0.5 wt %. In some embodiments, the activated carbon is F1600 activated carbon.

In some embodiments, T³ is from about 40° C. to about 110° C., from about 50° C. to about 100° C., or from about 60° C. to about 90° C. In some embodiments, T³ is from about 70° C. to about 80° C. (e.g., about 70° C., about 71° C., about 72° C., about 73° C., about 74° C., about 75° C., about 76° C., about 77° C., about 78° C., about 79° C., or about 80° C.). In some embodiments, T³ is from about 70° C. to about 75° C.

In some embodiments, the process further comprises after step 8: step 8-1: stirring the third solution for a duration t⁸⁻¹.

In some embodiments, t⁸⁻¹ is from about 1 h to about 4 h. In some embodiments, t⁸⁻¹ is from about 2 h to about 3 h. In some embodiments, t²⁻¹ is from about 2 h to about 8 h. In some embodiments, t⁻¹ is from about 4 h to about 6 h.

In some embodiments, t²⁻¹ is one hour or less. In some embodiments, t²⁻¹ is from about 10 min to about 40 min. In some embodiments, t²⁻¹ is from about 20 min to about 30 min.

In some embodiments, the process further comprises after step 8 or step 8-1: step 8-2: polish filtering the third solution. In some embodiments, step 8-2 further comprises rinsing the filter with the organic solvent of step 7, and re-heating the filtrate to the temperature T³.

In some embodiments, in step 8-2, the amount of the organic solvent used to rinse the filter is from about 0.5 V to about 5 V, from about 1 V to about 4 V, or from about 1 V to about 3 V. In some embodiments, in step 8-2, the amount of the organic solvent used to rinse the filter is about 2 V.

In some embodiments, T⁴ is from about 30° C. to about 90° C., from about 40° C. to about 80° C., or from about 50° C. to about 70° C., from about 50° C. to about 60° C., from about 55° C. to about 60° C. In some embodiments, T⁴ is about 55° C. In some embodiments, T⁴ is about 60° C.

In some embodiments, the seed crystal in step 6′ or 10 is a crystalline Form I of Compound I. In some embodiments, the seed crystal in step 6′ or 10 is a crystalline Form III of Compound I. In some embodiments, the seed crystal in step 6′ or 10 is a mixture of crystalline Form I of Compound I and crystalline Form III of Compound I. In some embodiments, the seed crystal is of the same crystalline form as the second purified crystalline form of Compound I.

In some embodiments, the seed crystal in step 6′ or 10 is added in an amount of from about 0.1 wt % to about 3 wt %, from about 0.1 wt % to about 2 wt %, from about 0.5 wt % to about 3 wt % or from about 0.5 wt % to about 2 wt %. In some embodiments, the seed crystal in step 6′ or 10 is added in an amount of about 1 wt %.

In some embodiments, the process further comprises after step 10: step 10-1: aging the seeded mixture for a duration t¹⁰⁻¹. In some embodiments, t¹⁰⁻¹ is 3 h or less. In some embodiments, t¹⁰⁻¹ is from about 10 min to about 3 h. In some embodiments, t¹⁰⁻¹ is from about 20 min to about 40 min. In some embodiments, t¹⁰⁻¹ is from about 30 min to about 40 min. In some embodiments, t¹⁰⁻¹ is about 30 min. In some embodiments, t¹⁰⁻¹ is from about 1 h to about 3 h. In some embodiments, t¹⁰⁻¹ is about 2 h.

In some embodiments, the process further comprises after step 10 or step 10-1: step 10-2: cooling the seeded mixture to a temperature T¹⁰⁻². In some embodiments, step 10-2 further comprises stirring the seeded mixture at temperature T¹⁰⁻² for 5-10 h.

In some embodiments, T¹⁰⁻² is from about 0° C. to about 50° C. In some embodiments, T¹⁰⁻² is from about 0° C. to about 40° C., from about 10° C. to about 40° C., from about 0° C. to about 30° C., from about 10° C. to about 30° C., or from about 20° C. to about 30° C. In some embodiments, T¹⁰⁻² is about 25° C.

In some embodiments, in step 10-2, the seeded mixture is cooled at a cooling rate of from about 5° C./h to about 20° C./h, or from about 5° C./h to about 15° C./h. In some embodiments, in step 10-2, the seeded mixture is cooled at a cooling rate of 10° C./h.

In some embodiments, the anti-solvent in step 7′ or 11 is added over a period of from about 2 h to about 10 h, from about 4 h to about 8 h, from about 4 h to about 6 h, or from about 5 h to about 7 h. In some embodiments, the anti-solvent in step 7′ or 11 is added over a period of about 6 h.

In some embodiments, the anti-solvent in step 7′ or 11 is selected from, isopropanol (IPA), n-propanol (n-PrOH), 2-butanol, acetone, ethyl acetate (EtOAc), isopropyl acetate (IPAc), methyl tert-butyl ether (MTBE), H₂O, acetonitrile (MeCN), heptane (e.g., n-heptane), and acetic acid (HOAc). In some embodiments, the anti-solvent in step 7′ or 11 is selected from ethyl acetate (EtOAc), acetonitrile (MeCN), and heptane (e.g., n-heptane). In some embodiments, the anti-solvent in step 7′ or 11 is heptane. In some embodiments, the anti-solvent in step 7′ or 11 is n-heptane.

In some embodiments, the amount of anti-solvent in step 7′ or 11 is from about 5 V to about 50 V, from about 10 V to about 50 V, from about 20 V to about 50 V, from about 20 V to about 40 V, or from about 30 V to about 40 V. In some embodiments, the amount of anti-solvent in step 7′ or 11 is from about 30 V to about 35 V. In some embodiments, the amount of anti-solvent in step 7′ or 11 is about 34 V. In some embodiments, the amount of anti-solvent in step 7′ or 11 is about 30 V.

In some embodiments, the anti-solvent in step 7′ or 11 is added over a period of from about 2 h to about 10 h, from about 4 h to about 8 h, from about 4 h to about 6 h, or from about 5 h to about 7 h. In some embodiments, the anti-solvent in step 7′ or 11 is added over a period of about 6 h.

In some embodiments, the process further comprises after step 11, step 11-1: cooling the fourth mixture to a temperature T¹¹⁻¹.

In some embodiments, T¹¹⁻¹ is from about −10° C. to about 15° C., from about −5° C. to about 15° C., from about −5° C. to about 10° C., or from about 0° C. to about 10° C. In some embodiments, T¹¹⁻¹ is about 5° C.

In some embodiments, in step 11-1, the fourth mixture is cooled at a cooling rate of from about 5° C./h to about 20° C./h, or from about 5° C./h to about 15° C./h. In some embodiments, in step 11-1, the fourth mixture is cooled at a cooling rate of 10° C./h.

In some embodiments, the process further comprises after step 11-1: step 11-2: stirring the cooled mixture of step 11-1 for a duration t¹¹⁻¹ at the temperature T¹¹⁻¹. In some embodiments, t¹¹⁻¹ is from about 5 h to about 20 h, from about 5 h to about 14 h, from about 8 h to about 14 h, from about 8 h to about 13 h, from about 10 h to about 13 h, or from about 11 h to about 12 h. In some embodiments, t¹¹⁻¹ is at least 8 h. In some embodiments, t¹¹⁻¹ is from about 8 h to about 16 h, from about 8 h to about 14 h, from about 8 h to about 13 h, or from about 8 h to about 12 h.

In some embodiments, step 8′ or 12 (i.e., isolating the first purified crystalline form) comprises filtering the fourth mixture. In some embodiments, the filtering further comprises rinsing the solid residue (i.e., the second purified crystalline form) with the anti solvent of step 7′ or 11, and/or drying the solid residue (i.e., the second purified crystalline form) under vacuum. In some embodiments, step 8′ or 12 comprises drying the solid residue (i.e., the first purified crystalline form) under vacuum at a temperature of from about 30° C. to about 40° C.

In some embodiments, in step 8′ or 12, the amount of the anti-solvent used to rinse the filter is from about 0.5 V to about 5 V, from about 1 V to about 4 V, or from about 1 V to about 3 V. In some embodiments, in step 8′ or 12, the amount of the anti-solvent used to rinse the filter is about 2 V.

In some embodiments, the second purified crystalline form of Compound I is Form I of Compound I. In some embodiments, the second purified crystalline form of Compound I is Form III of Compound I. In some embodiments, the second purified crystalline form of Compound I is a mixture of crystalline Form I of Compound I and crystalline Form III of Compound I. In some embodiments, the second purified crystalline form of Compound I is Form II of Compound I. In some embodiments, the second purified crystalline form of Compound I is Form IV of Compound I. In some embodiments, the second purified crystalline form of Compound I is Form V of Compound I.

In some embodiments, the crystal transformation to arrive at the crystalline Form III of Compound I (e.g., a Form III crystalline solid form, e.g., an anhydrous Form III crystalline solid form) comprises:Step ct-1: combining the first or second purified crystalline form of Compound I (i.e., the purified crystalline form of Compound I obtained in the first or second crystallization purification process) and methanol to form a mixture A;

Step ct-2: adding a seed crystal to mixture A to form a mixture B; wherein step ct-2 is after step ct-1;

Step ct-3: adding an anti-solvent to mixture B to form mixture C; wherein step ct-3 is after step ct-2; and

Step ct-4: isolating the crystalline Form III of Compound I from mixture C; wherein step ct-4 is after step ct-3.

In some embodiments, the crystal transformation to arrive at the crystalline Form III of Compound I (e.g., a Form III crystalline solid form, e.g., an anhydrous Form III crystalline solid form) comprises: Step ct-1: combining the first or second purified crystalline form of Compound I (i.e., the purified crystalline form of Compound I obtained in the first or second crystallization purification process) and methanol to form a mixture A;

Step ct-1a: adding a first portion of an anti-solvent to mixture A to form mixture A′; wherein step ct-1a is after step ct-1;

Step ct-2′: adding a seed crystal to mixture A′ to form a mixture B; wherein step ct-2 is after step ct-1a′;

Step ct-3: adding an anti-solvent to mixture B to form mixture C; wherein step ct-3 is after step ct-2′; and

Step ct-4: isolating the crystalline Form III of Compound I from mixture C; wherein step ct-4 is after step ct-3.

In some embodiments, the crystal transformation to arrive at the crystalline Form III of Compound I (e.g., a Form III crystalline solid form, e.g., an anhydrous Form III crystalline solid form) comprises:

Step 13: combining the second purified crystalline form of Compound I (i.e., the purified crystalline form of Compound I obtained in the second crystallization purification process, e.g., steps 7-12) and methanol to form a fifth mixture;

Step 14: heating the fifth mixture to a temperature T⁵ to form a third solution; wherein step 14 is after step 13;

Step 15: cooling the third solution to a temperature T⁶; wherein step 15 is after step 14;

Step 16: adding a first portion of an anti-solvent to the third solution to form a sixth mixture; wherein step 16 is after step 15;

Step 17: adding a seed crystal to the sixth mixture; wherein step 17 is after step 16;

Step 18: adding a second portion of an anti-solvent to the seeded mixture of step 17 to form a seventh mixture; wherein step 18 is after step 17;

Step 19: cooling the seventh mixture to a temperature T⁷; wherein step 19 is after step 18; and

Step 20: isolating the crystalline Form III of Compound I from the seventh mixture; wherein step 20 is after step 19.

In some embodiments, the amount of the methanol of step 13 is from about 10 V to about 40 V, from about 15 V to about 35 V, or from about 20 V to about 35 V. In some embodiments, the amount of the methanol of step 13 is about 28 V.

In some embodiments, T⁵ is from about 30° C. to about 90° C., from about 40° C. to about 80° C., or from about 50° C. to about 70° C., from about 40° C. to about 60° C., from about 50° C. to about 60° C., or from about 55° C. to about 60° C. In some embodiments, T⁵ is about 55° C.

In some embodiments, the process further comprises after step 14: step 14-1: filtering the third solution. In some embodiments, step 14-1 further comprises rinsing the filter with the methanol of step 13.

In some embodiments, in step 14-1, the amount of the methanol used to rinse the filter is from about 0.5 V to about 5 V, from about 1 V to about 4 V, or from about 1 V to about 3 V. In some embodiments, in step 14-1, the amount of the methanol used to rinse the filter is about 2 V.

In some embodiments, T⁶ is from about 10° C. to about 60° C., from about 20° C. to about 50° C., from about 30° C. to about 50° C., from about 40° C. to about 45° C., or from about 35° C. to about 45° C. In some embodiments, T⁶ is about 40° C.

In some embodiments, the anti-solvent in step ct-1a or 16 is H₂O. In some embodiments, in step ct-1a or 16, the first portion of the anti-solvent is an amount of from about 1 V to about 20 V, from about 1 V to about 15 V, from about 1 V to about 10 V, or from about 2 V to about 8 V. In some embodiments, in step ct-1a or 16, the first portion of the anti-solvent is an amount of about 5 V. In some embodiments, the anti-solvent in step ct-1a or 16 is added at the temperature T⁶.

In some embodiments, the seed crystal in step ct-2, ct-2′, or 17 is a crystalline Form III of Compound I.

In some embodiments, the seed crystal in step ct-2, ct-2′, or 17 is added in an amount of from about 0.1 wt % to about 3 wt %, from about 0.1 wt % to about 2 wt %, from about 0.5 wt % to about 3 wt %, or from about 0.5 wt % to about 2 wt %. In some embodiments, the seed crystal in step ct-2, ct-2′, or 17 is added in an amount of about 1 wt %.

In some embodiments, the process further comprises after step 17: step 17-1: aging the seeded mixture for a duration t¹⁷⁻¹. In some embodiments, t¹⁷⁻¹ is 3 h or less. In some embodiments, t¹⁷⁻¹ is from about 10 min to about 3 h. In some embodiments, t¹⁷⁻¹ is from about 20 min to about 40 min, or from about 30 min to about 40 min. In some embodiments, t¹⁷⁻¹ is about 30 min.

In some embodiments, the anti-solvent in step ct-3 or 18 is H₂O. In some embodiments, in step ct-1 or 18, the second portion of the anti-solvent is an amount of from about 1 V to about 30 V, from about 5 V to about 25 V, from about 5 V to about 20 V, or from about 5 V to about 15 V. In some embodiments, in step ct-3 or 18, the second portion of the anti-solvent is an amount of about 10 V. In some embodiments, the anti-solvent in step ct-3 or 18 is added at the temperature T⁵.

In some embodiments, the process further comprises after step 18: step 18-1: stirring the third solution for a duration t¹⁸⁻¹.

In some embodiments, t¹⁸⁻¹ is one hour or less. In some embodiments, t¹⁸⁻¹ is from about 10 min to about 40 min. In some embodiments, t¹⁸⁻¹ is from about 20 min to about 30 min.

In some embodiments, T⁷ is from about −5° C. to about 30° C., from about 0° C. to about 20° C., from about 0° C. to about 15° C., or from about 5° C. to about 15° C. In some embodiments, T⁷ is about 10° C.

In some embodiments, in step 19, the seventh mixture is cooled at a cooling rate of from about 10° C./h to about 30° C./h, or from about 15° C./h to about 25° C./h. In some embodiments, in step 19, the seventh mixture is cooled at a cooling rate of 20° C./h.

In some embodiments, the process further comprises after step 19: step 19-1: stirring the third solution for a duration t¹⁹⁻¹.

In some embodiments, t¹⁹⁻¹ is from about 5 h to about 20 h, from about 5 h to about 14 h, from about 8 h to about 14 h, from about 8 h to about 13 h, from about 10 h to about 13 h, from about 11 h to about 12 h, or from about 14 h to about 16 h. In some embodiments, t¹⁹⁻¹ is at least 8 h. In some embodiments, t¹⁹⁻¹ is from about 8 h to about 16 h, from about 8 h to about 14 h, or from about 8 h to about 12 h.

In some embodiments, the anti-solvent in step ct-1a, ct-3,16 or 18 is added dropwise.

In some embodiments, step 20 (i.e., isolating the crystalline Form III of Compound I) comprises filtering the seventh mixture. In some embodiments, step ct-4 (i.e., isolating the crystalline Form III of Compound I) comprises filtering mixture C. In some embodiments, the filtering further comprises rinsing the solid residue (i.e., the crystalline Form III of Compound I) with a mixture of methanol and H₂O, and/or drying the solid residue (i.e., the crystalline Form III of Compound I) under vacuum. In some embodiments, step ct-4 or 20 comprises drying the solid residue (i.e., the crystalline Form III of Compound I) under vacuum at a temperature of from about 30° C. to about 40° C.

In some embodiments, in step ct-4 or 20, the amount of the mixture of methanol and H₂O used to rinse the filter is from about 0.5 V to about 5 V, from about 1 V to about 4 V, or from about 1 V to about 3 V. In some embodiments, in step ct-4 or 20, the amount of the mixture of methanol and H₂O used to rinse the filter is about 2 V. In some embodiments, the step ct-4 or 20, the mixture of methanol and H₂O comprises methanol and H₂O in a ratio of from about 0.5:1 to about 4:1 or from about 1:1 to about 3:1. In some embodiments, the step ct-4 or 20, the mixture of methanol and H₂O is a 2:1 mixture of methanol and H₂O.

In some embodiments, the crude crystalline form of Compound is obtained by a process comprising extracting celastrol from thunder god vine. In some embodiments, extracting celastrol from thunder god vine comprises extracting celastrol from the crude plant extract using ethanol.

Without wishing to be bound by theory and as demonstrated in Example 5, in some embodiments, Compound 1 Form I was found to present interesting characteristics for development (e.g., highly crystalline, non hygroscopic, thermally stable). However, in some embodiments, inclusion of ethanol led to high levels of residual ethanol. In addition, process development work performed on Form I indicated that removal of ethanol from Form I was not possible. Further, as demonstrated in Example 8, both Form I and Form II contained residual solvent in amounts exceeding the ICH limit. See e.g., Table 19 in Example 8. Without wishing to be bound by theory, crude Compound I (celastrol) is generally obtained via extraction from thunder god vine with ethanol. Further, without wishing to be bound by theory, the extraction from thunder god vine with ethanol was found to result in Form I of Compound I (see Example 14). Similarly, as demonstrated in Example 7 (Table 16) Form III of Compound I in ethanol transitions towards Form I of Compound I. Thus, without wishing to be bound by theory, extraction of Compound I from thunder good vine is expected to result in at least some amounts of crystalline Form I of Compound I, i.e., a form of Compound I comprising high levels of residual ethanol.

Without wishing to be bound by theory, and as demonstrated in Example 7, Compound 1 Form III was obtained at high chemical purity and yield (99.9% (HPLC) and more than 90% respectively), as well as low levels of residual ethanol. In some embodiments, as demonstrated in Example 10, Compound 1 Form III may exhibit degradation during the concentration step in some processes for making Compound 1 Form III (e.g., when Form III is made by dissolving a crude Form I of Compound I in methanol and crystallizing from methanol). In some embodiments, retreating the batch by slurry with 15 volumes of MeOH:Water at a ratio of 67:33 (V:V) led to a batch of Compound 1 Form III being obtained with a purity (HPLC) of 99.7% and a yield of 92%.

In some embodiments, a chromatographic purification step was introduced into the process for making Compound 1 Form III prior to the final crystallization step to improve robustness of the process by preventing degradation in solution. In addition, in some examples slight adjustments were made to ensure that this change did not impact the final quality of Compound 1 Form III.

Without wishing to be bound by theory, and as demonstrated in Example 13, Compound 1 Form III was obtained at high chemical purity (>99.9% (HPLC).

In another aspect, a pharmaceutical composition is provided including any of the solid forms of Compound I, including embodiments thereof, and a pharmaceutically acceptable excipient.

In another aspect, a method of treating obesity in a subject in need thereof is provided. The method includes administering to the subject an effective amount of the any of the solid forms of Compound I, including any embodiments thereof.

In one aspect, the invention provides a composition, wherein the composition is used to treat an obesity-related disease or disorder or a psychiatric or mental health disorder, e.g., such as a psychiatric/mental health disorder characterized by compulsive behaviors such as compulsive eating. The obesity-related disease or disorder is selected from a group comprising obesity, pre-obesity, morbid obesity, Prader-Willi Syndrome, Hypothalamic Injury Associated Obesity, non-alcoholic steatohepatitis, hyperlipidemia, hypertension, diabetes, lipodystrophy, fatty liver, Bardet-Biedl Syndrome, Cohen Syndrome, cardiovascular disease, arthritis, stroke, gallstones, gallbladder disease, gout, sleep apnea, asthma, metabolic syndrome and MOMO (Macrosomia Obesity Macrocephaly Ocular abnormalities) Syndrome.

In another aspect, the invention provides a method of treating an obesity-related disease or disorder comprising administering to a subject suffering from or at risk of suffering from an obesity-related disease or disorder one or more compositions of Formula (I), which is disclosed in U.S. Patent Application Publication No. 2015-0250753, published Sep. 10, 2015, which is incorporated herein by reference in its entirety. The obesity-related disease or disorder is selected from the group comprising obesity, pre-obesity, morbid obesity, Prader-Willi Syndrome, Hypothalamic Injury Associated Obesity, Non-alcoholic steatohepatitis, hyperlipidemia, hypertension, diabetes, lipodystrophy, fatty liver, Bardet-Biedl Syndrome, Cohen Syndrome, cardiovascular disease, arthritis, stroke, gallstones, gallbladder disease, gout, sleep apnea, asthma, metabolic syndrome and MOMO Syndrome.

In another aspect of the invention, the composition of any of the solid forms of Compound I, including embodiments thereof, is administered in combination with another therapy.

In other aspects of the invention, administering further comprises oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

In another aspect, the composition of any of the solid forms of Compound I, including embodiments thereof, is administered in a form selected from the group comprising pills, capsules, tablets, granules, powders, salts, crystals, liquid, serums, syrups, suspensions, gels, creams, pastes, films, patches, and vapors.

In another aspect of the invention, the subject is a mammal. Furthermore, the subject is a human. In still another aspect, the subject is a human with a body mass index (BMI) greater than 30 kg/m².

In another aspect, a method of treating a malignancy in a subject in need thereof is provided. The method includes administering to the subject an effective amount of a crystalline form of Compound I of the disclosure.

In one aspect, the invention provides a composition, wherein the composition is used to treat a malignancy-related disease or disorder. The malignancy-related disease or disorder is selected from the group comprising gastric cancer, multiple myeloma, melanoma, leukemia, lymphoma, renal cell carcinoma, hepatocellular carcinoma, breast cancer, prostate cancer, head and neck cancer, non-small cell lung carcinoma, brain cancer, and glioblastoma multiforme (GBM).

In another aspect, the invention provides a method of treating a malignancy-related disease or disorder comprising administering to a subject suffering from or at risk of suffering from a malignancy-related disease or disorder one or more compositions of Formula (I). The malignancy-related disease or disorder is selected from the group comprising gastric cancer, multiple myeloma, melanoma, leukemia, lymphoma, renal cell carcinoma, hepatocellular carcinoma, breast cancer, prostate cancer, head and neck cancer, non-small cell lung carcinoma, brain cancer, and glioblastoma multiforme (GBM).

### III. Methods

The compounds (e.g., solid forms and salts) described herein can be used in treating a disease or condition in a patient in need thereof as described in U.S. Patent Application Publication No. 2015-0250753, published Sep. 10, 2015, which is incorporated herein by reference in its entirety.

The compounds described above, can be used in the treatment of obesity in a subject in need thereof includes administering to the subject an effective amount of the compounds of Formula (I). Obesity may be the primary cause of a disease and/or disorder or may be caused as a result of a disease and/or disorder.

In some cases, an effective amount of a crystalline form of Compound I of the disclosure may be administered as a method of treating weight gain in pre-obese, obese or morbidly obese patients.

In some cases, a method of reducing body fat in pre-obese, obese or morbidly obese patients includes administering an effective amount of a crystalline form of Compound I of the disclosure. Body mass or body fat may be creased by 5, 10, 15, 20, 25, 30, 35, 40, 45, or 50%.

In some cases, a method of reducing food intake in pre-obese, obese or morbidly obese patients is accomplished by administering an effective amount of a crystalline form of Compound I of the disclosure. The average daily food intake (in terms of calories) may be reduced by at least 5, 10, 15, 20, 25, 30, 35, 40, 45, 50% or higher.

In some cases, an effective amount of a crystalline form of Compound I of the disclosure may be administered to reduce the body mass index (BMI) of a patient suffering from obesity. The BMI of a patient may be reduced to a value of <30 kg m⁻² (normal BMI=20-25 kg m²).

Lastly, a method of improving glucose homeostatis in pre-obese, obese, or morbidly obese patients may be accomplished by administering a crystalline form of Compound I of the disclosure. The average fasting plasma blood glucose levels may be reduced by at least 10, 12, 15, 18, 20, 25, 30, 35, 40% or higher.

### Hypothalamic Injury Associated Obesity

Hypothalamic obesity is a complicated medical condition that can occur from the growth of rare brain tumors and from other types of injury to the hypothalamus. Craniopharyngioma is one of the tumors that can cause hypothalamic injury associated obesity. Damage to the hypothalamus disrupts the communication between the gut and the brain, causing a constant feeling of hunger.

The hypothalamus and pituitary gland are tightly integrated. Damage to the hypothalamus will impact the responsiveness and normal functioning of the pituitary. Hypothalamic disease may cause insufficient or inhibited signaling to the pituitary leading to deficiencies of one or more of the following hormones: thyroid-stimulating hormone, adrenocorticotropic hormone, beta-endorphin, luteinizing hormone, follicle-stimulating hormone, and melanocyte-stimulating hormones. Treatment for hypopituitarism involves hormone replacement therapy (Pinkney, Pituitary News 17, 2000).

Thyroid hormones are responsible for metabolic activity. Insufficient production of thyroid hormones result in suppressed metabolic activity and weight gain. Hypothalamic disease may therefore have implications for obesity (Pinkney, Pituitary News 17, 2000); (Ling, Trends in Obesity Research, 2004).

### Obesity

Obesity is a medical condition in which excess body fat has accumulated to the extent that it may have a negative effect on health, leading to reduced life expectancy and/or increased health problems (Haslam et al., Lancet (Review) 366 (9492): 1197-209, 2005). In Western countries, people are considered obese when their body mass index (BMI), a measurement obtained by dividing a person's weight by the square of the person's height, exceeds 30 kg/m², with the range 25-30 kg/m² defined as overweight. Obesity increases the likelihood of various diseases, particularly heart disease, type 2 diabetes, obstructive sleep apnea, certain types of cancer, and osteoarthritis (Haslam et al., Lancet (Review) 366 (9492): 1197-209, 2005). Obesity is most commonly caused by a combination of excessive food energy intake, lack of physical activity, and genetic susceptibility, although a few cases are caused primarily by genes, endocrine disorders, medications, or psychiatric illness. Evidence to support the view that some obese people eat little yet gain weight due to a slow metabolism is limited. On average, obese people have a greater energy expenditure than their thin counterparts due to the energy required to maintain an increased body mass (Kushner, Treatment of the Obese Patient, 2007).

Obesity is a medical condition in which excess body fat has accumulated to the extent that it may have an adverse effect on health. It is defined by body mass index (BMI) and further evaluated in terms of fat distribution via the waist-hip ratio and total cardiovascular risk factors (Sweeting et al., Nutr. J. 6 (1): 32, 2007). BMI is closely related to both percentage body fat and total body fat (Gray et al., J. Clin. Epidemiol. 44 (6): 545-50, 1991).

BMI is defined as the subject's weight divided by the square of their height. BMI is usually expressed in kilograms per square meter, resulting when weight is measured in kilograms and height in meters. Some modifications to the definitions have been made where the surgical literature breaks down obesity into further categories whose exact values are still disputed (Sturm et al., Public Health 121 (7): 492-6, 2007). Any BMI≥35 or 40 kg/m² is severe obesity. A BMI of ≥35 kg/m² and experiencing obesity-related health conditions or ≥40-44.9 kg/m² is morbid obesity. A BMI of ≥45 or 50 kg/m² is super obesity. The World Health Organization (WHO) regards a BMI of less than 18.5 as underweight and may indicate malnutrition, an eating disorder, or other health problems, while a BMI equal to or greater than 25 is considered overweight and above 30 is considered obese (World Health Organization, Global Database on Body Mass Index (2006)). A summary of the WHO BMI classification scheme is outlined in the Table A below.

TABLE A
Body Mass Index Classification Scheme

| CATEGORY | BMI range-kg/m² |
|---|---|
| Very severely underweight | less than 15 |
| Severely underweight | from 15.0 to 16.0 |
| Underweight | from 16.0 to 18.5 |
| Normal (healthy weight) | from 18.5 to 25 |
| Overweight | from 25 to 30 |
| Obese Class I (Moderately | from 30 to 35 |
| obese) | |
| Obese Class II (Severely | from 35 to 40 |
| obese) | |
| Obese Class III (Very severely | over 40 |
| obese) | |

### Fatty Liver/NASH

Non-alcoholic fatty liver disease (NAFLD) is one of the causes of fatty liver, occurring when fat is deposited (steatosis) in the liver due to causes other than excessive alcohol use. NAFLD is related to insulin resistance and the metabolic syndrome and may respond to treatments originally developed for other insulin-resistant states (e.g. diabetes mellitus type 2) such as weight loss, metformin and thiazolidinediones (Adams et al. Postgrad. Med. J. 82 (967): 315-22, 2006). Non-alcoholic steatohepatitis (NASH) is the most extreme form of NAFLD, and is regarded as a major cause of cirrhosis of the liver of unknown cause (Clark et al. JAMA 289 (22):3000-4, 2003).

Most people with NAFLD have few or no symptoms. Patients may complain of fatigue, malaise, and dull right-upper-quadrant abdominal discomfort. Mild jaundice may be noticed although this is rare. More commonly NAFLD is diagnosed following abnormal liver function tests during routine blood tests. NAFLD is associated with insulin resistance and metabolic syndrome (obesity, combined hyperlipidemia, diabetes mellitus (type II) and high blood pressure) (Adams et al. Postgrad Med J. 82 (967): 315-22, 2006).

Common findings are elevated liver enzymes and a liver ultrasound showing steatosis. An ultrasound may also be used to exclude gallstone problems (cholelithiasis). A liver biopsy (tissue examination) is the only test widely accepted as definitively distinguishing NASH from other forms of liver disease and can be used to assess the severity of the inflammation and resultant fibrosis (Adams et al. Postgrad Med J. 82 (967): 315-22, 2006).

Other diagnostic tests are available. Relevant blood tests include erythrocyte sedimentation rate, glucose, albumin, and renal function. Because the liver is important for making proteins used in coagulation some coagulation related studies are often carried out especially the INR (international normalized ratio). Blood tests (serology) are usually used to rule out viral hepatitis (hepatitis A, B, C and herpes viruses like EBV or CMV), rubella, and autoimmune related diseases. Hypothyroidism is more prevalent in NASH patients which would be detected by determining the TSH (Liangpunsakul et al. J. Clin. Gastroenterol. 37(4):340-3, 2003).

### Metabolic Syndrome

Metabolic syndrome is a disorder of energy utilization and storage, diagnosed by a co-occurrence of three out of five of the following medical conditions: abdominal (central) obesity, elevated blood pressure, elevated fasting plasma glucose, high serum triglycerides, and low high-density lipoprotein (HDL) levels. Metabolic syndrome increases the risk of developing cardiovascular disease and diabetes (Kaur, Cardiology Research and Practice, 2014) (Felizola, “Ursolic acid in experimental models and human subjects: potential as an anti-obesity/overweight treatment?” ResearchGate, 2015).

The main sign of metabolic syndrome is central obesity (also known as visceral, male-pattern or apple-shaped adiposity), overweight with adipose tissue accumulation particularly around the waist and trunk. Other signs of metabolic syndrome include high blood pressure, decreased fasting serum HDL cholesterol, elevated fasting serum triglyceride level (VLDL triglyceride), impaired fasting glucose, insulin resistance, or prediabetes. Associated conditions include hyperuricemia, fatty liver (especially in concurrent obesity) progressing to nonalcoholic fatty liver disease, polycystic ovarian syndrome (in women), erectile dysfunction (in men), and Acanthosis nigricans.

A joint interim statement of the International Diabetes Federation Task Force on Epidemiology and Prevention; National Heart, Lung, and Blood Institute; American Heart Association; World Heart Federation; International Atherosclerosis Society; and International Association for the Study of Obesity published a guideline to harmonize the definition of the metabolic syndrome (Alberti et al., Circulation 120 (16): 1640-5, 2009). This definition recognizes that the risk associated with a particular waist measurement will differ in different populations.

### Stroke

Stroke, also known as cerebrovascular accident (CVA), cerebrovascular insult (CVI), or brain attack, is when poor blood flow to the brain results in cell death. There are two main types of stroke: ischemic due to lack of blood flow and hemorrhagic due to bleeding. They result in part of the brain not functioning properly. Signs and symptoms of a stroke may include an inability to move or feel on one side of the body, problems understanding or speaking, feeling like the world is spinning, or loss of vision to one side among others (Donnan et al., Lancet 371 (9624): 1612-23, 2008). Signs and symptoms often appear soon after the stroke has occurred. If symptoms last less than one or two hours it is known as a transient ischemic attack (TIA). Hemorrhagic strokes may also be associated with a severe headache.

The main risk factor for stroke is high blood pressure. Other risk factors include tobacco smoking, obesity, high blood cholesterol, diabetes mellitus, previous TIA, and atrial fibrillation among others (Donnan et al., Lancet 371 (9624): 1612-23, 2008). An ischemic stroke is typically caused by blockage of a blood vessel. A hemorrhagic stroke is caused by bleeding either directly into the brain or into the space surrounding the brain. (Feigin et al., Stroke 36 (12): 2773-80, 2005). Bleeding may occur due to a brain aneurysm. Diagnosis is typically with medical imaging such as a CT scan or MRI scan along with a physical exam. Other tests such as an electrocardiogram (ECG) and blood tests are done to determine risk factors and rule out other possible causes.

Stroke is diagnosed through several techniques: a neurological examination (such as the NIHSS), CT scans (most often without contrast enhancements) or MRI scans, Doppler ultrasound, and arteriography. The diagnosis of stroke itself is clinical, with assistance from the imaging techniques. Imaging techniques also assist in determining the subtypes and cause of stroke. There is yet no commonly used blood test for the stroke diagnosis itself, though blood tests may be of help in finding out the likely cause of stroke (Hill et al., Clin. Chem. 51 (11): 2001-2, 2005).

### Cardiovascular Disease

Cardiovascular disease (CVD) is a class of diseases that involve the heart or blood vessels. Cardiovascular disease includes coronary artery diseases (CAD) such as angina and myocardial infarction (commonly known as a heart attack) (Shanthi et al., Global Atlas on Cardiovascular Disease Prevention and Control 3-18, 2011). Other CVDs are stroke, hypertensive heart disease, rheumatic heart disease, cardiomyopathy, atrial fibrillation, congenital heart disease, endocarditis, aortic aneurysms, and peripheral artery disease

The underlying mechanisms vary depending on the disease in question. Coronary artery disease, stroke, and peripheral artery disease involve atherosclerosis. This may be caused by high blood pressure, smoking, diabetes, lack of exercise, obesity, high blood cholesterol, poor diet, and excessive alcohol consumption, among others. High blood pressure results in 13% of CVD deaths, while tobacco results in 9%, diabetes 6%, lack of exercise 6% and obesity 5%. Rheumatic heart disease may follow untreated strep throat (Shanthi et al., Global Atlas on Cardiovascular Disease Prevention and Control 3-18, 2011).

Standard tests for cardiovascular disease include: coronary artery calcification, carotid total plaque area, elevated low-density lipoprotein-p, and elevated blood levels of brain natriuretic peptide (also known as B-type) (BNP) (Bertazzo et al., Nat. Mat. 12, 576-583, 2013) (Inaba et al., Atherosclerosis 220 (1): 128-33, 2012) (J. Clin. Lipidol. Dec; 1(6) 583-92, 2007) (Wang et al., N. Engl. J. Med. 350(7): 655-63, 2004).

### Diabetes

Diabetes mellitus (DM), commonly referred to as diabetes, is a group of metabolic diseases in which there are high blood sugar levels over a prolonged period. Symptoms of high blood sugar include frequent urination, increased thirst, and increased hunger. If left untreated, diabetes can cause many complications (Diabetes Fact sheet N^{o} 312″. WHO, 2013). Acute complications include diabetic ketoacidosis and nonketotic hyperosmolar coma (Kitabchi, et al., Diabetes Care 32 (7): 1335-43, 2009). Serious long-term complications include cardiovascular disease, stroke, chronic kidney failure, foot ulcers, and damage to the eyes.

Diabetes is due to either the pancreas not producing enough insulin or the cells of the body not responding properly to the insulin produced (Shoback, Greenspan's Basic & Clinical Endocrinology (9th ed.) (2011)).

Diabetes mellitus is characterized by recurrent or persistent high blood sugar, and is diagnosed by demonstrating any one of the following: Fasting plasma glucose level ≥7.0 mmol/l (126 mg/dl); Plasma glucose ≥11.1 mmol/l (200 mg/dl) two hours after a 75 g oral glucose load as in a glucose tolerance test; Symptoms of high blood sugar and casual plasma glucose ≥11.1 mmol/l (200 mg/dl); and Glycated hemoglobin (HbA1C) ≥48 mmol/mol (≥6.5 DCCT %) (National Diabetes Clearinghouse (NDIC): National Diabetes Statistics 2011” U.S. Department of Health and Human Services, 2011) (“Diabetes Care” American Diabetes Association, 2010).

A positive result, in the absence of unequivocal high blood sugar, should be confirmed by a repeat of any of the above methods on a different day. It is preferable to measure a fasting glucose level because of the ease of measurement and the considerable time commitment of formal glucose tolerance testing, which takes two hours to complete and offers no prognostic advantage over the fasting test (Saydah et al., Diabetes Care 24 (8): 1397-402, 2001). According to the current definition, two fasting glucose measurements above 126 mg/dl (7.0 mmol/l) is considered diagnostic for diabetes mellitus.

Per the World Health Organization, people with fasting glucose levels from 6.1 to 6.9 mmol/l (110 to 125 mg/dl) are considered to have impaired fasting glucose; people with plasma glucose at or above 7.8 mmol/l (140 mg/dl), but not over 11.1 mmol/l (200 mg/dl), two hours after a 75 g oral glucose load are considered to have impaired glucose tolerance (Definition and diagnosis of diabetes mellitus and intermediate hyperglycemia: report of a WHO/IDF consultation. World Health Organization p. 21, 2006). Of these two prediabetic states, the latter in particular is a major risk factor for progression to full-blown diabetes mellitus, as well as cardiovascular disease. The American Diabetes Association since 2003 uses a slightly different range for impaired fasting glucose of 5.6 to 6.9 mmol/l (100 to 125 mg/dl) (Bartoli et al., Eur. J. Int. Med. 22 (1): 8-12, 2011). Glycated hemoglobin is better than fasting glucose for determining risks of cardiovascular disease and death from any cause (Selvin et al., N. Engl. J. Med. 362 (9): 800-11, 2010).

The rare disease diabetes insipidus has similar symptoms to diabetes mellitus, but without disturbances in the sugar metabolism (insipidus means “without taste” in Latin) and does not involve the same disease mechanisms. Diabetes is a part of the wider condition known as metabolic syndrome.

### Hypertension

Hypertension is diagnosed on the basis of a persistently high blood pressure. Traditionally, the National Institute of Clinical Excellence recommends three separate sphygmomanometer measurements at one monthly intervals. The American Heart Association recommends at least three measurements on at least two separate health care visits (Aronow et al., J. Am. Soc. Hypertension: JASH 5 (4): 259-352, 2011). An exception to this is those with very high blood pressure readings especially when there is poor organ function. Initial assessment of the hypertensive people should include a complete history and physical examination. With the availability of 24-hour ambulatory blood pressure monitors and home blood pressure machines, the importance of not wrongly diagnosing those who have white coat hypertension has led to a change in protocols. In the United Kingdom, current best practice is to follow up a single raised clinic reading with ambulatory measurement, or less ideally with home blood pressure monitoring over the course of 7 days. Pseudohypertension in the elderly or non-compressibility artery syndrome may also require consideration. This condition is believed to be due to calcification of the arteries resulting in abnormally high blood pressure readings with a blood pressure cuff while intra-arterial measurements of blood pressure are normal (Franklin et al., Hypertension 59 (2): 173-8, 2012). Orthostatic hypertension is when blood pressure increases upon standing.

### Hyperlipidemia

Hyperlipidemia involves abnormally elevated levels of any or all lipids and/or lipoproteins in the blood (Dorland's Medical Dictionary for Health Consumers, 2007). It is the most common form of dyslipidemia (which includes any abnormal lipid levels). Lipids (fat-soluble molecules) are transported in a protein capsule. The size of that capsule, or lipoprotein, determines its density. The lipoprotein density and type of apolipoproteins it contains determines the fate of the particle and its influence on metabolism.

Hyperlipidemias are divided into primary and secondary subtypes. Primary hyperlipidemia is usually due to genetic causes (such as a mutation in a receptor protein), while secondary hyperlipidemia arises due to other underlying causes such as diabetes. Lipid and lipoprotein abnormalities are common in the general population, and are regarded as a modifiable risk factor for cardiovascular disease due to their influence on atherosclerosis. In addition, some forms may predispose to acute pancreatitis.

Hyperlipidemia is a group of disorders characterized by an excess of serum cholesterol, especially excess LDL-C and/or excess triglycerides. Hypercholesterolemia is generally asymptomatic. Hypertriglyceridemia is generally asymptomatic until triglyceride levels are sustained above 1000 mg/dL—symptoms then include dermatologic manifestations, such as eruptive xanthomas, and gastrointestinal manifestations, such as pancreatitis. Hyperlipidemias are most often genetically determined, but can be caused or amplified by abnormal diet, drugs, and certain disease conditions. Drugs associated with hyperlipidemias include: immunosuppressive therapy, thiazide diuretics, progestins, retinoids, anabolic steroids, glucocorticoids, HIV protease inhibitors, alcohol, retinoic acid, and beta-blockers. Diseases associated with secondary hyperlipidemias include: diabetes mellitus (type I and type II), hypothyroidism, Cushing's syndrome, chronic kidney disease, nephrotic syndrome, and cholestatic disorders. Hyperlipidemia is a major modifiable risk factor for atherosclerosis and cardiovascular disease, including coronary heart disease (Dorland's Medical Dictionary for Health Consumers, 2007).

### Prader-Willi Syndrome

Prader-Willi Syndrome affects approximately 1 in 10,000 to 1 in 25,000 newborns (Killeen, Principles of Molecular Pathology 2004). There are more than 400,000 people who live with Prader-Willi Syndrome around the world (Tweed, AOL Health, September 2009). It is traditionally characterized by hypotonia, short stature, hyperphagia, obesity, behavioral issues (specifically OCD-like behaviors), small hands and feet, hypogonadism, and mild intellectual disability (Killeen, Principles of Molecular Pathology 2004). Like autism, Prader-Willi Syndrome is a spectrum disorder and symptoms can range from mild to severe and may change throughout the person's lifetime.

Traditionally, Prader-Willi syndrome was diagnosed by clinical presentation. Currently, the syndrome is diagnosed through genetic testing; testing is recommended for newborns with pronounced hypotonia. Early diagnosis of Prader-Willi Syndrome allows for early intervention. The mainstay of diagnosis is genetic testing, specifically DNA-based methylation testing to detect the absence of the paternally contributed Prader-Willi syndrome/Angelman syndrome (PWS/AS) region on chromosome 15q11-q13. Such testing detects over 97% of cases. Methylation-specific testing is important to confirm the diagnosis of PWS in all individuals, but especially those who are too young to manifest sufficient features to make the diagnosis on clinical grounds or in those individuals who have atypical findings (Buiting et al., Nat. Genet. 9(4):395-400, 1995).

### Bardet-Biedl Syndrome

The Bardet-Biedl syndrome (BBS) is a ciliopathic human genetic disorder that produces many effects and affects many body systems. It is characterized principally by obesity, retinitis pigmentosa, polydactyly, hypogonadism, and renal failure in some cases (Beales et al., J. Med. Genet. 36(6):437-46, 1999).

Bardet-Biedl syndrome is a pleiotropic disorder with variable expressivity and a wide range of clinical variability observed both within and between families. The main clinical features are rod-cone dystrophy, with childhood-onset visual loss preceded by night blindness; postaxial polydactyly; truncal obesity that manifests during infancy and remains problematic throughout adulthood; specific learning difficulties in some but not all individuals; male hypogenitalism and complex female genitourinary malformations; and renal dysfunction, a major cause of morbidity and mortality. There is a wide range of secondary features that are sometimes associated with BBS including: speech disorder/delay, strabismus/cataracts/astigmatism, brachydactyly/syndactyly of both the hands and feet, partial syndactyl (most usually between the second and third toes), developmental delay, polyuria/polydipsia (nephrogenic diabetes insipidus), ataxia/poor coordination/imbalance, mild hypertonia (especially lower limbs), diabetes mellitus, dental crowding/hypodontia/small dental roots; high-arched palate, cardiovascular anomalies, hepatic involvement, anosmia, auditory deficiencies, and Hirschsprung disease (Ross et al. The Clinical, Molecular, and Functional Genetics of Bardet-Biedl Syndrome, in Genetics of Obesity Syndromes, 2008).

### Cohen Syndrome

This syndrome is believed to be a gene mutation in chromosome 8 at locus 8q22 gene COH1 (Kolehmainen et al, Am. J. Hum. Genet. 72(6):1359-69, 2003). Cohen syndrome has several characteristics such as obesity, mental retardation and craniofacial dysmorphism. It has an autosomal recessive transmission with variable expression (Kivitie-Kallio et al. Am. J. Med. Genet. 102(2):125-35, 2001).

Cohen syndrome is diagnosed by clinical examination, but often difficult due to variation in expression. Ocular complications, though rare, are listed as optic atrophy, microphthalmia, pigmentary chorioretinitis, hemeralopia (decreased vision in bright light), myopia, strabismus, nystagmus and iris/retinal coloboma. General appearance is obesity with thin/elongated arms and legs. Micrognathia, short philtrum, and high vaulted palate are common. Variable mental retardation with occasional seizure and deafness also is characteristic of Cohen syndrome.

### MOMO Syndrome

MOMO syndrome is an extremely rare genetic disorder which belongs to the overgrowth syndromes and has been diagnosed in only six cases around the world, and occurs in 1 in 100 million births. The name is an acronym of the four primary aspects of the disorder: Macrosomia (excessive birth weight), Obesity, Macrocephaly (excessive head size) and Ocular abnormalities (Moretti-Ferreira et al. Am. J. Med. Genet. 46(5):555-8, 1993). There are also other common symptoms: a downward slant of the forehead, delayed bone maturation, mental retardation. The ocular abnormalities are generally retinal coloboma and nystagmus.

### Cancer

Cancer, also known as a malignancy, malignant neoplasm, or malignant tumor, is a group of diseases involving abnormal cell growth with the potential to invade or spread to other parts of the body (Cancer Fact sheet N^{o} 297. World Health Organization. February 2014; Defining Cancer. National Cancer Institute. 2014). Not all tumors are cancerous as benign tumors do not spread (Defining Cancer. National Cancer Institute. 2014). Possible signs and symptoms include: a new lump, abnormal bleeding, a prolonged cough, unexplained weight loss, and a change in bowel movements among others (Cancer—Signs and symptoms. NHS Choices. 2014). While these symptoms may indicate cancer, they may also occur due to other issues (Cancer—Signs and symptoms. NHS Choices. 2014). There are over 100 different known cancers that affect humans (Defining Cancer. National Cancer Institute. 2014). Cancers are a large family of diseases that involve abnormal cell growth with the potential to invade or spread to other parts of the body (Cancer Fact sheet N^{o} 297. World Health Organization. February 2014; Defining Cancer. National Cancer Institute. 2014). A neoplasm or tumor is a group of cells that have undergone unregulated growth, and will often form a mass or lump, but may be distributed diffusely (Cancer Glossary. cancer.org. American Cancer Society. 2013; What is cancer?cancer.gov. National Cancer Institute. 2013). Proposed characteristics of cancer include: 1) insensitivity to anti-growth signals; 2) self-sufficiency in growth signaling; 3) induction and sustainment of angiogenesis; 4) evasion of apoptosis; 5) enabling of a limitless replicative potential; and 6) activation of metastasis and invasion of tissue (Hanahan, Douglas; Weinberg, Robert A. (Jan. 7, 2000). “The hallmarks of cancer”. Cell 100 (1): 57-70). Malignant progression is the multi-step process that takes normal cells to cells that can form a discernible mass to cancer (Hanahan, Douglas; Weinberg, Robert A. (Jan. 7, 2000). “The hallmarks of cancer”. Cell 100 (1): 57-70; Hanahan, Douglas; Weinberg, Robert A. (2011). “Hallmarks of Cancer: The Next Generation”. Cell 144 (5): 646-74).

Cancer is a disease of tissue growth regulation failure. Genes that regulate cell growth and differentiation must be altered for a normal cell to become cancerous (Croce C M (January 2008). “Oncogenes and cancer”. N. Engl. J. Med. 358 (5): 502-11). The affected genes are divided into two broad categories—tumor suppressor genes and oncogenes. Tumor suppressor genes inhibit cell division and survival. Oncogenes promote cell growth and reproduction. Tumor suppressor genes are genes that inhibit cell division and survival. Malignant transformation can occur through: the under-expression or disabling of tumor suppressor genes, the inappropriate over-expression of normal oncogenes, or formation of novel oncogenes (Knudson A G (November 2001). “Two genetic hits (more or less) to cancer”. Nature Reviews Cancer 1 (2): 157-62). Cancer is driven by progressive genetic abnormalities that include mutations in oncogenes, tumor-suppressor genes chromosomal abnormalities and epigenetic alterations (Baylin S B, Ohm J E (February 2006). “Epigenetic gene silencing in cancer—a mechanism for early oncogenic pathway addiction?”. Nature Reviews Cancer 6 (2): 107-16).

Most cancers are initially recognized either because of the appearance of signs or symptoms or through screening. A definitive diagnosis requires the examination of a tissue sample by a pathologist. Patients with suspected cancer are subjected to diagnostic tests which include CT scans, blood tests, endoscopy and X-rays.

Malignant cancers treated by the methods and compositions described herein include gastric cancer, multiple myeloma, leukemia, lymphoma, hepatocellular carcinoma, renal cell carcinoma, prostate cancer, brain cancer, glioblastoma, melanoma, breast cancer, head and neck cancer, and non-small cell lung carcinoma.

Glioblastoma, also known as glioblastoma multiforme (GBM) and grade IV astrocytoma, is the most common and most aggressive malignant primary brain tumor. It involves glial cells and accounting for 52% of all brain tissue tumor cases and 20% of all tumors inside the skull (“Glioblastoma and Malignant Astrocytoma”. American Brain Tumour Association (ABTA) 2014). About 50% of the people diagnosed with GBM die within one year, while 90% within three years. Treatment can involve chemotherapy, radiation and surgery. Median survival with standard-of-care radiation and chemotherapy with temozolomide is 15 months (Johnson, Derek R.; O'Neill, Brian Patrick (2011). “Glioblastoma survival in the United States before and during the temozolomide era”. Journal of Neuro-Oncology 107 (2): 359-64). Median survival without treatment is 412 months. Although no randomized controlled trials have been done, surgery remains the standard of care (Van Meir, E. G.; Hadjipanayis, C. G.; Norden, A. D.; Shu, H. K.; Wen, P. Y.; Olson, J. J. (2010). “Exciting New Advances in Neuro-Oncology: The Avenue to a Cure for Malignant Glioma”. CA: A Cancer Journal for Clinicians 60 (3): 166-93).

Although common symptoms of the disease include seizure, nausea and vomiting, headache, memory loss, and hemiparesis, the single most prevalent symptom is a progressive memory, personality, or neurological deficit due to temporal and frontal lobe involvement. The kind of symptoms produced depends highly on the location of the tumor, more so than on its pathological properties. The tumor can start producing symptoms quickly, but occasionally is an asymptomatic condition until it reaches an enormous size.

When viewed with MRI, glioblastomas often appear as ring-enhancing lesions. The appearance is not specific, however, as other lesions such as abscess, metastasis, tumefactive multiple sclerosis, and other entities may have a similar appearance (Smirniotopoulos, J. G.; Murphy, F. M.; Rushing, E. J.; Rees, J. H.; Schroeder, J. W. (2007). “From the Archives of the AFIP: Patterns of Contrast Enhancement in the Brain and Meninges”. Radiographics 27 (2): 525-51). Definitive diagnosis of a suspected GBM on CT or MRI requires a stereotactic biopsy or a craniotomy with tumor resection and pathologic confirmation. Because the tumor grade is based upon the most malignant portion of the tumor, biopsy or subtotal tumor resection can result in undergrading of the lesion. Imaging of tumor blood flow using perfusion MRI and measuring tumor metabolite concentration with MR spectroscopy will add value to standard MRI in the diagnosis of glioblastoma by showing increased relative cerebral blood volume and increased choline peak respectively, but pathology remains the gold standard (Weerakkody, Yuranga; Gaillard, Frank. “Glioblastoma”. Radiopaedia.org. 2014).

The diagnosis of glioblastoma depends on distinguishing primary glioblastoma from secondary glioblastoma. These tumors occur spontaneously (de novo) or have progressed from a lower-grade glioma, respectively (Bleeker, FE; Molenaar, RJ; Leenstra, S (May 2012). “Recent advances in the molecular understanding of glioblastoma.”. Journal of neuro-oncology 108 (1): 11-27). Primary glioblastomas have a worse prognosis, different tumor biology may have a different response to therapy, which makes this a critical evaluation to determine patient prognosis and therapy (Weerakkody, Yuranga; Gaillard, Frank. “Glioblastoma”. Radiopaedia.org 2014). Over 80% of secondary glioblastoma carries a mutation in IDH1, whereas this mutation is rare in primary glioblastoma (5-10%). Thus, IDH1 mutations may become a useful tool to distinguish primary and secondary glioblastomas in the future, since histopathologically they are very similar and the distinction without molecular biomarkers is unreliable (The driver and passenger effects of isocitrate dehydrogenase 1 and 2 mutations in oncogenesis and survival prolongation.”. Biochim Biophys Acta 1846 (2): 326-41. December 2014).

### IV. Pharmaceutical Compositions

The weight loss agents described above, can be formulated into pharmaceutical compositions suitable for use in the present methods. Such compositions include the active agent (compounds of Formula (I)) together with a pharmaceutically acceptable carrier, excipient or diluent.

In some embodiments, a pharmaceutical composition includes those described in International Publication No. WO 2014/052583, herein incorporated by reference in its entirety.

In some cases, a pharmaceutical composition includes the compounds of Formula (I), a pharmaceutically acceptable salt or prodrug thereof and a combination with one or more pharmaceutically acceptable excipients.

Pharmaceutical compositions provided by the present invention include compositions wherein the active ingredient is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., induce weight loss. Determination of a therapeutically effective amount of compounds of the invention is well within the capabilities of those skilled in the art, especially in light of the detailed disclosure herein.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, that may also act as diluents, flavoring agents, binders, preservatives, tablet disintegrating agents, or an encapsulating material.

In powders, the carrier is a finely divided solid in a mixture with the finely divided active component (e.g. a compound provided herein). In tablets, the active component is mixed with the carrier having the necessary binding properties in suitable proportions and compacted in the shape and size desired. The powders and tablets preferably contain from 5% to 70% of the active compound.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragees cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the salts of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The salts of the invention can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, PA) and WO 96/05309, the teachings of both of which are hereby incorporated by reference.

Aqueous solutions suitable for oral use can be prepared by dissolving the active salt (e.g. compounds described herein, including embodiments, and examples) in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, cyclodextrin, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g. to induce weight loss).

The compounds of the invention can be administered in a variety of routes including but not limited to: oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

The compounds of the present invention can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The salts of the present invention can also be administered by injection, that is, intravenously, intramuscularly, intracutaneously, subcutaneously, intraduodenally, or intraperitoneally. Also, the salts described herein can be administered by inhalation, for example, intranasally. Additionally, the salts of the present invention can be administered transdermally. It is also envisioned that multiple routes of administration (e.g., intramuscular, oral, transdermal) can be used to administer the salts of the invention. Accordingly, the present invention also provides pharmaceutical compositions comprising a pharmaceutically acceptable excipient and one or more salts of the invention.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 μg to 10000 μg, more typically 1.0 μg to 1000 μg, most typically 50 μg to 1000 μg, according to the particular application and the potency of the active component. Example of single unit doses may be 50, 100, 200, 300, 400, 500, 600, 700, 800, 900, 1000, 2000, or 5000 μg. The composition can, if desired, also contain other compatible therapeutic agents. Multiple unit doses may be administered within a 24 hour time period. Doses may be administered orally but other routes of administration may also be used depending on the severity of the disease/disorder of the patient.

Some compounds may have limited solubility in water and therefore may require a surfactant or other appropriate co-solvent in the composition. Such co-solvents include: Polysorbate 20, 60 and 80; Pluronic F-68, F-84 and P-103; cyclodextrin; polyoxyl 35 castor oil; or other agents known to those skilled in the art. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., emphysema, asthma, ARDS including oxygen toxicity, pneumonia, chronic obstructive pulmonary disease (COPD), emphysema, cystic fibrosis, bronchopulmonary dysplasia, chronic sinusitis, pulmonary fibrosis), kind of concurrent treatment, complications from the disease being treated or other health-related problems. The disease may be a primary cause for a weight gain disease and/or disorder. The disease may be a caused by a primary weight gain disorder and/or disorder. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In some embodiments, the dosage range is 0.001% to 10% w/v. In another embodiment, the dosage range is 0.1% to 5% w/v.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

The ratio between toxicity and therapeutic effect for a particular compound is its therapeutic index and can be expressed as the ratio between LD50 (the amount of compound lethal in 50% of the population) and ED50 (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED50 with little or no toxicity. The dosage may vary within this range depending upon the dosage form employed and the route of administration utilized. See, e.g. Fingl et al., In: The Pharmacological Basis of Therapeutics, Ch.1, p. 1, 1975. The exact formulation, route of administration and dosage can be chosen by the individual physician in view of the patient's condition and the particular method in which the compound is used.

### General Process for Preparation of a Highly Pure Form III of Compound I (Celastrol)

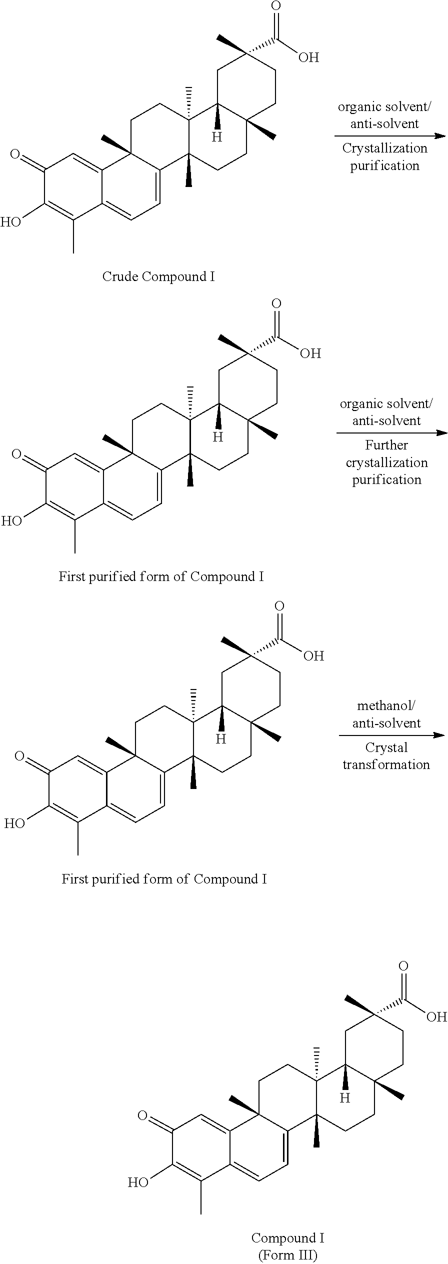

Crude Compound I (i.e., crude celastrol, e.g., crystalline Form I of Compound I or a mixture of crystalline Form I and Form III of Compound I) is dissolved in an organic solvent (e.g., 2-MeTHF) e.g., by combining the crude Compound I and the organic solvent and heating the resulting mixture (e.g., to a temperature of about 75° C.). The resulting solution may be cooled before proceeding with the reaction. Then, the solution of crude Compound I and the organic solvent is seeded and a first purified form is precipitated (e.g., by addition of an anti-solvent, e.g., n-heptane, followed by cooling of the resulting mixture). Then, the first purified form is dissolved in an organic solvent (e.g., 2-MeTHF), e.g., by combining the first purified form and the organic solvent and heating the resulting mixture (e.g., to a temperature of about 75° C.). The resulting solution may be cooled before proceeding with the reaction. Then, the solution of the first purified form and the organic solvent is seeded and a second purified form is precipitated (e.g., by addition of an anti-solvent, e.g., n-heptane, followed by cooling of the resulting mixture). Lastly, the Form III of Compound I (e.g., a highly pure polymorph of Form III of Compound I) is crystallized from methanol. For example, the crystallization from methanol is achieved by dissolving the second purified form of Compound I in the methanol, e.g., by combining the second purified form and the methanol and heating the resulting mixture (e.g., to a temperature of about 55° C.), and precipitating the Form III of Compound I from the resulting solution (e.g., by addition of an anti-solvent, e.g., H₂O, to the seeded solution followed by cooling of the resulting mixture).

### V. Examples

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### List of Abbreviations

API Active Pharmaceutical Ingredient
DCM Dichloromethane
DMF Dimethylformamide
DSC Differential Scanning Calorimetry
DVS Dynamic Vapor Sorption
Eq. Equivalent
HPLC High Performance Liquid Chromatography
MeOH Methanol
RH Relative Humidity
SEM Scanning Electron Microscopy
TGA ThermoGravimetric Analysis
XRPD X-Ray Powder Diffraction
SMS Surface measurement system

### Example 1. Salt Screening and Selection

A salt screen of celastrol was conducted

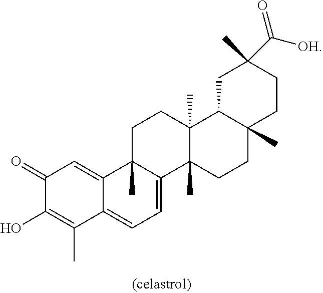

The crystallization experiments were conducted on 5 solvents at different concentrations and stochiometries (1.2 and 2.2 eq). The solvents were chosen according to their physico-chemical properties. The starting material for the screen (i.e., the free base) was Form I of celastrol. The free base was also tested in the same solvents at two concentrations. The counter-ions were selected among pharmaceutically acceptable ones and according to their respective pKa. The pKa of the free compound were calculated in-silico and found to be 4.8 (apparent) for the carboxylic acid function and 8.7 (apparent) for the phenol function.

Among the whole set of salt screening experiments tested, one condition led to the formation of a salt, which was obtained from methanol with 2.2 equivalents of trometamol. The salt formed had a 1 to 1 stoichiometry.

### 1.1 Free Celastrol

The physico-chemical properties of free Celastrol are summarized as follows:
Appearance: Red powder, platelet habit with presence of agglomerates
XRPD (crystallinity): Crystalline phase with high diffraction intensities
Thermal analysis (DSC): endothermic peat at around 156° C. (onset)
Thermal analysis (TGA): decrease in weight about 4.8% during endothermic peak
DVS(hygroscopicity): Non-hygroscopic compound
XPRD under temperature and relative humidity: no new crystalline phase until 100° C. and no new crystalline phase from 10 to 90% RH
Solubility in water at 37° C.: very slightly soluble (<1 g/L)

Table 1 provides a summary of experiments using free celastrol. Further, no polymorphism was observed from free API experiments except by evaporation in dichloromethane where in a mixture was obtained.

TABLE 1
API Experiments

| | Conc. | | | Observation | |
|---|---|---|---|---|---|
| No. | (g/L) | Technique | Solvent | (XRPD) | Conclusion |
| 1 | 50-150 | cooling | water | API only | No |
| | | | | | polymorphism |
| 2 | 50-150 | evaporation | water | Not enough solid | |
| | | | | isolated | |
| 3 | 50-150 | cooling | ethanol | API only | No |
| | | | | | polymorphism |
| 4 | 50 | evaporation | ethanol | API only | No |
| | | | | | polymorphism |
| 5 | 150 | evaporation | ethanol | API only | No |
| | | | | | polymorphism |
| 6 | 50 | cooling | methanol | no solid obtained | |
| 7 | 150 | cooling | methanol | API only | No |
| | | | | | polymorphism |
| 8 | 50 | evaporation | methanol | amorphous | |
| 9 | 150 | evaporation | methanol | API only | No |
| | | | | | polymorphism |
| 10 | 50 | cooling | DMF | no solid obtained | |
| 11 | 150 | cooling | DMF | no solid obtained | |
| 12 | 50 | evaporation | DMF | amorphous | |
| 13 | 150 | evaporation | DMF | amorphous | |
| 14 | 50 | cooling | acetone | API only | No |
| | | | | | polymorphism |
| 15 | 150 | cooling | acetone | API only | No |
| | | | | | polymorphism |
| 16 | 50 | evaporation | acetone | API only (very low | No |
| | | | | cry stallinity) | polymorphism |
| 17 | 150 | evaporation | acetone | Not enough solid | |
| | | | | isolated | |
| 18 | 120 | cooling | dichloromethane | Not enough solid | |
| | | | | isolated | |
| 19 | 120 | evaporation | dichloromethane | New diffraction | Probably |
| | | | | pattern | polymorph of the |
| | | | | | API |

1.2 Treatment of Celastrol with NaOH

Table 2 provides a summary of experiments where celastrol (i.e., the API) was treated with sodium hydroxide (NaOH), which has a pKa of 14. Sodium hydroxide experiments led either to amorphous powders or to 8 new diffraction patterns. HPLC analyses on the solids presenting new diffraction patterns demonstrated the degradation of the API. No salt formation was observed with sodium hydroxide.

TABLE 2
Treatment of Celastrol with NaOH

| | Conc. | | | Observation | |
|---|---|---|---|---|---|
| No. | (g/L) | Technique | Solvent | (XRPD) | Conclusion |
| 1 | 50 | 1.2 | water | amorphous | no salt formation |
| 2 | 150 | 1.2 | water | amorphous | no salt formation |
| 3 | 50 | 2.2 | water | amorphous | no salt formation |
| 4 | 150 | 2.2 | water | amorphous | no salt formation |
| 5 | 50 | 1.2 | ethanol | amorphous | no salt formation |
| 6 | 150 | 1.2 | ethanol | new diffraction | degradation |
| | | | | pattern | |
| 7 | 50 | 2.2 | ethanol | amorphous | no salt formation |
| 8 | 150 | 2.2 | ethanol | amorphous | no salt formation |
| 9 | 50 | 1.2 | methanol | amorphous | no salt formation |
| 10 | 150 | 1.2 | methanol | amorphous | no salt formation |
| 11 | 50 | 2.2 | methanol | new diffraction | degradation |
| | | | | pattern | |
| 12 | 150 | 2.2 | methanol | new diffraction | degradation |
| | | | | pattern | |
| 13 | 50 | 1.2 | DMF | amorphous | no salt formation |
| 14 | 150 | 1.2 | DMF | amorphous | no salt formation |
| 15 | 50 | 2.2 | DMF | amorphous | no salt formation |
| 16 | 150 | 2.2 | DMF | new diffraction | degradation |
| | | | | pattern | |
| 17 | 50 | 1.2 | acetone | new diffraction | degradation |
| | | | | pattern | |
| 18 | 150 | 1.2 | acetone | new diffraction | degradation |
| | | | | pattern | |
| 19 | 50 | 2.2 | acetone | new diffraction | degradation |
| | | | | pattern | |
| 20 | 150 | 2.2 | acetone | new diffraction | degradation |
| | | | | pattern | |

1.3 Treatment of Celastrol with KOH

Table 3 provides a summary of experiments where celastrol was treated with potassium hydroxide (KOH), which has a pKa of 14. Potassium hydroxide experiments led either to amorphous powders or to 7 new diffraction patterns. TPLC analyses on the solids presenting new diffraction patterns demonstrated the degradation of the API No salt formation was observed with potassium hydroxide.

TABLE 3
Treatment of Celastrol with KOH

| | Conc. | | | Observation | |
|---|---|---|---|---|---|
| No. | (g/L) | Technique | Solvent | (XRPD) | Conclusion |
| 1 | 50 | 1.2 | water | amorphous | no salt formation |
| 2 | 150 | 1.2 | water | amorphous | no salt formation |
| 3 | 50 | 2.2 | water | amorphous | no salt formation |
| 4 | 150 | 2.2 | water | amorphous | no salt formation |
| 5 | 50 | 1.2 | ethanol | amorphous | no salt formation |
| 6 | 150 | 1.2 | ethanol | amorphous | no salt formation |
| 7 | 50 | 2.2 | ethanol | new diffraction pattern | degradation |
| 8 | 150 | 2.2 | ethanol | new diffraction pattern | degradation |
| 9 | 50 | 1.2 | methanol | amorphous | no salt formation |
| 10 | 150 | 1.2 | methanol | new diffraction pattern | degradation |
| 11 | 50 | 2.2 | methanol | new diffraction pattern | degradation |
| 12 | 150 | 2.2 | methanol | new diffraction pattern | degradation |
| 13 | 50 | 1.2 | DMF | amorphous | no salt formation |
| 14 | 150 | 1.2 | DMF | new diffraction pattern | degradation |
| 15 | 50 | 2.2 | DMF | amorphous | no salt formation |
| 16 | 150 | 2.2 | DMF | amorphous | no salt formation |
| 17 | 50 | 1.2 | acetone | API only | no salt formation |
| 18 | 150 | 1.2 | acetone | API + supplementary peak | degradation |
| | | | | at 4.6° | |
| 19 | 50 | 2.2 | acetone | API only | no salt formation |
| 20 | 150 | 2.2 | acetone | amorphous | no salt formation |

1.4 Treatment of Celastrol with Calcium Hydroxide

Table 4 provides a summary of experiments where celastrol was treated with calcium hydroxide (Ca(OH)₂), which has a pKa of 14. Calcium hydroxide experiments led to 3 new diffraction patterns. HPLC analyses on the solids presenting new diffraction patterns demonstrated the degradation of the API. No salt formation was observed with calcium hydroxide.

TABLE 4
Treatment of Celastrol with Calcium Hydroxide

| | Conc. | | | Observation | |
|---|---|---|---|---|---|
| No. | (g/L) | Technique | Solvent | (XRPD) | Conclusion |
| 1 | 50 | 1.2 | water | API + co-former | no salt formation |
| 2 | 150 | 1.2 | water | API | no salt formation |
| 3 | 50 | 2.2 | water | API + co-former | no salt formation |
| 4 | 150 | 2.2 | water | co-former | no salt formation |
| 5 | 50 | 1.2 | ethanol | API + co-former | no salt formation |
| 6 | 150 | 1.2 | ethanol | API + co-former | no salt formation |
| 7 | 50 | 2.2 | ethanol | API + co-former | no salt formation |
| 8 | 150 | 2.2 | ethanol | API + co-former | no salt formation |
| 9 | 50 | 1.2 | methanol | API + co-former | no salt formation |
| 10 | 150 | 1.2 | methanol | API + co-former | no salt formation |
| 11 | 50 | 2.2 | methanol | co-former | no salt formation |
| 12 | 150 | 2.2 | methanol | API + co-former | no salt formation |
| 13 | 50 | 1.2 | DMF | new diffraction pattern | degradation |
| 14 | 150 | 1.2 | DMF | amorphous | no salt formation |
| 15 | 50 | 2.2 | DMF | new diffraction pattern | degradation |
| 16 | 150 | 2.2 | DMF | new diffraction pattern | degradation |
| 17 | 50 | 1.2 | acetone | API + co-former | no salt formation |
| 18 | 150 | 1.2 | acetone | API + co-former | no salt formation |
| 19 | 50 | 2.2 | acetone | API + co-former | no salt formation |
| 20 | 150 | 2.2 | acetone | API + co-former | no salt formation |

1.5 Treatment of Celastrol with L(+)-Lysine

Table 5 provides a summary of experiments where celastrol was treated with lysine. The pKa of L-Lysine are 2.16 (A), 9.18 (B) and 10.79 (B). Experiments with L(+) Lysine did not lead to any salt formation: only API, co-former or amorphous powders were obtained.

TABLE 5
Treatment of Celastrol with L(+)-Lysine

| | Conc. | | | Observation | |
|---|---|---|---|---|---|
| No. | (g/L) | Technique | Solvent | (XRPD) | Conclusion |
| 1 | 50 | 1.2 | water | API only | no salt formation |
| 2 | 150 | 1.2 | water | API only | no salt formation |
| 3 | 50 | 2.2 | water | API only | no salt formation |
| 4 | 150 | 2.2 | water | API only | no salt formation |
| 5 | 50 | 1.2 | ethanol | co-former | no salt formation |
| 6 | 150 | 1.2 | ethanol | co-former | no salt formation |
| 7 | 50 | 2.2 | ethanol | co-former | no salt formation |
| 8 | 150 | 2.2 | ethanol | co-former | no salt formation |
| 9 | 50 | 1.2 | methanol | amorphous | no salt formation |
| 10 | 150 | 1.2 | methanol | amorphous | no salt formation |
| 11 | 50 | 2.2 | methanol | amorphous | no salt formation |
| 12 | 150 | 2.2 | methanol | amorphous | no salt formation |
| 13 | 50 | 1.2 | DMF | co-former | no salt formation |
| 14 | 150 | 1.2 | DMF | co-former | no salt formation |
| 15 | 50 | 2.2 | DMF | co-former | no salt formation |
| 16 | 150 | 2.2 | DMF | co-former | no salt formation |
| 17 | 50 | 1.2 | acetone | co-former | no salt formation |
| 18 | 150 | 1.2 | acetone | co-former | no salt formation |
| 19 | 50 | 2.2 | acetone | co-former | no salt formation |
| 20 | 150 | 2.2 | acetone | co-former | no salt formation |

1.6 Treatment of Celastrol with Trometamol

Table 6 provides a summary of experiments where celastrol was treated with trometamol. The pKa of trometamol is 8.2 (B). Experiments with trometamol led to 8 new diffraction patterns. HPLC analyses on the solids presenting new diffraction patterns demonstrated the degradation of the API in most of the cases with a noticeable exception: in methanol, and at 2.2 equivalents of trometamol, the formation of a salt was achieved. This was confirmed by HPLC and elemental analysis. The salt formed had a 1 to 1 stoichiometry.

TABLE 6
Treatment of Celastrol with Trometamol

| | Conc. | | | Observation | |
|---|---|---|---|---|---|
| No. | (g/L) | Technique | Solvent | (XRPD) | Conclusion |
| 1 | 50 | 1.2 | water | API only | no salt formation |
| 2 | 150 | 1.2 | water | API + supplementary peak | degradation |
| | | | | at 11.6° | |
| 3 | 50 | 2.2 | water | API only | no salt formation |
| 4 | 150 | 2.2 | water | API only | no salt formation |
| 5 | 50 | 1.2 | ethanol | new diffraction pattern | degradation |
| 6 | 150 | 1.2 | ethanol | new diffraction pattern | degradation |
| 7 | 50 | 2.2 | ethanol | new diffraction pattern | degradation |
| 8 | 150 | 2.2 | ethanol | new diffraction pattern | degradation |
| 9 | 50 | 1.2 | methanol | amorphous | no salt formation |
| 10 | 150 | 1.2 | methanol | amorphous | no salt formation |
| 11 | 50 | 2.2 | methanol | new diffraction pattern | salt formation |
| 12 | 150 | 2.2 | methanol | co-former only | no salt formation |
| 13 | 50 | 1.2 | DMF | co-former only | no salt formation |
| 14 | 150 | 1.2 | DMF | new diffraction pattern | degradation |
| 15 | 50 | 2.2 | DMF | co-former only | no salt formation |
| 16 | 150 | 2.2 | DMF | co-former + 1 peak | degradation |
| 17 | 50 | 1.2 | acetone | API + co-former | no salt formation |
| 18 | 150 | 1.2 | acetone | API + co-former | no salt formation |
| 19 | 50 | 2.2 | acetone | API + co-former | no salt formation |
| 20 | 150 | 2.2 | acetone | API + co-former | no salt formation |

### Example 2. Trometamol Salt

The trometamol salt of Compound I (Compound 2), was further studied:

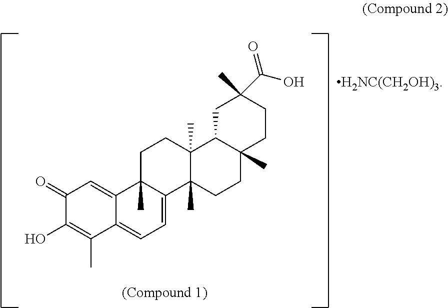

### 2.1 Scale Up Of Trometamol Salt

The process used for the manufacture of trometamol salt Compound 2 is summarized in Table 7 below.

TABLE 7
Synthesis of Compound 2

| | | | Equiv. | | |
|---|---|---|---|---|---|
| Scale | Solvent | Concentration | Acid | Manufacturing process | Yield |
| 4 g | MeOH | 900 g/L | 1.1 eq | Dissolution of API in 2.2 V of MeOH | 56% |
| | | | | at 50° C. (Tjacket) | |
| | | | | Addition of 1.1 eq of Trometamol | |
| | | | | Addition of 1.1 V of MeOH | |
| | | | | Cooling then seeding at 25° C. | |
| | | | | Cooling at 10° C. | |
| | | | | Stirring 2 h | |
| | | | | Filtration under vaccuum | |

Only 1.1 eq. of Trometamol was used to get the salt formation compared to 2.2eq. used during the salt screening. Around 2.2 g of salt was obtained by this process.

### 2.2 Characterization of Trometamol Salt

### 2.2.1 Appearance (Optical Microscope and Scanning Electron Microscope)

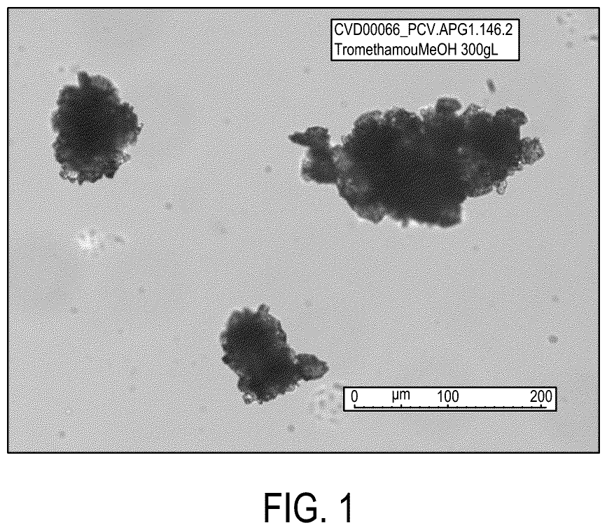

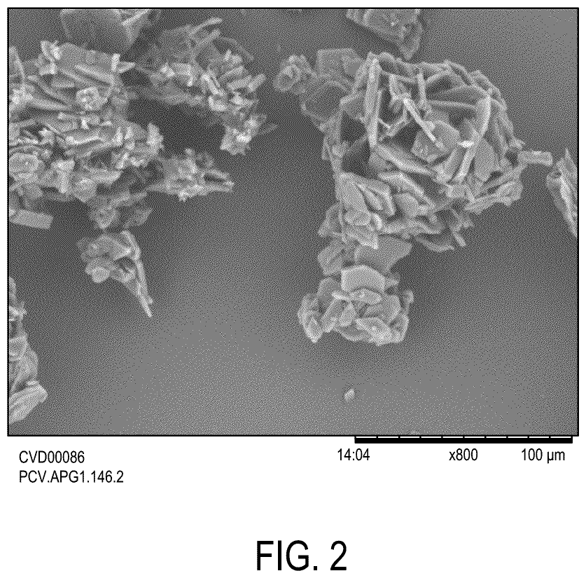

Photos by optical microscopy (FIG. 1; obj.×10) and SEM (FIG. 2; G×800) were obtained of the trometamol salt (Compound 2). The product presents itself as a red powder. It exhibits a platelet habit with the presence of agglomerates.

### 2.2.2 Crystallinity (XRPD)

The analysis was performed on a PHILIPS PANalytical, X'Pert Pro Diffractometer. The experimental conditions are the following:
Configuration Bragg-Brentano: 9-9
Source: Cu with Kα1 at 1=1.5406 Å.
The power supply works at 45 kV and 40 mA.
Detector: PSD X'Celerator in scanning mode
Sample temperature: room temperature
Angular variation: from 20=3° to 450
Step size [° 29]: 0.170
Scan step time (in s): 54.8054
Duration: 10 min

A few mg of sample was prepared on a silicon plate without any special treatment other than a slight pressure to obtain a flat and homogeneous surface for reproducible and repeatable results. The diffraction pattern obtained for trometamol salt Compound 2 is provided in FIG. 3. The trometamol salt was found crystalline with low diffraction intensity.

### 2.2.3 Thermal Analysis (DSC TGA)

### 2.2.3.1.1 Differential Scanning Calorimetry (DSC) and Thermogravimetric Analysis (TGA)

A different scanning calorimeter (Model Mettler Toldedo DSC822) was used. The experimental conditions are summarized below.
Accurate amount of compound (mg scale) was packed in aluminum pans (40 L), crimped with lids having pinholes.
Heating rate 20.00° C./min under nitrogen purge (40 mL/min)
Heating range from 30 to 400° C.

A thermogravimetric analyzer (Model Mettler Toldedo TGA/SDTA851) was used. The experimental conditions are similar to the one used by DSC in order to compare both techniques.

The results of these thermal analyses of trometamol salt Compound 2 are presented in FIG. 4. The DSC experiments showed multiple endothermic and exothermic peaks. The main peak occurred at 120° C. (onset) with an enthalpy of energy of −34 J/g. The TGA results showed a loss in weight of 0.7% during the endothermic peak of compound.

### 2.2.4 Hygroscopicity (DVS)

The hygroscopicity experiments were carried out with a DVS Intrinsic from S.M.S. This technique is designed to accurately measure a sample's change in mass as it sorbs precisely controlled concentrations of water vapors in an air carrier gas. The method used for DVS experiments (at 37° C.) is summarized below.
Equilibrating 10 min at 50% RH
Decreasing to 0% RH in 60 min
Equilibrating 10 min at 0% RH
Increasing to 95% RH in 280 min
Equilibrating 10 min at 95% RH
Decreasing to 50% RH in 100 min
Equilibrating 20 min at 50% RH

The water sorption/desorption of trometamol salt Compound 2 is shown in FIG. 5. The mass decreased by ˜3.8% w/w as the RH was lowered to 0%. The mass then increased by 11% w/w as the RH was increased to 95%. Hysteresis was observed with the final mass at 50% RH (˜3% w/w). The water sorption/desorption isotherms at 37° C. showed a hygropscopic compound (2 ˜15% w/w).

### 2.2.5 XRPD Under Temperature and Relative Humidity

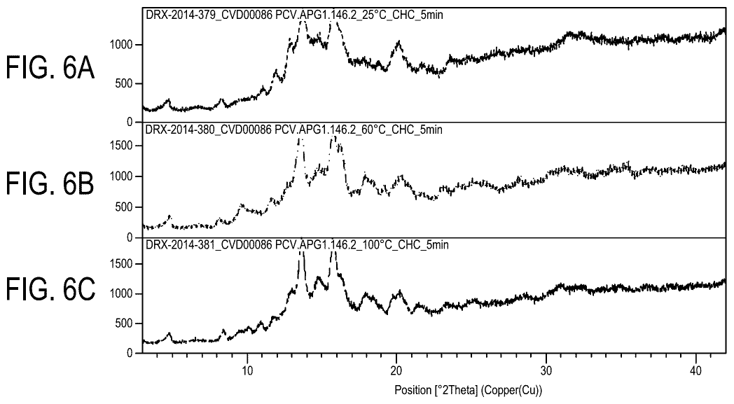

Method 1: influence of temperature at ambient RH. RH was set at 30% in order to simulate a RH near from the ambient RH, and the temperature studied were 25-60-100° C. FIG. 6 shows the XRPD pattern representing the influence of temperature on the trometamol salt Compound 2 (FIG. 6A=25° C.; FIG. 6B=60° C.; and FIG. 6C=100° C.).

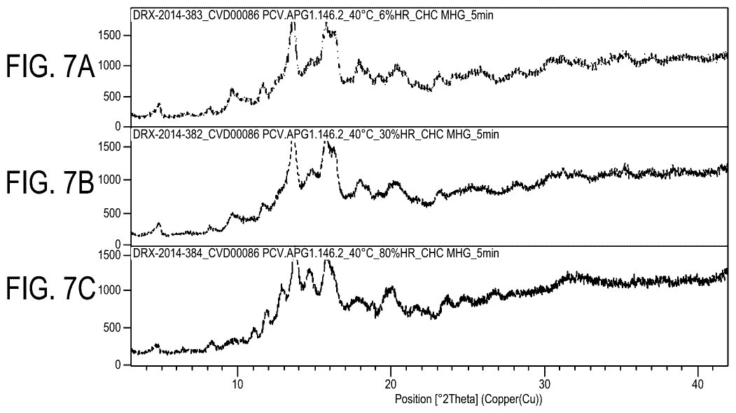

Method 2: influence on RH at 40° C. The RH studied was at 10-30-80%. FIG. 7 shows the XRPD pattern representing the influence of RH at 40° C. on trometamol salt Compound 2 (FIG. 7A=10%; FIG. 7B=30%; and FIG. 7C=80%).

No impact of temperature (from 25 to 100° C.) was observed on the trometamol salt Compound 2 in the conditions tested. An impact of relative humidity at 40° C. at 80% RH was observed by the formation of a new crystalline pattern (new crystalline form or degradation).

### 2.2.5.1 Solubility in Water at 37° C.

The solubility was measured in water at 37° C. and the trometamol salt Compound 2 was found slightly soluble (<1.5 g/L).

### 2.3 Summary

The physico-chemical properties of the trometamol salt Compound 2 and the free compound were compared. The results are gathered in the table below: The free compound was found highly crystalline, thermally stable, physically stable under relative humidity and non hygroscopic. The Trometamol salt was found less crystalline, less thermally stable (lower endothermic peak), less stable physically under relative humidity (new diffraction pattern from 80% RH) and hygroscopic. In addition to this, the aqueous solubility was similar to the one of the free compound.

TABLE 8
Summary of solid state characterization of Trometamol Salt Compound 2 and Free Compound 1

| Tests | Free compound | Trometamol salt |
|---|---|---|
| Appearance | Red powder | Red powder |
| (OM/SEM) | Platelet habit with presence | Platelet habit with presence of |
| | of agglomerates | agglomerates |
| XRPD (crystallinity) | Crystalline phase with high | Crystalline phase with low diffraction |
| | diffraction | intensities |
| | intensities | |
| Thermal analyses | DSC: | DSC: |
| (DSC/TGA) | Endothermic peak at 156° C. | Main endothermic peak at 120° C. (onset) |
| | (onset) | TGA: |
| | TGA: | 0.7% during endothermic peak |
| | 4.8% weight loss at 150° C. | |
| DVS (hygroscopicity) | Non hygroscopic | Hygroscopic |
| XRPD under | Under Temperature: | Under Temperature: |
| temperature | 25° C., 60° C. and 100° C. | 25° C., 60° C. and 100° C. |
| and RH | =>No new crystalline phase until | =>No new crystalline phase until 100° C. |
| | 100° C. | Under relative humidity (RH) at 40° C.: |
| | Under relative humidity (RH) at | 10%, 30% and 80% RH |
| | 40° C.: 10%, 30% and 90% RH | =>New crystaline phase from 80% RH |
| | =>No new crystalline phase from | |
| | 30 to 90% RH | |
| Purity by HPLC | 94.4% (scouting gradient method) | 94.3% (scouting gradient method) |
| (245 nm) | | |
| Solvent content | Does not comply | Does comply |
| (ICH standard) | (Ethanol ca. 5%) | MeOH: 0.13% |
| | | EtOH: 0.21% |
| Solublity in Water | (s < 1 g/L) | (s < 1.5 g/L) |
| at 37° C. | | |

### 2.5 Conclusion

Among the whole set of salt screening experiments tested, only one condition led to the formation of a salt. It was obtained from methanol with 2.2 equivalents of Trometamol. The salt formed had a 1 to 1 stoichiometry. Many experiments led to the degradation of the API or to amorphous powders.

The free compound presented itself as crystalline, physically stable under the conditions tested (temperature and RH).

### Example 3. Neutral Solid Form (Compound 1)

The free compound (Compound 1) presented itself as crystalline, physically stable under the conditions tested (temperature and RH).

### 3.1 Microscopy Studies

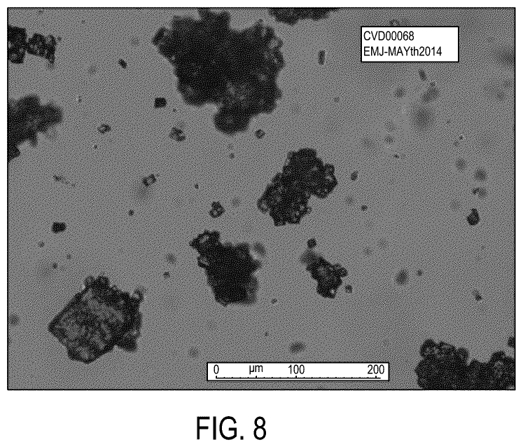

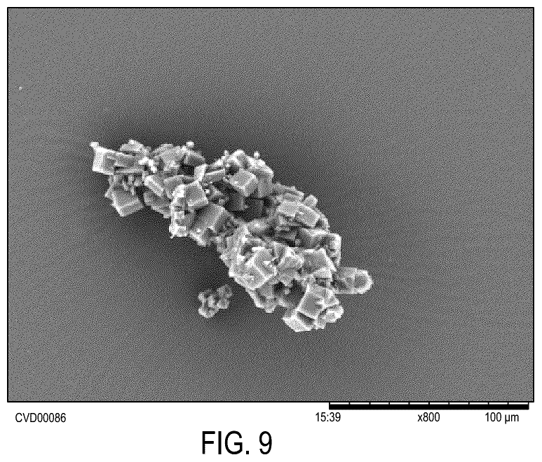

Microscopy studies were conducted using optical microscopy (FIG. 8; obj×10) and SEM (FIG. 9; G×800). The product presents itself as a red powder. It exhibits a platelet habit with the presence of agglomerates and fine particles, presuming a bimodal particle size distribution. 3.2 Crystallinity (XRPD)

The analysis was performed on a PHILIPS PANalytical, X'Pert Pro Diffractometer. The experimental conditions are the following:
Configuration Bragg-Brentano: θ-θ
Source: Cu with Kα₁ at λl=1.5406 Å.
The power supply works at 45 kV and 40 mA.
Detector: PSD X'Celerator in scanning mode
Sample temperature: room temperature
Angular variation: from 2θ=3° to 45°
Step size [° 29]: 0.170
Scan step time (ins): 54.8054
Duration: 10 min

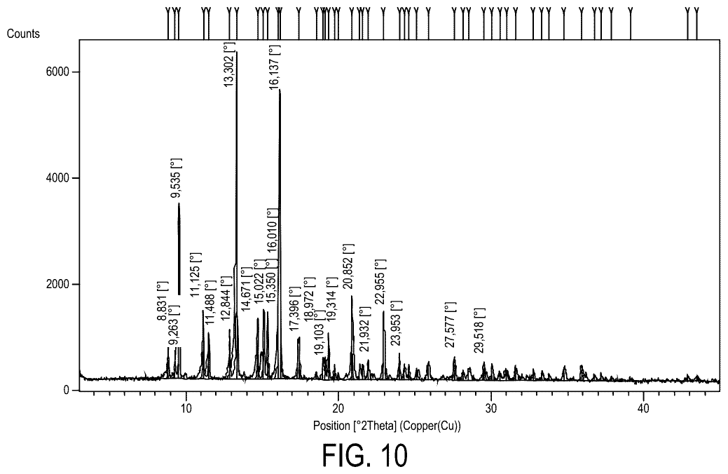

A few mg of sample was prepared on a silicon plate without any special treatment other than a slight pressure to obtain a flat homogeneous surface for reproducible and repeatable results. FIG. 10 provides the diffraction pattern of neutral Compound 1.

### 3.3 Thermal Analysis (DSC/TGS)

The combination of both techniques DSC/TGS allows determining the thermal behavior. The melting point can be determined by DSC, the decomposition of TGA.

### 3.3.1 Differential Scanning Calorimetry (DSC)

A differential scanning calorimeter (Model Mettler Toledo DSC822°) was used. The experimental conditions are summarized herein. Accurate amount of compound (mg scale) was packed in aluminum pans (40 L), crimped with lids having pinholes, and a heating rate 20.00° C./min under nitrogen purge (40 mL/min). Heating was ranged from 30 to 400° C.

### 3.3.2 Thermogravimetric Analysis (TGA)

A thermogravimetric analyzer (Model Mettler Toldedo TGA/SDTA851) was used. The experimental conditions are similar to the one used by DSC in order to compare both techniques.

### 3.3.3 Results

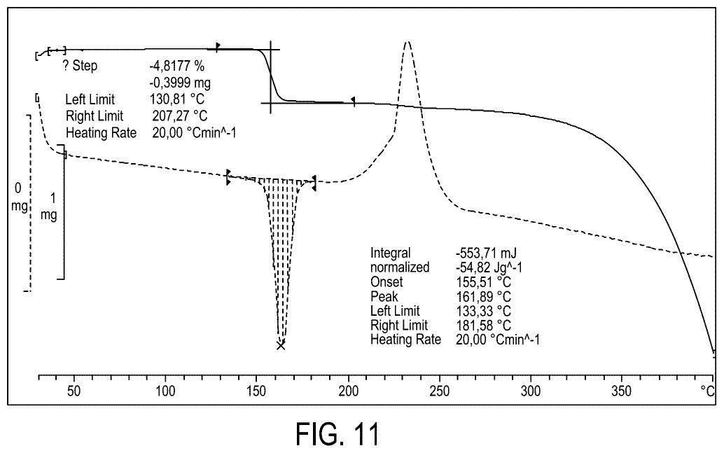

FIG. 11 shows thermal analysis (DSC and TGA) of neutral Compound 1. The DSC experiments showed an endothermic peak (onset) at 156° C. with an enthalphy of energy of −55 J/g followed by an exothermic peak. The TGA results showed a loss in weight of 4.8% during the endothermic peak of compound. FIG. 26A shows the DSC experiments showing an endothermic peak (onset) at 157° C. with an enthalphy of energy of −50 J/g followed by an exothermic peak. An exothermic peak occurred at 210° C. (onset). The TGA results showed a loss in weight of 4.6% during the endothermic peak of compound.

### 3.4 Hygroscopicity (DVS)

The hygroscopic experiments were carried out with a DVS Intrinsic from S.M.S. This technique is designed to accurately measure a sample's change in mass as it sorbs precisely controlled concentrations of water vapors in an air carrier gas. All the methods used for DVS experiments are summarized below. It should be noticed that the temperature and relative humidity (RH) are pre-set according to the initial temperature and RH.

### Method (37° C.):

Equilibrating 120 min at 50% RH
Decreasing to 0% RH in 60 min
Equilibrating 150 min at 0% RH
Increasing to 95% RH in 200 min
Equilibrating 360 min at 95% RH
Decreasing to 50% RH in 100 min
Equilibrating 150 min at 50% RH

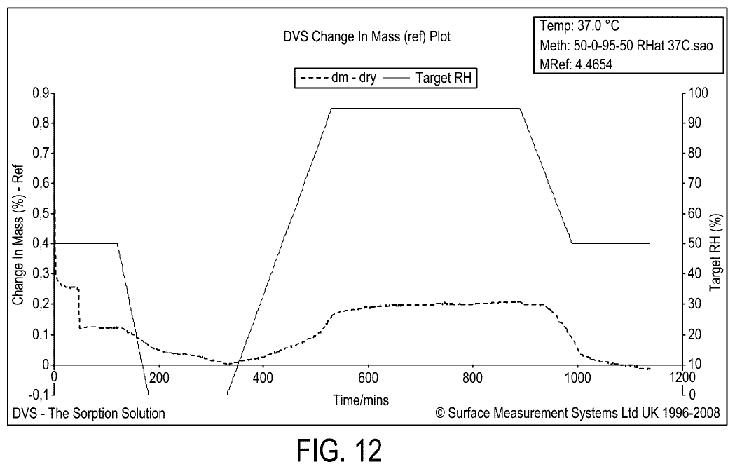

FIG. 12 shows the water sorption/desorption of neutral Compound 1 at 37° C. from 0% to 95% then to 0%. The mass decreased by ˜0.1% w/w as the RH was lowered to 0%. The mass increased by ˜0.2% w/w as the RH was increased to 95%. A slight hysteresis was observed with the final mass at 50% RH (˜0.1 wt-%). The water sorption/desorption isotherms at 37° C. showed a non-hygroscopic compound (<0.2% wt-%).

### 3.5 XRPD Under Temperature and Relative Humidity

### 3.5.1 Method 1

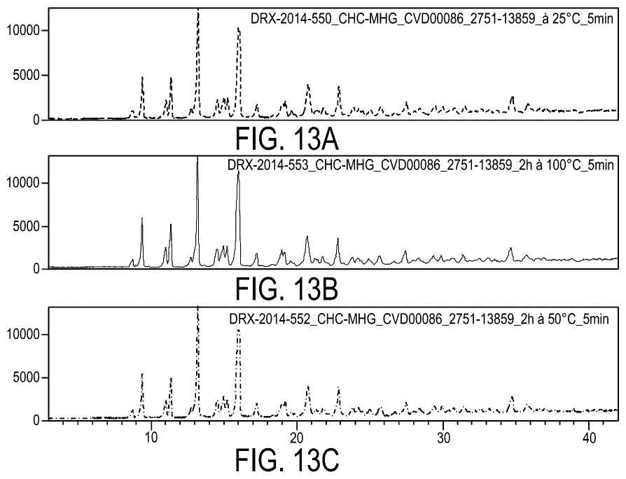

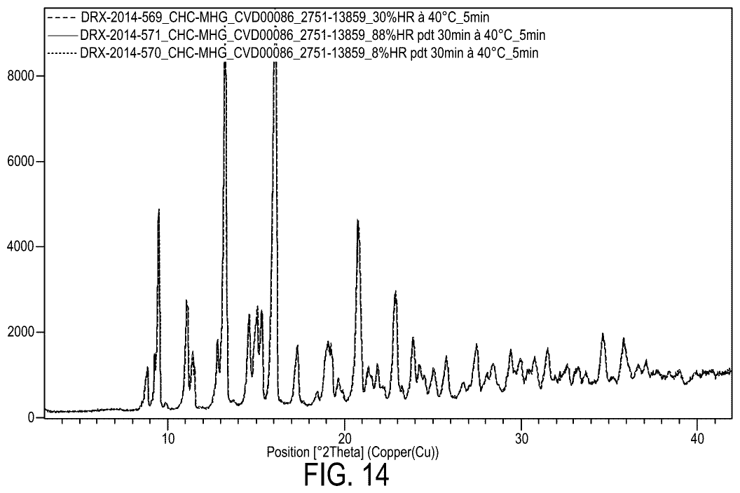

These experiments studied the influence of temperature at ambient RH. RH was wet at 30% in order to simulate a RH near from the ambient RH, and the temperatures studied were 25-50-100° C. FIG. 13 shows the XRPD pattern representing the influence of temperature: FIG. 13A relates to studies at 25° C.; FIG. 13B relates to studies at 100° C.; and FIG. 13C relates to studies at 50° C.

### 3.5.2 Method 2:

These experiments studied the influence on RH at 40° C. The RH studied were 10-30-88%. FIG. 14 shows the XRPD pattern representing the influence of RH at 40° C. No impact of temperature (from 25 to 100° C.) and relative humidity were observed on the free compound.

### 3.6 Solubility

The solubility of neutral Compound 1 in water at 37° C. was found below 1 g/l. This compound can be considered as very slightly soluble (<1 g/L).

### Example 4—Polymorph Screening

### 4.2 General Principles

The formation of polymorphs is influenced mainly by three parameters, in descending order:
1. Interfacial energy 7 between the nucleus and the solution; the chemical nature of the solvent and the presence of impurities largely influence its value
2. Supersaturation ratio; and
3. Temperature T.

The first step consisted in modifying the interfacial energy 7 by selecting different solvents covering all ranges of polarity and proticity (see Table 9 below).

TABLE 9
Solvents Used in Polymorph Screening

| Solvent | T_{boiling} (° C.) |
|---|---|
| dichoromethane | 43 |
| acetone | 56 |
| methanol | 65 |
| MTBE | 69 |
| ethyl acetate | 77 |
| ethanol | 78 |
| acetonitrile | 81 |
| propan-2-ol | 82 |
| heptane | 98 |
| water | 100 |

The impact of supersaturation was assessed during the generation of any crystals obtained by evaporation, cooling and anti-solvent experiments. Two opposite conditions (weak and strong supersaturation) were selected to generate the solid.

The temperature was the last parameter investigated (low and high), since the nucleation kinetics radically changes with temperature.

The slurry experiments were performed to detect solvent mediated phase transition in the range of temperature studied.

Solid-solid transition was studied by cycling temperature by DSC.

Any isolated solid was analyzed by XRPD. The diffraction patterns were compared with the starting material (Compound 1 Form III). Any new crystalline form (either pure or mixture) was numbered for identification.

### 4.3 Cooling Experiments

### 4.3.1 Methodology

Solutions of Compound 1 Form III were prepared at respectively 50 and 150 g/L in each solvent. Heat was applied when necessary to get a solution. Once a solution was obtained, the vials were cooled down to 10° C. Table 10 summarizes the results of the cooling experiments.

TABLE 10
Summary of Cooling Experiments

| | | | Form (Suspension |
|---|---|---|---|
| [C.] (g/L) | Solvent | Result | Analysis) |
| 50 | water | not soluble | Form III |
| 150 | dichloromethane | no solid | No solid |
| 50 | ethanol | not soluble | Form I |
| 50 | methanol | not soluble | Form III |
| 50 | acetone | not soluble | Form IV |
| 50 | ethyl acetate | not soluble | Form III |
| 50 | MTBE | not soluble | Form III |
| 50 | acetonitrile | not soluble | Form III |
| 50 | heptane | not soluble | Form III |
| 50 | propan-2-ol | not soluble | Form V |

### 4.3.2 Results

Four crystalline phases (pure or mixtures) were obtained by cooling experiments: Form I, Form III, Form IV and Form V. Forms IV and V, being new crystalline phases, they could likely be explained by solvent inclusion. With the exception of dichloromethane, Form III exhibited low solubility even at high temperature in all the solvents tested.

### 4.4 Evaporation Experiments

Solutions of Compound 1 Form III were prepared at respectively 50 and 150 g/L in each solvent. Heat was applied when necessary to get a solution. The evaporation rate was determined according to the behavior of the tested compound under evaporation. The main results of the evaporation experiments are gathered in Table 11 below. No new crystalline phase was obtained by evaporation experiments due to the low solubility of the compound. The only soluble solvent (dichloromethane) led to amorphous solids.

TABLE 11
Summary of Evaporation Experiments

| [C.] (g/L) | Solvent | Result |
|---|---|---|
| 50 | water | not soluble |
| 50 | dichloromethane | amorphous |
| 150 | | |
| 50 | ethanol | not soluble |
| 50 | methanol | not soluble |
| 50 | acetone | not soluble |
| 50 | ethyl acetate | not soluble |
| 50 | MTBE | not soluble |
| 50 | acetonitrile | not soluble |
| 50 | heptane | not soluble |
| 50 | propan-2-ol | not soluble |

### 4.5 Anti-Solvent Experiments

### 4.5.1 Methodology

Solutions of Compound 1 Form III were prepared at 100 g/L in dichoromethane at ambient temperature. An anti-solvent was selected according to its miscibility and then added until the formation of crystals. The addition was pursued even if phase separation occurred. If no solution was achieved, the experiment was stopped. When necessary, the solutions were further cooled down to 10° C. or below to obtain sufficient amounts of solid for characterization by XRPD.

### 4.5.2 Results

The main results of the anti-solvent experiments are gathered in Table 12 below. Two crystalline phases Forms II and IV (pure or mixture) were obtained by anti-solvent experiments including a new one (Form IV).

TABLE 12
Summary of Anti-Solvent Experiments

| [C.] (g/L) | Solvent | Anti-Solvent | Form |
|---|---|---|---|
| 100 | dichloromethane | heptane | Form II |
| 100 | dichloromethane | water | no solid |
| 100 | dichloromethane | MTBE | Form II |
| 100 | dichloromethane | acetonitrile | Form II |
| 100 | dichloromethane | ethyl acetate | Form IV |
| 100 | dichloromethane | acetone | Form IV |
| 100 | dichloromethane | propan-2-ol | no solid |

### 4.6 Slurry Experiments

### 4.6.1 Methodology

Suspensions of Compound 1 Form III were prepared at 100 g/L in each solvent at ambient temperature. The suspensions were stirred at least 2 weeks at room temperature. At the end of this period, the solids obtained were analyzed by XRPD.

### 4.6.2 Results

The main results of the slurry experiments were gathered in Table 13 below. Four crystalline phases (pure or mixtures) were obtained by slurry experiments: Forms I, III, IV and a new one VI (coming from MTBE).

TABLE 13
Summary of Slurry Experiments

| [C.] (g/L) | Solvent | Result |
|---|---|---|
| 50 | water | Form III |
| 50 | dichloromethane | no solid |
| 150 | | |
| 50 | ethanol | Form I |
| 50 | methanol | Form III |
| 50 | acetone | Form IV |
| 50 | ethyl acetate | Form III |
| 50 | MTBE | Form VI |
| 50 | acetonitrile | Form III |
| 50 | heptane | Form III |
| 50 | propan-2-ol | Form V |

### 4.7 Temperature Cycling Experiments (DSC)

Due to concomitant melting and decomposition phenomena, temperature cycling experiments were not relevant: temperature is usually cycled below and above the melting point. Different heating ramps were tested to separate both phenomena but without success.

### 4.8 Summary

Table 14 below summarizes the different forms observed during the polymorph screening.

TABLE 14
Summary of crystalline forms for Compound 1

| Crystalline | Crystalline Purity | Solvation State |
|---|---|---|
| Form | (DSC/XRPD) | (TGA/NMR) |
| I | may be a mixture | solvent inclusion |
| II | may be a mixture | solvent inclusion |
| III | pure | anhydrous |
| IV | may be a mixture | solvent inclusion |
| V | may be a mixture | solvent inclusion |
| VI | may be a mixture | solvent inclusion |

The diffraction patterns, TGA and NMR spectra corresponding to each form were gathered in assessment.

The six crystalline forms were characterized by XRPD and are described below.

FIG. 15 shows the XRPD data for Compound 1 Form I.

FIG. 16 shows the XRPD data for Compound 1 Form II.

FIG. 17 shows the XRPD data for Compound 1 Form III.

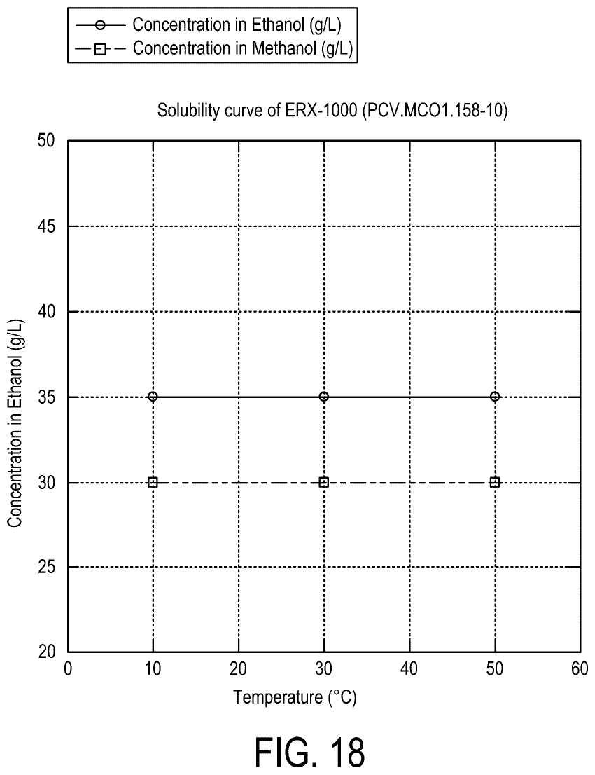

FIG. 18A shows the XRPD data for Compound 1 Form IV and FIG. 22B shows the NMR spectra of Compound 1 Form IV produced by dissolving in dichloromethane and some ethyl acetate as anti-solvent. The solvent content (dichloromethane) after drying was 7.4%.

FIG. 19A shows the XRPD data for Compound 1 Form V and FIG. 23B shows the NMR spectra of Compound 1 Form V produced by slurry in propan-2-ol. The solvent content (propan-2-ol) after drying was 6.1%.

FIG. 20A shows the XRPD data for Compound 1 Form VI and FIG. 24B shows the NMR spectra of Compound 1 Form VI produced by slurry in MTBE. The solvent content (MTBE) after drying was 8.3%.

### Example 5. Solid state characterization of Compound 1 Form I

### 5.1 Appearance (Optical Microscope and Scanning Electron Microscope)

The product presents itself as a red powder as shown in FIG. 25A. It exhibits a platelet habit with the presence of agglomerates.

### 5.2 Crystallinity (XRPD)

FIG. 21B shows the XRPD pattern of Compound 1 Form I. The diffraction pattern is representative of a crystalline powder with high diffraction intensities.

### 5.3 Thermal Analysis (DSC/TGA)

FIG. 26A shows the DSC experiments showing an endothermic peak (onset) at 157° C. with an enthalphy of energy of −50 J/g followed by an exothermic peak. An exothermic peak occurred at 210° C. (onset). The TGA results showed a loss in weight of 4.6% during the endothermic peak of compound. FIG. 22B shows data from DSC and TGA analyses of Compound 1 Form I. The DSC curve exhibited an endothermic peak (onset) at 155° C. This endothermic peak could correspond to a first degradation of the compound accompanied by a slight loss in weight (solvent inclusion). An exothermic peak was observed at 222° C. which could be explained by a recrystallization. The TGA curve showed a loss in weight during the endothermic peak (concomitant phenomena) of around 4.7% w/w. A second higher loss in weight corresponding to 20% occurred above 300° C. This should correspond to the main degradation of the compound.

### 5.4 Hygroscopicity (DVS)

The hygroscopicity experiments were carried out with a DVS Intrinsic from S.M.S. This technique is designed to accurately measure a sample's change in mass as it sorbs precisely controlled concentrations of water vapors in an air carrier gas. All the methods used for DVS experiments are summarized below, and it should be noticed that the temperature and relative humidity (RH) are pre-set according to the initial temperature and RH.

### Method (37° C.)

Equilibrating 120 min at 50% RH
Decreasing to 0% RH in 60 min
Equilibrating 150 min at 0% RH
Increasing to 95% RH in 200 min
Equilibrating 360 min at 95% RH
Decreasing to 50% RH in 100 min
Equilibrating 150 min at 50% RH

FIG. 27A shows the water sorption/desorption of Compound 1 Form I at 37° C. from 0% to 95% then to 50%. The mass decreased by about 0.06% w/w as RH was lowered to 0% and then increased by about 0.09% w/w as the RH was increased to 95%. No hysteresis was observed with the final mass at 50% RH. Thus, Compound 1 of Form I is a non-hygroscopic compound (water uptake <0.2% w/w). FIG. 23B also shows the water sorption/desorption of Compound 1 Form I at 37° C. from 0% to 95% then to 50%. The water sorption/desorption isotherms at 37° C. showed a non-hygroscopic compound (<0.2% w/w). The mass decreased by 0.12% w/w as the RH was lowered to 0%. The mass increased by ˜0.19% w/w as the RH was increased to 95%. A slight hysteresis was observed with the final mass at 50% RH. 5.5 XRPD Chamber under relative humidity and temperature

Using Method 1, the influence of temperature at ambient RH was studied. RH was set at 30% in order to simulate a RH near from the ambient RH, and the temperatures studied were 25-50-100° C. FIG. 24 provides the XRPD pattern representing the influence of temperature on Compound 1 Form I.

Using Method 2, the influence on RH at 40° C. was studied. The RH studied were 10-30-90%. FIG. 25 shows the XRPD pattern representing the influence of RH at 40° C. on Compound 1 Form I. No impact of temperature (from 25 to 100° C.) and relative humidity at 40° C. (from 10% to 90% RH) was observed on this batch, which indicated a good stability of this crystalline phase.

### 5.6 Solubility Study

The solubility of Compound I Form I in various solvents and temperature systems are shown in the following Table 15.

TABLE 15
Solubility Study of Compound I and Form I

| Solvent | Temperature (° C.) | Solubility (g/L) |
|---|---|---|
| CH₂Cl₂ | 25 | S > 120 |
| CH₂Cl₂/EtOH 90/10 | 10 | S >> 40 |
| CH₂Cl₂/EtOH 70/30 | 10 | S >> 40 |
| CH₂Cl₂/EtOH 50/50 | 10 | S >> 40 |
| CH₂Cl₂/EtOH 30/70 | 10 | 25 < S < 45 |
| CH₂Cl₂/EtOH 10/90 | 10 | 11 < S < 15 |
| CH₂Cl₂/EtOH 10/90 | 40 | S < 25 |
| CH₂Cl₂-2/EtOH 20/80 | 40 | 31 < S < 40 |
| Ethyl acetate | 10 | 20 < S < 40 |
| Propan-2-ol | 10 | S < 25 |
| Propan-2-ol | 50 | 25 < S < 40 |
| Ethanol | 25 | 9 < S < 14 |
| MeOH | 50 | S~36 |
| MeOH | 10 | 8 < S < 11 |
| MeOH/Water 67/33/V | 10 | S < 2 |
| Water/EtOH 50/50 | 10 | S < 11 |
| Water/EtOH 50/50 | 25 | S < 11 |

### 5.7 Summary

The physico-chemical properties of Compound I Form I are summarized as follows:
Appearance: Red powder, platelet habit with presence of agglomerates
XRPD (crystallinity): Crystalline phase with high diffraction intensities
Thermal analysis (DSC): endothermic peat at around 157° C. (onset)
Thermal analysis (TGA): decrease in weight about 4.600 during endothermic peak
DVS (hygroscopicity): Non-hygroscopic compound
XPRD under temperature and relative humidity: no new crystalline phase until 100° C. and no new crystalline phase from 10 to 90% RH

### Example 6. Solid State Characterization of Compound 1 Form II

### 6.1 Appearance (Optical Microscope and Scanning Electron Microscope)

The product presents itself as a red powder as shown in FIG. 30A. It exhibits a platelet habit with the presence of agglomerates.

### 6.2 Crystallinity (XRPD)

FIG. 26B shows the XRPD pattern of Compound 1 Form II. The diffraction pattern is representative of a crystalline powder with high diffraction intensities.

### 6.3 Thermal Analysis (DSC/TGA)

FIG. 27 shows data for the thermal analyses (DSC, TGA) of Compound 1 Form II. The DSC curve exhibited an endothermic peak (onset) at 155° C. This endothermic peak could correspond to a first degradation of the compound accompanied by a slight loss in weight (solvent inclusion). An exothermic peak was observed at 213° C. which could be explained by a recrystallization. The TGA curve showed a loss in weight during the endothermic peak (concomitant phenomena) of around 7.2% w/w. A second higher loss in weight corresponding to 16% occurred above 300° C. This should correspond to the main degradation of the compound. 6.4 Hygroscopicity (DVS)

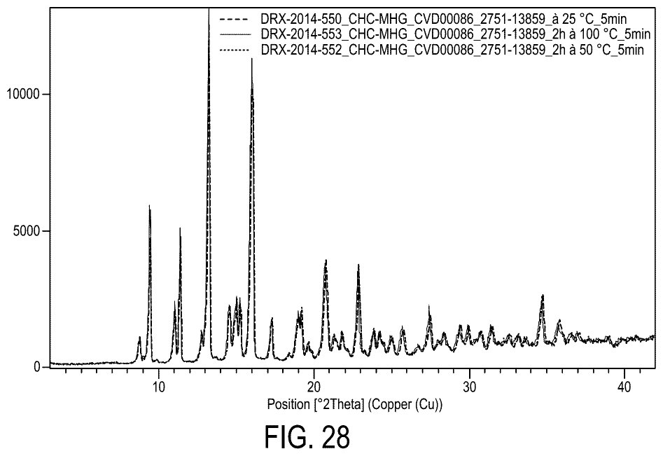

FIG. 28 shows the water sorption/desorption of Compound 1 Form II at 37° C. from 0% to 95% then to 0%. The water sorption/desorption isotherms at 37° C. showed a non-hygroscopic compound (<0.2% w/w). The mass decreased by ˜0.08% w/w as the RH was lowered to 0%. The mass increased by about 0.13% w/w as the RH was increased to 95%. No hysteresis was observed with the final mass at 50% RH.

### 6.5 XRPD Chamber Under Relative Humidity and Temperature

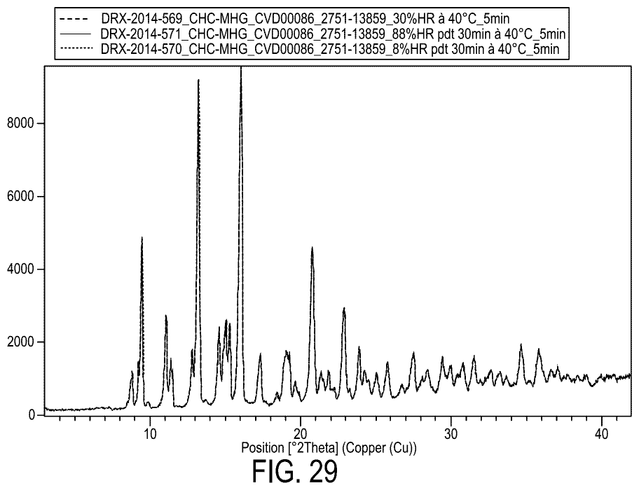

Using Method 1, the influence of temperature at ambient RH was studied. RH was set at 30% in order to simulate a RH near from the ambient RH. Temperature studied: 25-50-100° C. FIG. 29 shows the XRPD pattern representing the influence of temperature on Compound 1 Formula II.

Using Method 2, the influence on RH at 40° C. was studied at 10-30-90%. FIG. 30 shows the XRPD pattern representing the influence of RH at 40° C. on Compound 1 Form II. No impact of temperature (from 25 to 100° C.) and relative humidity at 40° C. (from 10% to 90% RH) was observed on this batch, which indicated a good stability of this crystalline phase.

### Example 7. Characterization of Form III

### 7.1 Starting Material

The starting material for the present experiments is Compound 1, Form III.

### 7.1.1 Solid State Characterization (Appearance)

The product presents itself as a red powder as shown in FIG. 15A. It exhibited a platelet habit with the presence of agglomerates.

### 7.1.2 Crystallinity (XRPD)

The analysis was performed on a PHILIPS PANalytical, X'Pert Pro Diffractometer. The experimental conditions are the following:
Configuration Bragg-Brentano: 0-0
Source: Cu with Kα1 at 1=1.5406 Å.
The power supply works at 45 kV and 40 mA.
Detector: PSD X'Celerator in scanning mode
Sample temperature: room temperature
Angular variation: from 20=3° to 450
Step size [° 2θ]: 0.080
Scan step time (in s): 13.35
Duration: 10 min

A few mg of sample was prepared on a silicon plate without any special treatment other than a slight pressure to obtain a flat and homogeneous surface for reproducible and repeatable results. The diffraction pattern shown in FIG. 31B is representative of a crystalline powder with high diffraction intensities.

### 7.1.3 Thermal Analysis (DSC, TGA)

### 7.1.3.1 Methodology

The DSC analyses were performed on a Mettler Toledo DSC822e and the TGA analyses on a Mettler Toledo TGA/SDTA851. The methodology used for the characterization is described below. A differential scanning calorimeter (Model Mettler Toledo DSC822) was used. The experimental conditions are summarized below.
Accurate amount of compound (mg scale) was packed in aluminum pans (40 μL), crimped with lids having pinholes.
Heating rate 20.00° C./min under nitrogen purge (40 mL/min)
Heating range from 30 to 400° C.

### 7.1.3.2 Results

FIG. 32 provides thermal analyses (DSC and TGA) of Compound 1 Form III. The DSC experiments showed an exothermic peak (onset) at 214.80° C. with an enthalpy of energy of −114 J/g followed by an exothermic peak. The TGA results showed a loss in weight of 0.5% during the exothermic peak of compound.

### 7.1.4 Hygroscopicity (DVS)

The hygroscopicity experiments were carried out with a DVS Intrinsic from S.M.S. This technique is designed to accurately measure a sample's change in mass as it sorbs precisely controlled concentrations of water vapors in an air carrier gas. All the methods used for DVS experiments are summarized below. It should be noticed that the temperature and relative humidity (RH) are pre-set according to the initial temperature and RH.

### Method (37° C.):

Equilibrating 120 min at 50% RH
Decreasing to 0% RH in 60 min
Equilibrating 150 min at 0% RH
Increasing to 95% RH in 200 min
Equilibrating 360 min at 95% RH
Decreasing to 50% RH in 100 min
Equilibrating 150 min at 50% RH

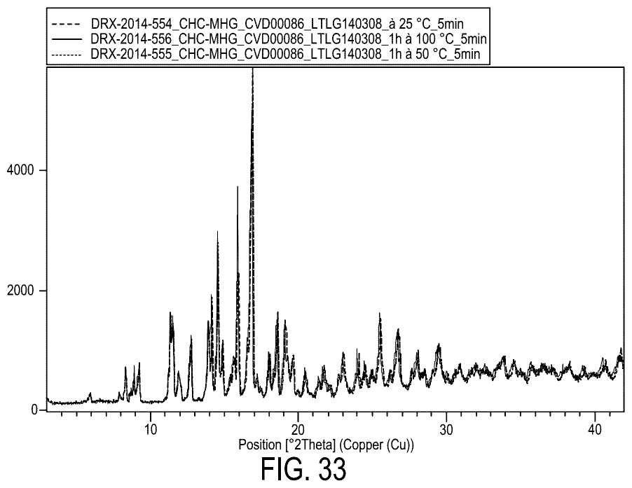

FIG. 33 shows the water sorption/desorption of Compound 1 Form III at 37° C. from 0% to 95% then to 0%. The water sorption/desorption isotherms at 37° C. showed a non-hygroscopic compound (<0.2%). The mass decreased by ˜0.06% as RH was lowered to 0% and then increased by ˜0.09% as RH was increased to 95%. No hysteresis was observed with the final mass at 50% RH.

### 7.1.5 Solubility Profile

The solubility of Compound 1 Form III was measured in the following solvents: water, ethanol and methanol. The determination of the solubility profile was performed with a Thermomixer Eppendorf driving the temperature and the mixing. The stock solutions were prepared according to the USP (United States Pharmacopeia), and then about 1 mL of these stock solutions was added to the API in tubes of 2 mL. The results obtained are gathered in the Table 16 below.

TABLE 16
Solubility Profile

| Solvents | Temperature (° C.) | Solubility (g/L) | Observation |
|---|---|---|---|
| water | 10 | <1 | no color |
| | 30 | | |
| | 50 | | |
| ethanol | 10 | 33-38 | XRPD analysis; phase |
| | 30 | 33-38 | transition towards |
| | 50 | 33-38 | Form I |
| methanol | 10 | 27-<33 | no color |
| | 30 | 27-33 | |
| | 50 | 27-33 | |

The solubility curves of Compound 1 Form III exhibit a linear solubility versus the temperature whether in methanol or ethanol.

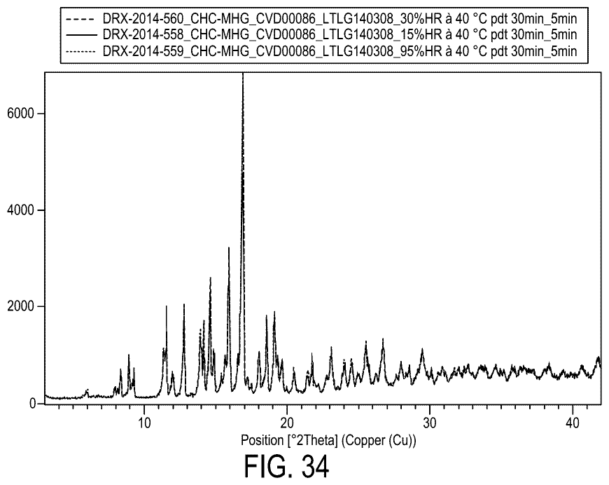

The solubility curves corresponding to the 2 solvents ethanol and methanol are shown in FIG. 34.

### 7.1.6 pH Solubility Profile at 37° C.

All the buffer solutions were prepared according to the U.S.P namely hydrochloric acid buffer for pH=2.0, acid phthalate buffer for pH=4.0, phosphate buffer for pH=6.0, alkaline borate buffer for pH=8.0, which correspond to the native pH. At the end of the solubility study, the pH of the solutions were controlled again (final pH) to determine any variation. The solubility was measured at 37° C. The maximum concentration limit was set at 100 g/L max to minimize consumption of the tested compound.

Table 17 shows the pH solubility profile at 37° C. of Compound 1 Form III. Compound 1 Form III exhibits a very slight solubility from pH=2.0 to 8.0. It should be noticed an orange coloration at pH=8.0 which could indicate a degradation or a very partial solubilization of the tested compound.

TABLE 17
pH Solubility Profile

| | | Solubility | | | |
|---|---|---|---|---|---|
| Temperature | Description | range | Native | Final | |
| (° C.) | form⁽¹⁾ | (g/L) | pH | pH | Color |
| 37 | Very slightly | <1 | 2.0 | 1.90 | colorless |
| | soluble | | 4.0 | 4.03 | colorless |
| | | | 6.0 | 6.03 | colorless |
| | | | 8.0 | 8.09 | Orange |

### 7.1.7 Summary of Compound I Form II Characterization

The physico-chemical properties of an initial batch of Form III are gathered in Table 18 below.

TABLE 18
pH Solubility Profile Tests

| | Observation |
|---|---|
| Appearance (OM/SEM) | Red powder |
| | Platelet habit with presence of agglomerates |
| XRPD (crystallinity) | Crystalline phase with high diffraction |
| | intensities |
| Thermal analyses | DSC: Exothermic peak at 214° C. (onset) |
| (DSC/TGA) | TGA: Loss in weight −0.5% during |
| | exothermic peak |
| DVS (hygroscopicity) | Non hygroscopic compound |
| Solubility profile | Water: Very slightly soluble |
| | Ethanol: Soluble |
| | Methanol: Sparingly Soluble |
| pH profile at 37° C. | From pH = 2 to pH = 8: very slightly soluble |

### Example 8. Summary

Table 19 below summarizes the assessment of the crystalline solid Forms I, II, and II of Celastrol.

TABLE 19
Summary of Compound 1 Form I, Form II, and Form III assessment

| | FI | FII | FIII |
|---|---|---|---|
| XRPD | Highly Crystalline | Highly Crystalline | Highly Crystalline |
| OM | Red powder | Red powder | Red-orange powder |
| | Platelet habit | Platelet habit | Platelet habit |
| Thermal analyses | DSC: | DSC: | DSC: |
| (DSC/TGA) | Endothermic peak at | Endothermic peak at | No endothermic peak |
| | 155° C. (onset) | 155° C. (onset) | Exothermic peak around |
| | Exothermic peak around | Exothermic peak around | 210° C. (onset) |
| | 210° C. (onset) | 210° C. (onset) | TGA: |
| | TGA: | TGA: | 0.5% weight loss during |
| | 4.7% weight loss at 150° C. | 7% weight loss at 150° C. | exothermic peak |
| DVS | Not hygroscopic | Not hygroscopic | Not hygroscopic |
| (Hygroscopicity) | | | |
| XRPD under | Under Temperature: | Under Temperature: | Under Temperature: |
| temperature | 25° C., 50° C. | 25° C., 50° C. | 25° C., 50° C. |
| and RH | and 100° C. | and 100° C. | and 100° C. |
| | =>No new crystalline | =>No new crystalline | =>No new crystalline |
| | phase until 100° C. | phase until 100° C. | phase until 100° C. |
| | Under relative humidity | Under relative humidity | Under relative humidity |
| | (RH): | (RH): | (RH): |
| | 30%, 60% and 90% RH | 30%, 60% and 90% RH | 30%, 60% and 90% RH |
| | =>No new crystalline | =>No new crystalline | =>No new crystalline |
| | phase from 30 to 90% RH | phase from 30 to 90% RH | phase from 30 to 90% RH |
| Solvent content | >ICH limit for ethanol | >>ICH limit for | <ICH limit for methanol |
| | | dichloromethane | |

The crystalline form (Form III) exhibited properties such as highly crystalline, higher thermal stability (stable until 210° C.), nonhygroscopicity, stable under temperature from 25 to 100° C. and relative humidity from 30 to 95% RH, solvent content compliant with ICH standard (≤3000 ppm for methanol, ≤5000 ppm for ethanol, ≤600 ppm for dichloromethane).

### Example 9. Synthetic Process for Compound 1 Form I

### 8.1 Introduction

Initial work related to methods for the preparation of solid forms of Compound 1 Form I. Crude compound was obtained from Shanghai Standard Biotech (SSB), and the batches were also characterized by high levels of ethanol (ca. 5%). For Compound 1 Form I, the synthesis route was modified with the introduction of purification by chromatography prior to the crystallization step. The crystallization process was revised to improve its robustness in view of the production of material suitable for clinical uses.

### 8.2 Crystallization Process Development for Preclinical Batch Manufacture

As crude Compound 1 Form I from SSB presented high levels of residual ethanol, the process development work was started with the objective of reducing ethanol content to acceptable levels (ICH limit).

### 8.2.1 Solubility Study

The solubility of Compound 1 Form I was assessed in order to identify potential crystallization media. Compound 1 Form I was found very soluble in dichoromethane even at low temperature (10° C.). A dichoromethane/ethanol 50:50 (V/V) mixture provided low solubility at low temperature and relatively good solubility at high temperature.

Experiments were performed in a dichoromethane/ethanol 50:50 (V/V) mixture either by cooling or by evaporation ofdichloromethane followed by cooling. The following Table 20 below summarizes the most representative trials.

TABLE 20
Summary of Crystallization Processes For Compound 1 Form I

| Exper- | | |
|---|---|---|
| iment | Process/yield | Comments |
| 1 | Solubilization of ERX-1000 | Mixture Form I and Form II |
| | at 45° C. in | (see. Polymorph report) |
| | CH₂Cl₂/EtOH 50/50 (5 V/5 V) | Does not comply with solvent |
| | Concentration at 8 V | content ~4.5% |
| | Cooling at 10° C. | |
| | Yield ~60% | |
| 2 | Solubilization of ERX-1000 | Mixture Form I and Form II |
| | at 45° C. in | (see. Polymorph report) |
| | CH₂Cl₂/EtOH 50/50 (5 V/5 V) | Does not comply with solvent |
| | Concentration at 6 V | content ~4.5% |
| | Cooling at 25° C. | |
| | Yield ~42% | |
| 3 | Solubilization of ERX-1000 | Form I |
| | at 45° C. in | Does not comply with solvent |
| | CH₂Cl₂/EtOH 50/50 (5 V/5 V) | content (4.9%) |
| | Concentration at 3 V | |
| | Adding 1.2 V of EtOH | |
| | Cooling at 10° C. | |
| | Yield ~80% | |

Pure crystalline Compound 1 Form I was finally isolated with satisfactory purity and yield. However, residual solvent content was still above ICH limit. Drying conditions were pushed up to 120° C. which allowed the solvent content to be decreased to 0.65%. As some degradation was observed at this temperature, the further reduction of residual ethanol content did not seem possible by this mean. The different trials with Compound 1 Form I tend to show that we are in presence of solvent inclusion rather than an ethanol solvate: different residual ethanol contents were observed in the different trials without a change in the crystalline phase.

### Example 10. Synthetic Process for Compound 1 Form III

During the process development work on Compound 1 Form I, crystallization was obtained after addition of MeOH to an oil. XRPD analysis indicated the formation of Compound 1 Form III. A specific process to obtain this new form was developed and implemented in a 50 mL reactor. Sufficient amount of solid were isolated to further characterize this new form by DSC/TGA. Compound 1 Form III was found to be an anhydrous, non-solvated form.

### 10.1 Initial Studies

The crystallization process for obtaining Compound 1 Form III was further developed with the addition of water as non-solvent in order to increase the yield. The final process described below was applied for the manufacture of a first pre-clinical batch.

TABLE 21
Summary of Crystallization Processes For Compound
1 Form III - pre-clinical batch A

| Step | Observations |
|---|---|
| Dissolution of crude API (F I) | T_{jacket} = 90° C. |
| in 25 V of MeOH | |
| Concentration at 10 V of MeOH | Nucleation during the |
| | concentration |
| Cooling at 40° C. | |
| Addition of 5 V of Water | improves the yield |
| Cooling to 10° C. | |
| Step 15 h at 10° C. | Consumption of supersaturation |
| Filtration under vacuum | Fast filtration |
| Drying under vacuum at 50° C. | Yield: 90% with 99.8% purity (HPLC) |

Prior being used at bigger scale, this process was tested at small scale (from 2 to 20 g) and led to the desired form (Form III) and a good purity (99.8%) (pre-clinical batch A).

### 9.2 First Scale-Up

The crystallization process was successfully scaled-up twice at 400 g scale to produce a preclinical batch (batch size: 732 g). The material was of the intended crystalline form (Compound 1 Form III) with a satisfactory chemical purity 99.7% and compliant residual solvent content (MeOH=1162 ppm; dichloromethane <10 ppm, ethyl acetate <10 ppm, and EtOH <10 ppm). Yield was very high (around 90%).

### 9.3 Second Scale-Up

Further studies using additional batches led to the identification that degradation in solution during the crystallization process might occur. The purity of the mother liquor was verified by HPLC all along the process and several samples were taken during the concentration step. The objective was to determine whether or not some degradation could occur during the process in the mother liquor and explain the lower yield.

The experiment was performed on 20 g scale and the results are summarized in Table 22 below.

TABLE 22
Chemical Purity of Compound 1 Form III During Crystallization

| Time | Purity by | |
|---|---|---|
| points | HPLC (%) | Comments |
| T₀ | Σᵢₘₚ = 0.23 | From solution. Just |
| | | before the concentration |
| T₁ | Σᵢₘₚ = 1.7 | After 2 h at reflux |
| T₂ | Σᵢₘₚ = 3.4 | After 4 h at reflux |
| T₃ | Σᵢₘₚ = 11.1 | At the end of the |
| | | concentration |
| T₄ | Σᵢₘₚ = 92.4 | On mother liquor |
| T₅ | Σᵢₘₚ = 3.5 | Solid isolated |

This experiment revealed a significant degradation of Compound 1 Form III during the concentration step with up to 11.1% of impurities formed. With a final purity of the solid of 96.5%, it appeared that the crystallization process was quite effective at removing the degradation products.

Some experiments were first performed at small scale (2 g) by slurry in 15V of a mixture MeOH/Water 67:33 (V:V); this retreatment of the isolated solid from T₅ of Table 22 (which had 96.5% purity) led a compound of high purity (99.8%) with a satisfactory yield (92%) (pre-clinical batch B).

TABLE 23
Further Experiments for the Purification of
Compound 1 Form III - pre-clinical batch B

| Step | Observations |
|---|---|
| Addition of 15 V of a mixture | T_{jacket} = 20° C. |
| MeOH/Water 67/33 V/V | |
| Step 3 h at 20° C. | |
| Filtration under vacuum | Fast filtration |
| Drying under vacuum at 50° C. | Yield: 92% with 99.7% purity (HPLC) |

In another experiment, the volume of solvent used for the slurry was reduced to 3.7 V but this did not sufficiently improve the chemical purity (97.5%). Yield remained satisfactory though (93%). 330 g of Compound 1 Form III were obtained this way. Purity by HPLC was 99.7% and the yield was 92%.

### Example 11. Crystallization Process Development for Clinical Batch Manufacture

The main drawback of the existing crystallization process being the degradation of Compound 1 Form III during the concentration step, two alternatives were investigated: concentration under vacuum and crystallization only by cooling. The material used was crude Compound 1 Form I.

### 10.1 Crystallization by Concentration Under Vacuum.

A 2 g scale trial with concentration step under vacuum showed that degradation was significantly reduced by this mean: purity decreased from 99.8% to 99.4% during the concentration. Almost another ten experiments were performed with concentration under vacuum as per the process described below in Table 24.

TABLE 24
Crystallization Experiments

| Step | Observations |
|---|---|
| Solubilisation of ERX-1000 (FI) in at | |
| least 25 V of MeOH at Tₘₐₛₛ = 65° C. | |
| Concentration under vacuum at 10 V | |
| (Tₘₐₛₛ ~45° C. T_{jacket} = 55° C.) | |
| At the end of the concentration, cooling | |
| down to 40° C. | |
| Addition of 15 V of a Water (ratio | |
| MeOH/Water 67/33 V/V) | |
| Cooling down to 10° C. | |
| Step 3 h at 10° C. | |
| Filtration under vacuum | Fast filtration |
| Drying under vacuum at 50° C. | Yield: around 90% |

For 70% of the trials, this process led to the production of pure crystalline phase Compound 1 Form III. The remaining ones conducted to mixtures of Compound 1 Forms I and III. This could be explained by the fact that during the concentration under vacuum, Compound 1 Form I was the one which always crystallized first. Under vacuum, Compound 1 Form I remains the most stable form but when pressure is back to atmospheric, Compound 1 Form I transitions to Compound 1 Form III in presence of water. Should water not reach an amount of Compound 1 Form I solid (e.g. solid that had crystallized on the reactor walls), it will not transition to Compound 1 Form III and instead will lead to some Compound 1 Form I being present among Compound 1 Form III material.

Additional trials were conducted to make this process reproducible in particular be seeding. If seeding improved the number of experiments leading to Compound 1 Form III, it did not prevent the production in some cases of mixtures of Compound 1 Form I and Compound 1 Form III.

In conclusion, at this point, the process with concentration under vacuum led to good yield and purity but failed to isolate the desired crystalline form Compound 1 Form III) in a reproducible manner.

### 10.2 Crystallization by Cooling

The other alternative was to crystallize the Compound 1 Form I directly by cooling in a mixture of MeOH/Water 67/33 (V/V). Several trials were conducted and the representative process can be summarized as follows in Table 25.

TABLE 25
Crystallization by Cooling Experiments

| Step | Observations |
|---|---|
| Solubilization of ERX-1000 (FI) in at | |
| least 27 V of MeOH at Tₘₐₛₛ = 55° C. | |
| Filtration to remove mechanical | The glassware used for |
| impurities at 55° C. | filtration was previously |
| | heated at 55° C. |
| Washing with 1.7 V of MeOH the pipes | |
| Cooling down to 40° C. | |
| Addition of 15 V of Water (ratio | Apparition of orange |
| MeOH/Water 67/33 V/V) | suspension |
| Cooling ramp from 40 to 10° C. (−20° C./h) | |
| Step one night at 10° C. | |
| Filtration under vacuum | Fast filtration |
| | Anhydrous crystalline |
| | form III produced |
| Drying under vacuum at 35° C. | Yield: around 80% |
| | Purity similar to the |
| | starting material |

All experiments performed with this process led to the desired anhydrous crystalline form Compound 1 Form III. It has the advantage to avoid the degradation and save time due to the absence of concentration step. Furthermore, this process is directly scalable to the Pilot Plant.

### Example 12. Introduction of a Purification Step by Chromatography

The introduction of a purification step by chromatography for the manufacture of a Compound 1 clinical batch required the verification of any impact on the final crystallization step. Representative samples were use-tested.

### 11.1 Use Test on Sample 1

The first sample was presenting a new crystalline phase. It was called Form VII and could be considered as an inclusion form cyclohexane, one of the solvents used to isolate Celastrol at the end of the purification by chromatography. The crystallization of this material as per the developed process led to the production of a mixture of crystalline phases Compound 1 Form III and Compound 1 Form VII.

The below represents a process that may be used to prepare a sample for recrystallization and preparation of Compound 1 Form III.

### Process A

Removal of cyclohexane and acetone by concentration under vacuum.
Addition of EtOH to get a solution.
Removal of cyclohexane traces by concentration under vacuum (azeotropic mixture).
Cooling the suspension to improve the yield.
Drying under vacuum.

### 11.2 Use Test on Sample 2

Sample 2 was obtained from a crude Celastrol batch where Process A was followed for its isolation. Its characterization by XRPD confirmed that it was Compound 1 Form I as intended.

The crystallization of this material as per the developed process led to the production of a pure anhydrous crystalline phase Compound 1 Form III with satisfactory yield (around 80%) and purity (100%).

Below are summarized the processes applied and associated purities from the raw material to the crystallized Form III as follows in Table 26.

TABLE 26
Use Test Samples genealogy

| | | Crude | Compound I Form III | Compound I Form III |
|---|---|---|---|---|
| | | celastrol | Chromatography | Crystallization by cooling |
| Process | | N/A | Purification by | Solubilization of ERX- |
| | | | chromatography column | 1000 (FI) in 27.5 V of |
| | | | to get a GMP material | MeOH (36 g/L) at |
| | | | (production of FVII). | Tₘₐₛₛ = 55° C. |
| | | | Retreatment in order to | Filtration to remove |
| | | | obtain the crystalline | mechanical impurities. |
| | | | phase Fl by distillation | Washing with 1.7 V of |
| | | | under vacuum. | MeOH the pipes. |
| | | | | Cooling down to 40° C. |
| | | | | Addition of 15 V of a |
| | | | | Water (ratio MeOH/Water |
| | | | | 67/33 V/V) |
| | | | | Cooling ramp to 10° C. |
| | | | | (−20° C./h) |
| | | | | Step one night at 10° C. |
| | | | | Filtration under vacuum |
| | | | | Drying under vacuum at |
| | | | | 35° C. |
| Yield | | N/A | N/A | 82% |
| Purity (%) | | 99.8 | >99.9 | >99.9 |
| Sum of impurities (%) | | RRT 0.95: 0.24% | <0.08 | <0.08 |
| Crystalline phase | | FI (solvate | FI (solvate | FIII (anhydrous phase) |
| | | of Ethanol) | of Ethanol) | |
| Solvent | Methanol | <10 | <10 | 60 |
| content | Ethanol | 49000 | ~57000 | 13 |
| (ppm) | Dichloro- | 120 | N/A | N/A |
| | methane | | | |
| | Ethyl | 1540 | N/A | N/A |
| | Acetate | | | |

| | | | |
|---|---|---|---|
| Water content (%) | 0.08 | N/A | 0.07 |

Purified Celastrol was of the expected form (Compound 1 Form I) with high chemical quality (RRT 0.95 impurity present in the crude Celastrol batch was significantly reduced).
The re-crystallization step led to the intended crystalline form (Compound 1 Form III) with a satisfactory yield (above 800%). The purity was found very high and the residual solvent contents were within the ICH limits of ≤3000 ppm for methanol, ≤5000 for ethanol, ≤600 for dichloromethane, and ≤5000 for ethyl acetate.
This process was implanted for the manufacture of a Celastrol clinical batch.

### Example 13—Process for Manufacture of Highly Pure Form III

Multiple processes for the purification of crystalline forms of Compound I (e.g., a polymorph of Form III of Compound I) were tested. The starting material was characterized by XRPD, PLM, TGA, DSC and HPLC and was identified as a mixture of Form I and Form III. DSC & TGA results showed a weight loss of ˜2.3% before 200° C. and exhibited an endothermic peak at 134.5° C. (onset). The assay and main purity of the starting material were 88.8% and 98.9%, respectively. Solvent screening results showed that the impurity was significantly rejected a 2-MeTHF/n-Heptane solvent/anti-solvent system, which was then further tested.

Preliminary trials in the 2-MeTHF/n-heptane system achieved significant reduction of impurities, but with a relatively low yield. Subsequently, the volume of anti-solvent was increased and the yield was effectively increased. Moreover, the recrystallization process was introduced to further increase the purity of the product. As a result, a product was obtained in the coupled process with purity as high as 100%. Several stress test experiments showed that the purification process was robust, including no seed loading, faster cooling rate, faster addition rate, etc.

In summary, the purification procedure had been successfully developed in the 2-MeTHF/n-Heptane system. Process demonstration was eventually conducted on 10 g scale, which reliably afforded qualified product in ˜91% yield (calculated by assay). Moreover, this purification process was validated with 1 kg GMP manufacture, in which the properties of the product were all qualified with the specifications.

Exemplary processes and the achieved results (including purity and residual solvent levels determined by HPLC and GCHS, respectively) are summarized in Tables 27-29, below.

TABLE 27
1^{st} purification in exemplary processes A-E

| Process A | Process B | Process C | Process D | Process E |
|---|---|---|---|---|
| Add 2-MeTHF to a | Add starting | Add starting | Add starting | Add starting |
| flask (7.5 V) | material to a flask | material to a flask | material to a flask | material to a flask |
| Add starting | Add 2-MeTHF to | Add 2-MeTHF to | Add 2-MeTHF to | Add 2-MeTHF to |
| material to the flask | the flask (7.5 V) | the flask (15 V) | the flask (7.5 V) | the flask (15 V) |
| Add 0.2 w/w | Add 0.2 w/w | | | |
| activated carbon | activated carbon | | | |
| (F1600) | (F1600) | | | |
| Add 2-MeTHF to | Add 2-MeTHF to | | | |
| wash wall of flask | wash wall of flask | | | |
| (7.5 V) | (7.5 V) | | | |
| Warm resulting | Warm resulting | Warm resulting | Warm resulting | Warm resulting |
| mixture to 75° C. | mixture to 75° C. | mixture to 75° C. | mixture to 75° C. | mixture to 75° C. |
| and stir at 75° C. | and stir at 75° C. | and stir at 75° C. | and stir at 75° C. | and stir at 75° C. |
| until clear solution | until clear solution | until clear solution | until clear solution | until clear solution |
| is obtained | is obtained | is obtained | is obtained | is obtained |
| | | Add 0.5 w/w | Add 0.5 w/w | |
| | | activated carbon | activated carbon | |
| | | (F1600) | (F1600) | |
| | | | Add 2-MeTHF to | |
| | | | wash wall of flask | |
| | | | (7.5 V) | |
| Stir at 75° C. | Stir at 75° C. | Stir at 75° C. | Stir at 75° C. | Stir at 75° C. |
| for ~18 h | for 4~6 h | for 4~6 h | for 2~3 h | for 20~30 min |
| Polish filtration | Polish filtration | Polish filtration | Polish filtration | |
| Rinse with | Rinse with | Rinse with | Rinse with | |
| 2-MeTHF (2 V) | 2-MeTHF (2 V) | 2-MeTHF (2 V) | 2-MeTHF (2 V) | |
| Warm resulting | Warm resulting | Warm resulting | Warm resulting | |
| mixture to 75° C. | mixture to 75° C. | mixture to 75° C. | mixture to 75° C. | |
| and stir at 75° C. | and stir at 75° C. | and stir at 75° C. | and stir at 75° C. | |
| until clear solution | until clear solution | until clear solution | until clear solution | |
| is obtained | is obtained | is obtained | is obtained | |
| Cool to 55° C. | Cool to 60° C. | Cool to 60° C. | Cool to 55~60° C. | Cool to 55~60° C. |
| Add 1 wt % seed | Add 1 wt % seed | Add 1 wt % seed | Add 1 wt % seed | Add 1 wt % seed |
| Age for 0.5 h | Age for 2 h | Age for 0.5 h | Age for 0.5 h | Age for 0.5 h |
| Cool to 25° C.; | Cool to 25° C.; | Cool to 25° C.; | Cool to 25° C.; | Cool to 25° C.; |
| (10° C./h) | (10° C./h) | (10° C./h) | (10° C./h) | (10° C./h) |
| | Stir at 25° C. | | | |
| | for 7.5 h | | | |
| Add n-Heptane | Add n-Heptane | Add n-Heptane | Add n-Heptane | Add n-Heptane |
| (34 V; 6 h) | (34 V; 6 h) | (34 V; 6 h) | (34 V; 6 h) | (34 V; 6 h) |
| Cool to 5° C.; | Cool to 5° C.; | Cool to 5° C.; | Cool to 5° C.; | Cool to 5° C.; |
| (10° C./h) | (10° C./h) | (10° C./h) | (10° C./h) | (10° C./h) |
| Stir at 5° C. | Stir at 5° C. | Stir at 5° C. | Stir at 5° C. | Stir at 5° C. |
| for >8 h | for 11 h | for 12 h | for 11 h | overnight |
| Filter, rinse filter | Filter, rinse filter | Filter, rinse filter | Filter, rinse filter | Filter, rinse filter |
| with n-Heptane (2 V) | with n-Heptane (2 V) | with n-Heptane (2 V) | with n-Heptane (2 V) | with n-Heptane (2 V) |
| Dry under vacuum | Dry under vacuum | Dry under vacuum | Dry under vacuum | Dry under vacuum |
| at 35° C. overnight | at 35° C. overnight | at 35° C. overnight | at 35° C. overnight | at 35° C. overnight |
| 1st purified | 1st purified | 1st purified | 1st purified | 1st purified |
| crystalline form: | crystalline form: | crystalline form: | crystalline form: | crystalline form: |
| Assay: 75.7% | Assay: 77.0% | Assay: 76.1% | Assay: 78.8% | Yield: 90.0% |
| Yield: 78.4% | Yield: 85.0% | Yield: 72.1% | Yield: 85.0% | Purity: 99.96% |
| Purity: 99.93% | Purity: 99.94% | Purity: 99.88% | Purity: 99.96% | 2-Me THF residue: |
| | THF residue: | THF residue: | 2-MeTHF residue: | 18.9% |
| | 19.2% | 18.1% | 17.7% | Heptane residue: |
| | Heptane residue: | Heptane residue: | Heptane residue: | 1.31% |
| | 1.2% | 1.5% | 1.26% | |

TABLE 28
2^{nd} purification in exemplary processes A-E

| Process A | Process B | Process C | Process D | Process E |
|---|---|---|---|---|
| Add 1st purified | Add 2-MeTHF to | Add 2-MeTHF to | Add 1st purified | Add 1st purified |
| crystalline form | the flask (7.5 V) | the flask (7.5 V) | crystalline form | crystalline form |
| from steps 1-6 to a | | | from steps 1-6 to a | from steps 1-6 to a |
| flask; | | | flask; | flask; |
| Add 2-MeTHF to | Add 1st purified | Add 1st purified | Add 2-MeTHF to | Add 2-MeTHF to |
| the flask (15 V) | crystalline form | crystalline form | the flask (15 V) | the flask (15 V) |
| | from steps 1-6 to | from steps 1-6 to | | |
| | a flask; | a flask; | | |
| | Add 0.5 w/w | Add 0.5 w/w | | |
| | activated carbon | activated carbon | | |
| | (F1600) | (F1600) | | |
| | Add 2-MeTHF to | Add 2-MeTHF to | | |
| | wash wall of | wash wall of | | |
| | flask (7.5 V) | flask (7.5 V) | | |
| Warm resulting | Warm resulting | Warm resulting | Warm resulting | Warm resulting |
| mixture to 75° C. | mixture to 75° C. | mixture to 75° C. | mixture to 75° C. | mixture to 75° C. |
| and stir at 75° C. | and stir at 75° C. | and stir at 75° C. | and stir at 75° C. | and stir at 75° C. |
| until clear solution | until clear solution | until clear solution | until clear solution | until clear solution |
| is obtained | is obtained | is obtained | is obtained | is obtained |
| | Stir at 75° C. for | Stir at 75° C. for | | |
| | 4~6 h | 4~6 h | | |
| | Polish filtration | Polish filtration | | |
| | Rinse with 2 V | Rinse with 2 V | | |
| | 2-MeTHF | 2-Me THF | | |
| | Warm resulting | Warm resulting | | |
| | mixture to 75° C. | mixture to 75° C. | | |
| | and stir at 75° C. | and stir at 75° C. | | |
| | until clear solution | until clear solution | | |
| | is obtained | is obtained | | |
| Cool to 55° C. | Cool to 60° C. | Cool to 60° C. | Cool to 55° C. | Cool to 55° C. |
| Add 1 wt % seed | Add 1 wt % seed | Add 1 wt % seed | Add 1 wt % seed | Add 1 wt % seed |
| Age for 0.5 h | Age for 0.5 h | Age for 0.5 h | Age for 0.5 h | Age for 0.5 h |
| Cool to 25° C.; | | | Cool to 25° C.; | Cool to 25° C.; |
| (10° C./h) | | | (10° C./h) | (10° C./h) |
| Add n-Heptane | Add n-Heptane | Add n-Heptane | Add n-Heptane | Add n-Heptane |
| (30 V; 6 h) | (34 V; 6 h) | (34 V; 6 h) | (30 V; 6 h) | (30 V; 6 h) |
| Cool to 5° C.; | Cool to 25° C.; | Cool to 25° C.; | Cool to 5° C.; | Cool to 5° C.; |
| (10° C./h) | (10° C./h) | (10° C./h) | (10° C./h) | (10° C./h) |
| Stir at 5° C. | Stir at 5° C. for | Stir at 5° C. for | Stir at 5° C. | Stir at 5° C. |
| for >8 h | overnight | overnight | for >8 h | for >8 h |
| Filter, rinse filter | Filter, rinse filter | Filter, rinse filter | Filter, rinse filter | Filter, rinse filter |
| with n-Heptane (2 V) | with n-Heptane (2 V) | with n-Heptane (2 V) | with n-Heptane (2 V) | with n-Heptane (2 V) |
| Dry under vacuum | Dry under | Dry under | Dry under | Dry under |
| at 35° C. for | vacuum at 35° C. | vacuum at 35° C. | vacuum at 35° C. | vacuum at 35° C. |
| overnight | for overnight | for overnight | for overnight | for overnight |
| 2nd purified | 2nd purified | 2nd purified | 2nd purified | 2nd purified |
| crystalline form: | crystalline form: | crystalline form: | crystalline form: | crystalline form: |
| Assay: 74.7% | Assay: 79.2% | Assay: 78.3% | Assay: 77.3% | Assay: 79.1% |
| Yield: 85.65% | Yield: 71.0% | Yield: 71.1% | Yield: 76.9% | Yield: 82.1% |
| Purity: 99.99% | THF residue: | Purity: 100.0% | Purity: 99.98% | Purity: 100.0% |
| 2-Me THF residue: | 18.5% | THF residue: | 2-Me THF residue: | 2-Me THF residue: |
| 16.84% | Heptane residue: | 18.8% | 20.69% | 17.50% |
| Heptane residue: | 0.82% | Heptane residue: | Heptane residue: | Heptane residue: |
| 1.70% | | 1.12% | 0.78% | 1.67% |

TABLE 29
Crystallization transformation in exemplary processes A-E

| Process A | Process B | Process C | Process D | Process E |
|---|---|---|---|---|
| Add 2nd purified | Add 2nd purified | Add 2nd purified | Add 2nd purified | Add 2nd purified |
| crystalline form to a | crystalline form to | crystalline form to | crystalline form to | crystalline form to |
| flask | a flask | a flask | a flask | a flask |
| Add MeOH to the | Add MeOH to the | Add MeOH to the | Add MeOH to the | Add MeOH to the |
| flask (27.5 V) | flask (27.5 V) | flask (27.5 V) | flask (27.5 V) | flask (27.5 V) |
| Warm resulting | Warm resulting | Warm resulting | Warm resulting | Warm resulting |
| mixture to 55° C. | mixture to 55° C. | mixture to 55° C. | mixture to 55° C. | mixture to 55° C. |
| and stir at 55° C. | and stir at 55° C. | and stir at 55° C. | and stir at 55° C. | and stir at 55° C. |
| until clear solution | until clear solution | until clear solution | until clear solution | until clear solution |
| is obtained | is obtained | is obtained | is obtained | is obtained |
| Filter the reaction | Filter the reaction | Filter the reaction | Filter the reaction | Filter the reaction |
| mixture at 55° C. | mixture at 55° C. | mixture at 55° C. | mixture at 55° C. | mixture at 55° C. |
| Rinse filter cake | Rinse filter cake | Rinse filter cake | Rinse filter cake | Rinse filter cake |
| with MeOH (1.7 V) | with MeOH (1.7 V) | with MeOH (1.7 V) | with MeOH (1.7 V) | with MeOH (1.7 V) |
| Cool to 40° C. | Cool to 40° C. | Cool to 40° C. | Cool to 40° C. | Cool to 40° C. |
| Slowly (dropwise) | Slowly (dropwise) | Slowly (dropwise) | Slowly (dropwise) | Slowly (dropwise) |
| add H₂O | add H₂O | add H₂O | add H₂O | add H₂O |
| 40~45° C. (5 V) | 40~45° C. (3.1 V) | 40~45° C. (3.1 V) | 40~45° C. (3.1 V) | 40~45° C. (3.1 V) |
| Add 1 wt % seed | Add 1 wt % seed | Add 1 wt % seed | Add 1 wt % seed | Add 1 wt % seed |
| Age for 0.5 h | Age for 0.5 h | Age for 0.5 h | Age for 0.5 h | Age for 0.5 h |
| Slowly (dropwise) | Slowly (dropwise) | Slowly (dropwise) | Slowly (dropwise) | Slowly (dropwise) |
| add H₂O at | add H₂O at | add H₂O at | add H₂O at | add H₂O at |
| 40~45° C. (10 V) | 40~45° C. (11.9 V) | 40~45° C. (11.9 V) | 40~45° C. (11.9 V) | 40~45° C. (11.9 V) |
| Cool to 10° C. | Cool to 10° C. | Cool to 10° C. | Cool to 10° C. | Cool to 10° C. |
| (20° C./h) | (20° C./h) | (20° C./h) | (20° C./h) | (20° C./h) |
| Stir at 10° C. | Stir at 10° C. | Stir at 10° C. | Stir at 10° C. | Stir at 10° C. |
| for 18 h | overnight | overnight | overnight | overnight |
| Filter, rinse filter | Filter, rinse filter | Filter, rinse filter | Filter, rinse filter | Filter, rinse filter |
| cake with | cake with | cake with | cake with | cake with |
| MeOH/H₂O = 2:1 (2 V) | MeOH/H₂O = 2:1 (2 V) | MeOH/H₂O = 2:1 (2 V) | MeOH/H₂O = 2:1 (2 V) | MeOH/H₂O = 2:1 (2 V) |
| Dry under vacuum at | Dry under vacuum | Dry under vacuum | Dry under vacuum | Dry under vacuum |
| 35° C. overnight | at 35° C. overnight | at 35° C. overnight | at 35° C. overnight | at 35° C. overnight |
| Product: | Product: | Product: | Product: | Product: |
| Assay: 98.9% | Assay: 99.5% | Assay: 98.1% | Assay: 98.8% | Assay: 100.2%, |
| Yield: 94.5% | Yield: 93.4% | Yield: 87.5% | Yield: 89.0% | Yield: 92.5% |
| Purity: 100.0% | Purity: 100.0% | Purity: 100.0% | Purity: 99.89% | Purity: 100.0% |
| MeOH residue: | MeOH residue: | MeOH residue: | H₂O content: 0.17% | H₂O: 0.20% |
| 706 ppm | 1095 ppm | 1318 ppm | MeOH residue: | MeOH residue: |
| 2-Me THF: 275 ppm | 2-Me THF: 154 cppm | 2-Me THF: 130 ppm | 1395 ppm | 1022 ppm |
| n-heptane: 65 ppm | n-heptane: 15 ppm | n-heptane: 13 ppm | 2-Me THF: 157 ppm | 2-Me THF: 105 ppm |
| | | | n-heptane: 9 ppm | n-heptane: 21 ppm |

Without wishing to be bound by theory, XRPD tests suggested that the products obtained in the 2-MeTHF or 2-MeTHF/n-Heptane systems (i.e., the first and second purified crystalline form) were crystalline Form IV of Compound I. The final products after crystallization from MeOH were confirmed by XRPD to be of Form III. Scale up using a process similar to the process described in Tables 27-29 yielded 1.2770 kg of Compound I Form III, with low residual solvent contents (methanol: 1248 ppm, n-heptane: <500 ppm and 2-MeTHF: <500 ppm). This was well within the acceptance (ICH) criteria of ≤300 ppm for methanol, ≤5000 ppm for n-Heptane; and ≤5000 ppm for MeTHF. No peaks were observed in the auto-scaled GCHS chromatogram for ethanol, acetone, cyclohexane, or toluene. Moreover, the Compound I Form III obtained in the 1.2770 kg scale process was tested for heavy metals and determined to contain <0.1 ppm of lead, <0.05 ppm of arsenic, <0.2 ppm of cobalt, <0.1 ppm of vanadium, <0.5 ppm of nickel and <1 ppm of titanium. Cadmium and mercury were not present in detectable levels.

FIG. 35 compares the XRPD patterns of Compound I Form III, Compound I Form IV, the crystalline form of Compound I obtained after one of the crystallization purification steps described above, and the crystalline form of Compound I obtained after the crystal transformation of the process described above.

### Comparative Example—Direct Crystallization of Form III from Starting Material (Crude Mixture of Crystalline Form I and Crystalline Form III of Compound I)

A direct crystallization of the starting material from MeOH/H₂O was also tested. The starting material (i.e., a mixture of crystalline Form I of Compound I and crystalline Form III of Compound I with an assay and main purity of t 88.8% and 98.9%, respectively) and MeOH (27.5 V) were added into three stand-up flask. The mixture was heated to about 50-60° C. (inner temperature 55° C.), and stirred for 10-30 min. The mixture was then filtered at 50-60° C., the filter cake was rinsed with MeOH (1.7 V) and the filtrate was cooled to 40-45° C. (inner temperature 40° C.). H₂O (15 V) was added dropwise to the flask at 40-45° C. (inner temperature 40° C.). The resulting mixture was cooled to 10° C. (cooling rate: −20° C./h), and stirred overnight at 10° C. Then, the reaction mixture was filtered and the solid residue (wet cake) was dried at 35° C. for 24 h. The resulting product was obtained with a purity of 89.9% (i.e., same as the starting material).

In sum, the mixed crystal form (Form I and Form III) can be converted to single crystal form (Form III) by crystallization from methanol, but this does not remove impurities. On the other hand, the process described herein, comprising two purification steps in a 2-MeTHF/n-heptane solvent/anti-solvent system, followed by crystallization from MeOH/H₂O, provided a crystalline Form III of Compound I with high purity and low residual solvent content (compliant with ICH requirements).

### Example 14: Extraction of Celastrol from Plant

An Example of an extraction process of celastrol from thunder god vine plant material is described herein:

### Harvesting Step: Thunder God Vine Plant Material

The thunder god vine root was collected, immediately sliced, dried packaged and stored.

### Preparation/Extraction Step: Celastrol Crude Plant Extract

Celastrol was extracted and isolated to give Celastrol Crude Plant Extract

### Extraction/Purification Step: Certified (‘Crude’) Celastrol (Form I)

Celastrol Crude Plant Extract was extracted with ethanol. The extracts were combined and concentrated and the crude celastrol extract then purified by normal phase chromatography (twice) followed by reverse phase chromatography and crystallization to give Certified (‘Crude’) Celastrol (Form I). Crude celastrol was characterized as summarized below:
Orange to red powder
Identification by XRPD: Form I
Related substances by HPLC: Total: Not more than 1.0%
Water: 0.08%
Residual solvents:
   a. Ethanol: 49453 ppm
   b. Dichloromethane: 120 ppm
   c. Ethyl acetate: 1538 ppm
Purity: 94.6%.

### Example 15—Intranasal Formulations Comprising Form III

### Spray Dried Formulations

Two feed solutions were prepared on a 3 g total solids scale, incorporating a celastrol drug substance (comprising a crystalline Form III of celastrol) at 2% w/w, alongside 30% w/w hydroxypropyl methyl cellulose (HPMC) and either hydroxypropyl-beta-cyclodextrin (HPBCD) or trehalose or sorbitol. The celastrol drug substance was initially dissolved in methanol to form a clear bright orange colored solution. HPMC was then slowly added to the celastrol drug substance-methanol solution and stirred until fully dispersed. Separately, trehalose or HP-beta-cyclodextrin were dissolved in the deionised water. The aqueous solution was then added dropwise to the celastrol drug substance-HPMC-methanol solution with stirring to form the final feed. The feed was spray dried immediately after the combination of the organic and aqueous phases.

The composition of the two feed solutions is summarized in Table 30.

TABLE 30
Feed solutions for spray drying

| | | | HPBCD | Anhydrous | | | Total |
|---|---|---|---|---|---|---|---|
| Feed | Celastrol | HPMC | (Kleptose | Trehalose | Methanol | De-ionised | Solids |
| Solution | DS (mg) | (mg) | HPB) (mg) | (mg) | (g) | water (g) | (mg) |
| A | 60 | 900 | 2040 | — | 27.0 | 3.0 | 3000 |
| B | 60 | 900 | — | 2040 | | | |

Both feed solutions were spray dried on a spray drier (ProCepT 4M8) fitted with an ultrasonic nozzle, an inlet temperature of 105° C. to achieve an outlet of ˜85° C., and a feed rate of 2 g/min. For both batches, the majority of powder deposited directly into the collection pot. The resultant powders were orange, free flowing powders. Comparable processing yields were seen for the two batches.

TABLE 31
Spray Dried Batches

| Spray Dried | | Yield | Yield |
|---|---|---|---|
| Batch | Formulation Composition | (g) | (%) |
| Dry A | Celastrol DS:HP-β-CD:HPMC | 2.57 | 85.8 |
| | (2:68:30 w/w/w) | | |
| Dry B | Celastrol DS:Trehalose:HPMC | 2.56 | 85.2 |
| | (2:68:30 w/w/w) | | |

### Particle Size Distribution

Spray dried powders were analyzed for particle size using a laser diffraction particle sizer (Sympatec) equipped with a dispersion unit (RODOS/ASPIROS) and an R3 lens at a dispersion pressure of 1 bar (n=3). Narrow particle size distributions were produced for both powders, with <2% of particles below 10 μm. The particle size distributions for both powders are summarized in Table 32.

TABLE 32
Particle size distribution of spray dried powders

| Spray Dried | | | | | VMD |
|---|---|---|---|---|---|
| Batch | % < 10 μm | X₁₀ (μm) | X₅₀ (μm) | X₉₀ (μm) | (μm) |
| Dry A | 1.79 | 20.71 | 37.04 | 59.27 | 36.68 |
| Dry B | 1.44 | 18.03 | 33.29 | 63.50 | 37.37 |

### Loss on Drying

The residual moisture/solvent content of the spray dried powders was determined (n=1) by loss of mass on drying at 60° C. using thermogravimetric analysis (TGA). Residual moisture content (RMC) values are summarized below alongside T=0 data.

TABLE 33
Residual moisture content of spray dried formulations

| Spray Dried Batch | Residual Moisture Content (%) |
|---|---|
| Dry A | 3.23 |
| Dry B | 2.43 |

### Assay and Related Substances

The celastrol assay of the spray dried powders were determined (n=2) by HPLC. Assay data indicates that the celastrol drug substance was fully retained in both spray dried powders.

TABLE 34
Assay of spray dried formulations

| Spray Dried Batch | Average Assay (%)* n = 2 |
|---|---|
| Dry A | 106.3 |
| Dry B | 100.4 |

### Liquid Formulation for Intranasal Administration

Stock solutions of polysorbate 20, benzalkonium chloride, a combination of microcrystalline cellulose and sodium carboxymethyl cellulose (MCC-NaCMC; Vivapur MCG 611P), and hydroxypropyl methylcellulose (HPMC; HPMC 60SH-4000), were prepared and combined in a 20 mL volumetric flask in the quantities summarized in Table 6, and diluted with ultrapure water and thoroughly mixed. Both suspensions produced were brightly orange colored suspensions.

TABLE 35
Preparation of liquid formulations

| | Liquid A | Liquid B |
|---|---|---|
| Celastrol drug substance (mg) | 400 | 400 |
| Polysorbate 20 Stock (100 mg/mL) | 5 mL (equiv. to 500 mg) | 5 mL (equiv. to 500 mg) |
| Benzalkonium Chloride Stock | 0.2 mL (equiv. to 2 mg) | 0.2 mL (equiv. to 2 mg) |
| (10 mg/mL) | | |
| MCC-NaCMC Stock (30 mg/mL) | — | 12 mL (equiv. to 360 mg) |
| HPMC Stock (2 mg/mL) | 10 mL (equiv. to 20 mg) | — |
| Total Volume (mL) | 20 mL | 20 mL |

TABLE 36
Composition of liquid Formulations

| Formulation | Formulation Composition (mg/mL) | |
|---|---|---|
| Liquid A | Celastrol drug substance:Polysorbate | 20:25:0.1:1 |
| | 20:Benzalkonium chloride:HPMC | |
| Liquid B | Celastrol drug substance:Polysorbate | 20:25:0.1:18 |
| | 20:Benzalkonium chloride:MCC-NaCMC | |

### pH & Viscosity of Liquid Formulations

The pH and viscosity of both suspensions were determined and is summarized in Table 37.

TABLE 37
pH and viscosity of liquid formulations

| Formulation | pH | Viscosity (cP)/Torque (%) |
|---|---|---|
| Liquid A | 6.11 | 2.43-2.39 cP/19-58% |
| Liquid B | 6.33 | 38-52 cP/42-93% |

### Assay and Related Substances

The celastrol drug substance assay of the liquid formulations was determined (n=2) by HPLC. Assay data indicates that celastrol drug substance was fully retained in both suspensions.

TABLE 38
Assay of liquid formulations

| Formulation | Average Assay (%) n = 2 |
|---|---|
| Liquid A | 103.5 |
| Liquid B | 105.7 |

### Intranasal Administration of Formulations of the Disclosure

Sprague Dawley rats aged 5-6 weeks and weighing approximately 150-250 g (N=8) were intranasally administered formulations Dry A, Dry B, Liquid A, and Liquid B, described in above, at a dosing volume of 0.1 mL/kg (corresponding to a dose concentration of 2 mg/kg). Additional rats (N=4) were administered the celastrol drug substance (comprising a crystalline Form III of celastrol) formulated in 0.01% w/v polysorbate 80, delivered via oral gavage at a dosing volume of 10 mL/kg.

Whole blood samples (˜100 μl) were obtained at 5 min, 15 min, 30 min, 1 h, and 4 h or at 5 min, 15 min, 30 min, 1 h, 2 h, 4 h, 8 h, and 24 h after administration and immediately placed back on wet ice. After centrifugation at 6000 rpm for 5 minutes at 4° C., plasma was collected and stored at −80° C. until analysis. The concentration of celastrol in the blood samples was determined by LC-MS. Table 39 summarizes the results of the study.

TABLE 39
Pharmacokinetic parameters of Celastrol

| Formu- | | Cₘₐₓ | Tₘₐₓ | AUC₀₋ₗₐₛₜ |
|---|---|---|---|---|
| lation | Measured at | (ng/mL) | (h) | (ng*hr/mL) |
| Dry A | 5 min, 15 min, 30 min, 1 h, and 4 h | 0.114 | 0.250 | 0.0143 |
| Dry A | 5 min, 15 min, 30 min, 1 h, and 4 h | 1.20 | 0.250 | 0.150 |
| Dry A | 5 min, 15 min, 30 min, 1 h, 2 h, 4 h, 8 h, and 24 h | 0.183 | 0.0830 | 0.00759 |
| Dry A | 5 min, 15 min, 30 min, 1 h, 2 h, 4 h, 8 h, and 24 h | 0.232 | 0.500 | 0.0580 |
| Dry B | 5 min, 15 min, 30 min, 1 h, and 4 h | NR | NR | NR |
| Dry B | 5 min, 15 min, 30 min, 1 h, and 4 h | NR | NR | NR |
| Dry B | 5 min, 15 min, 30 min, 1 h, 2 h, 4 h, 8 h, and 24 h | NR | NR | NR |
| Dry B | 5 min, 15 min, 30 min, 1 h, 2 h, 4 h, 8 h, and 24 h | 0.119 | 2.00 | 0.119 |
| Liquid A | 5 min, 15 min, 30 min, 1 h, and 4 h | 3.44 | 1.00 | 11.1 |
| Liquid A | 5 min, 15 min, 30 min, 1 h, and 4 h | 2.22 | 1.00 | 6.32 |
| Liquid A | 5 min, 15 min, 30 min, 1 h, 2 h, 4 h, 8 h, and 24 h | 4.17 | 1.00 | 19.1 |
| Liquid A | 5 min, 15 min, 30 min, 1 h, 2 h, 4 h, 8 h, and 24 h | 2.93 | 1.00 | 20.3 |
| Liquid B | 5 min, 15 min, 30 min, 1 h, and 4 h | 3.44 | 0.500 | 5.16 |
| Liquid B | 5 min, 15 min, 30 min, 1 h, and 4 h | 4.75 | 1.00 | 11.8 |
| Liquid B | 5 min, 15 min, 30 min, 1 h, 2 h, 4 h, 8 h, and 24 h | 5.65 | 1.00 | 48.6 |
| Liquid B | 5 min, 15 min, 30 min, 1 h, 2 h, 4 h, 8 h, and 24 h | 19.6 | 1.00 | 89.7 |
| Oral | 5 min, 15 min, 30 min, 1 h, and 4 h | 1.7 | 4.00 | 3.46 |
| Oral | 5 min, 15 min, 30 min, 1 h, and 4 h | 1.09 | 4.00 | 2.01 |
| Oral | 5 min, 15 min, 30 min, 1 h, 2 h, 4 h, 8 h, and 24 h | 3.57 | 4.00 | 53.9 |
| Oral | 5 min, 15 min, 30 min, 1 h, 2 h, 4 h, 8 h, and 24 h | 2.64 | 8.00 | 42.7 |

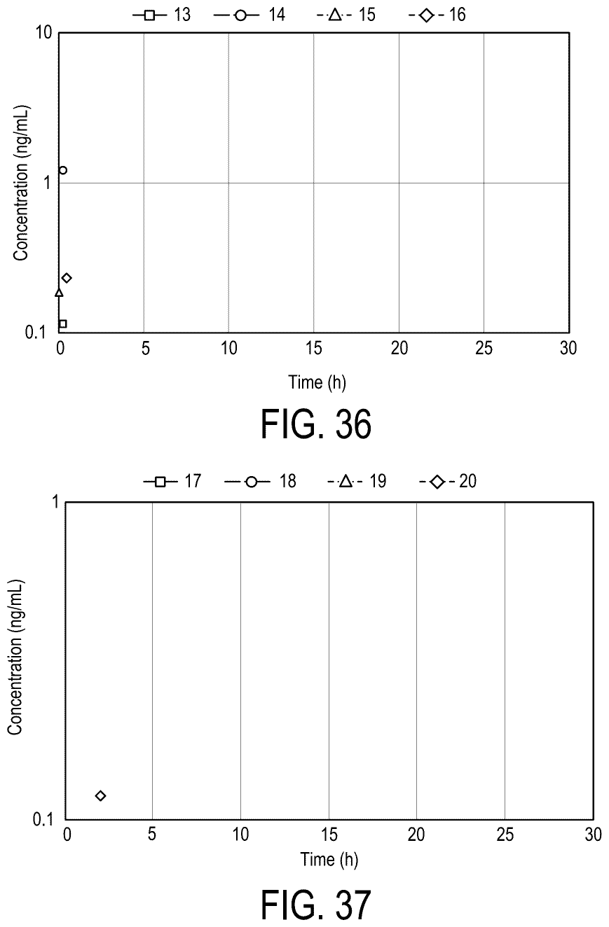

FIG. 36 shows the concentration of celastrol in plasma of male Sprague Dawley rats following intranasal dosing of a dry powder intranasal formulation (Dry A) at a dose concentration of 2 mg/kg.

FIG. 37 shows the concentration of celastrol in plasma of male Sprague Dawley rats following intranasal dosing of a dry powder intranasal formulation (Dry A a) at a dose concentration of 2 mg/kg.

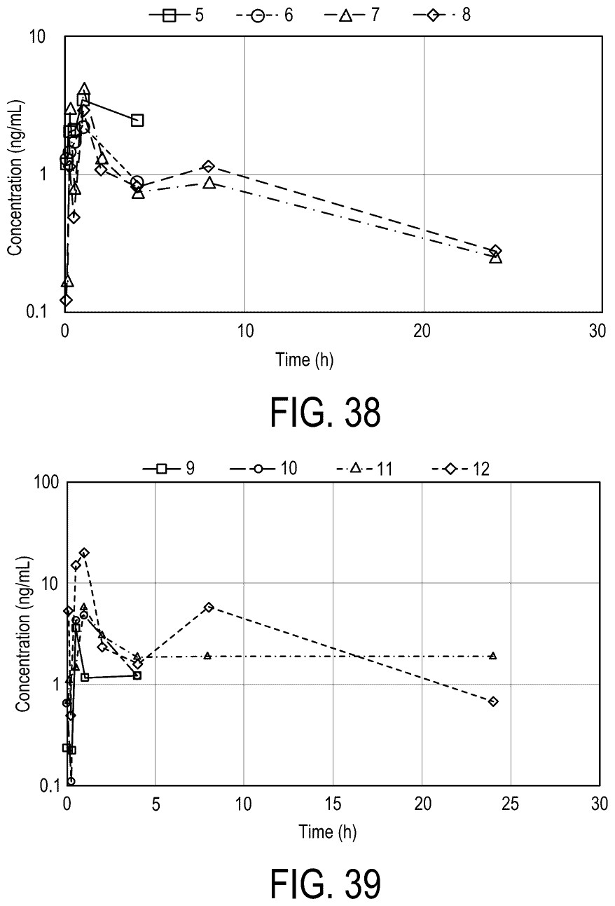

FIG. 38 shows the concentration of celastrol in plasma of male Sprague Dawley rats following intranasal dosing of the liquid intranasal formulation Liquid A at a dose concentration of 2 mg/kg.

FIG. 39 shows the concentration of celastrol in plasma of male Sprague Dawley rats following intranasal dosing of the liquid intranasal formulation Liquid B at a dose concentration of 2 mg/kg.

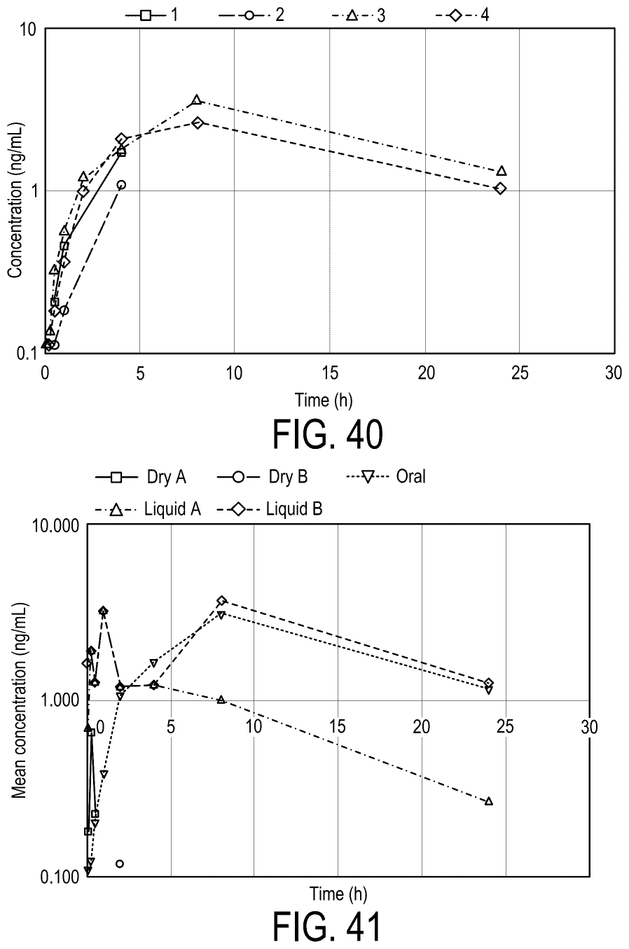

FIG. 40 shows the concentration of celastrol in plasma of male Sprague Dawley rats following oral dosing of celastrol at a dose concentration of 2 mg/kg.

FIG. 41 compares the mean concentrations of celastrol in plasma of male Sprague Dawley rats (average of tested rats) following intranasal administration of formulations Dry A, Dry B, Liquid A, and Liquid B, and oral dosing of celastrol at a dose concentration of 2 mg/kg.

As demonstrated above, intranasal administration using the tested liquid formulations resulted in plasma concentrations of celastrol that were comparable to or higher than plasma concentrations of celastrol administered orally. However, maximum plasma concentrations (Cₘₐₓ) were achieved faster following the intranasal administration than following oral administration (at about 1 h following the intranasal administration compared to 4 h following oral administration)

### Enumerated Embodiments

1. A process for preparing a crystalline Form III of 3-hydroxy-9β,13α-dimethyl-2-oxo-24,25,26-trinoroleana-1(10),3,5,7-tetraen-29-oic acid,

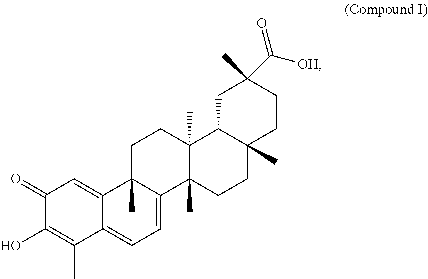

comprising: (i) a first crystallization purification to yield a first purified crystalline form of Compound I; and (ii) a crystal transformation to arrive at the crystalline Form III of Compound I.

2. A process for preparing a crystalline Form III of 3-hydroxy-9β,13α-dimethyl-2-oxo-24,25,26-trinoroleana-1(10),3,5,7-tetraen-29-oic acid,

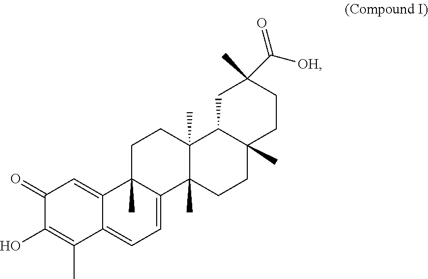

comprising: (i) a first crystallization purification to yield a first purified crystalline form of Compound I; (ii) a second crystallization purification to yield a second purified crystalline form of Compound I; and (iii) a crystal transformation to arrive at the crystalline Form III of Compound I.

3. The process of any of the preceding embodiments, wherein the first crystallization purification comprises:
Step 1′: combining a crude crystalline form of Compound I (e.g., a crude crystalline Form I of Compound I or a crude mixture of a crystalline Form I and a crystalline Form III of Compound I) and an organic solvent to form a first mixture;
Step 2′: adding a seed crystal to the first mixture; wherein step 2′ is after step 1′;
Step 3′: adding an anti-solvent to the first mixture (i.e., the seeded mixture; of step 2′) to form a second mixture; wherein step 3′ is after step 2′; and
Step 4′: isolating a first purified crystalline form of Compound I from the second mixture; wherein step 4′ is after step 3′.

4. The process of any of the preceding embodiments, wherein the second crystallization purification comprises:
Step 5′: combining the first purified crystalline form of Compound I (i.e., the purified crystalline form of Compound I obtained in steps 1′-4′ of the first crystallization purification) and an organic solvent to form a third mixture;
Step 6′: adding a seed crystal to the third mixture; wherein step 6′ is after step 5′
Step 7′: adding an anti-solvent to the third mixture (i.e., the seeded solution of step 6′) to form a fourth mixture; wherein step 7′ is after step 8′; and
Step 8′: isolating a second purified crystalline form of Compound I from the fourth mixture; wherein step 8′ is after step 7′.

5. The process of any of the preceding embodiments, wherein the crystal transformation comprises:
Step ct-1: combining the first or second purified crystalline form of Compound I (i.e., the purified crystalline form of Compound I obtained in the first or second crystallization purification process) and methanol to form a mixture A;
Step ct-2: adding a seed crystal to mixture A to form a mixture B; wherein step ct-2 is after step ct-1;
Step ct-3: adding an anti-solvent to mixture B to form mixture C; wherein step ct-3 is after step ct-2; and
Step ct-4: isolating the crystalline Form III of Compound I from mixture C; wherein step ct-4 is after step ct-3.

6. The process of any of the preceding embodiments, wherein the crystal transformation comprises:
Step ct-1: combining the first or second purified crystalline form of Compound I (i.e., the purified crystalline form of Compound I obtained in the first or second crystallization purification process) and methanol to form a mixture A;
Step ct-1a: adding a first portion of an anti-solvent to mixture A to form mixture A′; wherein step ct-1a is after step ct-1;
Step ct-2′: adding a seed crystal to mixture A′ to form a mixture B; wherein step ct-2 is after step ct-1a′;
Step ct-3: adding an anti-solvent to mixture B to form mixture C; wherein step ct-3 is after step ct-2′.
Step ct-4: isolating the crystalline Form III of Compound I from mixture C; wherein step ct-4 is after step ct-3.

7. The process of any of the preceding embodiments, wherein the first crystallization purification comprises:
Step 1: combining a crude crystalline form of Compound I (e.g., a crude crystalline Form I of Compound I or a crude mixture of a crystalline Form I and a crystalline Form III of Compound I) and an organic solvent to form a first mixture;
Step 2: heating the first mixture to a temperature T¹ to form a first solution; wherein step 2 is after step 1;
Step 3: cooling the first solution to a temperature T²; wherein step 3 is after step 2;
Step 4: adding a seed crystal to the first solution (i.e., the cooled solution of step 3); wherein step 4 is after step 3;
Step 5: adding an anti-solvent to the first solution (i.e., the seeded solution of step 4) to form a second mixture; wherein step 5 is after step 4; and
Step 6: isolating a first purified crystalline form of Compound I from the second mixture; wherein step 6 is after step 5.

8. The process of any of the preceding embodiments, wherein the second crystallization purification comprises:
Step 7: combining the first purified crystalline form of Compound I (i.e., the purified crystalline form of Compound I obtained in steps 1-6 of the first crystallization purification) and an organic solvent to form a third mixture;
Step 8: heating the third mixture to a temperature T³ to form a second solution; wherein step 8 is after step 7;
Step 9: cooling the second solution to a temperature T⁴; wherein step 9 is after step 8;
Step 10: adding a seed crystal to the second solution (i.e., the cooled solution of step 9); wherein step 10 is after step 9;
Step 11: adding an anti-solvent to the second solution (i.e., the seeded solution of step 10) to form a fourth mixture; wherein step 11 is after step 10; and
Step 12: isolating a second purified crystalline form of Compound I from the fourth mixture; wherein step 12 is after step 11.

9. The process of any of the preceding embodiments, wherein the crystal transformation comprises:
Step 13: combining the second purified crystalline form of Compound I (i.e., the purified crystalline form of Compound I obtained in the second crystallization purification process, e.g., steps 7-12) and methanol to form a fifth mixture;
Step 14: heating the fifth mixture to a temperature T⁵ to form a third solution; wherein step 14 is after step 13;
Step 15: cooling the third solution to a temperature T⁶; wherein step 15 is after step 14;
Step 16: adding a first portion of an anti-solvent to the third solution to form a sixth mixture; wherein step 16 is after step 15;
Step 17: adding a seed crystal to the sixth mixture; wherein step 17 is after step 16;
Step 18: adding a second portion of an anti-solvent to the sixth mixture (i.e., the seeded mixture of step 17) to form a seventh mixture; wherein step 18 is after step 17;
Step 19: cooling the seventh mixture to a temperature T⁷; wherein step 19 is after step 18; and
Step 20: isolating the crystalline Form III of Compound I from the seventh mixture; wherein step 20 is after step 19.

10. A process for preparing a Form III of Compound I comprising:
Step 1: combining a crude crystalline form of Compound I (e.g., a crude crystalline Form I of Compound I or a crude mixture of a crystalline Form I and a crystalline Form III of Compound I) and an organic solvent to form a first mixture;
Step 2: heating the first mixture to a temperature T¹ to form a first solution; wherein step 2 is after step 1;
Step 3: cooling the first solution to a temperature T²; wherein step 3 is after step 2;
Step 4: adding a seed crystal to the first solution (i.e., the cooled solution of step 3); wherein step 4 is after step 3;
Step 5: adding an anti-solvent to the first solution (i.e., the seeded solution of step 4) to form a second mixture; wherein step 5 is after step 4;
Step 6: isolating a first purified crystalline form of Compound I from the second mixture; wherein step 6 is after step 5;
Step 7: combining the first purified crystalline form of Compound I (i.e., the purified crystalline form of Compound I obtained in steps 1-6 of the first crystallization purification) and an organic solvent to form a third mixture;
Step 8: heating the third mixture to a temperature T³ to form a second solution; wherein step 8 is after step 7;
Step 9: cooling the second solution to a temperature T⁴; wherein step 9 is after step 8;
Step 10: adding a seed crystal to the second solution (i.e., the cooled solution of step 9); wherein step 10 is after step 9;
Step 11: adding an anti-solvent to the second solution (i.e., the seeded solution of step 10) to form a fourth mixture; wherein step 11 is after step 10;
Step 12: isolating a second purified crystalline form of Compound I from the fourth mixture; wherein step 12 is after step 11;
Step 13: combining the second purified crystalline form of Compound I (i.e., the purified crystalline form of Compound I obtained in the second crystallization purification process, e.g., steps 7-12) and methanol to form a fifth mixture;
Step 14: heating the fifth mixture to a temperature T⁵ to form a third solution; wherein step 14 is after step 13;
Step 15: cooling the third solution to a temperature T⁶; wherein step 15 is after step 14;
Step 16: adding a first portion of an anti-solvent to the third solution to form a sixth mixture; wherein step 16 is after step 15;
Step 17: adding a seed crystal to the sixth mixture; wherein step 17 is after step 16;
Step 18: adding a second portion of an anti-solvent to the seeded mixture of step 17 to form a seventh mixture; wherein step 18 is after step 17;
Step 19: cooling the seventh mixture to a temperature T⁷; wherein step 19 is after step 18; and
Step 20: isolating the crystalline Form III of Compound I from the seventh mixture; wherein step 20 is after step 19.

11. The process of any one of the preceding embodiments, wherein the crude crystalline form of Compound I is a crude mixture of a crystalline Form I and a crystalline Form III of Compound I.

12. The process of any one of the preceding embodiments, wherein the crude crystalline form of Compound I is a crude crystalline Form I of Compound I.

13. The process of any one of the preceding embodiments, wherein the crude crystalline form of Compound I has a purity of about 99.0% or less.

14. The process of any one of the preceding embodiments, wherein the crude crystalline form of Compound I has a purity of about 99.0% or less, about 98.9% or less, about 98.8% or less, about 98.7%, or less, about 98.6% or less, about 98.5% or less, about 98.4% or less, about 98.3% or less, about 98.2% or less, about 98.1% or less, or about 98.0% or less.

15. The process of any one of the preceding embodiments, wherein the crude crystalline form of Compound I has a purity of about 99% or less, about 98% or less, about 97% or less, about 96%, or less, about 95% or less, about 94% or less, about 93% or less, about 92% or less, about 91% or less, or about 90% or less.

16. The process of any one of the preceding embodiments, wherein the organic solvent of step 1, 1′, 5′, or step 7 is selected from methyl ethyl ketone (MEK), methyl isobutyl ketone (MIBK), tetrahydrofuran (THF), 2-methyltetrahydrofuran (2-MeTHF), 1,4-dioxane, dimethylsulfoxide (DMSO), dichloromethane (DCM), anisole, dimethylformamide (DMF), dimethylacetamide (DMAc), and N-methylpyrrolidone (NMP).

17. The process of any one of the preceding embodiments, wherein the organic solvent of step 1, 1′, 5′, or step 7 is selected from tetrahydrofuran (THF), 2-methyltetrahydrofuran (2-MeTHF), 1,4-dioxane, dimethylsulfoxide (DMSO), dichloromethane (DCM), anisole, dimethylformamide (DMF), dimethylacetamide (DMAc), and N-methylpyrrolidone (NMP).

18. The process of any one of the preceding embodiments, wherein the organic solvent of step 1, 1′, 5′, or step 7 is tetrahydrofuran (THF).

19. The process of any one of the preceding embodiments, wherein the organic solvent of step 1, 1′, 5′, or step 7 is 2-methyltetrahydrofuran (2-MeTHF).

20. The process of any one of the preceding embodiments, wherein the process further comprises after step 1: step 1-1: adding activated carbon to the first mixture.

21. The process of any one of the preceding embodiments, wherein the process further comprises after step 1-1: step 1-2: adding an additional amount of the organic solvent.

22. The process of any one of the preceding embodiments, wherein the amount of the organic solvent in step 1 is from about 5V to about 20 V.

23. The process of any one of the preceding embodiments, wherein the amount of the organic solvent in step 1 is from about 5V to about 10 V.

24. The process of any one of the preceding embodiments, wherein the amount of the organic solvent in step 1 is about 8 V.

25. The process of any one of the preceding embodiments, wherein the organic solvent in step 1 is from about 10 V to about 20 V.

26. The process of any one of the preceding embodiments, wherein the amount of the organic solvent in step 1 is about 15 V.

27. The process of any one of the preceding embodiments, wherein the amount of the organic solvent in step 1-2 is from about 5V to about 20 V.

28. The process of any one of the preceding embodiments, wherein the amount of the organic solvent in step 1-2 is from about 5V to about 10 V.

29. The process of any one of the preceding embodiments, wherein the amount of the organic solvent in step 1-2 is about 8 V.

30. The process of any one of the preceding embodiments, wherein the organic solvent in step 1-2 is from about 10 V to about 20 V.

31. The process of any one of the preceding embodiments, wherein the amount of the organic solvent in step 1-2 is about 15 V.

32. The process of any one of the preceding embodiments, wherein step 2 further includes adding activated carbon to the first solution.

33. The process of any one of the preceding embodiments, wherein the activated carbon in step 1, 2, 7, or 8 is added in an amount of from about 0.01 wt % to about 1 wt %.

34. The process of any one of the preceding embodiments, wherein the activated carbon in step 1, 2, 7, or 8 is added in an amount of from about 0.01 wt % to about 0.5 wt %, or from about 0.05 wt % to about 0.5 wt %.

35. The process of any one of the preceding embodiments, wherein the activated carbon in step 11, 2, 7, or 8 is added in an amount of about 0.1 wt % to about 0.4 wt %.

36. The process of any one of the preceding embodiments, wherein the activated carbon in step 1, 2, 7, or 8 is added in an amount of about 0.2 wt %.

37. The process of any one of the preceding embodiments, wherein the activated carbon in step 1, 2, 7, or 8 is added in an amount of about 0.5 wt %.

38. The process of any one of the preceding embodiments, wherein the activated carbon in step 1, 2, 7, or 8 is F1600 activated carbon.

39. The process of any one of the preceding embodiments, wherein T¹ is from about 40° C. to about 110° C., from about 50° C. to about 100° C., or from about 60° C. to about 90° C.

40. The process of any one of the preceding embodiments, wherein T¹ is from about 70° C. to about 80° C. (e.g., about 70° C., about 71° C., about 72° C., about 73° C., about 74° C., about 75° C., about 76° C., about 77° C., about 78° C., about 79° C., or about 80° C.).

41. The process of any one of the preceding embodiments, wherein T¹ is from about 70° C. to about 75° C.

42. The process of any one of the preceding embodiments, wherein the process further comprises after step 2: step 2-1: stirring the first solution for a duration t²⁻¹.

43. The process of any one of the preceding embodiments, wherein t²⁻¹ is from about 12 h to about 24 h (e.g., about 12 h, about 13 h, about 14 h, about 15 h, about 16 h, about 17 h, about 18 h, about 19 h, about 20 h, about 21 h, about 22 h, about 23 h, or about 24 h).

44. The process of any one of the preceding embodiments, wherein t²⁻¹ is from about 16 h to about 20 h.

45. The process of any one of the preceding embodiments, wherein t²⁻¹ is from about 1 h to about 8 h (e.g., about 1 h, about 2 h, about 3 h, about 4 h, about 5 h, about 6 h, about 7 h, or about 8 h).

46. The process of any one of the preceding embodiments, wherein t²⁻¹ is from about 1 h to about 4 h.

47. The process of any one of the preceding embodiments, wherein t²⁻¹ is from about 2 h to about 3 h.

48. The process of any one of the preceding embodiments, wherein t²⁻¹ is from about 2 h to about 8 h.

49. The process of any one of the preceding embodiments, wherein t²⁻¹ is from about 4 h to about 6 h.

50. The process of any one of the preceding embodiments, wherein t²⁻¹ is one hour or less.

51. The process of any one of the preceding embodiments, wherein t²⁻¹ is from about 10 min to about 40 min.

52. The process of any one of the preceding embodiments, wherein t²⁻¹ is from about 20 min to about 30 min.

53. The process of any one of the preceding embodiments, wherein the process further comprises after step 2 or step 2-1: step 2-2: polish filtering the first solution.

54. The process of any one of the preceding embodiments, wherein step 2-2 further comprises rinsing the filter with the organic solvent of step 1, and re-heating the filtrate to the temperature T¹. 55. The process of any one of the preceding embodiments, wherein in step 2-2, the amount of the organic solvent used to rinse the filter is from about 0.5 V to about 5 V, from about 1 V to about 4 V, or from about 1 V to about 3 V.

56. The process of any one of the preceding embodiments, wherein in step 2-2, the amount of the organic solvent used to rinse the filter is about 2 V.

57. The process of any one of the preceding embodiments, wherein T² is from about 30° C. to about 90° C., from about 40° C. to about 80° C., or from about 50° C. to about 70° C., from about 50° C. to about 60° C., or from about 55° C. to about 60° C.

58. The process of any one of the preceding embodiments, wherein T² is about 55° C.

59. The process of any one of the preceding embodiments, wherein T² is about 60° C.

60. The process of any one of the preceding embodiments, wherein the seed crystal in step 2′, 4, 6′, or 10 is a crystalline Form I of Compound I.

61. The process of any one of the preceding embodiments, wherein the seed crystal in step 2′, 4, 6′, or 10 is a crystalline Form III of Compound I.

62. The process of any one of the preceding embodiments, wherein the seed crystal in step 2′, 4, 6′, or 10 is a mixture of crystalline Form I of Compound I and crystalline Form III of Compound I.

63. The process of any one of the preceding embodiments, wherein the seed crystal in step 2′ or 4 is of the same crystalline form as the first purified crystalline form of Compound I.

64. The process of any one of the preceding embodiments, wherein the seed crystal in step 6′ 10 is of the same crystalline form as the second purified crystalline form of Compound I.

65. The process of any one of the preceding embodiments, wherein the process further comprises after step 4: step 4-1: aging the seeded mixture for a duration t⁴⁻¹.

66. The process of any one of the preceding embodiments, wherein t⁴⁻¹ is 3 h or less.

67. The process of any one of the preceding embodiments, wherein t⁴⁻¹ is from about 10 min to about 3 h.

68. The process of any one of the preceding embodiments, wherein t⁴⁻¹ is from about 20 min to about 40 min.

69. The process of any one of the preceding embodiments, wherein t⁴⁻¹ is about 30 min.

70. The process of any one of the preceding embodiments, wherein t⁴⁻¹ is from about 1 h to about 3 h. The process of any one of the preceding embodiments, wherein t⁴⁻¹ is about 2 h.

71. The process of any one of the preceding embodiments, wherein the process further comprises after step 4 or step 4-1: step 4-2: cooling the seeded mixture to a temperature T⁴⁻². The process of any one of the preceding embodiments, wherein step 4-2 further comprises stirring the seeded mixture at temperature T⁴⁻² for 5-10 h.

72. The process of any one of the preceding embodiments, wherein T⁴⁻² is from about 0° C. to about 50° C.

73. The process of any one of the preceding embodiments, wherein T⁴⁻² is from about 0° C. to about 40° C., from about 10° C. to about 40° C., from about 0° C. to about 30° C., from about 10° C. to about 30° C., or from about 20° C. to about 30° C.

74. The process of any one of the preceding embodiments, wherein T⁴⁻² is about 25° C.

75. The process of any one of the preceding embodiments, wherein in step 4-2, the seeded mixture is cooled at a cooling rate of from about 5° C./h to about 20° C./h, or from about 5° C./h to about 15° C./h.

76. The process of any one of the preceding embodiments, wherein in step 4-2, the seeded mixture is cooled at a cooling rate of 10° C./h.

77. The process of any one of the preceding embodiments, wherein the anti-solvent in step 3′ 5, 7′ or 11 is added over a period of from about 2 h to about 10 h, from about 4 h to about 8 h, or from about 4 h to about 6 h

78. The process of any one of the preceding embodiments, wherein the anti-solvent in step 3′, 5, 7′, or 11 is added over a period of from about 5 h to about 7 h (e.g., 6 h).

79. The process of any one of the preceding embodiments, wherein the anti-solvent in step 3′, 5, 7′, or 11 is selected from, isopropanol (IPA), n-propanol (n-PrOH), 2-butanol, acetone, ethyl acetate (EtOAc), isopropyl acetate (IPAc), methyl tert-butyl ether (MTBE), H₂O, acetonitrile (MeCN), heptane (e.g., n-heptane), and acetic acid (HOAc).

80. The process of any one of the preceding embodiments, wherein the anti-solvent in step 3′, 5, 7′, or 11 is selected from ethyl acetate (EtOAc), acetonitrile (MeCN), and heptane (e.g., n-heptane).

81. The process of any one of the preceding embodiments, wherein the anti-solvent in step 3′, 5, 7′, or 11 is heptane.

82. The process of any one of the preceding embodiments, wherein the anti-solvent in step 3′, 5, 7′, or 11 is n-heptane.

83. The process of any one of the preceding embodiments, wherein the amount of anti-solvent in step 3′, 5, 7′, or 11 is from about 5 V to about 50 V, from about 10 V to about 50 V, from about 20 V to about 50 V, from about 20 V to about 40 V, or from about 30 V to about 40 V. The process of any one of the preceding embodiments, wherein the amount of anti-solvent in step 3′, 5, 7′, or 11 is from about 30 V to about 35 V.

84. The process of any one of the preceding embodiments, wherein the amount of anti-solvent in step 3′, 5, 7′, or 11 is about 34 V.

85. The process of any one of the preceding embodiments, wherein the amount of anti-solvent in step 3′, 5, 7′, or 11 is about 30 V.

86. The process of any one of the preceding embodiments, wherein the process further comprises after step 5, step 5-1: cooling the second mixture to a temperature T⁵⁻¹.

87. The process of any one of the preceding embodiments, wherein T⁵⁻¹ is from about −10° C. to about 15° C., from about −5° C. to about 15° C., from about −5° C. to about 10° C., or from about 0° C. to about 10° C.

88. The process of any one of the preceding embodiments, wherein T⁵⁻¹ is about 5° C.

89. The process of any one of the preceding embodiments, wherein in step 5-1, the second mixture is cooled at a cooling rate of from about 5° C./h to about 20° C./h, or from about 5° C./h to about 15° C./h.

90. The process of any one of the preceding embodiments, wherein in step 5-1, the second mixture is cooled at a cooling rate of 10° C./h.

91. The process of any one of the preceding embodiments, wherein the process further comprises after step 5-1: step 5-2: stirring the cooled mixture of step 5-1 for a duration t⁵⁻¹ at the temperature T⁵⁻¹.

92. The process of any one of the preceding embodiments, wherein t⁵⁻¹ is from about 5 h to about 20 h, from about 5 h to about 14 h, from about 8 h to about 14 h, from about 8 h to about 13 h, from about 10 h to about 13 h, or from about 11 h to about 12 h.

93. The process of any one of the preceding embodiments, wherein t⁵⁻¹ is at least 8 h.

94. The process of any one of the preceding embodiments, wherein t⁵⁻¹ is from about 8 h to about 16 h, from about 8 h to about 14 h, from about 8 h to about 13 h, or from about 8 h to about 12 h.

95. The process of any one of the preceding embodiments, wherein step 4′ or 6 (i.e., isolating the first purified crystalline form) comprises filtering the second mixture.

96. The process of any one of the preceding embodiments, wherein the filtering further comprises rinsing the solid residue (i.e., the first purified crystalline form) with the anti-solvent of step 3′ or 5, and/or drying the solid residue (i.e., the first purified crystalline form) under vacuum.

97. The process of any one of the preceding embodiments, wherein step 4′ or 6 comprises drying the solid residue (i.e., the first purified crystalline form) under vacuum at a temperature of from about 30° C. to about 40° C.

98. The process of any one of the preceding embodiments, wherein in step 4′ or 6, the amount of the anti-solvent used to rinse the filter is from about 0.5 V to about 5 V, from about 1V to about 4 V, or from about 1V to about 3 V.

99. The process of any one of the preceding embodiments, wherein in step 4′ or 6, the amount of the anti-solvent used to rinse the filter is about 2V.

100. The process of any one of the preceding embodiments, wherein the first purified crystalline form of Compound I is Form I of Compound I.

101. The process of any one of the preceding embodiments, wherein the first purified crystalline form of Compound I is Form III of Compound I.

102. The process of any one of the preceding embodiments, wherein the first purified crystalline form of Compound I is a mixture of crystalline Form I of Compound I and crystalline Form III of Compound I.

103. The process of any one of the preceding embodiments, wherein the first purified crystalline form of Compound I is Form II of Compound I.

104. The process of any one of the preceding embodiments, wherein the first purified crystalline form of Compound I is Form IV of Compound I.

105. The process of any one of the preceding embodiments, wherein the first purified crystalline form of Compound I is Form V of Compound I.

106. The process of any one of the preceding embodiments, wherein the process further comprises after step 7: step 7-1: adding activated carbon to the third mixture.

107. The process of any one of the preceding embodiments, wherein the process further comprises after step 7-1: step 7-2: adding an additional amount of the organic solvent.

108. The process of any one of the preceding embodiments, wherein the amount of the organic solvent in step 7 is from about 5 V to about 20 V.

109. The process of any one of the preceding embodiments, wherein the amount of the organic solvent in step 7 is from about 5 V to about 10 V.

110. 1. The process of any one of the preceding embodiments, wherein the amount of the organic solvent in step 7 is about 8 V.

111. The process of any one of the preceding embodiments, wherein the amount of the organic solvent in step 7 is from about 10 V to about 20 V.

112. The process of any one of the preceding embodiments, wherein the amount of the organic solvent in step 7 is about 15 V.

113. The process of any one of the preceding embodiments, wherein the additional amount of the organic solvent in step 7-2 is from about 5 V to about 20 V.

114. The process of any one of the preceding embodiments, wherein the additional amount of the organic solvent in step 7-2 is from about 5 V to about 10 V.

115. The process of any one of the preceding embodiments, wherein the additional amount of the organic solvent in step 7-2 is about 8 V.

116. The process of any one of the preceding embodiments, wherein the additional amount of the organic solvent in step 7-2 is from about 10 V to about 20 V.

117. The process of any one of the preceding embodiments, wherein the additional amount of the organic solvent in step 7-2 is about 15 V.

118. The process of any one of the preceding embodiments, wherein step 8 further includes adding activated carbon to the third solution.

119. The process of any one of the preceding embodiments, wherein T³ is from about 40° C. to about 110° C., from about 50° C. to about 100° C., or from about 60° C. to about 90° C.

120. The process of any one of the preceding embodiments, wherein T³ is from about 70° C. to about 80° C. (e.g., about 70° C., about 71° C., about 72° C., about 73° C., about 74° C., about 75° C., about 76° C., about 77° C., about 78° C., about 79° C., or about 80° C.).

121. The process of any one of the preceding embodiments, wherein T³ is from about 70° C. to about 75° C.

122. The process of any one of the preceding embodiments, wherein the process further comprises after step 8: step 8-1: stirring the third solution for a duration t⁸⁻¹.

123. The process of any one of the preceding embodiments, wherein t⁸⁻¹ is from about 1 h to about 4 h.

124. The process of any one of the preceding embodiments, wherein t⁸⁻¹ is from about 2 h to about 3 h.

125. The process of any one of the preceding embodiments, wherein t²⁻¹ is from about 2 h to about 8 h.

126. The process of any one of the preceding embodiments, wherein t⁸⁻¹ is from about 4 h to about 6 h.

127. The process of any one of the preceding embodiments, wherein t²⁻¹ is one hour or less.

128. The process of any one of the preceding embodiments, wherein t²⁻¹ is from about 10 min to about 40 min.

129. The process of any one of the preceding embodiments, wherein t²⁻¹ is from about 20 min to about 30 min.

130. The process of any one of the preceding embodiments, wherein the process further comprises after step 8 or step 8-1: step 8-2: polish filtering the third solution.

131. The process of any one of the preceding embodiments, wherein step 8-2 further comprises rinsing the filter with the organic solvent of step 7, and re-heating the filtrate to the temperature T³⁻¹.

132. The process of any one of the preceding embodiments, wherein in step 8-2, the amount of the organic solvent used to rinse the filter is from about 0.5 V to about 5 V, from about 1 V to about 4 V, or from about 1 V to about 3 V.

133. The process of any one of the preceding embodiments, wherein in step 8-2, the amount of the organic solvent used to rinse the filter is about 2 V.

134. The process of any one of the preceding embodiments, wherein T⁴ is from about 30° C. to about 90° C., from about 40° C. to about 80° C., or from about 50° C. to about 70° C., from about 50° C. to about 60° C., from about 55° C. to about 60° C.

135. The process of any one of the preceding embodiments, wherein T⁴ is about 55° C.

136. The process of any one of the preceding embodiments, wherein T⁴ is about 60° C.

137. The process of any one of the preceding embodiments, wherein the process further comprises after step 10: step 10-1: aging the seeded mixture for a duration t¹⁰⁻¹.

138. The process of any one of the preceding embodiments, wherein t¹⁰⁻¹ is 3 h or less.

139. The process of any one of the preceding embodiments, wherein t¹⁰⁻¹ is from about 10 min to about 3 h. The process of any one of the preceding embodiments, wherein t¹⁰⁻¹ is from about 20 min to about 40 min.

140. The process of any one of the preceding embodiments, wherein t¹⁰⁻¹ is from about 30 min to about 40 min.

141. The process of any one of the preceding embodiments, wherein t¹⁰⁻¹ is about 30 min.

142. The process of any one of the preceding embodiments, wherein t¹⁰⁻¹ is from about 1 h to about 3 h.

143. The process of any one of the preceding embodiments, wherein t¹⁰⁻¹ is about 2 h.

144. The process of any one of the preceding embodiments, wherein the process further comprises after step 10 or step 10-1: step 10-2: cooling the seeded mixture to a temperature T¹⁰⁻²

145. The process of any one of the preceding embodiments, wherein step 10-2 further comprises stirring the seeded mixture at temperature T¹⁰⁻² for 5-10 h.

146. The process of any one of the preceding embodiments, wherein T¹⁰⁻² is from about 0° C. to about 50° C. The process of any one of the preceding embodiments, wherein T¹⁰⁻² is from about 0° C. to about 40° C., from about 10° C. to about 40° C., from about 0° C. to about 30° C., from about 10° C. to about 30° C., or from about 20° C. to about 30° C.

147. The process of any one of the preceding embodiments, wherein T¹⁰⁻² is about 25° C.

148. The process of any one of the preceding embodiments, wherein in step 10-2, the seeded mixture is cooled at a cooling rate of from about 5° C./h to about 20° C./h, or from about 5° C./h to about 15° C./h.

149. The process of any one of the preceding embodiments, wherein in step 10-2, the seeded mixture is cooled at a cooling rate of 10° C./h.

150. The process of any one of the preceding embodiments, wherein the process further comprises after step 11, step 11-1: cooling the fourth mixture to a temperature T¹¹⁻¹.

151. The process of any one of the preceding embodiments, wherein T¹¹⁻¹ is from about −10° C. to about 15° C., from about −5° C. to about 15° C., from about −5° C. to about 10° C., or from about 0° C. to about 10° C.

152. The process of any one of the preceding embodiments, wherein T¹¹⁻¹ is about 5° C.

153. The process of any one of the preceding embodiments, wherein in step 11-1, the fourth mixture is cooled at a cooling rate of from about 5° C./h to about 20° C./h, or from about 5° C./h to about 15° C./h.

154. The process of any one of the preceding embodiments, wherein in step 11-1, the fourth mixture is cooled at a cooling rate of 10° C./h.

155. The process of any one of the preceding embodiments, wherein the process further comprises after step 11-1: step 11-2: stirring the cooled mixture of step 11-1 for a duration t¹¹⁻¹ at the temperature T¹¹⁻¹.

156. The process of any one of the preceding embodiments, wherein t¹¹⁻¹ is from about 5 h to about 20 h, from about 5 h to about 14 h, from about 8 h to about 14 h, from about 8 h to about 13 h, from about 10 h to about 13 h, or from about 11 h to about 12 h.

157. The process of any one of the preceding embodiments, wherein t¹¹⁻¹ is at least 8 h.

158. The process of any one of the preceding embodiments, wherein t¹¹⁻¹ is from about 8 h to about 16 h, from about 8 h to about 14 h, from about 8 h to about 13 h, or from about 8 h to about 12 h.

159. The process of any one of the preceding embodiments, wherein step 8′ or 12 (i.e., isolating the second purified crystalline form) comprises filtering the fourth mixture.

160. The process of any one of the preceding embodiments, wherein the filtering further comprises rinsing the solid residue (i.e., the first purified crystalline form) with the anti-solvent of step 7′ or 11, and/or drying the solid residue (i.e., the first purified crystalline form) under vacuum.

161. The process of any one of the preceding embodiments, wherein step 8′ or 12 comprises drying the solid residue (i.e., the second purified crystalline form) under vacuum at a temperature of from about 30° C. to about 40° C.

162. The process of any one of the preceding embodiments, wherein in step 8′ or 12, the amount of the anti-solvent used to rinse the filter is from about 0.5 V to about 5 V, from about 1 V to about 4 V, or from about 1 V to about 3 V.

163. The process of any one of the preceding embodiments, wherein in step 8′ or 12, the amount of the anti-solvent used to rinse the filter is about 2 V.

164. The process of any one of the preceding embodiments, wherein the second purified crystalline form of Compound I is Form I of Compound I.

165. The process of any one of the preceding embodiments, wherein the second purified crystalline form of Compound I is Form III of Compound I.

166. The process of any one of the preceding embodiments, wherein the second purified crystalline form of Compound I is a mixture of crystalline Form I of Compound I and crystalline Form III of Compound I.

167. The process of any one of the preceding embodiments, wherein the second purified crystalline form of Compound I is Form II of Compound I.

168. The process of any one of the preceding embodiments, wherein the second purified crystalline form of Compound I is Form IV of Compound I. The process of any one of the preceding embodiments, wherein the second purified crystalline form of Compound I is Form V of Compound I.

169. The process of any one of the preceding embodiments, wherein the amount of the methanol of step 13 is from about 10 V to about 40 V, from about 15 V to about 35 V, or from about 20 V to about 35 V.

170. The process of any one of the preceding embodiments, wherein the amount of the methanol of step 13 is about 28 V.

171. The process of any one of the preceding embodiments, wherein T⁵ is from about 30° C. to about 90° C., from about 40° C. to about 80° C., or from about 50° C. to about 70° C., from about 40° C. to about 60° C., from about 50° C. to about 60° C., or from about 55° C. to about 60° C.

172. The process of any one of the preceding embodiments, wherein T⁵ is about 55° C.

173. The process of any one of the preceding embodiments, wherein the process further comprises after step 14: step 14-1: filtering the third solution.

174. The process of any one of the preceding embodiments, wherein step 14-1 further comprises rinsing the filter with the methanol of step 13.

175. The process of any one of the preceding embodiments, wherein in step 14-1, the amount of the methanol used to rinse the filter is from about 0.5 V to about 5 V, from about 1 V to about 4 V, or from about 1 V to about 3 V.

176. The process of any one of the preceding embodiments, wherein in step 14-1, the amount of the methanol used to rinse the filter is about 2 V.

177. The process of any one of the preceding embodiments, wherein T⁶ is from about 10° C. to about 60° C., from about 20° C. to about 50° C., from about 30° C. to about 50° C., from about 40° C. to about 45° C., or from about 35° C. to about 45° C.

178. The process of any one of the preceding embodiments, wherein T⁶ is about 40° C.

179. The process of any one of the preceding embodiments, wherein the anti-solvent in step ct-1a or 16 is H₂O.

180. The process of any one of the preceding embodiments, wherein in step ct-1a or 16, the first portion of the anti-solvent is an amount of from about 1 V to about 20 V, from about 1 V to about 15 V, from about 1 V to about 10 V, or from about 2 V to about 8 V.

181. The process of any one of the preceding embodiments, wherein in step ct-1a or 16, the first portion of the anti-solvent is an amount of about 5 V.

182. The process of any one of the preceding embodiments, wherein the anti-solvent in step ct-1a or 16 is added at the temperature T⁶.

183. The process of any one of the preceding embodiments, wherein the seed crystal in step ct-2, ct-2′, or 17 is a crystalline Form III of Compound I.

184. The process of any one of the preceding embodiments, wherein the seed crystal in step 2′, 4, 6′, 10, ct-2, ct-2′, or 17 is added in an amount of from about 0.1 wt % to about 3 wt %, from about 0.1 wt % to about 2 wt %, from about 0.5 wt % to about 3 wt %, or from about 0.5 wt % to about 2 wt %.

185. The process of any one of the preceding embodiments, wherein the seed crystal in step 2′ 4, 6′, 10, ct-2, ct-2′, or 17 is added in an amount of about 1 wt %.

186. The process of any one of the preceding embodiments, wherein the process further comprises after step 17: step 17-1: aging the seeded mixture for a duration t¹⁷⁻¹.

187. The process of any one of the preceding embodiments, wherein t¹⁷⁻¹ is 3 h or less.

188. The process of any one of the preceding embodiments, wherein t¹⁷⁻¹ is from about 10 min to about 3 h.

189. The process of any one of the preceding embodiments, wherein t¹⁷⁻¹ is from about 20 min to about 40 min, or from about 30 min to about 40 min.

190. The process of any one of the preceding embodiments, wherein t¹⁷⁻¹ is about 30 min.

191. The process of any one of the preceding embodiments, wherein the anti-solvent in step ct-3 or 18 is H₂O.

192. The process of any one of the preceding embodiments, wherein in step ct-3 or 18, the second portion of the anti-solvent is an amount of from about 1 V to about 30 V, from about 5 V to about 25 V, from about 5 V to about 20 V, or from about 5 V to about 15 V.

193. The process of any one of the preceding embodiments, wherein in step ct-3 or 18, the second portion of the anti-solvent is an amount of about 10 V.

194. The process of any one of the preceding embodiments, wherein the anti-solvent in step ct-3 or 18 is added at the temperature T⁵.

195. The process of any one of the preceding embodiments, wherein the process further comprises after step 18: step 18-1: stirring the third solution for a duration t¹⁸⁻¹.

196. The process of any one of the preceding embodiments, wherein t¹⁸⁻¹ is one hour or less.

197. The process of any one of the preceding embodiments, wherein t¹⁸⁻¹ is from about 10 min to about 40 min.

198. The process of any one of the preceding embodiments, wherein t¹⁸⁻¹ is from about 20 min to about 30 min.

199. The process of any one of the preceding embodiments, wherein T⁷ is from about −5° C. to about 30° C., from about 0° C. to about 20° C., from about 0° C. to about 15° C., or from about 5° C. to about 15° C.

200. The process of any one of the preceding embodiments, wherein T⁷ is about 10° C.

201. The process of any one of the preceding embodiments, wherein in step 19, the seventh mixture is cooled at a cooling rate of from about 10° C./h to about 30° C./h, or from about 15° C./h to about 25° C./h.

202. The process of any one of the preceding embodiments, wherein in step 19, the seventh mixture is cooled at a cooling rate of 20° C./h.

203. The process of any one of the preceding embodiments, wherein the process further comprises after step 19: step 19-1: stirring the third solution for a duration t¹⁹⁻¹.

204. The process of any one of the preceding embodiments, wherein t¹⁹⁻¹ is from about 5 h to about 20 h, from about 5 h to about 14 h, from about 8 h to about 14 h, from about 8 h to about 13 h, from about 10 h to about 13 h, from about 11 h to about 12 h, or from about 14 h to about 16 h.

205. The process of any one of the preceding embodiments, wherein t¹⁹⁻¹ is at least 8 h.

206. The process of any one of the preceding embodiments, wherein t¹⁹⁻¹ is from about 8 h to about 16 h, from about 8 h to about 14 h, or from about 8 h to about 12 h.

207. The process of any one of the preceding embodiments, wherein the anti-solvent in step ct-1a, ct-3, 16 or 18 is added dropwise.

208. The process of any one of the preceding embodiments, wherein step 20 (i.e., isolating the crystalline Form III of Compound I) comprises filtering the seventh mixture.

209. The process of any one of the preceding embodiments, wherein step ct-4 (i.e., isolating the crystalline Form III of Compound I) comprises filtering mixture C.

210. The process of any one of the preceding embodiments, wherein the filtering further comprises rinsing the solid residue (i.e., the crystalline Form III of Compound I) with a mixture of methanol and H₂O, and/or drying the solid residue (i.e., the crystalline Form III of Compound I) under vacuum.

211. The process of any one of the preceding embodiments, wherein step ct-4 or 20 comprises drying the solid residue (i.e., the crystalline Form III of Compound I) under vacuum at a temperature of from about 30° C. to about 40° C.

212. The process of any one of the preceding embodiments, wherein in step ct-4 or 20, the amount of the mixture of methanol and H₂O used to rinse the filter is from about 0.5 V to about 5 V, from about 1 V to about 4 V, or from about 1 V to about 3 V.

213. The process of any one of the preceding embodiments, wherein in step ct-4 or 20, the amount of the mixture of methanol and H₂O used to rinse the filter is about 2 V.

214. The process of any one of the preceding embodiments, wherein the step ct-4 or 20, the mixture of methanol and H₂O comprises methanol and H₂O in a ratio of from about 0.5:1 to about 4:1 or from about 1:1 to about 3:1.

215. The process of any one of the preceding embodiments, wherein the step ct-4 or 20, the mixture of methanol and H₂O is a 2:1 mixture of methanol and H₂O

216. The process of any one of the preceding embodiments, wherein the crude crystalline form of Compound I is obtained by a process comprising extracting celastrol from thunder god vine.

217. A crystalline Form III of Compound I, made by a process of any one of the preceding embodiments.

218. A method of purifying Compound I or a crystalline form of Compound I, comprising the making a crystalline Form III of Compound I by a process of any one of embodiments 1-216.

219. A crystalline Form III of 3-hydroxy-9β,13α-dimethyl-2-oxo-24,25,26-trinoroleana-1(10),3,5,7-tetraen-29-oic acid,

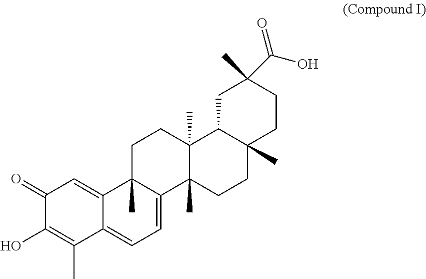

characterized by an x-ray powder diffraction pattern comprising peaks at 2θ angles (±0.2) of 9.51°, 14.81°, and 16.84°.

220. The crystalline form of embodiment 219, characterized by an X-ray powder diffraction pattern further comprising one or more peaks at 2θ angles (±0.2) selected from the group consisting of 13.60°, 14.65°, 18.74°, and 19.10°.

221. The crystalline form of embodiment 219, characterized by an X-ray powder diffraction pattern further comprising one peak at 2θ angles (±0.2) selected from the group consisting of 13.60°, 14.65°, 18.74°, and 19.10°.

222. The crystalline form of embodiment 219, characterized by an X-ray powder diffraction pattern further comprising two peaks at 2θ angles (±0.2) selected from the group consisting of 13.60°, 14.65°, 18.74°, and 19.10°.

223. The crystalline form of embodiment 219, characterized by an X-ray powder diffraction pattern further comprising three peaks at 2θ angles (±0.2) selected from the group consisting of 13.60°, 14.65°, 18.74°, and 19.10°.

224. The crystalline form of embodiment 219, characterized by an X-ray powder diffraction pattern further comprising peaks at 2θ angles (±0.2) of 13.60°, 14.65°, 18.74°, and 19.10°. 225. A crystalline Form III of 3-hydroxy-9β,13α-dimethyl-2-oxo-24,25,26-trinoroleana-1(10),3,5,7-tetraen-29-oic acid,

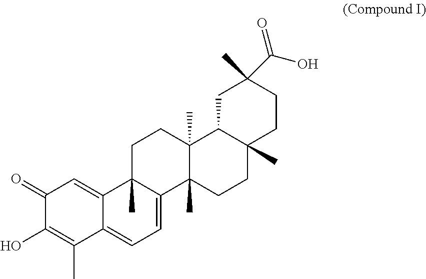

characterized by an x-ray powder diffraction pattern comprising two or more characteristic peaks at 2θ angles selected from the group consisting of 9.51°, 13.60°, 14.65°, 14.81°, 16.84°, 18.74°, and 19.10°.

226. The crystalline form of embodiment 225, characterized by an x-ray powder diffraction pattern comprising three, four, five, or six characteristic peaks at 2θ angles selected from the group consisting of 9.51°, 13.60°, 14.65°, 14.81°, 16.84°, 18.74°, and 19.10°.

227. The crystalline form of embodiment 225, characterized by an x-ray powder diffraction pattern comprising three characteristic peaks at 2θ angles selected from the group consisting of 9.51°, 13.60°, 14.65°, 14.81°, 16.84°, 18.74°, and 19.10°.

228. The crystalline form of embodiment 225, characterized by an x-ray powder diffraction pattern comprising four characteristic peaks at 2θ angles selected from the group consisting of 9.51°, 13.60°, 14.65°, 14.81°, 16.84°, 18.74°, and 19.10°.

229. The crystalline form of embodiment 225, characterized by an x-ray powder diffraction pattern comprising five characteristic peaks at 2θ angles selected from the group consisting of 9.51°, 13.60°, 14.65°, 14.81°, 16.84°, 18.74°, and 19.10°.

230. The crystalline form of embodiment 225, characterized by an x-ray powder diffraction pattern comprising six characteristic peaks at 2θ angles selected from the group consisting of 9.51°, 13.60°, 14.65°, 14.81°, 16.84°, 18.74°, and 19.10°.

231. A crystalline Form III of 3-hydroxy-9β,13α-dimethyl-2-oxo-24,25,26-trinoroleana-1(10),3,5,7-tetraen-29-oic acid,

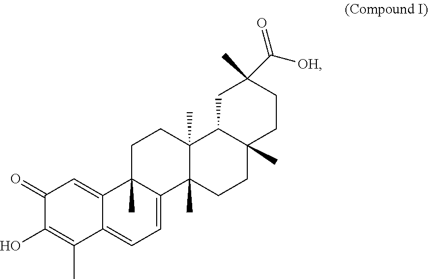

characterized by an X-ray powder diffraction pattern comprising one or more peaks at 2θ angles (±0.2) selected from the group consisting of 13.60°, 14.65°, 18.74°, and 19.10°.
232. A crystalline Form III of 3-hydroxy-9β,13α-dimethyl-2-oxo-24,25,26-trinoroleana-1(10),3,5,7-tetraen-29-oic acid,

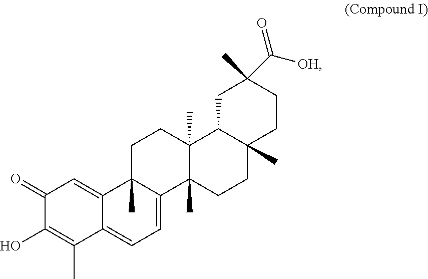

characterized by an X-ray powder diffraction pattern comprising characterized by an X-ray powder diffraction pattern comprising one or more peaks at 2θ angles (±0.2) selected from the group consisting of 9.51°, 14.81°, and 16.84°.

233. The crystalline form of embodiment 232 comprising two peaks at 2θ angles (±0.2) selected from the group consisting of 9.51°, 14.81°, and 16.84°.

234. A crystalline Form III of 3-hydroxy-9β,13α-dimethyl-2-oxo-24,25,26-trinoroleana-1(10),3,5,7-tetraen-29-oic acid,

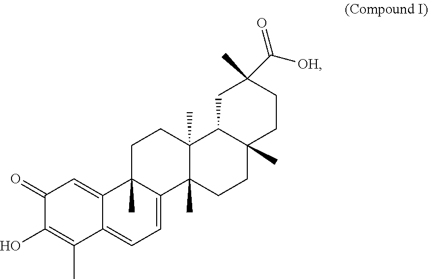

characterized by an X-ray powder diffraction pattern comprising one or more peaks (e.g., one, two, three, or four peaks) at 2θ angles (±0.2) selected from the group consisting of 13.60°, 14.65°, 18.74°, and 19.10°.

235. The crystalline form of embodiment 234, comprising two peaks at 2θ angles (±0.2) selected from the group consisting of 13.60°, 14.65°, 18.74°, and 19.10°.

236. The crystalline form of embodiment 234, comprising three peaks at 2θ angles (±0.2) selected from the group consisting of 13.60°, 14.65°, 18.74°, and 19.10°.

237. The crystalline form of embodiment 234, comprising four peaks at 2θ angles (±0.2) selected from the group consisting of 13.60°, 14.65°, 18.74°, and 19.10°.

238. The crystalline form of any one of embodiments 217 and 219-237, characterized by a differential scanning calorimetry (DSC) profile substantially similar to FIG. 16.

239. The crystalline form of any one of embodiments 217 and 219-237, characterized by a DSC that is about the same or is the same as the profile shown in FIG. 16.

240. The crystalline form of any one of embodiments 217 and 219-239, characterized by a differential scanning calorimetry (DSC) profile with no endothermic peak and an exothermic peak at about 215° C. (onset).

241. The crystalline form of any one of embodiments 217 and 219-240, characterized by a thermal gravimetric analysis (TGA) profile with about 0.5% weight loss at about the exothermic peak at about 215° C. (onset).

242. The crystalline form any one of embodiments 217 and 219-241, wherein the crystalline form is anhydrous.

243. The crystalline form of any one of embodiments 217 and 219-242, wherein the crystalline form comprises about 5000 ppm or less of n-heptane.

244. The crystalline form of any one of embodiments 217 and 219-242, wherein the crystalline form comprises about 4000 ppm or less of n-heptane.

245. The crystalline form of any one of embodiments 217 and 219-242, wherein the crystalline form comprises about 3000 ppm or less of n-heptane.

246. The crystalline form of any one of embodiments 217 and 219-242, wherein the crystalline form comprises about 2000 ppm or less of n-heptane.

247. The crystalline form of any one of embodiments 217 and 219-242, wherein the crystalline form comprises about 1000 ppm or less of n-heptane.

248. The crystalline form of any one of embodiments 217 and 219-242, wherein the crystalline form comprises about 500 ppm or less of n-heptane.

249. The crystalline form of any one of embodiments 217 and 219-242, wherein the crystalline form comprises about 400 ppm or less of n-heptane.

250. The crystalline form of any one of embodiments 217 and 219-242, wherein the crystalline form comprises about 300 ppm or less of n-heptane.

251. The crystalline form of any one of embodiments 217 and 219-242, wherein the crystalline form comprises about 200 ppm or less of n-heptane.

252. The crystalline form of any one of embodiments 217 and 219-242, wherein the crystalline form comprises about 100 ppm or less of n-heptane.

253. The crystalline form of any one of embodiments 217 and 219-242, wherein the crystalline form comprises about 70 ppm or less of n-heptane.

254. The crystalline form of any one of embodiments 217 and 219-242, wherein the crystalline form comprises about 60 ppm or less of n-heptane.

255. The crystalline form of any one of embodiments 217 and 219-242, wherein the crystalline form comprises about 20 ppm or less of n-heptane.

256. The crystalline form of any one of embodiments 217 and 219-242, wherein the crystalline form comprises about 15 ppm or less of n-heptane.

257. The crystalline form of any one of embodiments 217 and 219-242, wherein the crystalline form comprises about 10 ppm or less of n-heptane.

258. The crystalline form of any one of embodiments 217 and 219-242, wherein the crystalline form is substantially free of n-heptane.

259. The crystalline form of any one of embodiments 217 and 219-242, wherein the crystalline form is free of n-heptane.

260. The crystalline form of any one of embodiments 217 and 219-259, wherein the crystalline form comprises about 5000 ppm or less of 2-methyltetrahydrofuran.

261. The crystalline form of any one of embodiments 217 and 219-259, wherein the crystalline form comprises about 4000 ppm or less of 2-methyltetrahydrofuran.

262. The crystalline form of any one of embodiments 217 and 219-259, wherein the crystalline form comprises about 3000 ppm or less of 2-methyltetrahydrofuran.

263. The crystalline form of any one of embodiments 217 and 219-259, wherein the crystalline form comprises about 2000 ppm or less of 2-methyltetrahydrofuran.

264. The crystalline form of any one of embodiments 217 and 219-259, wherein the crystalline form comprises about 1000 ppm or less of 2-methyltetrahydrofuran.

265. The crystalline form of any one of embodiments 217 and 219-259, wherein the crystalline form comprises about 500 ppm or less of 2-methyltetrahydrofuran.

266. The crystalline form of any one of embodiments 217 and 219-259, wherein the crystalline form comprises about 400 ppm or less of 2-methyltetrahydrofuran.

267. The crystalline form of any one of embodiments 217 and 219-259, wherein the crystalline form comprises about 300 ppm or less of 2-methyltetrahydrofuran.

268. The crystalline form of any one of embodiments 217 and 219-259, wherein the crystalline form comprises about 200 ppm or less of 2-methyltetrahydrofuran.

269. The crystalline form of any one of embodiments 217 and 219-259, wherein the crystalline form comprises about 100 ppm or less of 2-methyltetrahydrofuran.

270. The crystalline form of any one of embodiments 217 and 219-259, wherein the crystalline form comprises about 70 ppm or less of 2-methyltetrahydrofuran.

271. The crystalline form of any one of embodiments 217 and 219-259, wherein the crystalline form comprises about 60 ppm or less of 2-methyltetrahydrofuran.

272. The crystalline form of any one of embodiments 217 and 219-259, wherein the crystalline form comprises about 20 ppm or less of 2-methyltetrahydrofuran.

273. The crystalline form of any one of embodiments 217 and 219-259, wherein the crystalline form comprises about 15 ppm or less of 2-methyltetrahydrofuran.

274. The crystalline form of any one of embodiments 217 and 219-259, wherein the crystalline form comprises about 10 ppm or less of 2-methyltetrahydrofuran.

275. The crystalline form of any one of embodiments 217 and 219-259, wherein the crystalline form is substantially free of 2-methyltetrahydrofuran.

276. The crystalline form of any one of embodiments 217 and 219-259, wherein the crystalline form is free of 2-methyltetrahydrofuran.

277. The crystalline form of any one of embodiments 217 and 219-276, wherein the crystalline form comprises about 5000 ppm or less of ethyl acetate.

278. The crystalline form of any one of embodiments 217 and 219-276, wherein the crystalline form comprises about 4000 ppm or less of ethyl acetate.

279. The crystalline form of any one of embodiments 217 and 219-276, wherein the crystalline form comprises about 3000 ppm or less of ethyl acetate.

280. The crystalline form of any one of embodiments 217 and 219-276, wherein the crystalline form comprises about 2000 ppm or less of ethyl acetate.

281. The crystalline form of any one of embodiments 217 and 219-276, wherein the crystalline form comprises about 1000 ppm or less of ethyl acetate.

282. The crystalline form of any one of embodiments 217 and 219-276, wherein the crystalline form comprises about 500 ppm or less of ethyl acetate.

283. The crystalline form of any one of embodiments 217 and 219-276, wherein the crystalline form comprises about 400 ppm or less of ethyl acetate.

284. The crystalline form of any one of embodiments 217 and 219-276, wherein the crystalline form comprises about 300 ppm or less of ethyl acetate.

285. The crystalline form of any one of embodiments 217 and 219-276, wherein the crystalline form comprises about 200 ppm or less of ethyl acetate.

286. The crystalline form of any one of embodiments 217 and 219-276, wherein the crystalline form comprises about 100 ppm or less of ethyl acetate.

287. The crystalline form of any one of embodiments 217 and 219-276, wherein the crystalline form comprises about 80 ppm or less of ethyl acetate.

288. The crystalline form of any one of embodiments 217 and 219-276, wherein the crystalline form comprises about 60 ppm or less of ethyl acetate.

289. The crystalline form of any one of embodiments 217 and 219-276, wherein the crystalline form comprises about 40 ppm or less of ethyl acetate.

290. The crystalline form of any one of embodiments 217 and 219-276, wherein the crystalline form comprises about 10 ppm or less of ethyl acetate.

291. The crystalline form of any one of embodiments 217 and 219-276, wherein the crystalline form is substantially free of ethyl acetate.

292. The crystalline form of any one of embodiments 217 and 219-276, wherein the crystalline form is free of ethyl acetate.

293. The crystalline form of any one of embodiments 217 and 219-292, wherein the crystalline form comprises 3000 ppm or less of methanol.

294. The crystalline form of any one of embodiments 217 and 219-292, wherein the crystalline form comprises 2000 ppm or less of methanol.

295. The crystalline form of any one of embodiments 217 and 219-292, wherein the crystalline form comprises 1,500 ppm or less of methanol.

296. The crystalline form of any one of embodiments 217-292, wherein the crystalline form comprises 1,400 ppm or less of methanol.

297. The crystalline form of any one of embodiments 217 and 219-292, wherein the crystalline form comprises 1,300 ppm or less of methanol.

298. The crystalline form of any one of embodiments 217 and 219-292, wherein the crystalline form comprises 1,250 ppm or less of methanol.

299. The crystalline form of any one of embodiments 217 and 219-292, wherein the crystalline form comprises 1,200 ppm or less of methanol.

300. The crystalline form of any one of embodiments 217 and 219-292, wherein the crystalline form comprises 1,100 ppm or less of methanol.

301. The crystalline form of any one of embodiments 217 and 219-292, wherein the crystalline form comprises 1,000 ppm or less of methanol.

302. The crystalline form of any one of embodiments 217 and 219-292, wherein the crystalline form comprises 800 ppm or less of methanol.

303. The crystalline form of any one of embodiments 217 and 219-292, wherein the crystalline form comprises about 600 ppm or less of methanol.

304. The crystalline form of any one of embodiments 217 and 219-292, wherein the crystalline form comprises about 400 ppm or less of methanol.

305. The crystalline form of any one of embodiments 217 and 219-292, wherein the crystalline form comprises about 300 ppm or less of methanol.

306. The crystalline form of any one of embodiments 217 and 219-292, wherein the crystalline form comprises about 200 ppm or less of methanol.

307. The crystalline form of any one of embodiments 217 and 219-292, wherein the crystalline form comprises about 100 ppm or less of methanol.

308. The crystalline form of any one of embodiments 217 and 219-292, wherein the crystalline form comprises about 60 ppm or less of methanol.

309. The crystalline form of any one of embodiments 217 and 219-292, wherein the crystalline form is substantially free of methanol.

310. The crystalline form of any one of embodiments 217 and 219-292, wherein the crystalline form is free of methanol.

311. The crystalline form of embodiment any one of embodiments 217 and 219-310, wherein the crystalline form comprises about 5000 ppm or less of ethanol.

312. The crystalline form of embodiment any one of embodiments 217 and 219-310, wherein the crystalline form comprises about 4000 ppm or less of ethanol.

313. The crystalline form of embodiment any one of embodiments 217 and 219-310, wherein the crystalline form comprises about 3000 ppm or less of ethanol.

314. The crystalline form of embodiment any one of embodiments 217 and 219-310, wherein the crystalline form comprises about 2000 ppm or less of ethanol.

315. The crystalline form of embodiment any one of embodiments 217 and 219-310, wherein the crystalline form comprises about 1000 ppm or less of ethanol.

316. The crystalline form of embodiment any one of embodiments 217 and 219-310, wherein the crystalline form comprises about 800 ppm or less of ethanol.

317. The crystalline form of embodiment any one of embodiments 217 and 219-310, wherein the crystalline form comprises about 600 ppm or less of ethanol.

318. The crystalline form of embodiment any one of embodiments 217 and 219-310, wherein the crystalline form comprises about 400 ppm or less of ethanol.

319. The crystalline form of embodiment any one of embodiments 217 and 219-310, wherein the crystalline form comprises about 200 ppm or less of ethanol.

320. The crystalline form of embodiment any one of embodiments 217 and 219-310, wherein the crystalline form comprises about 100 ppm or less of ethanol.

321. The crystalline form of embodiment any one of embodiments 217 and 219-310, wherein the crystalline form comprises about 80 ppm or less of ethanol.

322. The crystalline form of embodiment any one of embodiments 217 and 219-310, wherein the crystalline form comprises about 60 ppm or less of ethanol.

323. The crystalline form of embodiment any one of embodiments 217 and 219-310, wherein the crystalline form comprises about 40 ppm or less of ethanol.

324. The crystalline form of embodiment any one of embodiments 217 and 219-310, wherein the crystalline form comprises about 20 ppm or less of ethanol.

325. The crystalline form of embodiment any one of embodiments 217 and 219-310, wherein the crystalline form comprises about 15 ppm or less of ethanol.

326. The crystalline form of embodiment any one of embodiments 217 and 219-310, wherein the crystalline form is substantially free of ethanol.

327. The crystalline form of embodiment any one of embodiments 217 and 219-310, wherein the crystalline form is free of ethanol.

328. The crystalline form of any one of embodiments 217 and 219-327, wherein the crystalline form comprises about 600 ppm or less of dichloromethane.

329. The crystalline form of any one of embodiments 217 and 219-327, wherein the crystalline form comprises about 500 ppm or less of dichloromethane.

330. The crystalline form of any one of embodiments 217 and 219-327, wherein the crystalline form comprises about 400 ppm or less of dichloromethane.

331. The crystalline form of any one of embodiments 217 and 219-327, wherein the crystalline form comprises about 300 ppm or less of dichloromethane.

332. The crystalline form of any one of embodiments 217 and 219-327, wherein the crystalline form comprises about 200 ppm or less of dichloromethane.

333. The crystalline form of any one of embodiments 217 and 219-327, wherein the crystalline form comprises about 100 ppm or less of dichloromethane.

334. The crystalline form of any one of embodiments 217 and 219-327, wherein the crystalline form comprises about 80 ppm or less of dichloromethane.

335. The crystalline form of any one of embodiments 217 and 219-327, wherein the crystalline form comprises about 60 ppm or less of dichloromethane.

336. The crystalline form of any one of embodiments 217 and 219-327, wherein the crystalline form comprises about 40 ppm or less of dichloromethane.

337. The crystalline form of any one of embodiments 217 and 219-327, wherein the crystalline form comprises about 10 ppm or less of dichloromethane.

338. The crystalline form of any one of embodiments 217 and 219-327, wherein the crystalline form is substantially free of dichloromethane.

339. The crystalline form of any one of embodiments 217 and 219-327, wherein the crystalline form is free of dichloromethane.

340. The crystalline form of any one of any of embodiments 217 and 219-339, wherein the crystalline form is substantially free of methanol, ethanol, and/or dichloromethane.

341. The crystalline form of any one of any of embodiments 217 and 219-339, wherein the crystalline form is free of methanol, ethanol, and/or dichloromethane.

342. The crystalline form of any one of any of embodiments 217 and 219-339, wherein the crystalline form is substantially free of methanol, ethanol, and dichloromethane.

343. The crystalline form of any one of embodiments 217 and 219-339, wherein the crystalline form is free of methanol, ethanol, and dichloromethane.

344. The crystalline form of any one of embodiments 217 and 219-343, wherein the crystalline form comprises about 5000 ppm or less of acetone.

345. The crystalline form of any one of embodiments 217 and 219-343, wherein the crystalline form is substantially free of acetone.

346. The crystalline form of any one of embodiments 217 and 219-343, wherein the crystalline form is free of acetone.

347. The crystalline form of any one of embodiments 217 and 219-346, wherein the crystalline form comprises about 890 ppm or less of toluene.

348. The crystalline form of any one of embodiments 217 and 219-346, wherein the crystalline form is substantially free of toluene.

349. The crystalline form of any one of embodiments 217 and 219-346, wherein the crystalline form is free of toluene.

350. The crystalline form of any one of embodiments 217 and 219-349, wherein the crystalline form comprises about 3880 ppm or less of cyclohexane.

351. The crystalline form of any one of embodiments 217 and 219-349, wherein the crystalline form is substantially free of cyclohexane.

352. The crystalline form of any one of embodiments 217 and 219-349, wherein the crystalline form is free of cyclohexane.

353. The crystalline form of embodiment any one of embodiments 217 and 219-352, wherein the crystalline form comprises about 5000 ppm or less of residual solvents.

354. The crystalline form of embodiment any one of embodiments 217 and 219-352, wherein the crystalline form comprises about 4000 ppm or less of residual solvents.

355. The crystalline form of embodiment any one of embodiments 217 and 219-352, wherein the crystalline form comprises about 3000 ppm or less of residual solvents.

356. The crystalline form of embodiment any one of embodiments 217 and 219-352, wherein the crystalline form comprises about 2000 ppm or less of residual solvents.

357. The crystalline form of embodiment any one of embodiments 217 and 219-352, wherein the crystalline form comprises about 1000 ppm or less of residual solvents.

358. The crystalline form of embodiment any one of embodiments 217 and 219-352, wherein the crystalline form comprises about 800 ppm or less of residual solvents.

359. The crystalline form of embodiment any one of embodiments 217 and 219-352, wherein the crystalline form comprises about 600 ppm or less of residual solvents.

360. The crystalline form of embodiment any one of embodiments 217 and 219-352, wherein the crystalline form comprises about 400 ppm or less of residual solvents.

361. The crystalline form of embodiment any one of embodiments 217 and 219-352, wherein the crystalline form comprises about 200 ppm or less of residual solvents.

362. The crystalline form of embodiment any one of embodiments 217 and 219-352, wherein the crystalline form comprises about 100 ppm or less of residual solvents.

363. The crystalline form of embodiment any one of embodiments 217 and 219-352, wherein the crystalline form comprises about 50 ppm or less of residual solvents.

364. The crystalline form of embodiment any one of embodiments 217 and 219-352, wherein the crystalline form is substantially free of residual solvents.

365. The crystalline form of embodiment any one of embodiments 217 and 219-352, wherein the crystalline form is free of residual solvents.

366. The crystalline form of any one of embodiments 217 and 219-365, wherein the crystalline form has at least 99.0% purity.

367. The crystalline form of any one of embodiments 217 and 219-365, wherein the crystalline form has at least 99.1% purity.

368. The crystalline form of any one of embodiments 217 and 219-365, wherein the crystalline form has at least 99.2% purity.

369. The crystalline form of any one of embodiments 217 and 219-365, wherein the crystalline form has at least 99.3% purity.

370. The crystalline form of any one of embodiments 217 and 219-365, wherein the crystalline form has at least 99.4% purity.

371. The crystalline form of any one of embodiments 217 and 219-365, wherein the crystalline form has at least 99.5% purity.

372. The crystalline form of any one of embodiments 217 and 219-365, wherein the crystalline form has at least 99.6% purity.

373. The crystalline form of any one of embodiments 217 and 219-365, wherein the crystalline form has at least 99.7% purity.

374. The crystalline form of any one of embodiments 217 and 219-365, wherein the crystalline form has at least 99.8% purity.

375. The crystalline form of any one of embodiments 217 and 219-365, wherein the crystalline form has at least 99.9% purity.

376. The crystalline form of any one of embodiments 217 and 219-365, wherein the crystalline form has 100% purity.

377. The crystalline form of any one of embodiments 217 and 219-376, wherein the crystalline form is a crystalline Form III and comprises about 20 ppm or less of heavy metals.

378. The crystalline form of any one of embodiments 217 and 219-376, wherein the crystalline form is a crystalline Form III and comprises about 15 ppm or less of heavy metals.

379. The crystalline form of any one of embodiments 217 and 219-376, wherein the crystalline form is a crystalline Form III and comprises about 10 ppm or less of heavy metals.

380. The crystalline form of any one of embodiments 217 and 219-376, wherein the crystalline form is a crystalline Form III and comprises about 5 ppm or less of heavy metals.

381. The crystalline form of any one of embodiments 217 and 219-376, wherein the crystalline form is a crystalline Form III and comprises about 2 ppm or less of heavy metals.

382. The crystalline form of any one of embodiments 217 and 219-381, wherein the crystalline form is a crystalline Form III and comprises about 20 ppm or less of lead.

383. The crystalline form of any one of embodiments 217 and 219-381, wherein the crystalline form is a crystalline Form III and comprises about 15 ppm or less of lead.

384. The crystalline form of any one of embodiments 217 and 219-381, wherein the crystalline form is a crystalline Form III and comprises about 10 ppm or less of lead.

385. The crystalline form of any one of embodiments 217 and 219-381, wherein the crystalline form is a crystalline Form III and comprises about 5 ppm or less of lead.

386. The crystalline form of any one of embodiments 217 and 219-381, wherein the crystalline form is a crystalline Form III and comprises about 2 ppm or less of lead.

387. The crystalline form of any one of embodiments 217 and 219-381, wherein the crystalline form is a crystalline Form III and comprises about 1 ppm or less of lead.

388. The crystalline form of any one of embodiments 217 and 219-381, wherein the crystalline form is a crystalline Form III and comprises about 0.5 ppm or less of lead.

389. The crystalline form of any one of embodiments 217 and 219-381, wherein the crystalline form is a crystalline Form III and comprises about 0.1 ppm or less of lead.

390. The crystalline form of any one of embodiments 217 and 219-389, wherein the crystalline form is a crystalline Form III and comprises about 20 ppm or less of arsenic.

391. The crystalline form of any one of embodiments 217 and 219-389, wherein the crystalline form is a crystalline Form III and comprises about 15 ppm or less of arsenic.

392. The crystalline form of any one of embodiments 217 and 219-389, wherein the crystalline form is a crystalline Form III and comprises about 10 ppm or less of arsenic.

393. The crystalline form of any one of embodiments 217 and 219-389, wherein the crystalline form is a crystalline Form III and comprises about 5 ppm or less of arsenic.

394. The crystalline form of any one of embodiments 217 and 219-389, wherein the crystalline form is a crystalline Form III and comprises about 2 ppm or less of arsenic.

395. The crystalline form of any one of embodiments 217 and 219-389, wherein the crystalline form is a crystalline Form III and comprises about 1 ppm or less of arsenic.

396. The crystalline form of any one of embodiments 217 and 219-389, wherein the crystalline form is a crystalline Form III and comprises about 0.5 ppm or less of arsenic.

397. The crystalline form of any one of embodiments 217 and 219-389, wherein the crystalline form is a crystalline Form III and comprises about 0.1 ppm or less of arsenic.

398. The crystalline form of any one of embodiments 217 and 219-389, wherein the crystalline form is a crystalline Form III and comprises about 0.05 ppm or less of arsenic.

399. The crystalline form of any one of embodiments 217 and 219-398, wherein the crystalline form is a crystalline Form III and comprises about 20 ppm or less of cobalt.

400. The crystalline form of any one of embodiments 217 and 219-398, wherein the crystalline form is a crystalline Form III and comprises about 15 ppm or less of cobalt.

401. The crystalline form of any one of embodiments 217 and 219-398, wherein the crystalline form is a crystalline Form III and comprises about 10 ppm or less of cobalt.

402. The crystalline form of any one of embodiments 217 and 219-398, wherein the crystalline form is a crystalline Form III and comprises about 5 ppm or less of cobalt.

403. The crystalline form of any one of embodiments 217 and 219-398, wherein the crystalline form is a crystalline Form III and comprises about 2 ppm or less of cobalt.

404. The crystalline form of any one of embodiments 217 and 219-398, wherein the crystalline form is a crystalline Form III and comprises about 1 ppm or less of cobalt.

405. The crystalline form of any one of embodiments 217 and 219-398, wherein the crystalline form is a crystalline Form III and comprises about 0.5 ppm or less of cobalt.

406. The crystalline form of any one of embodiments 217 and 219-398, wherein the crystalline form is a crystalline Form III and comprises about 0.1 ppm or less of cobalt.

407. The crystalline form of any one of embodiments 217 and 219-406, wherein the crystalline form is a crystalline Form III and comprises about 20 ppm or less of vanadium.

408. The crystalline form of any one of embodiments 217 and 219-406, wherein the crystalline form is a crystalline Form III and comprises about 15 ppm or less of vanadium.

409. The crystalline form of any one of embodiments 217 and 219-406, wherein the crystalline form is a crystalline Form III and comprises about 10 ppm or less of vanadium.

410. The crystalline form of any one of embodiments 217 and 219-406, wherein the crystalline form is a crystalline Form III and comprises about 5 ppm or less of vanadium.

411. The crystalline form of any one of embodiments 217 and 219-406, wherein the crystalline form is a crystalline Form III and comprises about 2 ppm or less of vanadium.

412. The crystalline form of any one of embodiments 217 and 219-406, wherein the crystalline form is a crystalline Form III and comprises about 1 ppm or less of vanadium.

413. The crystalline form of any one of embodiments 217 and 219-406, wherein the crystalline form is a crystalline Form III and comprises about 0.5 ppm or less of vanadium.

414. The crystalline form of any one of embodiments 217 and 219-406, wherein the crystalline form is a crystalline Form III and comprises about 0.1 ppm or less of vanadium.

415. The crystalline form of any one of embodiments 217 and 219-414, wherein the crystalline form is a crystalline Form III and comprises about 20 ppm or less of nickel.

416. The crystalline form of any one of embodiments 217 and 219-414, wherein the crystalline form is a crystalline Form III and comprises about 15 ppm or less of nickel.

417. The crystalline form of any one of embodiments 217 and 219-414, wherein the crystalline form is a crystalline Form III and comprises about 10 ppm or less of nickel.

418. The crystalline form of any one of embodiments 217 and 219-414, wherein the crystalline form is a crystalline Form III and comprises about 5 ppm or less of nickel.

419. The crystalline form of any one of embodiments 217 and 219-414, wherein the crystalline form is a crystalline Form III and comprises about 2 ppm or less of nickel.

420. The crystalline form of any one of embodiments 217 and 219-414, wherein the crystalline form is a crystalline Form III and comprises about 1 ppm or less of nickel.

421. The crystalline form of any one of embodiments 217 and 219-414, wherein the crystalline form is a crystalline Form III and comprises about 0.5 ppm or less of nickel.

422. The crystalline form of any one of embodiments 217 and 219-421, wherein the crystalline form is a crystalline Form III and comprises about 20 ppm or less of titanium.

423. The crystalline form of any one of embodiments 217 and 219-421, wherein the crystalline form is a crystalline Form III and comprises about 15 ppm or less of titanium.

424. The crystalline form of any one of embodiments 217 and 219-421, wherein the crystalline form is a crystalline Form III and comprises about 10 ppm or less of titanium.

425. The crystalline form of any one of embodiments 217 and 219-421, wherein the crystalline form is a crystalline Form III and comprises about 5 ppm or less of titanium.

426. The crystalline form of any one of embodiments 217 and 219-421, wherein the crystalline form is a crystalline Form III and comprises about 2 ppm or less of titanium.

427. The crystalline form of any one of embodiments 217 and 219-421, wherein the crystalline form is a crystalline Form III and comprises about 1 ppm or less of titanium.

428. The crystalline form of any one of embodiments 217 and 219-427, wherein the crystalline form is a crystalline Form III and comprises about 20 ppm or less of mercury.

429. The crystalline form of any one of embodiments 217 and 219-427, wherein the crystalline form is a crystalline Form III and comprises about 5 ppm or less of mercury.

430. The crystalline form of any one of embodiments 217 and 219-427, wherein the crystalline form is a crystalline Form III and comprises about 1 ppm or less of mercury.

431. The crystalline form of any one of embodiments 217 and 219-427, wherein the crystalline form is a crystalline Form III is substantially free of mercury.

432. The crystalline form of any one of embodiments 217 and 219-431, wherein the crystalline form is a crystalline Form III and comprises about 20 ppm or less of cadmium.

433. The crystalline form of any one of embodiments 217 and 219-431, wherein the crystalline form is a crystalline Form III and comprises about 5 ppm or less of cadmium.

434. The crystalline form of any one of embodiments 217 and 219-431, wherein the crystalline form is a crystalline Form III and comprises about 1 ppm or less of cadmium.

435. The crystalline form of any one of embodiments 217 and 219-431, wherein the crystalline form is a crystalline Form III is substantially free of cadmium.

436. The crystalline form of any one of embodiments 217 and 219-435, wherein the crystalline form is a crystalline Form III and comprises less than 15% of other crystalline forms of Compound I.

437. The crystalline form of any one of embodiments 217 and 219-435, wherein the crystalline form is a crystalline Form III and comprises less than 10% of other crystalline forms of Compound I.

438. The crystalline form of any one of embodiments 217 and 219-435, wherein the crystalline form is a crystalline Form III and comprises less than 5% of other crystalline forms of Compound I.

439. The crystalline form of any one of embodiments 217 and 219-435, wherein the crystalline form is a crystalline Form III and comprises less than 4% of other crystalline forms of Compound I.

440. The crystalline form of any one of embodiments 217 and 219-435, wherein the crystalline form is a crystalline Form III and comprises less than 3% of other crystalline forms of Compound I.

441. The crystalline form of any one of embodiments 217 and 219-435, wherein the crystalline form is a crystalline Form III and comprises less than 2% of other crystalline forms of Compound I.

442. The crystalline form of any one of embodiments 217 and 219-435, wherein the crystalline form is a crystalline Form III and comprises less than 1% of other crystalline forms of Compound I.

443. The crystalline form of any one of embodiments 217 and 219-435, wherein the crystalline form is a crystalline Form III and comprises less than 0.5% of other crystalline forms of Compound I.

444. The crystalline form of any one of embodiments 217 and 219-435, wherein the crystalline form is a crystalline Form III and does not contain any other crystalline forms of Compound I.

445. A high purity form of Compound I, wherein the high purity form of Compound I is the crystalline Form III of Compound I of any one of embodiments 217 and 219-444.

446. A crystalline Form I of 3-hydroxy-9β,13α-dimethyl-2-oxo-24,25,26-trinoroleana-1(10),3,5,7-tetraen-29-oic acid,

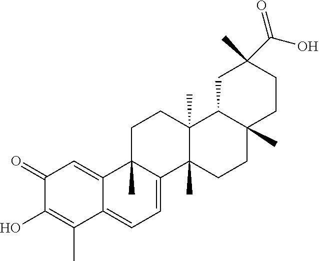

characterized by an x-ray powder diffraction pattern comprising peaks at 2θ angles (±0.2) of 9.53°, 13.29°, and 16.13°.

447. The crystalline form of embodiment 446, characterized by an X-ray powder diffraction pattern comprising one or more peaks at 2θ angles (±0.2) selected from the group consisting of 8.34°, 11.12°, 15.08°, 15.35°, 20.86°, and 22.95°.

448. The crystalline form of embodiment 446, characterized by a differential scanning calorimetry (DSC) profile with an endothermic peak at about 155° C. (onset) and an exothermic peak at about 222° C. (onset).

449. The crystalline form of embodiment 446, characterized by a thermal gravimetric analysis (TGA) profile with about 4.7% weight loss at about 155° C.

450. A crystalline Form II of 3-hydroxy-9β,13α-dimethyl-2-oxo-24,25,26-trinoroleana-1(10),3,5,7-tetraen-29-oic acid,

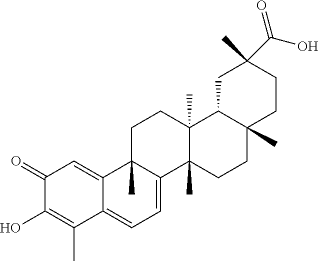

characterized by an X-ray powder diffraction pattern comprising peaks at 2θ angles (±0.2) of 11.68°, 16.07°, and 17.04°.

451. The crystalline form of embodiment 450, characterized by an X-ray powder diffraction pattern comprising one or more peaks at 2θ angles (±0.2) selected from the group consisting of 8.48°, 14.05°, 14.68°, 19.30°, 24.18, and 26.87°.

452. The crystalline form of embodiment 450, characterized by a differential scanning calorimetry (DSC) profile with an endothermic peak at about 155° C. (onset) and an exothermic peak at about 213° C. (onset).

453. The crystalline form of embodiment 450, characterized by a thermal gravimetric analysis (TGA) profile with about 7% weight loss at about 155° C.

454. A crystalline Form VI of 3-hydroxy-9β,13α-dimethyl-2-oxo-24,25,26-trinoroleana-1(10),3,5,7-tetraen-29-oic acid,

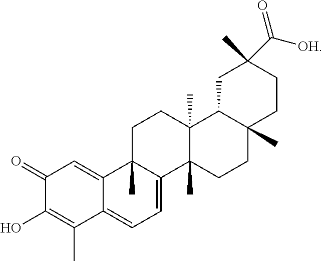

455. The crystalline form of embodiment 454, characterized by an X-ray powder diffraction pattern comprising peaks at 2θ angles (±0.2) of 13.4°, 14.6°, and 17.4°.

456. The crystalline form of embodiment 454, characterized by an X-ray powder diffraction pattern comprising peaks at 2θ angles (±0.2) of 11.1°, 16.2°, and 17.4°.

457. The crystalline form of embodiment 454, characterized by an X-ray powder diffraction pattern comprising peaks at 2θ angles (±0.2) of 11.5°, 16.2°, and 17.4°.

458. The crystalline form of embodiment 454, characterized by an X-ray powder diffraction pattern comprising peaks at 2θ angles (±0.2) of 11.1°, 13.4°, 15.0°, 16.2°, and 17.4°.

459. The crystalline form of embodiment 454, characterized by an X-ray powder diffraction pattern comprising peaks at 2θ angles (±0.2) of 11.5°, 13.4°, 15.0°, 16.2°, and 17.4°.

460. The crystalline form of embodiment 454, characterized by an X-ray powder diffraction pattern comprising one or more peaks (e.g., one, two, three, four, five, or six peaks) at 2θ angles (±0.2) selected from the group consisting of 11.1°, 13.4°, 15.0°, 16.2°, and 17.4°.

461. The crystalline form of embodiment 454, characterized by an X-ray powder diffraction pattern comprising two peaks at 2θ angles (±0.2) selected from the group consisting of 11.1°, 13.4°, 15.0°, 16.2°, and 17.4°.

462. The crystalline form of embodiment 454, characterized by an X-ray powder diffraction pattern comprising three peaks at 2θ angles (±0.2) selected from the group consisting of 11.1°, 13.4°, 15.0°, 16.2°, and 17.4°.

463. The crystalline form of embodiment 454, characterized by an X-ray powder diffraction pattern comprising four peaks at 2θ angles (±0.2) selected from the group consisting of 11.1°, 13.4°, 15.0°, 16.2°, and 17.4°.

464. The crystalline form of embodiment 454, characterized by an X-ray powder diffraction pattern comprising five peaks at 2θ angles (±0.2) selected from the group consisting of 11.1°, 13.4°, 15.0°, 16.2°, and 17.4°.

465. The crystalline form of embodiment 454, characterized by an X-ray powder diffraction pattern comprising one or more peaks (e.g., one, two, three, four, five, or six peaks) at 2θ angles (±0.2) selected from the group consisting of 11.5°, 13.4°, 15.0°, 16.2°, and 17.4°.

466. The crystalline form of embodiment 454, characterized by an X-ray powder diffraction pattern comprising two peaks at 2θ angles (±0.2) selected from the group consisting of 11.5°, 13.4°, 15.0°, 16.2°, and 17.4°.

467. The crystalline form of embodiment 454, characterized by an X-ray powder diffraction pattern comprising three peaks at 2θ angles (±0.2) selected from the group consisting of 11.5°, 13.4°, 15.0°, 16.2°, and 17.4°.

468. The crystalline form of embodiment 454, characterized by an X-ray powder diffraction pattern comprising four peaks at 2θ angles (±0.2) selected from the group consisting of 11.5°, 13.4°, 15.0°, 16.2°, and 17.4°.

469. The crystalline form of embodiment 454, characterized by an X-ray powder diffraction pattern comprising five peaks at 2θ angles (±0.2) selected from the group consisting of 11.5°, 13.4°, 15.0°, 16.2°, and 17.4°.

470. The crystalline form of embodiment 454, characterized by an X-ray powder diffraction pattern comprising two peaks at 2θ angles (±0.2) selected from the group consisting of 11.5°, 13.4°, 15.0°, 16.2°, and 17.4°.

471. The crystalline form of embodiment 454, characterized by an X-ray powder diffraction pattern comprising three peaks at 2θ angles (±0.2) selected from the group consisting of 11.5°, 13.4°, 15.0°, 16.2°, and 17.4°.

472. The crystalline form of embodiment 454, characterized by an X-ray powder diffraction pattern comprising four peaks at 2θ angles (±0.2) selected from the group consisting of 11.5°, 13.4°, 15.0°, 16.2°, and 17.4°.

473. The crystalline form of embodiment 454, characterized by an X-ray powder diffraction pattern comprising five peaks at 2θ angles (±0.2) selected from the group consisting of 11.5°, 13.4°, 15.0°, 16.2°, and 17.4°.

474. The crystalline form of embodiment 454, characterized by an X-ray powder diffraction pattern comprising peaks at 2θ angles (±0.2) of 9.5, 11.1°, 11.5°, 13.4°, 14.6°, 15.0°, 15.2°, 16.2°, 17.4°, 19.0°, and 19.3°.

475. The crystalline form of embodiment 454, characterized by an X-ray powder diffraction pattern comprising one or more peaks (e.g., one, two, three, four, five, or six peaks) at 2θ angles (±0.2) selected from the group consisting of 9.5, 11.1°, 11.5°, 13.4°, 14.6°, 15.0°, 15.2°, 16.2°, 17.4°, 19.0°, and 19.3°.

476. The crystalline form of embodiment 454, characterized by an X-ray powder diffraction pattern comprising one or more peaks two peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

477. The crystalline form of embodiment 454, characterized by an X-ray powder diffraction pattern comprising one or more peaks three peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

478. The crystalline form of embodiment 454, characterized by an X-ray powder diffraction pattern comprising one or more peaks four peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

479. The crystalline form of embodiment 454, characterized by an X-ray powder diffraction pattern comprising one or more peaks five peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

480. The crystalline form of embodiment 454, characterized by an X-ray powder diffraction pattern comprising one or more peaks six peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

481. The crystalline form of embodiment 454, characterized by an X-ray powder diffraction pattern comprising two peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

482. The crystalline form of embodiment 454, characterized by an X-ray powder diffraction pattern comprising three peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

483. The crystalline form of embodiment 454, characterized by an X-ray powder diffraction pattern comprising four peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

484. The crystalline form of embodiment 454, characterized by an X-ray powder diffraction pattern comprising five peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

485. The crystalline form of embodiment 454, characterized by an X-ray powder diffraction pattern comprising six peaks at 2θ angles (±0.2) selected from the group consisting of 9.5, 11.1°, 11.5°, 13.4°, 14.6°, 15.0°, 15.2°, 16.2°, 17.4°, 19.0°, and 19.3°.

486. The crystalline form of embodiment 454, characterized by an X-ray powder diffraction pattern comprising peaks at 2θ angles (±0.2) of 9.5, 11.1°, 11.5°, 13.4°, 14.6°, 15.0°, 15.2°, 16.2°, 17.4°, 19.0°, 19.3°, 20.9°, and 23.0°.

487. The crystalline form of embodiment 454, characterized by an X-ray powder diffraction pattern comprising one or more peaks (e.g., one, two, three, four, five, or six peaks) at 2θ angles (±0.2) selected from the group consisting of 9.5, 11.1°, 11.5°, 13.4°, 14.6°, 15.0°, 15.2°, 16.2°, 17.4°, 19.0°, 19.3°, 20.9°, and 23.0°.

488. The crystalline form of embodiment 454, characterized by an X-ray powder diffraction pattern comprising two peaks at 2θ angles (±0.2) selected from the group consisting of 9.5, 11.1°, 11.5°, 13.4°, 14.6°, 15.0°, 15.2°, 16.2°, 17.4°, 19.0°, 19.3°, 20.9°, and 23.0°.

489. The crystalline form of embodiment 454, characterized by an X-ray powder diffraction pattern comprising three peaks at 2θ angles (±0.2) selected from the group consisting of 9.5, 11.1°, 11.5°, 13.4°, 14.6°, 15.0°, 15.2°, 16.2°, 17.4°, 19.0°, 19.3°, 20.9°, and 23.0°.

490. The crystalline form of embodiment 454, characterized by an X-ray powder diffraction pattern comprising four peaks at 2θ angles (±0.2) selected from the group consisting of 9.5, 11.1°, 11.5°, 13.4°, 14.6°, 15.0°, 15.2°, 16.2°, 17.4°, 19.0°, 19.3°, 20.9°, and 23.0°.

491. The crystalline form of embodiment 454, characterized by an X-ray powder diffraction pattern comprising five peaks at 2θ angles (±0.2) selected from the group consisting of 9.5, 11.1°, 11.5°, 13.4°, 14.6°, 15.0°, 15.2°, 16.2°, 17.4°, 19.0°, 19.3°, 20.9°, and 23.0°.

492. The crystalline form of embodiment 454, characterized by an X-ray powder diffraction pattern comprising six peaks at 2θ angles (±0.2) selected from the group consisting of 9.5, 11.1°, 11.5°, 13.4°, 14.6°, 15.0°, 15.2°, 16.2°, 17.4°, 19.0°, 19.3°, 20.9°, and 23.0°.

493. The crystalline form of embodiment 454, characterized by an X-ray powder diffraction pattern comprising peaks at 2θ angles (±0.2) of 9.5°, 11.1°, 11.5°, 13.4°, 14.6°, 15.0°, 15.2°, 16.2°, 17.4°, 19.0°, 19.3°, 20.0°, 20.9°, 21.4°, 22.0°, 23.0°, 24.0°, 24.5°, 25.9°, and 26.6°.

494. The crystalline form of any one of embodiments 454-493, characterized by an X-ray powder diffraction pattern substantially the same as FIG. 22.

495. The crystalline form of embodiment 454, characterized by an X-ray powder diffraction pattern comprising peaks at 2θ angles (±0.2) of 13.2°, 14.9, 16.1, and 17.2°.

496. The crystalline form of embodiment 454, characterized by an X-ray powder diffraction pattern comprising peaks at 2θ angles (±0.2) of 10.9°, 13.2°, 14.9, 16.1, and 17.2°.

497. The crystalline form of embodiment 454, characterized by one or more peaks (e.g., one, two, three, four, five, or six peaks) at 2θ angles (±0.2) selected from the group consisting of 10.0°, 10.9°, 18.5°, and 20.5°.

498. The crystalline form of embodiment 454, characterized by two peaks at 2θ angles (±0.2) selected from the group consisting of 10.0°, 10.9°, 18.5°, and 20.5°.

499. The crystalline form of embodiment 454, characterized by three peaks at 2θ angles (±0.2) selected from the group consisting of 10.0°, 10.9°, 18.5°, and 20.5°.

500. The crystalline form of embodiment 454, characterized by four peaks at 2θ angles (±0.2) selected from the group consisting of 10.0°, 10.9°, 18.5°, and 20.5°.

501. The crystalline form of embodiment 454, characterized by one or more peaks (e.g., one, two, three, four, five, or six peaks) at 2θ angles (±0.2) selected from the group consisting of 10.0°, 10.9°, 11.6°, 13.2°, 16.1°, and 20.5°.

502. The crystalline form of embodiment 454, characterized by two peaks at 2θ angles (±0.2) selected from the group consisting of 10.0°, 10.9°, 11.6°, 13.2°, 16.1°, and 20.5°.

503. The crystalline form of embodiment 454, characterized by three peaks at 2θ angles (±0.2) selected from the group consisting of 10.0°, 10.9°, 11.6°, 13.2°, 16.1°, and 20.5°.

504. The crystalline form of embodiment 454, characterized by four peaks at 2θ angles (±0.2) selected from the group consisting of 10.0°, 10.9°, 11.6°, 13.2°, 16.1°, and 20.5°.

505. The crystalline form of embodiment 454, characterized by five peaks at 2θ angles (±0.2) selected from the group consisting of 10.0°, 10.9°, 11.6°, 13.2°, 16.1°, and 20.5°.

506. The crystalline form of embodiment 454, characterized by six peaks at 2θ angles (±0.2) selected from the group consisting of 10.0°, 10.9°, 11.6°, 13.2°, 16.1°, and 20.5°.

507. The crystalline form of embodiment 454, characterized by one or more peaks (e.g., one, two, three, four, five, or six peaks) at 2θ angles (±0.2) selected from the group consisting of 10.0°, 10.9°, 11.6°, 13.2°, 14.9°, 16.1°, 17.2°, 18.5°, and 20.5°.

508. The crystalline form of embodiment 454, characterized by two peaks at 2θ angles (±0.2) selected from the group consisting of 10.0°, 10.9°, 11.6°, 13.2°, 14.9°, 16.1°, 17.2°, 18.5°, and 20.5°.

509. The crystalline form of embodiment 454, characterized by three peaks at 2θ angles (±0.2) selected from the group consisting of 10.0°, 10.9°, 11.6°, 13.2°, 14.9°, 16.1°, 17.2°, 18.5°, and 20.5°.

510. The crystalline form of embodiment 454, characterized by four peaks at 2θ angles (±0.2) selected from the group consisting of 10.0°, 10.9°, 11.6°, 13.2°, 14.9°, 16.1°, 17.2°, 18.5°, and 20.5°.

511. The crystalline form of embodiment 454, characterized by five, six peaks at 2θ angles (±0.2) selected from the group consisting of 10.0°, 10.9°, 11.6°, 13.2°, 14.9°, 16.1°, 17.2°, 18.5°, and 20.5°.

512. The crystalline form of embodiment 454, characterized by six peaks at 2θ angles (±0.2) selected from the group consisting of 10.0°, 10.9°, 11.6°, 13.2°, 14.9°, 16.1°, 17.2°, 18.5°, and 20.5°.

513. The crystalline form of embodiment 454, characterized by one or more peaks (e.g., one, two, three, four, five, or six peaks) at 2θ angles (±0.2) selected from the group consisting of 6.3°, 7.40, 10.00, 10.90, 11.60, 13.20, 14.9°, 16.1°, 17.2°, 18.5°, and 20.5°.

514. The crystalline form of embodiment 454, characterized by two, three, four, five, six peaks at 2θ angles (±0.2) selected from the group consisting of 6.3°, 7.4°, 10.0°, 10.9°, 11.6°, 13.20, 14.9°, 16.1°, 17.2°, 18.5°, and 20.5°.

515. The crystalline form of embodiment 454, characterized by three peaks at 2θ angles (±0.2) selected from the group consisting of 6.3°, 7.4°, 10.0°, 10.9°, 11.6°, 13.2°, 14.9°, 16.1°, 17.2°, 18.5°, and 20.5°.

516. The crystalline form of embodiment 454, characterized by four peaks at 2θ angles (±0.2) selected from the group consisting of 6.3°, 7.4°, 10.0°, 10.9°, 11.6°, 13.2°, 14.9°, 16.1°, 17.2°, 18.5°, and 20.5°.

517. The crystalline form of embodiment 454, characterized by five peaks at 2θ angles (±0.2) selected from the group consisting of 6.3°, 7.4°, 10.0°, 10.9°, 11.6°, 13.2°, 14.9°, 16.1°, 17.2°, 18.5°, and 20.5°.

518. The crystalline form of embodiment 454, characterized by six peaks at 2θ angles (±0.2) selected from the group consisting of 6.3°, 7.4°, 10.0°, 10.9°, 11.6°, 13.2°, 14.9°, 16.1°, 17.2°, 18.5°, and 20.5°.

519. The crystalline form of any one of embodiments 454 and 495-518, characterized by an X-ray powder diffraction pattern substantially the same as the second pattern from the bottom of FIG. 35.

520. The crystalline form of any one of embodiments 454 and 495-518, characterized by an X-ray powder diffraction pattern substantially the same as the bottom pattern FIG. 35.

521. A pharmaceutical composition comprising:
(i) a pharmaceutical agent, wherein the pharmaceutical agent is a crystalline form of Compound I of any one of embodiments 217 and 219-520;
(ii) an emulsifier or surfactant; and
(iii) a mucoadhesive or viscosifier;
wherein the pharmaceutical composition is in the form of a liquid suspension.

522. The pharmaceutical composition of embodiment 521, comprising:
(i) a pharmaceutical agent, wherein the pharmaceutical agent is a crystalline form of Compound I of any one of embodiments 217 and 219-520;
(ii) an emulsifier or surfactant;
(iii) a mucoadhesive or viscosifier; and
(iv) a preservative.

523. The pharmaceutical composition of embodiment 521, wherein the pharmaceutical composition comprises the emulsifier or surfactant at a concentration of from about 5 mg/mL to about 50 mg/mL.

524. The pharmaceutical composition of any one of embodiments 521-523, wherein the pharmaceutical composition comprises the emulsifier or surfactant at a concentration of from about 5 mg/mL to about 40 mg/mL, from about 5 mg/mL to about 30 mg/mL, from about 10 mg/mL to about 30 mg/mL, or from about 20 mg/mL to about 30 mg/mL.

525. The pharmaceutical composition of any one of embodiments 521-523, wherein the pharmaceutical composition comprises the emulsifier or surfactant at a concentration of about 25 mg/mL.

526. The pharmaceutical composition of any one of embodiments 521-523, wherein the pharmaceutical composition comprises the emulsifier or surfactant at a molar concentration of from about 5 mM to about 40 mM.

527. The pharmaceutical composition of any one of embodiments 521-523, wherein the pharmaceutical composition comprises the emulsifier or surfactant at a molar concentration of from about 10 mM to about 30 mM.

528. The pharmaceutical composition of any one of embodiments 521-523, wherein the pharmaceutical composition comprises the emulsifier or surfactant at a molar concentration of about 20 mM.

529. The pharmaceutical composition of any one of embodiments 521-528, wherein the pharmaceutical composition comprises the mucoadhesive or viscosifier at a concentration of from about 0.1 mg/mL to about 5 mg/mL.

530. The pharmaceutical composition of any one of embodiments 521-528, wherein the pharmaceutical composition comprises the mucoadhesive or viscosifier at a concentration of from about 0.1 mg/mL to about 4 mg/mL, from about 0.1 mg/mL to about 3 mg/mL, from about 0.1 mg/mL to about 2 mg/mL, or from about 0.5 mg/mL to about 1.5 mg/mL.

531. The pharmaceutical composition of any one of embodiments 521-528, wherein the pharmaceutical composition comprises the mucoadhesive or viscosifier at a concentration of about 1 mg/mL.

532. The pharmaceutical composition of any one of embodiments 521-528, wherein the pharmaceutical composition comprises the mucoadhesive or viscosifier at a concentration of from about 5 mg/mL to about 50 mg/mL.

533. The pharmaceutical composition of any one of embodiments 521-528, wherein the pharmaceutical composition comprises the mucoadhesive or viscosifier at a concentration of from about 5 mg/mL to about 40 mg/mL, from about 5 mg/mL to about 30 mg/mL, from about 10 mg/mL to about 30 mg/mL, from about 5 mg/mL to about 20 mg/mL, from about 10 mg/mL to about 20 mg/mL, or from about 15 mg/mL to about 20 mg/mL.

534. The pharmaceutical composition of any one of embodiments 521-528, wherein the pharmaceutical composition comprises the mucoadhesive or viscosifier at a concentration of about 18 mg/mL.

535. The pharmaceutical composition of any one of embodiments 521-534, wherein the surfactant is a nonionic surfactant.

536. The pharmaceutical composition of any one of embodiments 521-534, wherein the emulsifier or surfactant is a polysorbate.

537. The pharmaceutical composition of any one of embodiments 521-534, wherein the emulsifier or surfactant is selected from polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), and polyoxyethylene (20) sorbitan monooleate (polysorbate 80).

538. The pharmaceutical composition of any one of embodiments 521-534, wherein the emulsifier or surfactant is polyoxyethylene (20) sorbitan monolaurate (tween 20, polysorbate 20).

539. The pharmaceutical composition of any one of embodiments 521-538, wherein the mucoadhesive or viscosifier is selected from methylcellulose (MC), carboxymethylcellulose (CMC), sodium carboxymethylcellulose (Na-CMC), hydroxypropylmethylcellulose (HPMC), hydroxethylcellulose (HEC), microcrystalline cellulose (MCC), and combinations thereof.

540. The pharmaceutical composition of any one of embodiments 521-538, wherein the mucoadhesive or viscosifier is selected from methylcellulose (MC), microcrystalline cellulose (MCC), sodium carboxymethylcellulose (Na-CMC), hydroxypropylmethylcellulose (HPMC), and combinations thereof.

541. The pharmaceutical composition of any one of embodiments 521-540, wherein the mucoadhesive or viscosifier is selected from microcrystalline cellulose (MCC), sodium carboxymethyl cellulose (Na-CMC) and a combination thereof (MCC-NaCMC).

542. The pharmaceutical composition of any one of embodiments 521-540, wherein the mucoadhesive or viscosifier is hydroxypropylmethylcellulose (HPMC).

543. The pharmaceutical composition of any one of embodiments 521-540, wherein the mucoadhesive or viscosifier is a combination of microcrystalline cellulose (MCC) and sodium carboxymethyl cellulose (Na-CMC) (MCC-NaCMC).

544. A pharmaceutical composition comprising:
(i) a pharmaceutical agent, wherein the pharmaceutical agent is a crystalline form of Compound I of any one of embodiments 217 and 219-520;
(ii) a polysorbate; and
(iii) a cellulose;
wherein the pharmaceutical composition is in the form of a liquid suspension.

545. The pharmaceutical composition of embodiment 544, wherein the pharmaceutical composition comprises:
(i) a pharmaceutical agent, wherein the pharmaceutical agent is a crystalline form of Compound I of any one of embodiments 217 and 219-520;
(ii) a polysorbate;
(iii) a cellulose; and
(iv) a preservative.

546. The pharmaceutical composition of any one of embodiments 522-543 and 545, wherein the pharmaceutical composition comprises the preservative at a concentration of up to about 1 mg/mL.

547. The pharmaceutical composition of any one of embodiments 522-543 and 545, wherein the pharmaceutical composition comprises the preservative at a concentration of up to about 0.1 mg/mL, up to about 0.2 mg/mL, up to about 0.3 mg/mL, up to about 0.4 mg/mL, up to about 0.5 mg/mL, up to about 0.6 mg/mL, up to about 0.7 mg/mL, up to about 0.8 mg/mL, or up to about 0.9 mg/mL.

548. The pharmaceutical composition of any one of embodiments 522-543 and 545, wherein the pharmaceutical composition comprises the pharmaceutical composition comprises the preservative at a concentration of from about 0.01 mg/mL to 1.00 mg/mL.

549. The pharmaceutical composition of any one of embodiments 522-543 and 545, wherein the pharmaceutical composition comprises the pharmaceutical composition comprises the preservative an amount of from about 0.01 mg/mL to about 0.8 mg/mL, from about 0.01 mg/mL to about 0.6 mg/mL, from about 0.01 mg/mL to about 0.4 mg/mL, from about 0.01 mg/mL to about 0.2 mg/mL, from about 0.05 mg/mL to about 0.2 mg/mL, or from about 0.05 mg/mL to about 0.15 mg/mL.

550. The pharmaceutical composition of any one of embodiments 522-543 and 545, wherein the pharmaceutical composition comprises the preservative at a concentration of about 0.1 mg/mL.

551. The pharmaceutical composition of any one of embodiments 522-543 and 545, wherein the pharmaceutical composition comprises the preservative at a molar concentration of from about 0.1 mM to about 0.5 mM.

552. The pharmaceutical composition of any one of embodiments 522-543 and 545, wherein the pharmaceutical composition comprises the preservative at a molar concentration of from about 0.2 mM to about 0.4 mM.

553. The pharmaceutical composition of any one of embodiments 522-543 and 545, wherein the pharmaceutical composition comprises the preservative at a molar concentration of about 0.3 mM.

554. The pharmaceutical composition of any one of embodiments 544-553, wherein the pharmaceutical composition comprises the polysorbate at a molar concentration of from about 5 mg/mL to about 50 mg/mL.

555. The pharmaceutical composition of any one of embodiments 544-553, wherein the pharmaceutical composition comprises the polysorbate at a molar concentration of from about 5 mM to about 40 mM.

556. The pharmaceutical composition of any one of embodiments 544-553, wherein the pharmaceutical composition comprises the polysorbate at a molar concentration of from about 10 mM to about 30 mM.

557. The pharmaceutical composition of any one of embodiments 544-553, wherein the pharmaceutical composition comprises the polysorbate at a molar concentration of about 20 mM.

558. The pharmaceutical composition of any one of embodiments 544-557, wherein the pharmaceutical composition comprises the cellulose at a concentration of from about 0.1 mg/mL to about 5 mg/mL.

559. The pharmaceutical composition of any one of embodiments 544-557, wherein the pharmaceutical composition comprises the cellulose at a concentration of from about 5 mg/mL to about 50 mg/mL.

560. The pharmaceutical composition of any one of embodiments 544-557, wherein the pharmaceutical composition comprises cellulose at a concentration of about 18 mg/mL.

561. The pharmaceutical composition of any one of embodiments 521-560, wherein the pharmaceutical composition comprises the pharmaceutical agent at a concentration of from about 5 mg/mL to about 50 mg/mL.

562. The pharmaceutical composition of any one of embodiments 521-560, wherein the pharmaceutical composition comprises the pharmaceutical agent at a concentration of from about 5 mg/mL to about 40 mg/mL, from about 5 mg/mL to about 30 mg/mL, from about 10 mg/mL to about 30 mg/mL, or from about 15 mg/mL to about 25 mg/mL.

563. The pharmaceutical composition of any one of embodiments 521-560, wherein the pharmaceutical composition comprises the pharmaceutical agent at a concentration of about 20 mg/mL.

564. The pharmaceutical composition of any one of embodiments 521-560, wherein the pharmaceutical composition comprises the pharmaceutical agent at a molar concentration of from about 10 mM to about 70 mM.

565. The pharmaceutical composition of any one of embodiments 521-560, wherein the pharmaceutical composition comprises the pharmaceutical agent at a molar concentration of from about 20 mM to about 60 mM.

566. The pharmaceutical composition of any one of embodiments 521-560, wherein the pharmaceutical composition comprises the pharmaceutical agent at a molar concentration of from about 40 mM to about 50 mM.

567. The pharmaceutical composition of any one of embodiments 521-560, wherein the pharmaceutical composition comprises the pharmaceutical agent at a molar concentration of about 44 mM.

568. The pharmaceutical composition of any one of embodiments 522-543 and 545-567, wherein the preservative is selected from potassium sorbate, chlorobutanol, methyl paraben, propyl paraben, butyl paraben, benzethonium chloride, sodium benzoate, and sorbic acid, and benzalkonium chloride.

569. The pharmaceutical composition of any one of embodiments 522-543 and 545-567, wherein the preservative is benzalkonium chloride.

570. The pharmaceutical composition of any one of embodiments 544-569, wherein the cellulose is hydroxypropylmethylcellulose (HPMC) or a combination of microcrystalline cellulose (MCC) and sodium carboxymethyl cellulose (Na-CMC) (MCC-NaCMC).

571. The pharmaceutical composition of any one of embodiments 544-570, wherein the polysorbate is selected from polyoxyethylene (20) sorbitan monolaurate (polysorbate 20), polyoxyethylene (20) sorbitan monopalmitate (polysorbate 40), polyoxyethylene (20) sorbitan monostearate (polysorbate 60), and polyoxyethylene (20) sorbitan monooleate (polysorbate 80).

572. The pharmaceutical composition of any one of embodiments 544-570, wherein the polysorbate is polyoxyethylene (20) sorbitan monolaurate (tween 20, polysorbate 20).

573. A pharmaceutical composition comprising:
(i) a pharmaceutical agent, wherein the pharmaceutical agent is a crystalline form of Compound I of any one of embodiments 217 and 219-520 in an amount of from about 1 wt. % to about 5 wt. %;
(ii) hydroxypropyl-beta-cyclodextrin (HPBCD) or trehalose in an amount of from about 60 wt. % to about 70 wt. %; and
(iii) hydroxypropyl methyl cellulose (HPMC) in an amount of from about 10 wt. % to about 40 wt. %;
wherein the pharmaceutical composition is in the form of a powder.

574. The pharmaceutical composition of embodiment 573, wherein the pharmaceutical composition comprises the pharmaceutical agent in an amount of about 2 wt. %.

575. The pharmaceutical composition of embodiment 573 or 574, wherein the pharmaceutical composition comprises the HPBCD or trehalose in an amount in an amount of about 68 wt. %.

576. The pharmaceutical composition of any one of embodiments 573-575, wherein the pharmaceutical composition comprises the HPMC in an amount in an amount of about 30 wt. %.

577. The pharmaceutical composition of any one of embodiments 521-576, wherein the pharmaceutical composition is a fast-acting pharmaceutical composition.

578. The pharmaceutical composition of any one of embodiments 1-572 and 577, wherein the pharmaceutical composition is formulated for intranasal administration.

579. The pharmaceutical composition of any one of embodiments 1-572 and 577, wherein the pharmaceutical composition is in the form of a nasal spray or nose drops.

580. A synthetic intermediate for the preparation of Compound 1 Form III, wherein the synthetic intermediate is Compound 1 Form IV, of any one of embodiments 454-520.

581. The process of any one of embodiments 1-216, wherein the crystalline Form III of Compound I is a crystalline Form III of Compound I as described in embodiments 219-376.

582. A crystalline Form III of Compound I is a crystalline Form III of Compound I as described in embodiments 219-376, made by a process of any one of embodiments 1-216.

583. A pharmaceutical composition comprising the crystalline form of any one of embodiments 217-376 and 582, and a pharmaceutically acceptable excipient.

584. The pharmaceutical composition of embodiment 583, formulated into a tablet, capsule, suspension, dispersion, injectable, or other pharmaceutical form.

585. A method of treating obesity in a subject in need thereof comprising administering to the subject an effective amount of a crystalline form of any one of embodiments 217-520 and 582, or a pharmaceutical composition of any one of embodiments 521-579 and 583-584.

586. A method of treating an obesity-related disease or disorder comprising administering to a subject suffering from or at risk of suffering from an obesity-related disease or disorder a crystalline form of any one of embodiments 217-520 and 582, or a pharmaceutical composition of any one of embodiments 521-579 and 583-584.

587. The method of embodiment 586, wherein the obesity-related disease or disorder is selected from the group comprising obesity, pre-obesity, morbid obesity, Prader-Willi Syndrome, Hypothalamic Injury Associated Obesity, Non-alcoholic steatohepatitis, hyperlipidemia, hypertension, diabetes, lipodystrophy, fatty liver, Bardet-Biedl Syndrome, Cohen Syndrome, cardiovascular disease, arthritis, stroke, metabolic syndrome and MOMO Syndrome.

588. The method of any one of embodiments 585 to 587, wherein the composition is administered in combination with another therapy.

589. The method of any one of embodiments 585 to 588, wherein administering further comprises oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

590. The method of any one of embodiments 585 to 589, wherein the composition is administered in a form selected from the group comprising pills, capsules, tablets, granules, powders, salts, crystals, liquid, serums, syrups, suspensions, gels, creams, pastes, films, patches, and vapors.

591. The method of any one of embodiments 585 to 590, wherein is the subject is a mammal.

592. The method of any one of embodiments 585 to 591, wherein the subject is a human.

593. The method of embodiment 592, wherein the subject is a human with a body mass index (BMI) greater than 30 kg/m².

594. A method of treating a malignancy-related disease or disorder comprising administering to a subject suffering from or at risk of suffering from a malignancy-related disease or disorder a crystalline form of any one of embodiments 217-520 and 582, or a pharmaceutical composition of any one of embodiments 521-579 and 583-584.

595. The method of embodiment 594, wherein the malignancy-related disease or disorder is selected from the group comprising gastric cancer, multiple myeloma, melanoma, leukemia, lymphoma, renal cell carcinoma, hepatocellular carcinoma, breast cancer, prostate cancer, head and neck cancer, non-small cell lung carcinoma, brain cancer, and glioblastoma multiform (GBM).

596. The method of any one of embodiments 594 to 595, wherein the composition is administered in combination with another therapy.

597. The method of any one of embodiments 594 to 596, wherein administering further comprises oral administration, intravenous administration, topical administration, parenteral administration, intraperitoneal administration, intramuscular administration, intrathecal administration, intralesional administration, intracranial administration, intranasal administration, intraocular administration, intracardiac administration, intravitreal administration, intraosseous administration, intracerebral administration, intraarterial administration, intraarticular administration, intradermal administration, transdermal administration, transmucosal administration, sublingual administration, enteral administration, sublabial administration, insufflation administration, suppository administration, inhaled administration, or subcutaneous administration.

598. The method of any one of embodiments 594 to 597, wherein the composition is administered in a form selected from the group comprising pills, capsules, tablets, granules, powders, salts, crystals, liquid, serums, syrups, suspensions, gels, creams, pastes, films, patches, and vapors.

599. The method of any one of embodiments 594 to 598, wherein is the subject is a mammal.

600. The method of any one of embodiments 594 to 599, wherein the subject is a human.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims. All publications and patent applications cited in this specification are herein incorporated by reference as if each individual publication or patent application were specifically and individually indicated to be incorporated by reference.

1. A crystalline Form III of 3-hydroxy-9β,13α-dimethyl-2-oxo-24,25,26-trinoroleana-1(10),3,5,7-tetraen-29-oic acid, 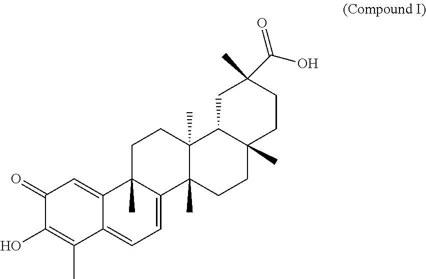
characterized by an x-ray powder diffraction pattern comprising peaks at 2θ angles (±0.2) of 9.51°, 14.81°, and 16.84°.

2. The crystalline form of claim 1, characterized by an X-ray powder diffraction pattern further comprising one or more peaks at 2θ angles (±0.2) selected from the group consisting of 13.60°, 14.65°, 18.74°, and 19.10°.

3. The crystalline form of claim 1, characterized by a differential scanning calorimetry (DSC) profile with no endothermic peak and an exothermic peak at about 215° C. (onset).

4. The crystalline form of claim 1, characterized by a thermal gravimetric analysis (TGA) profile with about 0.5% weight loss at about the exothermic peak at about 215° C. (onset).

5. The crystalline form claim 1, wherein the crystalline form is anhydrous.

6. The crystalline form of claim 1, wherein the crystalline form comprises about 5000 ppm or less of ethyl acetate.

7. The crystalline form of claim 1, wherein the crystalline form comprises about 20 ppm or less of ethyl acetate.

8. The crystalline form of claim 1, wherein the crystalline form comprises 3000 ppm or less of methanol.

9. The crystalline form of claim 1, wherein the crystalline form comprises 1500 ppm or less of methanol.

10. The crystalline form of claim 1, wherein the crystalline form comprises about 5000 ppm or less of ethanol.

11. The crystalline form of claim 1, wherein the crystalline form comprises about 600 ppm or less of ethanol.

12. The crystalline form of claim 1, wherein the crystalline form comprises about 20 ppm or less of ethanol.

13. The crystalline form of claim 1, wherein the crystalline form comprises about 600 ppm or less of dichloromethane.

14. The crystalline form of claim 1, wherein the crystalline form comprises about 20 ppm or less of dichloromethane.

15. The crystalline form of claim 1, wherein the crystalline form comprises about 4000 ppm or less of residual solvents.

16. The crystalline form of claim 1, wherein the crystalline form has at least 99.8% purity.

17. A crystalline Form III of 3-hydroxy-9β,13α-dimethyl-2-oxo-24,25,26-trinoroleana-1(10),3,5,7-tetraen-29-oic acid, 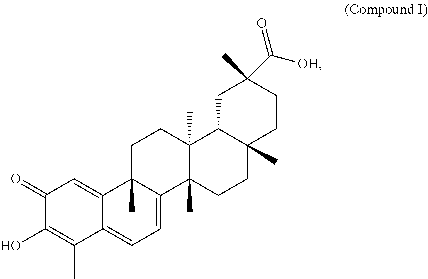
characterized by an x-ray powder diffraction pattern comprising peaks at 2θ angles (±0.2) of 9.51°, 14.81°, and 16.84°, wherein the crystalline form comprises about 5000 ppm or less of ethyl acetate, 5000 ppm or less of ethanol, 3000 ppm or less of methanol, and about 600 ppm or less of dichloromethane.

18. The crystalline form of claim 17, characterized by an X-ray powder diffraction pattern further comprising one or more peaks at 2θ angles (±0.2) selected from the group consisting of 13.60°, 14.65°, 18.74°, and 19.10°.

19. The crystalline form claim 17, wherein the crystalline form is anhydrous.

20. The crystalline form of claim 17, wherein the crystalline form comprises about 20 ppm or less of ethyl acetate.

21. The crystalline form of claim 17, wherein the crystalline form comprises 1500 ppm or less of methanol.

22. The crystalline form of claim 17, wherein the crystalline form comprises about 600 ppm or less of ethanol.

23. The crystalline form of claim 17, wherein the crystalline form comprises about 20 ppm or less of ethanol.

24. The crystalline form of claim 17, wherein the crystalline form comprises about 20 ppm or less of dichloromethane.

25. The crystalline form of claim 17, wherein the crystalline form comprises about 20 ppm or less of ethyl acetate, about 600 ppm or less of ethanol, about 1500 ppm or less of methanol, and about 20 ppm or less of dichloromethane.

26. The crystalline form of claim 25, wherein the crystalline form comprises about 20 ppm or less of ethanol.

27. The crystalline form of claim 17, wherein the crystalline form comprises about 500 ppm or less of n-heptane and about 500 ppm or less of 2-methyltetrahydrofuran.

28. The crystalline form of claim 17, wherein the crystalline form has at least 99.8% purity.

29. A pharmaceutical composition comprising the crystalline form of claim 1, and a pharmaceutically acceptable excipient.

30. A pharmaceutical composition comprising the crystalline form of claim 17, and a pharmaceutically acceptable excipient.
